SEQUENCE LISTING (1235 sequences)

SEQ ID NO: 1 - RNA, 20 nt, synthetic construct
  source 1..20: mol_type = other RNA
gatcctcgtcgtggtgctcg

SEQ ID NO: 2 - RNA, 20 nt, synthetic construct
  source 1..20: mol_type = other RNA
cctcgtcgtggtgctcgcgg

SEQ ID NO: 3 - RNA, 20 nt, synthetic construct
  source 1..20: mol_type = other RNA
gcatcgcgccaggacggtct

SEQ ID NO: 4 - RNA, 20 nt, synthetic construct
  source 1..20: mol_type = other RNA
tgtacttgacgcatcgcgcc

SEQ ID NO: 5 - RNA, 20 nt, synthetic construct
  source 1..20: mol_type = other RNA
caccgggctgaactcgcagt

SEQ ID NO: 6 - RNA, 20 nt, synthetic construct
  source 1..20: mol_type = other RNA
tcgcggtggtcgtcccgagg

SEQ ID NO: 7 - RNA, 20 nt, synthetic construct
  source 1..20: mol_type = other RNA
ccaccgcgagcaccacgacg

SEQ ID NO: 8 - RNA, 20 nt, synthetic construct
  source 1..20: mol_type = other RNA
catcgcgccaggacggtctc

SEQ ID NO: 9 - RNA, 20 nt, synthetic construct
  source 1..20: mol_type = other RNA
gacggtctcgggaaagcgct

SEQ ID NO: 10 - RNA, 20 nt, synthetic construct
  source 1..20: mol_type = other RNA
gcgctttcccgagaccgtcc

SEQ ID NO: 11 - RNA, 20 nt, synthetic construct
  source 1..20: mol_type = other RNA
cttgacgcatcgcgccagga

SEQ ID NO: 12 - RNA, 20 nt, synthetic construct
  source 1..20: mol_type = other RNA
tgcgagttcagcccggtgtc

SEQ ID NO: 13 - RNA, 20 nt, synthetic construct
  source 1..20: mol_type = other RNA
tgctcgcggtggtcgtcccg

SEQ ID NO: 14 - RNA, 20 nt, synthetic construct
  source 1..20: mol_type = other RNA
agggtttgtccccggacacc

SEQ ID NO: 15 - RNA, 20 nt, synthetic construct
  source 1..20: mol_type = other RNA
gcgagttcagcccggtgtcc

SEQ ID NO: 16 - RNA, 20 nt, synthetic construct
  source 1..20: mol_type = other RNA
ggtctcgggaaagcgcttgg

SEQ ID NO: 17 - RNA, 20 nt, synthetic construct
  source 1..20: mol_type = other RNA
cgagttcagcccggtgtccg

SEQ ID NO: 18 - RNA, 20 nt, synthetic construct
  source 1..20: mol_type = other RNA
ccggcagcagggtttgtccc

SEQ ID NO: 19 - RNA, 20 nt, synthetic construct
  source 1..20: mol_type = other RNA
gttgggctctcctagagagc

SEQ ID NO: 20 - RNA, 20 nt, synthetic construct
  source 1..20: mol_type = other RNA
cgagcaccacgacgaggatc

SEQ ID NO: 21 - RNA, 20 nt, synthetic construct
  source 1..20: mol_type = other RNA
ggccaactgcgagttcagcc

SEQ ID NO: 22 - RNA, 20 nt, synthetic construct
  source 1..20: mol_type = other RNA
tactgacgccaagacagagt

SEQ ID NO: 23 - RNA, 20 nt, synthetic construct
  source 1..20: mol_type = other RNA
tcgccaacccacctcatctc

SEQ ID NO: 24 - RNA, 20 nt, synthetic construct
  source 1..20: mol_type = other RNA
ccggggacaaaccctgctgc

SEQ ID NO: 25 - RNA, 20 nt, synthetic construct
  source 1..20: mol_type = other RNA
ggaaagcgcttggtggtgcc

SEQ ID NO: 26 - RNA, 20 nt, synthetic construct
  source 1..20: mol_type = other RNA
tctcctagagagccggcagc

SEQ ID NO: 27 - RNA, 20 nt, synthetic construct
  source 1..20: mol_type = other RNA
cgccagcagtggagcggtcc

SEQ ID NO: 28 - RNA, 20 nt, synthetic construct
  source 1..20: mol_type = other RNA
ctccactgctggcgccacct

SEQ ID NO: 29 - RNA, 20 nt, synthetic construct
  source 1..20: mol_type = other RNA
cagggtttgtccccggacac

SEQ ID NO: 30 - RNA, 20 nt, synthetic construct
  source 1..20: mol_type = other RNA
ctgtcttggcgtcagtatcc

SEQ ID NO: 31 - RNA, 20 nt, synthetic construct
  source 1..20: mol_type = other RNA
tgcccggaccgctccactgc

SEQ ID NO: 32 - RNA, 20 nt, synthetic construct
  source 1..20: mol_type = other RNA
ctcctagagagccggcagca

SEQ ID NO: 33 - RNA, 20 nt, synthetic construct
  source 1..20: mol_type = other RNA
cctggtcctgatcctcgtcg

SEQ ID NO: 34 - RNA, 20 nt, synthetic construct
  source 1..20: mol_type = other RNA
tcccgaggtggcgccagcag

SEQ ID NO: 35 - RNA, 20 nt, synthetic construct
  source 1..20: mol_type = other RNA
gcgccagcagtggagcggtc

SEQ ID NO: 36 - RNA, 20 nt, synthetic construct
  source 1..20: mol_type = other RNA
tccactgctggcgccacctc

SEQ ID NO: 37 - RNA, 20 nt, synthetic construct
  source 1..20: mol_type = other RNA
aggagagcccaactctgtct

SEQ ID NO: 38 - RNA, 20 nt, synthetic construct
  source 1..20: mol_type = other RNA
actgacgccaagacagagtt

SEQ ID NO: 39 - RNA, 20 nt, synthetic construct
  source 1..20: mol_type = other RNA
aaccctgctgccggctctct

SEQ ID NO: 40 - RNA, 20 nt, synthetic construct
  source 1..20: mol_type = other RNA
tatcagccactaatgaagtt

SEQ ID NO: 41 - RNA, 20 nt, synthetic construct
  source 1..20: mol_type = other RNA
tgaaagcatcccatacactt

SEQ ID NO: 42 - RNA, 20 nt, synthetic construct
  source 1..20: mol_type = other RNA
cccccaattaccttgttgca

SEQ ID NO: 43 - RNA, 20 nt, synthetic construct
  source 1..20: mol_type = other RNA
atgtagactgccaaagtgta

SEQ ID NO: 44 - RNA, 20 nt, synthetic construct
  source 1..20: mol_type = other RNA
aagtgtatgggatgctttca

SEQ ID NO: 45 - RNA, 20 nt, synthetic construct
  source 1..20: mol_type = other RNA
aattaccttgttgcaaggta

SEQ ID NO: 46 - RNA, 20 nt, synthetic construct
  source 1..20: mol_type = other RNA
tgagttcccaacttcattag

SEQ ID NO: 47 - RNA, 20 nt, synthetic construct
  source 1..20: mol_type = other RNA
tgtagactgccaaagtgtat

SEQ ID NO: 48 - RNA, 20 nt, synthetic construct
  source 1..20: mol_type = other RNA
agtgtatgggatgctttcaa

SEQ ID NO: 49 - RNA, 20 nt, synthetic construct
  source 1..20: mol_type = other RNA
ctatcagccactaatgaagt

SEQ ID NO: 50 - RNA, 20 nt, synthetic construct
  source 1..20: mol_type = other RNA
tcttcttcagtaatgttgca

SEQ ID NO: 51 - RNA, 20 nt, synthetic construct
  source 1..20: mol_type = other RNA
tctggcccatcagttcacac

SEQ ID NO: 52 - RNA, 20 nt, synthetic construct
  source 1..20: mol_type = other RNA
gcgggacatgttcaccctgg

SEQ ID NO: 53 - RNA, 20 nt, synthetic construct
  source 1..20: mol_type = other RNA
ccagcgggacatgttcaccc

SEQ ID NO: 54 - RNA, 20 nt, synthetic construct
  source 1..20: mol_type = other RNA
agcctagcagcgtgtcctcc

SEQ ID NO: 55 - RNA, 20 nt, synthetic construct
  source 1..20: mol_type = other RNA
tgaattcaccacaccatgtg

SEQ ID NO: 56 - RNA, 20 nt, synthetic construct
  source 1..20: mol_type = other RNA
atcagttcacacaggtccag

SEQ ID NO: 57 - RNA, 20 nt, synthetic construct
  source 1..20: mol_type = other RNA
gctgatgacctcacatggtg

SEQ ID NO: 58 - RNA, 20 nt, synthetic construct
  source 1..20: mol_type = other RNA
gcctagcagcgtgtcctcca

SEQ ID NO: 59 - RNA, 20 nt, synthetic construct
  source 1..20: mol_type = other RNA
accatgtgaggtcatcagca

SEQ ID NO: 60 - RNA, 20 nt, synthetic construct
  source 1..20: mol_type = other RNA
tcagttcacacaggtccagc

SEQ ID NO: 61 - RNA, 20 nt, synthetic construct
  source 1..20: mol_type = other RNA
ccagggtgaacatgtcccgc

SEQ ID NO: 62 - RNA, 20 nt, synthetic construct
  source 1..20: mol_type = other RNA
gctggacctgtgtgaactga

SEQ ID NO: 63 - RNA, 20 nt, synthetic construct
  source 1..20: mol_type = other RNA
accctggaggacacgctgct

SEQ ID NO: 64 - RNA, 20 nt, synthetic construct
  source 1..20: mol_type = other RNA
ctggacctgtgtgaactgat

SEQ ID NO: 65 - RNA, 20 nt, synthetic construct
  source 1..20: mol_type = other RNA
tctggaaaacggtttcccgc

SEQ ID NO: 66 - RNA, 20 nt, synthetic construct
  source 1..20: mol_type = other RNA
ctacttggtacttaccctgc

SEQ ID NO: 67 - RNA, 20 nt, synthetic construct
  source 1..20: mol_type = other RNA
acaaccctgtttcagtattc

SEQ ID NO: 68 - RNA, 20 nt, synthetic construct
  source 1..20: mol_type = other RNA
ttttccagaatactgaaaca

SEQ ID NO: 69 - RNA, 20 nt, synthetic construct
  source 1..20: mol_type = other RNA
gttttccagaatactgaaac

SEQ ID NO: 70 - RNA, 20 nt, synthetic construct
  source 1..20: mol_type = other RNA
gggatccattgagcatcaca

SEQ ID NO: 71 - RNA, 20 nt, synthetic construct
  source 1..20: mol_type = other RNA
catcacatggaccacatcac

SEQ ID NO: 72 - RNA, 20 nt, synthetic construct
  source 1..20: mol_type = other RNA
gtggtccatgtgatgctcaa

SEQ ID NO: 73 - RNA, 20 nt, synthetic construct
  source 1..20: mol_type = other RNA
agagaaggttcagacactag

SEQ ID NO: 74 - RNA, 20 nt, synthetic construct
  source 1..20: mol_type = other RNA
tctagtgtctgaaccttctc

SEQ ID NO: 75 - RNA, 20 nt, synthetic construct
  source 1..20: mol_type = other RNA
ggttcagacactagaggcct

SEQ ID NO: 76 - RNA, 20 nt, synthetic construct
  source 1..20: mol_type = other RNA
ccataatttgcaaccagaga

SEQ ID NO: 77 - RNA, 20 nt, synthetic construct
  source 1..20: mol_type = other RNA
ccttctctggttgcaaatta

SEQ ID NO: 78 - RNA, 20 nt, synthetic construct
  source 1..20: mol_type = other RNA
aggttcagacactagaggcc

SEQ ID NO: 79 - RNA, 20 nt, synthetic construct
  source 1..20: mol_type = other RNA
ctagaggcctgggtgataca

SEQ ID NO: 80 - RNA, 20 nt, synthetic construct
  source 1..20: mol_type = other RNA
ttttagcacttttgggagtg

SEQ ID NO: 81 - RNA, 20 nt, synthetic construct
  source 1..20: mol_type = other RNA
tcttccaccatgtatcaccc

SEQ ID NO: 82 - RNA, 20 nt, synthetic construct
  source 1..20: mol_type = other RNA
cttccccagagacttatgcc

SEQ ID NO: 83 - RNA, 20 nt, synthetic construct
  source 1..20: mol_type = other RNA
ttacctgtagatattcttgc

SEQ ID NO: 84 - RNA, 20 nt, synthetic construct
  source 1..20: mol_type = other RNA
gctcttttatggtgggatcc

SEQ ID NO: 85 - RNA, 20 nt, synthetic construct
  source 1..20: mol_type = other RNA
ggatcccaccataaaagagc

SEQ ID NO: 86 - RNA, 20 nt, synthetic construct
  source 1..20: mol_type = other RNA
cgattccagctcttttatgg

SEQ ID NO: 87 - RNA, 20 nt, synthetic construct
  source 1..20: mol_type = other RNA
gattccagctcttttatggt

SEQ ID NO: 88 - RNA, 20 nt, synthetic construct
  source 1..20: mol_type = other RNA
aatcgattccagctctttta

SEQ ID NO: 89 - RNA, 100 nt, synthetic construct
  source 1..100: mol_type = other RNA
gatcctcgtcgtggtgctcggttttagagctagaaatagcaagttaaaataaggctagtccgttatcaacttgaaaaagtggcaccgagtcggtgctttt

SEQ ID NO: 90 - RNA, 100 nt, synthetic construct
  source 1..100: mol_type = other RNA
cctcgtcgtggtgctcgcgggttttagagctagaaatagcaagttaaaataaggctagtccgttatcaacttgaaaaagtggcaccgagtcggtgctttt

SEQ ID NO: 91 - RNA, 100 nt, synthetic construct
  source 1..100: mol_type = other RNA
gcatcgcgccaggacggtctgttttagagctagaaatagcaagttaaaataaggctagtccgttatcaacttgaaaaagtggcaccgagtcggtgctttt

SEQ ID NO: 92 - RNA, 100 nt, synthetic construct
  source 1..100: mol_type = other RNA
tgtacttgacgcatcgcgccgttttagagctagaaatagcaagttaaaataaggctagtccgttatcaacttgaaaaagtggcaccgagtcggtgctttt

SEQ ID NO: 93 - RNA, 100 nt, synthetic construct
  source 1..100: mol_type = other RNA
caccgggctgaactcgcagtgttttagagctagaaatagcaagttaaaataaggctagtccgttatcaacttgaaaaagtggcaccgagtcggtgctttt

SEQ ID NO: 94 - RNA, 100 nt, synthetic construct
  source 1..100: mol_type = other RNA
tcgcggtggtcgtcccgagggttttagagctagaaatagcaagttaaaataaggctagtccgttatcaacttgaaaaagtggcaccgagtcggtgctttt

SEQ ID NO: 95 - RNA, 100 nt, synthetic construct
  source 1..100: mol_type = other RNA
ccaccgcgagcaccacgacggttttagagctagaaatagcaagttaaaataaggctagtccgttatcaacttgaaaaagtggcaccgagtcggtgctttt

SEQ ID NO: 96 - RNA, 100 nt, synthetic construct
  source 1..100: mol_type = other RNA
catcgcgccaggacggtctcgttttagagctagaaatagcaagttaaaataaggctagtccgttatcaacttgaaaaagtggcaccgagtcggtgctttt

SEQ ID NO: 97 - RNA, 100 nt, synthetic construct
  source 1..100: mol_type = other RNA
gacggtctcgggaaagcgctgttttagagctagaaatagcaagttaaaataaggctagtccgttatcaacttgaaaaagtggcaccgagtcggtgctttt

SEQ ID NO: 98 - RNA, 100 nt, synthetic construct
  source 1..100: mol_type = other RNA
gcgctttcccgagaccgtccgttttagagctagaaatagcaagttaaaataaggctagtccgttatcaacttgaaaaagtggcaccgagtcggtgctttt

SEQ ID NO: 99 - RNA, 100 nt, synthetic construct
  source 1..100: mol_type = other RNA
cttgacgcatcgcgccaggagttttagagctagaaatagcaagttaaaataaggctagtccgttatcaacttgaaaaagtggcaccgagtcggtgctttt

SEQ ID NO: 100 - RNA, 100 nt, synthetic construct
  source 1..100: mol_type = other RNA
tgcgagttcagcccggtgtcgttttagagctagaaatagcaagttaaaataaggctagtccgttatcaacttgaaaaagtggcaccgagtcggtgctttt

SEQ ID NO: 101 - RNA, 100 nt, synthetic construct
  source 1..100: mol_type = other RNA
tgctcgcggtggtcgtcccggttttagagctagaaatagcaagttaaaataaggctagtccgttatcaacttgaaaaagtggcaccgagtcggtgctttt

SEQ ID NO: 102 - RNA, 100 nt, synthetic construct
  source 1..100: mol_type = other RNA
agggtttgtccccggacaccgttttagagctagaaatagcaagttaaaataaggctagtccgttatcaacttgaaaaagtggcaccgagtcggtgctttt

SEQ ID NO: 103 - RNA, 100 nt, synthetic construct
  source 1..100: mol_type = other RNA
gcgagttcagcccggtgtccgttttagagctagaaatagcaagttaaaataaggctagtccgttatcaacttgaaaaagtggcaccgagtcggtgctttt

SEQ ID NO: 104 - RNA, 100 nt, synthetic construct
  source 1..100: mol_type = other RNA
ggtctcgggaaagcgcttgggttttagagctagaaatagcaagttaaaataaggctagtccgttatcaacttgaaaaagtggcaccgagtcggtgctttt

SEQ ID NO: 105 - RNA, 100 nt, synthetic construct
  source 1..100: mol_type = other RNA
cgagttcagcccggtgtccggttttagagctagaaatagcaagttaaaataaggctagtccgttatcaacttgaaaaagtggcaccgagtcggtgctttt

SEQ ID NO: 106 - RNA, 100 nt, synthetic construct
  source 1..100: mol_type = other RNA
ccggcagcagggtttgtcccgttttagagctagaaatagcaagttaaaataaggctagtccgttatcaacttgaaaaagtggcaccgagtcggtgctttt

SEQ ID NO: 107 - RNA, 100 nt, synthetic construct
  source 1..100: mol_type = other RNA
gttgggctctcctagagagcgttttagagctagaaatagcaagttaaaataaggctagtccgttatcaacttgaaaaagtggcaccgagtcggtgctttt

SEQ ID NO: 108 - RNA, 100 nt, synthetic construct
  source 1..100: mol_type = other RNA
cgagcaccacgacgaggatcgttttagagctagaaatagcaagttaaaataaggctagtccgttatcaacttgaaaaagtggcaccgagtcggtgctttt

SEQ ID NO: 109 - RNA, 100 nt, synthetic construct
  source 1..100: mol_type = other RNA
ggccaactgcgagttcagccgttttagagctagaaatagcaagttaaaataaggctagtccgttatcaacttgaaaaagtggcaccgagtcggtgctttt

SEQ ID NO: 110 - RNA, 100 nt, synthetic construct
  source 1..100: mol_type = other RNA
tactgacgccaagacagagtgttttagagctagaaatagcaagttaaaataaggctagtccgttatcaacttgaaaaagtggcaccgagtcggtgctttt

SEQ ID NO: 111 - RNA, 100 nt, synthetic construct
  source 1..100: mol_type = other RNA
tcgccaacccacctcatctcgttttagagctagaaatagcaagttaaaataaggctagtccgttatcaacttgaaaaagtggcaccgagtcggtgctttt

SEQ ID NO: 112 - RNA, 100 nt, synthetic construct
  source 1..100: mol_type = other RNA
ccggggacaaaccctgctgcgttttagagctagaaatagcaagttaaaataaggctagtccgttatcaacttgaaaaagtggcaccgagtcggtgctttt

SEQ ID NO: 113 - RNA, 100 nt, synthetic construct
  source 1..100: mol_type = other RNA
ggaaagcgcttggtggtgccgttttagagctagaaatagcaagttaaaataaggctagtccgttatcaacttgaaaaagtggcaccgagtcggtgctttt

SEQ ID NO: 114 - RNA, 100 nt, synthetic construct
  source 1..100: mol_type = other RNA
tctcctagagagccggcagcgttttagagctagaaatagcaagttaaaataaggctagtccgttatcaacttgaaaaagtggcaccgagtcggtgctttt

SEQ ID NO: 115 - RNA, 100 nt, synthetic construct
  source 1..100: mol_type = other RNA
cgccagcagtggagcggtccgttttagagctagaaatagcaagttaaaataaggctagtccgttatcaacttgaaaaagtggcaccgagtcggtgctttt

SEQ ID NO: 116 - RNA, 100 nt, synthetic construct
  source 1..100: mol_type = other RNA
ctccactgctggcgccacctgttttagagctagaaatagcaagttaaaataaggctagtccgttatcaacttgaaaaagtggcaccgagtcggtgctttt

SEQ ID NO: 117 - RNA, 100 nt, synthetic construct
  source 1..100: mol_type = other RNA
cagggtttgtccccggacacgttttagagctagaaatagcaagttaaaataaggctagtccgttatcaacttgaaaaagtggcaccgagtcggtgctttt

SEQ ID NO: 118 - RNA, 100 nt, synthetic construct
  source 1..100: mol_type = other RNA
ctgtcttggcgtcagtatccgttttagagctagaaatagcaagttaaaataaggctagtccgttatcaacttgaaaaagtggcaccgagtcggtgctttt

SEQ ID NO: 119 - RNA, 100 nt, synthetic construct
  source 1..100: mol_type = other RNA
tgcccggaccgctccactgcgttttagagctagaaatagcaagttaaaataaggctagtccgttatcaacttgaaaaagtggcaccgagtcggtgctttt

SEQ ID NO: 120 - RNA, 100 nt, synthetic construct
  source 1..100: mol_type = other RNA
ctcctagagagccggcagcagttttagagctagaaatagcaagttaaaataaggctagtccgttatcaacttgaaaaagtggcaccgagtcggtgctttt

SEQ ID NO: 121 - RNA, 100 nt, synthetic construct
  source 1..100: mol_type = other RNA
cctggtcctgatcctcgtcggttttagagctagaaatagcaagttaaaataaggctagtccgttatcaacttgaaaaagtggcaccgagtcggtgctttt

SEQ ID NO: 122 - RNA, 100 nt, synthetic construct
  source 1..100: mol_type = other RNA
tcccgaggtggcgccagcaggttttagagctagaaatagcaagttaaaataaggctagtccgttatcaacttgaaaaagtggcaccgagtcggtgctttt

SEQ ID NO: 123 - RNA, 100 nt, synthetic construct
  source 1..100: mol_type = other RNA
gcgccagcagtggagcggtcgttttagagctagaaatagcaagttaaaataaggctagtccgttatcaacttgaaaaagtggcaccgagtcggtgctttt

SEQ ID NO: 124 - RNA, 100 nt, synthetic construct
  source 1..100: mol_type = other RNA
tccactgctggcgccacctcgttttagagctagaaatagcaagttaaaataaggctagtccgttatcaacttgaaaaagtggcaccgagtcggtgctttt

SEQ ID NO: 125 - RNA, 100 nt, synthetic construct
  source 1..100: mol_type = other RNA
aggagagcccaactctgtctgttttagagctagaaatagcaagttaaaataaggctagtccgttatcaacttgaaaaagtggcaccgagtcggtgctttt

SEQ ID NO: 126 - RNA, 100 nt, synthetic construct
  source 1..100: mol_type = other RNA
actgacgccaagacagagttgttttagagctagaaatagcaagttaaaataaggctagtccgttatcaacttgaaaaagtggcaccgagtcggtgctttt

SEQ ID NO: 127 - RNA, 100 nt, synthetic construct
  source 1..100: mol_type = other RNA
aaccctgctgccggctctctgttttagagctagaaatagcaagttaaaataaggctagtccgttatcaacttgaaaaagtggcaccgagtcggtgctttt

SEQ ID NO: 128 - RNA, 100 nt, synthetic construct
  source 1..100: mol_type = other RNA
tatcagccactaatgaagttgttttagagctagaaatagcaagttaaaataaggctagtccgttatcaacttgaaaaagtggcaccgagtcggtgctttt

SEQ ID NO: 129 - RNA, 100 nt, synthetic construct
  source 1..100: mol_type = other RNA
tgaaagcatcccatacacttgttttagagctagaaatagcaagttaaaataaggctagtccgttatcaacttgaaaaagtggcaccgagtcggtgctttt

SEQ ID NO: 130 - RNA, 100 nt, synthetic construct
  source 1..100: mol_type = other RNA
cccccaattaccttgttgcagttttagagctagaaatagcaagttaaaataaggctagtccgttatcaacttgaaaaagtggcaccgagtcggtgctttt

SEQ ID NO: 131 - RNA, 100 nt, synthetic construct
  source 1..100: mol_type = other RNA
atgtagactgccaaagtgtagttttagagctagaaatagcaagttaaaataaggctagtccgttatcaacttgaaaaagtggcaccgagtcggtgctttt

SEQ ID NO: 132 - RNA, 100 nt, synthetic construct
  source 1..100: mol_type = other RNA
aagtgtatgggatgctttcagttttagagctagaaatagcaagttaaaataaggctagtccgttatcaacttgaaaaagtggcaccgagtcggtgctttt

SEQ ID NO: 133 - RNA, 100 nt, synthetic construct
  source 1..100: mol_type = other RNA
aattaccttgttgcaaggtagttttagagctagaaatagcaagttaaaataaggctagtccgttatcaacttgaaaaagtggcaccgagtcggtgctttt

SEQ ID NO: 134 - RNA, 100 nt, synthetic construct
  source 1..100: mol_type = other RNA
tgagttcccaacttcattaggttttagagctagaaatagcaagttaaaataaggctagtccgttatcaacttgaaaaagtggcaccgagtcggtgctttt

SEQ ID NO: 135 - RNA, 100 nt, synthetic construct
  source 1..100: mol_type = other RNA
tgtagactgccaaagtgtatgttttagagctagaaatagcaagttaaaataaggctagtccgttatcaacttgaaaaagtggcaccgagtcggtgctttt

SEQ ID NO: 136 - RNA, 100 nt, synthetic construct
  source 1..100: mol_type = other RNA
agtgtatgggatgctttcaagttttagagctagaaatagcaagttaaaataaggctagtccgttatcaacttgaaaaagtggcaccgagtcggtgctttt

SEQ ID NO: 137 - RNA, 100 nt, synthetic construct
  source 1..100: mol_type = other RNA
ctatcagccactaatgaagtgttttagagctagaaatagcaagttaaaataaggctagtccgttatcaacttgaaaaagtggcaccgagtcggtgctttt

SEQ ID NO: 138 - RNA, 100 nt, synthetic construct
  source 1..100: mol_type = other RNA
tcttcttcagtaatgttgcagttttagagctagaaatagcaagttaaaataaggctagtccgttatcaacttgaaaaagtggcaccgagtcggtgctttt

SEQ ID NO: 139 - RNA, 100 nt, synthetic construct
  source 1..100: mol_type = other RNA
tctggcccatcagttcacacgttttagagctagaaatagcaagttaaaataaggctagtccgttatcaacttgaaaaagtggcaccgagtcggtgctttt

SEQ ID NO: 140 - RNA, 100 nt, synthetic construct
  source 1..100: mol_type = other RNA
gcgggacatgttcaccctgggttttagagctagaaatagcaagttaaaataaggctagtccgttatcaacttgaaaaagtggcaccgagtcggtgctttt

SEQ ID NO: 141 - RNA, 100 nt, synthetic construct
  source 1..100: mol_type = other RNA
ccagcgggacatgttcacccgttttagagctagaaatagcaagttaaaataaggctagtccgttatcaacttgaaaaagtggcaccgagtcggtgctttt

SEQ ID NO: 142 - RNA, 100 nt, synthetic construct
  source 1..100: mol_type = other RNA
agcctagcagcgtgtcctccgttttagagctagaaatagcaagttaaaataaggctagtccgttatcaacttgaaaaagtggcaccgagtcggtgctttt

SEQ ID NO: 143 - RNA, 100 nt, synthetic construct
  source 1..100: mol_type = other RNA
tgaattcaccacaccatgtggttttagagctagaaatagcaagttaaaataaggctagtccgttatcaacttgaaaaagtggcaccgagtcggtgctttt

SEQ ID NO: 144 - RNA, 100 nt, synthetic construct
  source 1..100: mol_type = other RNA
atcagttcacacaggtccaggttttagagctagaaatagcaagttaaaataaggctagtccgttatcaacttgaaaaagtggcaccgagtcggtgctttt

SEQ ID NO: 145 - RNA, 100 nt, synthetic construct
  source 1..100: mol_type = other RNA
gctgatgacctcacatggtggttttagagctagaaatagcaagttaaaataaggctagtccgttatcaacttgaaaaagtggcaccgagtcggtgctttt

SEQ ID NO: 146 - RNA, 100 nt, synthetic construct
  source 1..100: mol_type = other RNA
gcctagcagcgtgtcctccagttttagagctagaaatagcaagttaaaataaggctagtccgttatcaacttgaaaaagtggcaccgagtcggtgctttt

SEQ ID NO: 147 - RNA, 100 nt, synthetic construct
  source 1..100: mol_type = other RNA
accatgtgaggtcatcagcagttttagagctagaaatagcaagttaaaataaggctagtccgttatcaacttgaaaaagtggcaccgagtcggtgctttt

SEQ ID NO: 148 - RNA, 100 nt, synthetic construct
  source 1..100: mol_type = other RNA
tcagttcacacaggtccagcgttttagagctagaaatagcaagttaaaataaggctagtccgttatcaacttgaaaaagtggcaccgagtcggtgctttt

SEQ ID NO: 149 - RNA, 100 nt, synthetic construct
  source 1..100: mol_type = other RNA
ccagggtgaacatgtcccgcgttttagagctagaaatagcaagttaaaataaggctagtccgttatcaacttgaaaaagtggcaccgagtcggtgctttt

SEQ ID NO: 150 - RNA, 100 nt, synthetic construct
  source 1..100: mol_type = other RNA
gctggacctgtgtgaactgagttttagagctagaaatagcaagttaaaataaggctagtccgttatcaacttgaaaaagtggcaccgagtcggtgctttt

SEQ ID NO: 151 - RNA, 100 nt, synthetic construct
  source 1..100: mol_type = other RNA
accctggaggacacgctgctgttttagagctagaaatagcaagttaaaataaggctagtccgttatcaacttgaaaaagtggcaccgagtcggtgctttt

SEQ ID NO: 152 - RNA, 100 nt, synthetic construct
  source 1..100: mol_type = other RNA
ctggacctgtgtgaactgatgttttagagctagaaatagcaagttaaaataaggctagtccgttatcaacttgaaaaagtggcaccgagtcggtgctttt

SEQ ID NO: 153 - RNA, 100 nt, synthetic construct
  source 1..100: mol_type = other RNA
tctggaaaacggtttcccgcgttttagagctagaaatagcaagttaaaataaggctagtccgttatcaacttgaaaaagtggcaccgagtcggtgctttt

SEQ ID NO: 154 - RNA, 100 nt, synthetic construct
  source 1..100: mol_type = other RNA
ctacttggtacttaccctgcgttttagagctagaaatagcaagttaaaataaggctagtccgttatcaacttgaaaaagtggcaccgagtcggtgctttt

SEQ ID NO: 155 - RNA, 100 nt, synthetic construct
  source 1..100: mol_type = other RNA
acaaccctgtttcagtattcgttttagagctagaaatagcaagttaaaataaggctagtccgttatcaacttgaaaaagtggcaccgagtcggtgctttt

SEQ ID NO: 156 - RNA, 100 nt, synthetic construct
  source 1..100: mol_type = other RNA
ttttccagaatactgaaacagttttagagctagaaatagcaagttaaaataaggctagtccgttatcaacttgaaaaagtggcaccgagtcggtgctttt

SEQ ID NO: 157 - RNA, 100 nt, synthetic construct
  source 1..100: mol_type = other RNA
gttttccagaatactgaaacgttttagagctagaaatagcaagttaaaataaggctagtccgttatcaacttgaaaaagtggcaccgagtcggtgctttt

SEQ ID NO: 158 - RNA, 100 nt, synthetic construct
  source 1..100: mol_type = other RNA
gggatccattgagcatcacagttttagagctagaaatagcaagttaaaataaggctagtccgttatcaacttgaaaaagtggcaccgagtcggtgctttt

SEQ ID NO: 159 - RNA, 100 nt, synthetic construct
  source 1..100: mol_type = other RNA
catcacatggaccacatcacgttttagagctagaaatagcaagttaaaataaggctagtccgttatcaacttgaaaaagtggcaccgagtcggtgctttt

SEQ ID NO: 160 - RNA, 100 nt, synthetic construct
  source 1..100: mol_type = other RNA
gtggtccatgtgatgctcaagttttagagctagaaatagcaagttaaaataaggctagtccgttatcaacttgaaaaagtggcaccgagtcggtgctttt

SEQ ID NO: 161 - RNA, 100 nt, synthetic construct
  source 1..100: mol_type = other RNA
agagaaggttcagacactaggttttagagctagaaatagcaagttaaaataaggctagtccgttatcaacttgaaaaagtggcaccgagtcggtgctttt

SEQ ID NO: 162 - RNA, 100 nt, synthetic construct
  source 1..100: mol_type = other RNA
tctagtgtctgaaccttctcgttttagagctagaaatagcaagttaaaataaggctagtccgttatcaacttgaaaaagtggcaccgagtcggtgctttt

SEQ ID NO: 163 - RNA, 100 nt, synthetic construct
  source 1..100: mol_type = other RNA
ggttcagacactagaggcctgttttagagctagaaatagcaagttaaaataaggctagtccgttatcaacttgaaaaagtggcaccgagtcggtgctttt

SEQ ID NO: 164 - RNA, 100 nt, synthetic construct
  source 1..100: mol_type = other RNA
ccataatttgcaaccagagagttttagagctagaaatagcaagttaaaataaggctagtccgttatcaacttgaaaaagtggcaccgagtcggtgctttt

SEQ ID NO: 165 - RNA, 100 nt, synthetic construct
  source 1..100: mol_type = other RNA
ccttctctggttgcaaattagttttagagctagaaatagcaagttaaaataaggctagtccgttatcaacttgaaaaagtggcaccgagtcggtgctttt

SEQ ID NO: 166 - RNA, 100 nt, synthetic construct
  source 1..100: mol_type = other RNA
aggttcagacactagaggccgttttagagctagaaatagcaagttaaaataaggctagtccgttatcaacttgaaaaagtggcaccgagtcggtgctttt

SEQ ID NO: 167 - RNA, 100 nt, synthetic construct
  source 1..100: mol_type = other RNA
ctagaggcctgggtgatacagttttagagctagaaatagcaagttaaaataaggctagtccgttatcaacttgaaaaagtggcaccgagtcggtgctttt

SEQ ID NO: 168 - RNA, 100 nt, synthetic construct
  source 1..100: mol_type = other RNA
ttttagcacttttgggagtggttttagagctagaaatagcaagttaaaataaggctagtccgttatcaacttgaaaaagtggcaccgagtcggtgctttt

SEQ ID NO: 169 - RNA, 100 nt, synthetic construct
  source 1..100: mol_type = other RNA
tcttccaccatgtatcacccgttttagagctagaaatagcaagttaaaataaggctagtccgttatcaacttgaaaaagtggcaccgagtcggtgctttt

SEQ ID NO: 170 - RNA, 100 nt, synthetic construct
  source 1..100: mol_type = other RNA
cttccccagagacttatgccgttttagagctagaaatagcaagttaaaataaggctagtccgttatcaacttgaaaaagtggcaccgagtcggtgctttt

SEQ ID NO: 171 - RNA, 100 nt, synthetic construct
  source 1..100: mol_type = other RNA
ttacctgtagatattcttgcgttttagagctagaaatagcaagttaaaataaggctagtccgttatcaacttgaaaaagtggcaccgagtcggtgctttt

SEQ ID NO: 172 - RNA, 100 nt, synthetic construct
  source 1..100: mol_type = other RNA
gctcttttatggtgggatccgttttagagctagaaatagcaagttaaaataaggctagtccgttatcaacttgaaaaagtggcaccgagtcggtgctttt

SEQ ID NO: 173 - RNA, 100 nt, synthetic construct
  source 1..100: mol_type = other RNA
ggatcccaccataaaagagcgttttagagctagaaatagcaagttaaaataaggctagtccgttatcaacttgaaaaagtggcaccgagtcggtgctttt

SEQ ID NO: 174 - RNA, 100 nt, synthetic construct
  source 1..100: mol_type = other RNA
cgattccagctcttttatgggttttagagctagaaatagcaagttaaaataaggctagtccgttatcaacttgaaaaagtggcaccgagtcggtgctttt

SEQ ID NO: 175 - RNA, 100 nt, synthetic construct
  source 1..100: mol_type = other RNA
gattccagctcttttatggtgttttagagctagaaatagcaagttaaaataaggctagtccgttatcaacttgaaaaagtggcaccgagtcggtgctttt

SEQ ID NO: 176 - RNA, 100 nt, synthetic construct
  source 1..100: mol_type = other RNA
aatcgattccagctcttttagttttagagctagaaatagcaagttaaaataaggctagtccgttatcaacttgaaaaagtggcaccgagtcggtgctttt

SEQ ID NO: 177-199 - skipped (23 entries)

SEQ ID NO: 200 - RNA, 22 nt, synthetic construct
  source 1..22: mol_type = other RNA
gttttagagctatgctgttttg

SEQ ID NO: 201 - RNA, 76 nt, synthetic construct
  source 1..76: mol_type = other RNA
gttttagagctagaaatagcaagttaaaataaggctagtccgttatcaacttgaaaaagtggcaccgagtcggtgc

SEQ ID NO: 202 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 203-299 - skipped (97 entries)

SEQ ID NO: 300 - RNA, 100 nt, synthetic construct
  source 1..100: mol_type = other RNA
  misc_feature 1..4: note = Phosphorothiate linkage
  variation 1..20: note = Any natural or non-natural nucleotide
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 69: mod_base = OTHER; note = 2' methyl adenosine
  modified_base 70: mod_base = cm
  modified_base 71..72: mod_base = um
  modified_base 73: mod_base = gm
  modified_base 74..78: mod_base = OTHER; note = 2' methyl adenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2' methyl adenosine
  modified_base 85..86: mod_base = cm
  modified_base 87: mod_base = gm
  modified_base 88: mod_base = OTHER; note = 2' methyl adenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  misc_feature 97..100: note = Phosphorothiate linkage
nnnnnnnnnnnnnnnnnnnngttttagagctagaaatagcaagttaaaataaggctagtccgttatcaacttgaaaaagtggcaccgagtcggtgctttt

SEQ ID NO: 301-404 - skipped (104 entries)

SEQ ID NO: 405 - RNA, 88 nt, synthetic construct
  source 1..88: mol_type = other RNA
nnnnnnnnnnnnnnnnnnnngttttagagctagaaatagcaagttaaaataaggctagtccgttatcaacttggcaccgagtcggtgc

SEQ ID NO: 406 - RNA, 88 nt, synthetic construct; identical to SEQ ID NO: 405
  source 1..88: mol_type = other RNA
  misc_feature 1..4: note = Phosphorothiate linkage
  variation 1..20: note = Any natural or non-natural nucleotide
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 86: mod_base = um
  modified_base 87: mod_base = gm
  modified_base 88: mod_base = cm
  misc_feature 85..88: note = Phosphorothiate linkage

SEQ ID NO: 407 - RNA, 90 nt, synthetic construct
  source 1..90: mol_type = other RNA
nnnnnnnnnnnnnnnnnnnngttttagagctagaaatagcaagttaaaataaggctagtccgttatcacgaaagggcaccgagtcggtgc

SEQ ID NO: 408 - RNA, 90 nt, synthetic construct; identical to SEQ ID NO: 407
  source 1..90: mol_type = other RNA
  misc_feature 1..4: note = Phosphorothiate linkage
  variation 1..20: note = Any natural or non-natural nucleotide
  modified_base 21: mod_base = gm
  modified_base 25: mod_base = OTHER; note = 2' fluoro uracil
  modified_base 28: mod_base = OTHER; note = 2' methyl adenosine
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 41..42: mod_base = OTHER; note = 2' methyl adenosine
  modified_base 45: mod_base = OTHER; note = 2' fluoro uracil
  modified_base 46: mod_base = OTHER; note = 2' methyl adenosine
  modified_base 47: mod_base = OTHER; note = 2' fluoro adenosine
  modified_base 48: mod_base = OTHER; note = 2' methyl adenosine
  modified_base 49: mod_base = OTHER; note = 2' fluoro adenosine
  modified_base 50: mod_base = um
  modified_base 52: mod_base = OTHER; note = 2' fluoro adenosine
  modified_base 53..54: mod_base = gm
  modified_base 55: mod_base = cm
  modified_base 56: mod_base = um
  modified_base 57: mod_base = OTHER; note = 2' methyl adenosine
  modified_base 60..61: mod_base = cm
  modified_base 64: mod_base = OTHER; note = 2' fluoro uracil
  modified_base 66: mod_base = um
  modified_base 67: mod_base = cm
  modified_base 69: mod_base = cm
  modified_base 70: mod_base = gm
  modified_base 71..73: mod_base = OTHER; note = 2' methyl adenosine
  modified_base 74..76: mod_base = gm
  modified_base 77: mod_base = cm
  modified_base 78: mod_base = OTHER; note = 2' methyl adenosine
  modified_base 79..80: mod_base = cm
  modified_base 81: mod_base = gm
  modified_base 82: mod_base = OTHER; note = 2' methyl adenosine
  modified_base 83: mod_base = gm
  modified_base 84: mod_base = um
  modified_base 85: mod_base = cm
  modified_base 86..87: mod_base = gm
  modified_base 88: mod_base = um
  modified_base 89: mod_base = gm
  modified_base 90: mod_base = cm
  misc_feature 87..90: note = Phosphorothiate linkage

SEQ ID NO: 409 - skipped

SEQ ID NO: 410 - RNA, 90 nt, synthetic construct; identical to SEQ ID NO: 407
  source 1..90: mol_type = other RNA
  misc_feature 1..4: note = Phosphorothiate linkage
  variation 1..20: note = Any natural or non-natural nucleotide
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 88: mod_base = um
  modified_base 89: mod_base = gm
  modified_base 90: mod_base = cm
  misc_feature 87..90: note = Phosphorothiate linkage

SEQ ID NO: 411 - RNA, 74 nt, synthetic construct
  source 1..74: mol_type = other RNA
aacagcatagcaagttaaaataaggctagtccgttatcaacttgaaaaagtggcaccgagtcggtgcttttttt

SEQ ID NO: 412 - RNA, 91 nt, synthetic construct
  source 1..91: mol_type = other RNA
  misc_feature 1..4: note = Phosphorothiate linkage
  variation 1..20: note = Any natural or non-natural nucleotide
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 88: mod_base = um
  modified_base 89: mod_base = gm
  modified_base 90: mod_base = cm
  modified_base 91: mod_base = um
  misc_feature 90..91: note = Phosphorothiate linkage
nnnnnnnnnnnnnnnnnnnngttttagagctagaaatagcaagttaaaataaggctagtccgttatcacgaaagggcaccgagtcggtgct

SEQ ID NO: 413 - RNA, 91 nt, synthetic construct
  source 1..91: mol_type = other RNA
cttgacgcatcgcgccaggagttttagagctagaaatagcaagttaaaataaggctagtccgttatcacgaaagggcaccgagtcggtgct

SEQ ID NO: 414 - RNA, 91 nt, synthetic construct; identical to SEQ ID NO: 412
  source 1..91: mol_type = other RNA
  misc_feature 1..4: note = Phosphorothiate linkage
  variation 1..20: note = Any natural or non-natural nucleotide
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 88: mod_base = um
  modified_base 89: mod_base = gm
  modified_base 90: mod_base = cm
  modified_base 91: mod_base = um
  misc_feature 88..91: note = Phosphorothiate linkage

SEQ ID NO: 415-799 - skipped (385 entries)

SEQ ID NO: 800 - protein, 1379 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 801 - DNA, 4140 bp, synthetic construct
  source 1..4140: mol_type = other DNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 802 - DNA, 4140 bp, synthetic construct
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 803 - RNA, 4197 nt, synthetic construct
  source 1..4197: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 804 - RNA, 4782 nt, synthetic construct
  source 1..4782: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 805 - RNA, 4839 nt, synthetic construct
  source 1..4839: mol_type = other RNA
atggaggcctcccccgcctccggcccccggcacctgatggacccccacatcttcacctccaacttcaacaacggcatcggccggcacaagacctacctgtgctacgaggtggagcggctggacaacggcacctccgtgaagatggaccagcaccggggcttcctgcacaaccaggccaagaacctgctgtgcggcttctacggccggcacgccgagctgcggttcctggacctggtgccctccctgcagctggaccccgcccagatctaccgggtgacctggttcatctcctggtccccctgcttctcctggggctgcgccggcgaggtgcgggccttcctgcaggagaacacccacgtgcggctgcggatcttcgccgcccggatctacgactacgaccccctgtacaaggaggccctgcagatgctgcgggacgccggcgcccaggtgtccatcatgacctacgacgagttcaagcactgctgggacaccttcgtggaccaccagggctgccccttccagccctgggacggcctggacgagcactcccaggccctgtccggccggctgcgggccatcctgcagaaccagggcaactccggctccgagacccccggcacctccgagtccgccacccccgagtccgacaagaagtactccatcggcctggccatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtccgagtccgccacccccgagtccgtgtccggctggcggctgttcaagaagatctcctga

SEQ ID NO: 806 - protein, 1593 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 807 - RNA, 291 nt, synthetic construct
  source 1..291: mol_type = other RNA
atgggaccgaagaagaagagaaaggtcggaggaggaagcacaaacctgtcggacatcatcgaaaaggaaacaggaaagcagctggtcatccaggaatcgatcctgatgctgccggaagaagtcgaagaagtcatcggaaacaagccggaatcggacatcctggtccacacagcatacgacgaatcgacagacgaaaacgtcatgctgctgacatcggacgcaccggaatacaagccgtgggcactggtcatccaggactcgaacggagaaaacaagatcaagatgctgtga

SEQ ID NO: 808 - RNA, 324 nt, synthetic construct
  source 1..324: mol_type = other RNA
atgaccaacctgtccgacatcatcgagaaggagaccggcaagcagctggtgatccaggagtccatcctgatgctgcccgaggaggtggaggaggtgatcggcaacaagcccgagtccgacatcctggtgcacaccgcctacgacgagtccaccgacgagaacgtgatgctgctgacctccgacgcccccgagtacaagccctgggccctggtgatccaggactccaacggcgagaacaagatcaagatgctgtccggcggctccaagcggaccgccgacggctccgagttcgagtcccccaagaagaagcggaaggtggagtga

SEQ ID NO: 809 - protein, 107 aa, synthetic construct
MTNLSDIIEKETGKQLVIQESILMLPEEVEEVIGNKPESDILVHTAYDESTDENVMLLTSDAPEYKPWALVIQDSNGENKIKMLSGGSKRTADGSEFESPKKKRKVE

SEQ ID NO: 810-899 - skipped (90 entries)

SEQ ID NO: 900 - protein, 16 aa, synthetic construct
SGSETPGTSESATPES

SEQ ID NO: 901 - protein, 12 aa, synthetic construct
SGSETPGTSESA

SEQ ID NO: 902 - protein, 21 aa, synthetic construct
SGSETPGTSESATPEGGSGGS

SEQ ID NO: 903-1000 - skipped (98 entries)

SEQ ID NO: 1001 - DNA, 3896 bp, synthetic construct
  source 1..3896: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttccgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaacatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgctagcgctaccggactcaatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgttagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatcagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 1002-1099 - skipped (98 entries)

SEQ ID NO: 1100 - RNA, 100 nt, synthetic construct
  source 1..100: mol_type = other RNA
  misc_feature 1..4: note = Phosphorothiate linkage
  modified_base 1: mod_base = cm
  modified_base 2: mod_base = um
  modified_base 3: mod_base = cm
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 69: mod_base = OTHER; note = 2' methyl adenosine
  modified_base 70: mod_base = cm
  modified_base 71..72: mod_base = um
  modified_base 73: mod_base = gm
  modified_base 74..78: mod_base = OTHER; note = 2' methyl adenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2' methyl adenosine
  modified_base 85..86: mod_base = cm
  modified_base 87: mod_base = gm
  modified_base 88: mod_base = OTHER; note = 2' methyl adenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  misc_feature 97..100: note = Phosphorothiate linkage
ctctcagctggtacacggcagttttagagctagaaatagcaagttaaaataaggctagtccgttatcaacttgaaaaagtggcaccgagtcggtgctttt

SEQ ID NO: 1101 - RNA, 100 nt, synthetic construct
  source 1..100: mol_type = other RNA
  misc_feature 1..4: note = Phosphorothiate linkage
  modified_base 1: mod_base = cm
  modified_base 2: mod_base = um
  modified_base 3: mod_base = cm
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 69: mod_base = OTHER; note = 2' methyl adenosine
  modified_base 70: mod_base = cm
  modified_base 71..72: mod_base = um
  modified_base 73: mod_base = gm
  modified_base 74..78: mod_base = OTHER; note = 2' methyl adenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2' methyl adenosine
  modified_base 85..86: mod_base = cm
  modified_base 87: mod_base = gm
  modified_base 88: mod_base = OTHER; note = 2' methyl adenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  misc_feature 97..100: note = Phosphorothiate linkage
ggcctcggcgctgacgatctgttttagagctagaaatagcaagttaaaataaggctagtccgttatcaacttgaaaaagtggcaccgagtcggtgctttt

SEQ ID NO: 1102 - RNA, 91 nt, synthetic construct; identical to SEQ ID NO: 413
  source 1..91: mol_type = other RNA
  misc_feature 1..4: note = Phosphorothiate linkage
  modified_base 1: mod_base = cm
  modified_base 2..3: mod_base = um
  modified_base 29: mod_base = gm
  modified_base 30: mod_base = cm
  modified_base 31: mod_base = um
  modified_base 32: mod_base = OTHER; note = 2' methyl adenosine
  modified_base 33: mod_base = gm
  modified_base 34..36: mod_base = OTHER; note = 2' methyl adenosine
  modified_base 37: mod_base = um
  modified_base 38: mod_base = OTHER; note = 2' methyl adenosine
  modified_base 39: mod_base = gm
  modified_base 40: mod_base = cm
  modified_base 88: mod_base = um
  modified_base 89: mod_base = gm
  modified_base 90: mod_base = cm
  modified_base 91: mod_base = um
  misc_feature 88..91: note = Phosphorothiate linkage

SEQ ID NO: 1103-1200 - skipped (98 entries)

SEQ ID NO: 1201 - protein, 1612 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGNSGSETPGTSESATPESDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 1202 - protein, 1398 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKVSESATPESVSGWRLFKKIS

SEQ ID NO: 1203 - RNA, 4140 nt, synthetic construct
  source 1..4140: mol_type = other RNA
atggacaagaagtacagcatcggcctggacatcggcacgaacagcgttggctgggctgtgatcacggacgagtacaaggttccctcaaagaagttcaaggtgctgggcaacacggaccggcacagcatcaagaagaatctcatcggtgcactgctgttcgacagcggtgagacggccgaagccacgcggctgaagcggacggcccgccggcggtacacgcggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaagtcgcctaccacgagaagtaccccaccatctaccacctgcggaagaagctggtggactcgactgacaaggccgacctgcggctgatctacctggcactggcccacatgataaagttccggggccacttcctgatcgagggcgacctgaaccctgacaacagcgacgtggacaagctgttcatccagctggtgcagacctacaaccagctgttcgaggagaaccccatcaacgccagcggcgtggacgccaaggccatcctcagcgcccgcctcagcaagagccggcggctggagaatctcatcgcccagcttccaggtgagaagaagaatgggctgttcggcaatctcatcgcactcagcctgggcctgactcccaacttcaagagcaacttcgacctggccgaggacgccaagctgcagctcagcaaggacacctacgacgacgacctggacaatctcctggcccagatcggcgaccagtacgccgacctgttcctggctgccaagaatctcagcgacgccatcctgctcagcgacatcctgcgggtgaacacagagatcacgaaggcccccctcagcgccagcatgataaagcggtacgacgagcaccaccaggacctgacgctgctgaaggcactggtgcggcagcagcttccagagaagtacaaggagatcttcttcgaccagagcaagaatgggtacgccgggtacatcgacggtggtgccagccaggaggagttctacaagttcatcaagcccatcctggagaagatggacggcacagaggagctgctggtgaagctgaacagggaggacctgctgcggaagcagcggacgttcgacaatgggagcatcccccaccagatccacctgggtgagctgcacgccatcctgcggcggcaggaggacttctaccccttcctgaaggacaacagggagaagatcgagaagatcctgacgttccggatcccctactacgttggccccctggcccgcggcaacagccggttcgcctggatgacgcggaagagcgaggagacgatcactccctggaacttcgaggaagtcgtggacaagggtgccagcgcccagagcttcatcgagcggatgacgaacttcgacaagaatcttccaaacgagaaggtgcttccaaagcacagcctgctgtacgagtacttcacggtgtacaacgagctgacgaaggtgaagtacgtgacagagggcatgcggaagcccgccttcctcagcggtgagcagaagaaggccatcgtggacctgctgttcaagacgaaccggaaggtgacggtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgacgctgacgctgttcgaggacagggagatgatagaggagcggctgaagacctacgcccacctgttcgacgacaaggtgatgaagcagctgaagcggcggcggtacacgggctggggccggctcagccggaagctgatcaatgggatccgagacaagcagagcggcaagacgatcctggacttcctgaagagcgacggcttcgccaaccggaacttcatgcagctgatccacgacgacagcctgacgttcaaggaggacatccagaaggcccaggtcagcggccagggcgacagcctgcacgagcacatcgccaatctcgccgggagccccgccatcaagaaggggatcctgcagacggtgaaggtggtggacgagctggtgaaggtgatgggccggcacaagccagagaacatcgtgatcgagatggccagggagaaccagacgactcaaaaggggcagaagaacagcagggagcggatgaagcggatcgaggagggcatcaaggagctgggcagccagatcctgaaggagcaccccgtggagaacactcaactgcagaacgagaagctgtacctgtactacctgcagaatgggcgagacatgtacgtggaccaggagctggacatcaaccggctcagcgactacgacgtggaccacatcgttccccagagcttcctgaaggacgacagcatcgacaacaaggtgctgacgcggagcgacaagaaccggggcaagagcgacaacgttccctcagaggaagtcgtgaagaagatgaagaactactggcggcagctgctgaacgccaagctgatcactcaacggaagttcgacaatctcacgaaggccgagcggggtggcctcagcgagctggacaaggccgggttcatcaagcggcagctggtggagacgcggcagatcacgaagcacgtggcccagatcctggacagccggatgaacacgaagtacgacgagaacgacaagctgatcagggaagtcaaggtgatcacgctgaagagcaagctggtcagcgacttccggaaggacttccagttctacaaggtgagggagatcaacaactaccaccacgcccacgacgcctacctgaacgctgtggttggcacggcactgatcaagaagtaccccaagctggagagcgagttcgtgtacggcgactacaaggtgtacgacgtgcggaagatgatagccaagagcgagcaggagatcggcaaggccacggccaagtacttcttctacagcaacatcatgaacttcttcaagacagagatcacgctggccaatggtgagatccggaagcggcccctgatcgagacgaatggtgagacgggtgagatcgtgtgggacaaggggcgagacttcgccacggtgcggaaggtgctcagcatgccccaggtgaacatcgtgaagaagacagaagtccagacgggtggcttcagcaaggagagcatccttccaaagcggaacagcgacaagctgatcgcccgcaagaaggactgggaccccaagaagtacggtggcttcgacagccccaccgtggcctacagcgtgctggtggtggccaaggtggagaaggggaagagcaagaagctgaagagcgtgaaggagctgctgggcatcacgatcatggagcggagcagcttcgagaagaaccccatcgacttcctggaagccaaggggtacaaggaagtcaagaaggacctgatcatcaagcttccaaagtacagcctgttcgagctggagaatgggcggaagcggatgctggccagcgccggtgagctgcagaaggggaacgagctggcacttccctcaaagtacgtgaacttcctgtacctggccagccactacgagaagctgaaggggagcccagaggacaacgagcagaagcagctgttcgtggagcagcacaagcactacctggacgagatcatcgagcagatcagcgagttcagcaagcgggtgatcctggccgacgccaatctcgacaaggtgctcagcgcctacaacaagcaccgagacaagcccatcagggagcaggccgagaacatcatccacctgttcacgctgacgaatctcggtgcccccgctgccttcaagtacttcgacacgacgatcgaccggaagcggtacacgtcgactaaggaagtcctggacgccacgctgatccaccagagcatcacgggcctgtacgagacgcggatcgacctcagccagctgggtggcgacggtggtggcagccccaagaagaagcggaaggtgtag

SEQ ID NO: 1204 - RNA, 4140 nt, synthetic construct
  source 1..4140: mol_type = other RNA
atggacaagaagtacagcatcggcctcgacatcggcaccaacagcgtcggctgggccgtcatcaccgacgagtacaaggtccccagcaagaagttcaaggtcctcggcaacaccgaccgccacagcatcaagaagaacctcatcggcgccctcctcttcgacagcggcgagaccgccgaggccacccgcctcaagcgcaccgcccgccgccgctacacccgccgcaagaaccgcatctgctacctccaggagatcttcagcaacgagatggccaaggtcgacgacagcttcttccaccgcctcgaggagagcttcctcgtcgaggaggacaagaagcacgagcgccaccccatcttcggcaacatcgtcgacgaggtcgcctaccacgagaagtaccccaccatctaccacctccgcaagaagctcgtcgacagcaccgacaaggccgacctccgcctcatctacctcgccctcgcccacatgatcaagttccgcggccacttcctcatcgagggcgacctcaaccccgacaacagcgacgtcgacaagctcttcatccagctcgtccagacctacaaccagctcttcgaggagaaccccatcaacgccagcggcgtcgacgccaaggccatcctcagcgcccgcctcagcaagagccgccgcctcgagaacctcatcgcccagctccccggcgagaagaagaacggcctcttcggcaacctcatcgccctcagcctcggcctcacccccaacttcaagagcaacttcgacctcgccgaggacgccaagctccagctcagcaaggacacctacgacgacgacctcgacaacctcctcgcccagatcggcgaccagtacgccgacctcttcctcgccgccaagaacctcagcgacgccatcctcctcagcgacatcctccgcgtcaacaccgagatcaccaaggcccccctcagcgccagcatgatcaagcgctacgacgagcaccaccaggacctcaccctcctcaaggccctcgtccgccagcagctccccgagaagtacaaggagatcttcttcgaccagagcaagaacggctacgccggctacatcgacggcggcgccagccaggaggagttctacaagttcatcaagcccatcctcgagaagatggacggcaccgaggagctcctcgtcaagctcaaccgcgaggacctcctccgcaagcagcgcaccttcgacaacggcagcatcccccaccagatccacctcggcgagctccacgccatcctccgccgccaggaggacttctaccccttcctcaaggacaaccgcgagaagatcgagaagatcctcaccttccgcatcccctactacgtcggccccctcgcccgcggcaacagccgcttcgcctggatgacccgcaagagcgaggagaccatcaccccctggaacttcgaggaggtcgtcgacaagggcgccagcgcccagagcttcatcgagcgcatgaccaacttcgacaagaacctccccaacgagaaggtcctccccaagcacagcctcctctacgagtacttcaccgtctacaacgagctcaccaaggtcaagtacgtcaccgagggcatgcgcaagcccgccttcctcagcggcgagcagaagaaggccatcgtcgacctcctcttcaagaccaaccgcaaggtcaccgtcaagcagctcaaggaggactacttcaagaagatcgagtgcttcgacagcgtcgagatcagcggcgtcgaggaccgcttcaacgccagcctcggcacctaccacgacctcctcaagatcatcaaggacaaggacttcctcgacaacgaggagaacgaggacatcctcgaggacatcgtcctcaccctcaccctcttcgaggaccgcgagatgatcgaggagcgcctcaagacctacgcccacctcttcgacgacaaggtcatgaagcagctcaagcgccgccgctacaccggctggggccgcctcagccgcaagctcatcaacggcatccgcgacaagcagagcggcaagaccatcctcgacttcctcaagagcgacggcttcgccaaccgcaacttcatgcagctcatccacgacgacagcctcaccttcaaggaggacatccagaaggcccaggtcagcggccagggcgacagcctccacgagcacatcgccaacctcgccggcagccccgccatcaagaagggcatcctccagaccgtcaaggtcgtcgacgagctcgtcaaggtcatgggccgccacaagcccgagaacatcgtcatcgagatggcccgcgagaaccagaccacccagaagggccagaagaacagccgcgagcgcatgaagcgcatcgaggagggcatcaaggagctcggcagccagatcctcaaggagcaccccgtcgagaacacccagctccagaacgagaagctctacctctactacctccagaacggccgcgacatgtacgtcgaccaggagctcgacatcaaccgcctcagcgactacgacgtcgaccacatcgtcccccagagcttcctcaaggacgacagcatcgacaacaaggtcctcacccgcagcgacaagaaccgcggcaagagcgacaacgtccccagcgaggaggtcgtcaagaagatgaagaactactggcgccagctcctcaacgccaagctcatcacccagcgcaagttcgacaacctcaccaaggccgagcgcggcggcctcagcgagctcgacaaggccggcttcatcaagcgccagctcgtcgagacccgccagatcaccaagcacgtcgcccagatcctcgacagccgcatgaacaccaagtacgacgagaacgacaagctcatccgcgaggtcaaggtcatcaccctcaagagcaagctcgtcagcgacttccgcaaggacttccagttctacaaggtccgcgagatcaacaactaccaccacgcccacgacgcctacctcaacgccgtcgtcggcaccgccctcatcaagaagtaccccaagctcgagagcgagttcgtctacggcgactacaaggtctacgacgtccgcaagatgatcgccaagagcgagcaggagatcggcaaggccaccgccaagtacttcttctacagcaacatcatgaacttcttcaagaccgagatcaccctcgccaacggcgagatccgcaagcgccccctcatcgagaccaacggcgagaccggcgagatcgtctgggacaagggccgcgacttcgccaccgtccgcaaggtcctcagcatgccccaggtcaacatcgtcaagaagaccgaggtccagaccggcggcttcagcaaggagagcatcctccccaagcgcaacagcgacaagctcatcgcccgcaagaaggactgggaccccaagaagtacggcggcttcgacagccccaccgtcgcctacagcgtcctcgtcgtcgccaaggtcgagaagggcaagagcaagaagctcaagagcgtcaaggagctcctcggcatcaccatcatggagcgcagcagcttcgagaagaaccccatcgacttcctcgaggccaagggctacaaggaggtcaagaaggacctcatcatcaagctccccaagtacagcctcttcgagctcgagaacggccgcaagcgcatgctcgccagcgccggcgagctccagaagggcaacgagctcgccctccccagcaagtacgtcaacttcctctacctcgccagccactacgagaagctcaagggcagccccgaggacaacgagcagaagcagctcttcgtcgagcagcacaagcactacctcgacgagatcatcgagcagatcagcgagttcagcaagcgcgtcatcctcgccgacgccaacctcgacaaggtcctcagcgcctacaacaagcaccgcgacaagcccatccgcgagcaggccgagaacatcatccacctcttcaccctcaccaacctcggcgcccccgccgccttcaagtacttcgacaccaccatcgaccgcaagcgctacaccagcaccaaggaggtcctcgacgccaccctcatccaccagagcatcaccggcctctacgagacccgcatcgacctcagccagctcggcggcgacggcggcggcagccccaagaagaagcgcaaggtctag

SEQ ID NO: 1205 - DNA, 4140 bp, synthetic construct
  source 1..4140: mol_type = other DNA
atggacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacggcggcggcagccccaagaagaagcggaaggtgtga

SEQ ID NO: 1206 - RNA, 100 nt, synthetic construct; identical to SEQ ID NO: 300
  source 1..100: mol_type = other RNA

SEQ ID NO: 1207 - RNA, 100 nt, synthetic construct; identical to SEQ ID NO: 300
  source 1..100: mol_type = other RNA

SEQ ID NO: 1208 - RNA, 88 nt, synthetic construct; identical to SEQ ID NO: 405
  source 1..88: mol_type = other RNA

SEQ ID NO: 1209 - RNA, 88 nt, synthetic construct; identical to SEQ ID NO: 405
  source 1..88: mol_type = other RNA

SEQ ID NO: 1210 - RNA, 90 nt, synthetic construct; identical to SEQ ID NO: 407
  source 1..90: mol_type = other RNA

SEQ ID NO: 1211 - RNA, 90 nt, synthetic construct; identical to SEQ ID NO: 407
  source 1..90: mol_type = other RNA

SEQ ID NO: 1212 - RNA, 89 nt, synthetic construct
  source 1..89: mol_type = other RNA
nnnnnnnnnnnnnnnnnnnngttttagagctagaaatagcaagttaaaataaggctagtccgttatcaaaaatggcaccgagtcggtgc

SEQ ID NO: 1213 - RNA, 89 nt, synthetic construct; identical to SEQ ID NO: 1212
  source 1..89: mol_type = other RNA

SEQ ID NO: 1214 - RNA, 88 nt, synthetic construct
  source 1..88: mol_type = other RNA
nnnnnnnnnnnnnnnnnnnngttttagagctagaaatagcaagttaaaataaggctagtccgttatcacaagggcaccgagtcggtgc

SEQ ID NO: 1215 - RNA, 88 nt, synthetic construct; identical to SEQ ID NO: 1214
  source 1..88: mol_type = other RNA

SEQ ID NO: 1216 - RNA, 88 nt, synthetic construct
  source 1..88: mol_type = other RNA
nnnnnnnnnnnnnnnnnnnngttttagagctagaaatagcaagttaaaataaggctagtccgttatcaaaatggcaccgagtcggtgc

SEQ ID NO: 1217 - RNA, 88 nt, synthetic construct; identical to SEQ ID NO: 1216
  source 1..88: mol_type = other RNA

SEQ ID NO: 1218 - RNA, 87 nt, synthetic construct
  source 1..87: mol_type = other RNA
nnnnnnnnnnnnnnnnnnnngttttagagcgcgaagcgcaagttaaaataaggctagtccgttatcaaaatggcaccgagtcggtgc

SEQ ID NO: 1219 - RNA, 87 nt, synthetic construct; identical to SEQ ID NO: 1218
  source 1..87: mol_type = other RNA

SEQ ID NO: 1220 - RNA, 91 nt, synthetic construct
  source 1..91: mol_type = other RNA
gcgccagcagtggagcggtcgttttagagctagaaatagcaagttaaaataaggctagtccgttatcacgaaagggcaccgagtcggtgct

SEQ ID NO: 1221 - RNA, 91 nt, synthetic construct; identical to SEQ ID NO: 413
  source 1..91: mol_type = other RNA

SEQ ID NO: 1222 - RNA, 91 nt, synthetic construct
  source 1..91: mol_type = other RNA
gacggtctcgggaaagcgctgttttagagctagaaatagcaagttaaaataaggctagtccgttatcacgaaagggcaccgagtcggtgct

SEQ ID NO: 1223 - RNA, 91 nt, synthetic construct
  source 1..91: mol_type = other RNA
gcgctttcccgagaccgtccgttttagagctagaaatagcaagttaaaataaggctagtccgttatcacgaaagggcaccgagtcggtgct

SEQ ID NO: 1224 - RNA, 91 nt, synthetic construct
  source 1..91: mol_type = other RNA
ggaaagcgcttggtggtgccgttttagagctagaaatagcaagttaaaataaggctagtccgttatcacgaaagggcaccgagtcggtgct

SEQ ID NO: 1225 - RNA, 91 nt, synthetic construct
  source 1..91: mol_type = other RNA
cgccagcagtggagcggtccgttttagagctagaaatagcaagttaaaataaggctagtccgttatcacgaaagggcaccgagtcggtgct

SEQ ID NO: 1226 - RNA, 4140 nt, synthetic construct
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 1227 - protein, 1379 aa, synthetic construct; identical to SEQ ID NO: 800

SEQ ID NO: 1228 - RNA, 4140 nt, synthetic construct
  source 1..4140: mol_type = other RNA
atggacaagaagtacagcatcggactggacatcggaacaaacagcgtcggatgggcagtcatcacagacgaatacaaggtcccgagcaagaagttcaaggtcctgggaaacacagacagacacagcatcaagaagaacctgatcggagcactgctgttcgacagcggagaaacagcagaagcaacaagactgaagagaacagcaagaagaagatacacaagaagaaagaacagaatctgctacctgcaggaaatcttcagcaacgaaatggcaaaggtcgacgacagcttcttccacagactggaagaaagcttcctggtcgaagaagacaagaagcacgaaagacacccgatcttcggaaacatcgtcgacgaagtcgcataccacgaaaagtacccgacaatctaccacctgagaaagaagctggtcgacagcacagacaaggcagacctgagactgatctacctggcactggcacacatgatcaagttcagaggacacttcctgatcgaaggagacctgaacccggacaacagcgacgtcgacaagctgttcatccagctggtccagacatacaaccagctgttcgaagaaaacccgatcaacgcaagcggagtcgacgcaaaggcaatcctgagcgcaagactgagcaagagcagaagactggaaaacctgatcgcacagctgccgggagaaaagaagaacggactgttcggaaacctgatcgcactgagcctgggactgacaccgaacttcaagagcaacttcgacctggcagaagacgcaaagctgcagctgagcaaggacacatacgacgacgacctggacaacctgctggcacagatcggagaccagtacgcagacctgttcctggcagcaaagaacctgagcgacgcaatcctgctgagcgacatcctgagagtcaacacagaaatcacaaaggcaccgctgagcgcaagcatgatcaagagatacgacgaacaccaccaggacctgacactgctgaaggcactggtcagacagcagctgccggaaaagtacaaggaaatcttcttcgaccagagcaagaacggatacgcaggatacatcgacggaggagcaagccaggaagaattctacaagttcatcaagccgatcctggaaaagatggacggaacagaagaactgctggtcaagctgaacagagaagacctgctgagaaagcagagaacattcgacaacggaagcatcccgcaccagatccacctgggagaactgcacgcaatcctgagaagacaggaagacttctacccgttcctgaaggacaacagagaaaagatcgaaaagatcctgacattcagaatcccgtactacgtcggaccgctggcaagaggaaacagcagattcgcatggatgacaagaaagagcgaagaaacaatcacaccgtggaacttcgaagaagtcgtcgacaagggagcaagcgcacagagcttcatcgaaagaatgacaaacttcgacaagaacctgccgaacgaaaaggtcctgccgaagcacagcctgctgtacgaatacttcacagtctacaacgaactgacaaaggtcaagtacgtcacagaaggaatgagaaagccggcattcctgagcggagaacagaagaaggcaatcgtcgacctgctgttcaagacaaacagaaaggtcacagtcaagcagctgaaggaagactacttcaagaagatcgaatgcttcgacagcgtcgaaatcagcggagtcgaagacagattcaacgcaagcctgggaacataccacgacctgctgaagatcatcaaggacaaggacttcctggacaacgaagaaaacgaagacatcctggaagacatcgtcctgacactgacactgttcgaagacagagaaatgatcgaagaaagactgaagacatacgcacacctgttcgacgacaaggtcatgaagcagctgaagagaagaagatacacaggatggggaagactgagcagaaagctgatcaacggaatcagagacaagcagagcggaaagacaatcctggacttcctgaagagcgacggattcgcaaacagaaacttcatgcagctgatccacgacgacagcctgacattcaaggaagacatccagaaggcacaggtcagcggacagggagacagcctgcacgaacacatcgcaaacctggcaggaagcccggcaatcaagaagggaatcctgcagacagtcaaggtcgtcgacgaactggtcaaggtcatgggaagacacaagccggaaaacatcgtcatcgaaatggcaagagaaaaccagacaacacagaagggacagaagaacagcagagaaagaatgaagagaatcgaagaaggaatcaaggaactgggaagccagatcctgaaggaacacccggtcgaaaacacacagctgcagaacgaaaagctgtacctgtactacctgcagaacggaagagacatgtacgtcgaccaggaactggacatcaacagactgagcgactacgacgtcgaccacatcgtcccgcagagcttcctgaaggacgacagcatcgacaacaaggtcctgacaagaagcgacaagaacagaggaaagagcgacaacgtcccgagcgaagaagtcgtcaagaagatgaagaactactggagacagctgctgaacgcaaagctgatcacacagagaaagttcgacaacctgacaaaggcagagagaggaggactgagcgaactggacaaggcaggattcatcaagagacagctggtcgaaacaagacagatcacaaagcacgtcgcacagatcctggacagcagaatgaacacaaagtacgacgaaaacgacaagctgatcagagaagtcaaggtcatcacactgaagagcaagctggtcagcgacttcagaaaggacttccagttctacaaggtcagagaaatcaacaactaccaccacgcacacgacgcatacctgaacgcagtcgtcggaacagcactgatcaagaagtacccgaagctggaaagcgaattcgtctacggagactacaaggtctacgacgtcagaaagatgatcgcaaagagcgaacaggaaatcggaaaggcaacagcaaagtacttcttctacagcaacatcatgaacttcttcaagacagaaatcacactggcaaacggagaaatcagaaagagaccgctgatcgaaacaaacggagaaacaggagaaatcgtctgggacaagggaagagacttcgcaacagtcagaaaggtcctgagcatgccgcaggtcaacatcgtcaagaagacagaagtccagacaggaggattcagcaaggaaagcatcctgccgaagagaaacagcgacaagctgatcgcaagaaagaaggactgggacccgaagaagtacggaggattcgacagcccgacagtcgcatacagcgtcctggtcgtcgcaaaggtcgaaaagggaaagagcaagaagctgaagagcgtcaaggaactgctgggaatcacaatcatggaaagaagcagcttcgaaaagaacccgatcgacttcctggaagcaaagggatacaaggaagtcaagaaggacctgatcatcaagctgccgaagtacagcctgttcgaactggaaaacggaagaaagagaatgctggcaagcgcaggagaactgcagaagggaaacgaactggcactgccgagcaagtacgtcaacttcctgtacctggcaagccactacgaaaagctgaagggaagcccggaagacaacgaacagaagcagctgttcgtcgaacagcacaagcactacctggacgaaatcatcgaacagatcagcgaattcagcaagagagtcatcctggcagacgcaaacctggacaaggtcctgagcgcatacaacaagcacagagacaagccgatcagagaacaggcagaaaacatcatccacctgttcacactgacaaacctgggagcaccggcagcattcaagtacttcgacacaacaatcgacagaaagagatacacaagcacaaaggaagtcctggacgcaacactgatccaccagagcatcacaggactgtacgaaacaagaatcgacctgagccagctgggaggagacggaggaggaagcccgaagaagaagagaaaggtctag

SEQ ID NO: 1229 - protein, 7 aa, synthetic construct
PKKKRKV

SEQ ID NO: 1230 - protein, 7 aa, synthetic construct
PKKKRRV

SEQ ID NO: 1231 - protein, 16 aa, synthetic construct
KRPAATKKAGQAKKKK

SEQ ID NO: 1232 - protein, 6 aa, synthetic construct
HHHHHH

SEQ ID NO: 1233 - protein, 8 aa, synthetic construct
HHHHHHHH

SEQ ID NO: 1234 - RNA, 42 nt, synthetic construct
  source 1..42: mol_type = other RNA
nnnnnnnnnnnnnnnnnnnngttttagagctatgctgttttg

SEQ ID NO: 1235 - RNA, 71 nt, synthetic construct
  source 1..71: mol_type = other RNA
gttttagagctagaaatagcaagttaaaataaggctagtccgttatcacgaaagggcaccgagtcggtgct

(19) United States
(12) Patent Application Publication
(10) Pub. No.: US 2025/0302874 A1
(43) Pub. Date: Oct. 2, 2025

(54) CD38 COMPOSITIONS AND METHODS FOR IMMUNOTHERAPY

(71) Applicant: Intellia Therapeutics, Inc., Cambridge, MA (US)

(72) Inventors: Ruan Oliveira, Cambridge, MA (US); Christian Dombrowski, Cambridge, MA (US)

(21) Appl. No.: 18/706,899

(22) PCT Filed: Nov. 2, 2022
(86) PCT No.: PCT/US2022/048691

§ 371 Date: May 2, 2024

Related U.S. Application Data
(60) Provisional application No. 63/275,431, filed on Nov. 3, 2021.

(51) Int. Cl. A61K 35/17; A61K 40/11; A61K 40/15; A61K 40/31; A61K 40/42; C07K 14/705; C12N 9/22; C12N 15/11

(52) U.S. Cl. CPC A61K 35/17; A61K 40/11; A61K 40/15; A61K 40/31; A61K 40/4222; C07K 14/70596; C12N 9/226; C12N 15/111; C12N 2310/20; C12N 2310/531

(57) ABSTRACT

Compositions and methods for editing, e.g., altering a DNA sequence, within a CD38 gene are provided. Compositions and methods for immunotherapy are provided.

### CROSS-REFERENCE TO RELATED APPLICATION

This application is a national stage filing under 35 U.S.C. § 371 of PCT/US2022/048691, filed Nov. 2, 2022, which claims the benefit of U.S. Provisional Application No. 63/275,431 filed on Nov. 3, 2021, the content of each of which is hereby incorporated by reference in its entirety.

### SEQUENCE LISTING

The application contains a Sequence Listing which has been submitted electronically in .XML format and is hereby incorporated by reference in its entirety. Said .XML copy, created on Apr. 30, 2025, is named “ILH-02501.xml” and is 572,086 bytes in size. The sequence listing contained in this .XML file is part of the specification and is hereby incorporated by reference herein in its entirety.

### BACKGROUND

Cyclic ADP ribose hydrolase (CD38) is an ectoenzyme expressed on the surface of certain immune cells that has been used as a biomarker to identify T cells and lymphocyte activation. It synthesizes second messengers cyclic adenosine 5′-diphosphate-ribose (cADP-ribose) and nicotinamide dinucleotide (NAD+). NAD+ is a second messenger for glucose-induced insulin secretion. Adenosine can be synthesized from NAD+, and adenosine has been implicated in immune suppression and in the immunomodulation of multiple myeloma and lung cancer. These findings have led to speculation that CD38 may function as an immune check point molecule. Additionally, CD38 has been implicated in aging and age-related dysfunction, responding to microbial infection, and hyperinflammatory disorders. Moreover, CD38 regulates antitumor T cell exhaustion.

CD38 is expressed on immune cells including T cells, B cells, circulating monocytes, dendritic cells, granulocytes, plasma cells, both resting and circulating NK cells, neutrophils, and granulocytes. CD38 can also function as a receptor on these cells, and this function can activate immune cells and is necessary for these cells to proliferate. On a T cell surface, CD38 interacts with its ligand, CD31, and elicits downstream effects that overlap with T cell receptor (TCR)/CD3 activation.

CD38, which has been associated with several hematological malignancies, plays a role in immune suppression in the tumor microenvironment. For example, chronic lymphocytic leukemia CD38+ clones have been shown to have a survival advantage over CD38− clones. CD38 is often overexpressed in multiple myeloma plasma cells that accumulate in the bone marrow and is involved in the metabolic reprogramming and cellular proliferation by upregulating the PI3K/AKT/mTOR pathway.

### SUMMARY

In certain aspects, provided herein are compositions and methods related to the preparation of engineered cells with genetic modifications (e.g., insertions, deletions, substitutions) in a CD38 gene sequence using the CRISPR/Cas system, as well as cells with genetic modifications in the CD38 gene sequence (e.g., modifications that reduce or eliminate CD38 expression by the cells) and their use in various methods, including, but not limited to, adoptive cell transfer therapy for cancers (e.g., CD38 expressing cancers).

In some embodiments, the engineered cells provided herein are genetically modified T cells or natural killer (NK) cells. In certain embodiments, the engineered cells are cells that have been modified to express a chimeric antigen receptor (CAR), such as a CAR specific for CD38 polypeptides (i.e., a full-length CAR protein or a fragment thereof, including, for example, an MHC-presented CD38 peptide). In certain embodiments, the engineered cells express a recombinant T cell receptor (TCR), such as a recombinant TCR specific for a CD38 polypeptide. In some embodiments, the engineered cells may include other genetic modifications in additional genomic sequences including, at the T-cell receptor (TCR) loci, e.g., TRAC or TRBC loci, to reduce and/or eliminate TCR expression; at genomic loci that reduce and/or eliminate expression of one or more MHC class I molecules, e.g., B2M and HLA-A loci; genomic loci that reduce and/or eliminate expression of one or more MHC class II molecules, e.g., CIITA loci; and/or at one or more checkpoint inhibitor loci, e.g., CD244 (2B4) loci, TIM3 loci, LAG3, and PD-1 loci. In some embodiments, such cells are used to treat a cancer in a subject (e.g., CD38 expressing cancer in a subject). In some embodiments, such genetically modified cells are used in a combination therapy that also includes administration of a CD38-targeting therapeutic, such as a CD38-specific monoclonal antibody (e.g., daratumumab, isatuximab) to the subject.

In some embodiments, the present disclosure relates to populations of cells, including cells with genetic modification of their CD38 gene sequence, and optionally other genomic loci disclosed herein. In certain embodiments, such populations of cells may be used in adoptive cell (e.g., T cell, NK cell) transfer therapies. In some embodiments, the present disclosure relates to compositions and uses of the cells with genetic modification of the CD38 sequence for use in therapy, e.g., cancer therapy and immunotherapy.

In certain aspects, provided herein is an engineered cell comprising a genetic modification in a human CD38 sequence, such as a genetic modification within the genomic coordinates of chr4:15766497-15871496.

Also disclosed is the use of a composition and/or formulation of a cell of any of the foregoing embodiments for the preparation of a medicament for treating a subject. The subject may be human or animal (e.g. human or non-human animal, e.g., cynomolgus monkey). In certain embodiments, the subject is human.

In some aspects, disclosed are any of the foregoing compositions or formulations for use in producing a genetic modification (e.g., an insertion, a substitution, or a deletion) within a CD38 gene sequence, e.g., using a CRISPR/Cas system. In some embodiments, provided herein are gRNA molecules, CRISPR systems, cells, and methods useful for genome editing of cells. In certain embodiments, the genetic modification within the CD38 gene sequence results in a change in the nucleic acid sequence that prevents translation of a full-length CD38 protein, e.g., by forming a frameshift or nonsense mutation, such that translation is terminated prematurely. In some embodiments, the genetic modification can include insertion, substitution, or deletion at a splice site, i.e., a splice acceptor site or a splice donor site, such that the abnormal splicing results in a frameshift mutation, nonsense mutation, or truncated mRNA, such that translation is terminated prematurely. In some embodiments, genetic modifications can also disrupt translation or folding of the encoded protein resulting in premature translation termination. In certain embodiments, compositions and methods provided herein for use in producing a genetic modification within a CD38 sequence that results in reduced expression of a CD38 protein (e.g., cell surface expression of the CD38 protein, from the CD38 sequence).

In certain aspects, provided herein are methods of providing an immunotherapy to a subject, the method including administering to the subject an effective amount of a cell as described herein (e.g., a genetically modified T cell or NK cell described herein). In some embodiments, the immunotherapy is for the treatment of a cancer in a subject. In certain embodiments, the cancer is a CD38-expressing cancer. In some embodiments, the therapy also includes administration of a CD38-targeting therapeutic, such as a CD38-specific monoclonal antibody (e.g., daratumumab, isatuximab), to the subject. In certain embodiments, the modification of the CD38 gene sequence in the cells is such that the cells are resistant to targeting by a CD38-targeting therapeutic (e.g., another CD38-targeting adoptively transferred cell and/or a CD38-specific therapeutic, such as an anti-CD38 monoclonal antibody). In certain embodiments, the resistance to targeting is a result of a reduced expression of CD38 on the cells. In some embodiments, the resistance to targeting is the result of a modification of the expressed CD38 protein that eliminates an epitope recognized by the CD38-targeting therapeutic.

In embodiments, the immunotherapy method includes lymphodepletion prior to administering a cell or population of cells described herein. In some embodiments, the method includes administering a lymphodepleting agent or immunosuppressant prior to administering to the subject an effective amount of the cell as described herein, for example, a cell of any of the aforementioned cell aspects and embodiments. In certain embodiments, the therapeutic method includes preparing cells (e.g., a population of cells) using a method provided herein such that they have reduced and/or eliminated CD38 expression prior to administration to the subject.

In another aspect, provided herein is a method of preparing cells (e.g., a population of cells, such a T cells or NK cells) for immunotherapy, the method including: (a) modifying cells by reducing or eliminating expression of CD38 protein and, optionally, one or more or all components of a T-cell receptor (TCR), for example, by introducing into said cells a gRNA molecule (as described herein), or more than one gRNA molecule, as disclosed herein; and (b) expanding said cells. Cells provided herein are suitable for further engineering, e.g., by introduction of a heterologous sequence or heterologous sequences coding for a targeting receptor, e.g., a protein that mediates TCR/CD3 zeta chain signalling. In some embodiments, the protein is a targeting receptor selected from a non-endogenous TCR or CAR sequence (e.g., sequences encoding TCRs or CARs specific for CD38 polypeptides). In some embodiments, the protein is a wild-type or variant TCR. Cells provided herein may also be suitable for further engineering by introduction of a heterologous sequence coding for an alternative antigen binding moiety, e.g., by introduction of a heterologous sequence coding for an alternative (non-endogenous) T cell receptor, e.g., a chimeric antigen receptors (CAR) engineered to target a specific protein (e.g., CD38). CARs are also known as chimeric immunoreceptors, chimeric T cell receptors or artificial T cell receptors).

In another aspect, provided herein is a method of treating a subject that includes administering cells (e.g., a population of cells, such as a population of T cells or NK cells) prepared by a method described herein (e.g., a method that results in a reduction and/or elimination of CD38 protein expression). In some embodiments, the method further comprises administering an additional therapeutic agent to the subject. The additional therapeutic agent can be a CD38-targeting therapy such as an anti-CD38 antibody (e.g., daratumumab, isatuximab), small molecule inhibitor of CD38, an NAD+ analog, a flavonoid, or a cell comprising a chimeric antigen receptor that specifically binds to CD38. In some embodiments, the subject is treated for a cancer, an infection, and/or an aging disorder. The cancer can be a solid tumor or a hematological cancer. In some embodiments, the cancer is a CD38 expressing cancer. In some embodiments, the cancer is multiple myeloma, chronic lymphocytic leukemia, lung cancer, prostate cancer, or melanoma.

Further embodiments are provided throughout and described in the claims and Figures.

### BRIEF DESCRIPTION OF THE DRAWINGS

FIG. 1 is a graph showing the percentage of NK cells without CD38 surface expression after treatment with LNPs delivering Cas9 mRNA and a gRNA as indicated in Table 5 targeting CD38.

FIG. 2 is a graph showing the percentage of NK cells with or without CD38 surface expression and with or without GFP expression.

FIG. 3 shows the editing frequencies of T cells harvested 4 days post-LNP treatment with a fixed dose of BC22 mRNA and uracil glycosylase inhibitor (UGI) mRNA and a decreasing dose of CD38 sgRNA in the 100-mer or 91-mer formats.

FIG. 4 shows the percentage of CD8+ T cells that are negative for CD38 surface receptors following treatment with a fixed dose of BC22 mRNA and UGI mRNA and a decreasing dose of B2M and CD38 sgRNAs in the 100-mer or 91-mer formats.

FIG. 5A shows mean percent CD38 negative NK cells as assessed by flow cytometry after editing with various guide concentrations.

FIG. 5B shows mean percent CD38 negative NK cells as assessed by flow cytometry after editing with various mRNA concentrations.

FIG. 6 shows mean percent CD38 KO as assessed by flow cytometry after gene editing.

### DETAILED DESCRIPTION

Reference will now be made in detail to certain embodiments disclosed herein. The present teaching also encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “a conjugate” includes a plurality of conjugates and reference to “a cell” includes a plurality of cells (e.g., a population of cells) and the like.

Numeric ranges are inclusive of the numbers defining the range. Measured and measurable values are understood to be approximate, taking into account significant digits and the error associated with the measurement. In some embodiments a population of cells refers to a population of at least 10³, 10⁴, 10⁵ or 10⁶ cells, preferably 10⁷, 2×10⁷, 5×10⁷, or 10⁸ cells.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The term “or” is used in an inclusive sense in the specification, i.e., equivalent to “and/or,” unless the context clearly indicates otherwise.

The term “about”, when used before a list, modifies each member of the list. The term “about” is understood to encompass tolerated variation or error within the art, e.g., 2 standard deviations from the mean, or the sensitivity of the method used to take a measurement. When “about” is present before the first value of a series, it can be understood to modify each value in the series.

Ranges are understood to include the numbers at the end of the range and all logical values therebetween. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, ranges include both the upper and lower limit.

In the event of a conflict between a sequence in the application and an indicated accession number or position in an accession number, the sequence in the application predominates.

In the event of a conflict between a chemical name and a structure, the structure predominates.

As used herein, “detecting an analyte” and the like is understood as performing an assay in which the analyte can be detected, if present, wherein the analyte is present in an amount above the level of detection of the assay.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

As used herein, “eliminate” is understood to mean reducing a level to below the detection threshold of an assay.

The section headings used herein are for organizational purposes only and are not to be construed as limiting the desired subject matter in any way. In the event that any material incorporated by reference contradicts any term defined in this specification or any other express content of this specification, this specification controls.

### Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. An RNA may comprise one or more deoxyribose nucleotides, e.g. as modifications, and similarly a DNA may comprise one or more ribonucleotides. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases, and linkages or can include both conventional components and substitutions (e.g., conventional nucleosides with 2′ methoxy substituents or polymers containing both conventional nucleosides and one or more nucleoside analogs). Nucleic acids include “locked nucleic acids” (LNA) and analogues containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA,” “gRNA,” and simply “guide” are used herein interchangeably to refer to, for example, either a single guide RNA or the combination of a crRNA and a trRNA (also known as tracrRNA). The crRNA and trRNA may be associated as a single RNA molecule (as a single guide RNA, sgRNA) or, for example, in two separate RNA strands (dual guide RNA, dgRNA). “Guide RNA” or “gRNA” refers to each type. The trRNA may be a naturally-occurring sequence or a trRNA sequence with modifications or variations.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs: 1-88. In some embodiments, the target sequence is in a gene or on a chromosome, for example, and is complementary to the guide sequence. In some embodiments, the degree of complementarity or identity between a guide sequence and its corresponding target sequence is at least 75%, 80%, 85%, 90%, 95%, or 100%. For example, in some embodiments, the guide sequence comprises a sequence with at least 75%, 80%, 85%, 90%, 95%, or 100% identity to at least 17, 18, 19, or 20 contiguous nucleotides of a sequence selected from SEQ ID NOs: 1-88. In some embodiments, the guide sequence and the target region may be 100% complementary or identical. In other embodiments, the guide sequence and the target region may contain at least one mismatch, i.e., one nucleotide that is not identical or not complementary, depending on the reference sequence. For example, the guide sequence and the target sequence may contain 1, 2, 3, or 4 mismatches, where the total length of the target sequence is 17, 18, 19, 20, or more nucleotides. In some embodiments, the guide sequence and the target region may contain 1-4 mismatches where the guide sequence comprises at least 17, 18, 19, 20, or more nucleotides. In some embodiments, the guide sequence and the target region may contain 1, 2, 3, or 4 mismatches where the guide sequence comprises 20 nucleotides. That is, the guide sequence and the target region may form a duplex region having 17, 18, 19, 20 or more base pairs. In certain embodiments, the duplex region may include 1, 2, 3, or 4 mismatches such that guide strand and target sequence are not fully complementary. For example, a guide strand and target sequence may be complementary over a 20 nucleotide region, including 2 mismatches, such that the guide sequence and target sequence are 90% complementary providing a duplex region of 18 base pairs out of 20.

Target sequences for RNA-guided DNA binding agents include both the positive and negative strands of genomic DNA (i.e., the sequence given and the reverse compliment of the sequence), as a nucleic acid substrate for an RNA-guided DNA binding agent is a double stranded nucleic acid. Accordingly, where a guide sequence is said to be “complementary to a target sequence,” it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence. Unless otherwise indicated, nucleotides in guide RNA sequences provided herein that are identified using a capital letter are RNA nucleotide wit a 2′-OH.

As used herein, an “RNA guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease,” as used herein, encompasses Cas cleavases, Cas nickases, and dCas DNA binding agents. The dCas DNA binding agent may be a dead nuclease comprising non-functional nuclease domains (RuvC or HNH domain). In some embodiments the Cas cleavase or Cas nickase encompasses a dCas DNA binding agent modified to permit DNA cleavage, e.g. via fusion with a FokI domain. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, the term “editor” refers to an agent comprising a polypeptide that is capable of making a modification within a DNA sequence. In some embodiments, the editor is a cleavase, such as a Cas9 cleavase. In some embodiments, the editor is capable of deaminating a base within a DNA molecule. In some embodiments, the editor is capable of deaminating a cytosine (C) in DNA. In some embodiments, the editor is a fusion protein comprising an RNA-guided nickase fused to a cytidine deaminase. In some embodiments, the editor is a fusion protein comprising an RNA-guided nickase fused to an APOBEC3A deaminase (A3A). In some embodiments, the editor comprises a Cas9 nickase fused to an APOBEC3A deaminase (A3A). In some embodiments, the editor is a fusion protein comprising an RNA-guided nickase fused to a cytidine deaminase and a uracil glycosylase inhibitor (UGI). In some embodiments, the editor lacks a UGI.

As used herein, a “cytidine deaminase” means a polypeptide or complex of polypeptides that is capable of cytidine deaminase activity; that is catalyzing the hydrolytic deamination of cytidine or deoxycytidine, typically resulting in uridine or deoxyuridine. Cytidine deaminases encompass enzymes in the cytidine deaminase superfamily, and in particular, enzymes of the APOBEC family (APOBEC1, APOBEC2, APOBEC4, and APOBEC3 subgroups of enzymes), activation-induced cytidine deaminase (AID or AICDA) and CMP deaminases (see, e.g., Conticello et al., Mol. Biol. Evol. 22:367-77, 2005; Conticello, Genome Biol. 9:229, 2008; Muramatsu et al., J. Biol. Chem. 274: 18470-6, 1999); Carrington et al., Cells 9:1690 (2020)).

As used herein, the term “APOBEC3” refers to a APOBEC3 protein, such as an APOBEC3 protein expressed by any of the seven genes (A3A-A3H) of the human APOBEC3 locus. The APOBEC3 may have catalytic DNA or RNA editing activity. An amino acid sequence of APOBEC3A has been described (UniPROT accession ID: p31941). In some embodiments, the APOBEC3 protein is a mammalian, e.g., human wild-type APOBEC3 protein or a variant protein. Variants include proteins having a sequence that differs from wild-type APOBEC3 protein by one or several mutations (i.e., substitutions, deletions, insertions), such as one or several single point substitutions. For instance, a shortened APOBEC3 sequence could be used, e.g. by deleting several N-term or C-term amino acids, preferably one to four amino acids at the C-terminus of the sequence. As used herein, the term “variant” refers to allelic variants, splicing variants, and natural or artificial mutants, which are homologous to a APOBEC3 reference sequence. The variant is “functional” in that it shows a catalytic activity of DNA or RNA editing. In some embodiments, an APOBEC3 (such as a human APOBEC3A) has a wild-type amino acid position 57 (as numbered in the wild-type sequence). In some embodiments, an APOBEC3 (such as a human APOBEC3A) has an asparagine at amino acid position 57 (as numbered in the wild-type sequence).

As used herein, a “nickase” is an enzyme that creates a single-strand break (also known as a “nick”) in double strand DNA, i.e., cuts one strand but not the other of a DNA double helix. As used herein, an “RNA-guided DNA nickase” means a polypeptide or complex of polypeptides having DNA nickase activity, wherein the DNA nickase activity is sequence-specific and depends on the sequence of the RNA. Exemplary RNA-guided DNA nickases include Cas nickases. Cas nickases include nickase forms of a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. Class 2 Cas nickases include variants in which only one of the two catalytic domains is inactivated, which have RNA-guided DNA nickase activity. Class 2 Cas nickases include, for example, Cas9 (e.g., H840A, D10A, or N863A variants of SpyCas9), Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like protein domain. Cpf1 sequences of Zetsche are incorporated by reference in their entirety. See, e.g., Zetsche, Tables S1 and S3. “Cas9” encompasses S. pyogenes (Spy) Cas9, the variants of Cas9 listed herein, and equivalents thereof. See, e.g., Makarova et al., Nat Rev Microbiol, 13(11): 722-36 (2015); Shmakov et al., Molecular Cell, 60:385-397 (2015).

As used herein, the term “fusion protein” refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

The term “linker,” as used herein, refers to a chemical group or a molecule linking two adjacent molecules or moieties. Typically, the linker is positioned between, or flanked by, two groups, molecules, or other moieties and connected to each one via a covalent bond. In some embodiments, the linker is an amino acid or a plurality of amino acids (e.g., a peptide or protein) such as a 16-amino acid residue “XTEN” linker or a variant thereof (see, e.g., the Examples and Schellenberger et al. A recombinant polypeptide extends the in vivo half-life of peptides and proteins in a tunable manner. Nat. Biotechnol. 27, 1186-1190 (2009)). In some embodiments, the XTEN linker comprises the sequence SGSETPGTSESATPES (SEQ ID NO: 900), SGSETPGTSESA (SEQ ID NO: 901), or SGSETPGTSESATPEGGSGGS (SEQ ID NO: 902).

As used herein, the term “uracil glycosylase inhibitor” or “UGI” refers to a protein that is capable of inhibiting a uracil-DNA glycosylase (UDG) base-excision repair enzyme.

Exemplary nucleotide and polypeptide sequences of Cas9 molecules are provided below. Methods for identifying alternate nucleotide sequences encoding Cas9 polypeptide sequences, including alternate naturally occurring variants, are known in the art. Sequences with at least 75%, 80%, 85%, 90%, 95%, 96%, 97%, 98%, or 99% identity to any of the Cas9 nucleic acid sequences, amino acid sequences, or nucleic acid sequences encoding the amino acid sequences provided herein are also contemplated.

Exemplary open reading frame for Cas9
AUGGACAAGAAGUACUCCAUCGGCCUGGACAUCGGCACCAACUCCGUGGGCUGGGCCGUGAU
CACCGACGAGUACAAGGUGCCCUCCAAGAAGUUCAAGGUGCUGGGCAACACCGACCGGCACU
CCAUCAAGAAGAACCUGAUCGGCGCCCUGCUGUUCGACUCCGGCGAGACCGCCGAGGCCACC
CGGCUGAAGCGGACCGCCCGGCGGCGGUACACCCGGCGGAAGAACCGGAUCUGCUACCUGC
AGGAGAUCUUCUCCAACGAGAUGGCCAAGGUGGACGACUCCUUCUUCCACCGGCUGGAGGAG
UCCUUCCUGGUGGAGGAGGACAAGAAGCACGAGCGGCACCCCAUCUUCGGCAACAUCGUGGA
CGAGGUGGCCUACCACGAGAAGUACCCCACCAUCUACCACCUGCGGAAGAAGCUGGUGGACU
CCACCGACAAGGCCGACCUGCGGCUGAUCUACCUGGCCCUGGCCCACAUGAUCAAGUUCCGG
GGCCACUUCCUGAUCGAGGGCGACCUGAACCCCGACAACUCCGACGUGGACAAGCUGUUCAU
CCAGCUGGUGCAGACCUACAACCAGCUGUUCGAGGAGAACCCCAUCAACGCCUCCGGCGUGG
ACGCCAAGGCCAUCCUGUCCGCCCGGCUGUCCAAGUCCCGGCGGCUGGAGAACCUGAUCGC
CCAGCUGCCCGGCGAGAAGAAGAACGGCCUGUUCGGCAACCUGAUCGCCCUGUCCCUGGGC
CUGACCCCCAACUUCAAGUCCAACUUCGACCUGGCCGAGGACGCCAAGCUGCAGCUGUCCAA
GGACACCUACGACGACGACCUGGACAACCUGCUGGCCCAGAUCGGCGACCAGUACGCCGACC
UGUUCCUGGCCGCCAAGAACCUGUCCGACGCCAUCCUGCUGUCCGACAUCCUGCGGGUGAAC
ACCGAGAUCACCAAGGCCCCCCUGUCCGCCUCCAUGAUCAAGCGGUACGACGAGCACCACCA
GGACCUGACCCUGCUGAAGGCCCUGGUGCGGCAGCAGCUGCCCGAGAAGUACAAGGAGAUC
UUCUUCGACCAGUCCAAGAACGGCUACGCCGGCUACAUCGACGGCGGCGCCUCCCAGGAGGA
GUUCUACAAGUUCAUCAAGCCCAUCCUGGAGAAGAUGGACGGCACCGAGGAGCUGCUGGUGA
AGCUGAACCGGGAGGACCUGCUGCGGAAGCAGCGGACCUUCGACAACGGCUCCAUCCCCCAC
CAGAUCCACCUGGGCGAGCUGCACGCCAUCCUGCGGCGGCAGGAGGACUUCUACCCCUUCC
UGAAGGACAACCGGGAGAAGAUCGAGAAGAUCCUGACCUUCCGGAUCCCCUACUACGUGGGC
CCCCUGGCCCGGGGCAACUCCCGGUUCGCCUGGAUGACCCGGAAGUCCGAGGAGACCAUCA
CCCCCUGGAACUUCGAGGAGGUGGUGGACAAGGGCGCCUCCGCCCAGUCCUUCAUCGAGCG
GAUGACCAACUUCGACAAGAACCUGCCCAACGAGAAGGUGCUGCCCAAGCACUCCCUGCUGU
ACGAGUACUUCACCGUGUACAACGAGCUGACCAAGGUGAAGUACGUGACCGAGGGCAUGCGG
AAGCCCGCCUUCCUGUCCGGCGAGCAGAAGAAGGCCAUCGUGGACCUGCUGUUCAAGACCAA
CCGGAAGGUGACCGUGAAGCAGCUGAAGGAGGACUACUUCAAGAAGAUCGAGUGCUUCGACU
CCGUGGAGAUCUCCGGCGUGGAGGACCGGUUCAACGCCUCCCUGGGCACCUACCACGACCU
GCUGAAGAUCAUCAAGGACAAGGACUUCCUGGACAACGAGGAGAACGAGGACAUCCUGGAGG
ACAUCGUGCUGACCCUGACCCUGUUCGAGGACCGGGAGAUGAUCGAGGAGCGGCUGAAGAC
CUACGCCCACCUGUUCGACGACAAGGUGAUGAAGCAGCUGAAGCGGCGGCGGUACACCGGCU
GGGGCCGGCUGUCCCGGAAGCUGAUCAACGGCAUCCGGGACAAGCAGUCCGGCAAGACCAU
CCUGGACUUCCUGAAGUCCGACGGCUUCGCCAACCGGAACUUCAUGCAGCUGAUCCACGACG
ACUCCCUGACCUUCAAGGAGGACAUCCAGAAGGCCCAGGUGUCCGGCCAGGGCGACUCCCUG
CACGAGCACAUCGCCAACCUGGCCGGCUCCCCCGCCAUCAAGAAGGGCAUCCUGCAGACCGU
GAAGGUGGUGGACGAGCUGGUGAAGGUGAUGGGCCGGCACAAGCCCGAGAACAUCGUGAUC
GAGAUGGCCCGGGAGAACCAGACCACCCAGAAGGGCCAGAAGAACUCCCGGGAGCGGAUGAA
GCGGAUCGAGGAGGGCAUCAAGGAGCUGGGCUCCCAGAUCCUGAAGGAGCACCCCGUGGAG
AACACCCAGCUGCAGAACGAGAAGCUGUACCUGUACUACCUGCAGAACGGCCGGGACAUGUA
CGUGGACCAGGAGCUGGACAUCAACCGGCUGUCCGACUACGACGUGGACCACAUCGUGCCCC
AGUCCUUCCUGAAGGACGACUCCAUCGACAACAAGGUGCUGACCCGGUCCGACAAGAACCGG
GGCAAGUCCGACAACGUGCCCUCCGAGGAGGUGGUGAAGAAGAUGAAGAACUACUGGCGGCA
GCUGCUGAACGCCAAGCUGAUCACCCAGCGGAAGUUCGACAACCUGACCAAGGCCGAGCGGG
GCGGCCUGUCCGAGCUGGACAAGGCCGGCUUCAUCAAGCGGCAGCUGGUGGAGACCCGGCA
GAUCACCAAGCACGUGGCCCAGAUCCUGGACUCCCGGAUGAACACCAAGUACGACGAGAACG
ACAAGCUGAUCCGGGAGGUGAAGGUGAUCACCCUGAAGUCCAAGCUGGUGUCCGACUUCCGG
AAGGACUUCCAGUUCUACAAGGUGCGGGAGAUCAACAACUACCACCACGCCCACGACGCCUA
CCUGAACGCCGUGGUGGGCACCGCCCUGAUCAAGAAGUACCCCAAGCUGGAGUCCGAGUUCG
UGUACGGCGACUACAAGGUGUACGACGUGCGGAAGAUGAUCGCCAAGUCCGAGCAGGAGAUC
GGCAAGGCCACCGCCAAGUACUUCUUCUACUCCAACAUCAUGAACUUCUUCAAGACCGAGAUC
ACCCUGGCCAACGGCGAGAUCCGGAAGCGGCCCCUGAUCGAGACCAACGGCGAGACCGGCGA
GAUCGUGUGGGACAAGGGCCGGGACUUCGCCACCGUGCGGAAGGUGCUGUCCAUGCCCCAG
GUGAACAUCGUGAAGAAGACCGAGGUGCAGACCGGCGGCUUCUCCAAGGAGUCCAUCCUGCC
CAAGCGGAACUCCGACAAGCUGAUCGCCCGGAAGAAGGACUGGGACCCCAAGAAGUACGGCG
GCUUCGACUCCCCCACCGUGGCCUACUCCGUGCUGGUGGUGGCCAAGGUGGAGAAGGGCAA
GUCCAAGAAGCUGAAGUCCGUGAAGGAGCUGCUGGGCAUCACCAUCAUGGAGCGGUCCUCCU
UCGAGAAGAACCCCAUCGACUUCCUGGAGGCCAAGGGCUACAAGGAGGUGAAGAAGGACCUG
AUCAUCAAGCUGCCCAAGUACUCCCUGUUCGAGCUGGAGAACGGCCGGAAGCGGAUGCUGGC
CUCCGCCGGCGAGCUGCAGAAGGGCAACGAGCUGGCCCUGCCCUCCAAGUACGUGAACUUCC
UGUACCUGGCCUCCCACUACGAGAAGCUGAAGGGCUCCCCCGAGGACAACGAGCAGAAGCAG
CUGUUCGUGGAGCAGCACAAGCACUACCUGGACGAGAUCAUCGAGCAGAUCUCCGAGUUCUC
CAAGCGGGUGAUCCUGGCCGACGCCAACCUGGACAAGGUGCUGUCCGCCUACAACAAGCACC
GGGACAAGCCCAUCCGGGAGCAGGCCGAGAACAUCAUCCACCUGUUCACCCUGACCAACCUG
GGCGCCCCCGCCGCCUUCAAGUACUUCGACACCACCAUCGACCGGAAGCGGUACACCUCCAC
CAAGGAGGUGCUGGACGCCACCCUGAUCCACCAGUCCAUCACCGGCCUGUACGAGACCCGGA
UCGACCUGUCCCAGCUGGGCGGCGACGGCGGCGGCUCCCCCAAGAAGAAGCGGAAGGUGUG
A
Exemplary amino acid sequence for Cas9
MDKKYSIGLDIGTNSVGWAVITDEYKVPSKKFKVLGNTDRHSIKKNLIGALLFDSGETAEATRLKRTAR
RRYTRRKNRICYLQEIFSNEMAKVDDSFFHRLEESFLVEEDKKHERHPIFGNIVDEVAYHEKYPTIYHL
RKKLVDSTDKADLRLIYLALAHMIKFRGHFLIEGDLNPDNSDVDKLFIQLVQTYNQLFEENPINASGVDA
KAILSARLSKSRRLENLIAQLPGEKKNGLFGNLIALSLGLTPNFKSNFDLAEDAKLQLSKDTYDDDLDNL
LAQIGDQYADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSIPHQIHLG
ELHAILRRQEDFYPFLKDNREKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRKPAFLSGEQKKAIVDLLFKT
NRKVTVKQLKEDYFKKIECFDSVEISGVEDRFNASLGTYHDLLKIIKDKDFLDNEENEDILEDIVLTLTLF
EDREMIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKPENIVIE
MARENQTTQKGQKNSRERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKKMKNYWRQLLNAKLITQRK
FDNLTKAERGGLSELDKAGFIKRQLVETRQITKHVAQILDSRMNTKYDENDKLIREVKVITLKSKLVSDF
RKDFQFYKVREINNYHHAHDAYLNAVVGTALIKKYPKLESEFVYGDYKVYDVRKMIAKSEQEIGKATA
KYFFYSNIMNFFKTEITLANGEIRKRPLIETNGETGEIVWDKGRDFATVRKVLSMPQVNIVKKTEVQTG
GFSKESILPKRNSDKLIARKKDWDPKKYGGFDSPTVAYSVLVVAKVEKGKSKKLKSVKELLGITIMERS
SFEKNPIDFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIREQAENIIH
LFTLTNLGAPAAFKYFDTTIDRKRYTSTKEVLDATLIHQSITGLYETRIDLSQLGGDGGGSPKKKRKV
Exemplary open reading frame for Cas9
AUGGACAAGAAGUACAGCAUCGGACUGGACAUCGGAACAAACAGCGUCGGAUGGGCAGUCAU
CACAGACGAAUACAAGGUCCCGAGCAAGAAGUUCAAGGUCCUGGGAAACACAGACAGACACAG
CAUCAAGAAGAACCUGAUCGGAGCACUGCUGUUCGACAGCGGAGAAACAGCAGAAGCAACAAG
ACUGAAGAGAACAGCAAGAAGAAGAUACACAAGAAGAAAGAACAGAAUCUGCUACCUGCAGGA
AAUCUUCAGCAACGAAAUGGCAAAGGUCGACGACAGCUUCUUCCACAGACUGGAAGAAAGCUU
CCUGGUCGAAGAAGACAAGAAGCACGAAAGACACCCGAUCUUCGGAAACAUCGUCGACGAAGU
CGCAUACCACGAAAAGUACCCGACAAUCUACCACCUGAGAAAGAAGCUGGUCGACAGCACAGA
CAAGGCAGACCUGAGACUGAUCUACCUGGCACUGGCACACAUGAUCAAGUUCAGAGGACACU
UCCUGAUCGAAGGAGACCUGAACCCGGACAACAGCGACGUCGACAAGCUGUUCAUCCAGCUG
GUCCAGACAUACAACCAGCUGUUCGAAGAAAACCCGAUCAACGCAAGCGGAGUCGACGCAAAG
GCAAUCCUGAGCGCAAGACUGAGCAAGAGCAGAAGACUGGAAAACCUGAUCGCACAGCUGCC
GGGAGAAAAGAAGAACGGACUGUUCGGAAACCUGAUCGCACUGAGCCUGGGACUGACACCGA
ACUUCAAGAGCAACUUCGACCUGGCAGAAGACGCAAAGCUGCAGCUGAGCAAGGACACAUAC
GACGACGACCUGGACAACCUGCUGGCACAGAUCGGAGACCAGUACGCAGACCUGUUCCUGGC
AGCAAAGAACCUGAGCGACGCAAUCCUGCUGAGCGACAUCCUGAGAGUCAACACAGAAAUCAC
AAAGGCACCGCUGAGCGCAAGCAUGAUCAAGAGAUACGACGAACACCACCAGGACCUGACACU
GCUGAAGGCACUGGUCAGACAGCAGCUGCCGGAAAAGUACAAGGAAAUCUUCUUCGACCAGA
GCAAGAACGGAUACGCAGGAUACAUCGACGGAGGAGCAAGCCAGGAAGAAUUCUACAAGUUC
AUCAAGCCGAUCCUGGAAAAGAUGGACGGAACAGAAGAACUGCUGGUCAAGCUGAACAGAGAA
GACCUGCUGAGAAAGCAGAGAACAUUCGACAACGGAAGCAUCCCGCACCAGAUCCACCUGGG
AGAACUGCACGCAAUCCUGAGAAGACAGGAAGACUUCUACCCGUUCCUGAAGGACAACAGAGA
AAAGAUCGAAAAGAUCCUGACAUUCAGAAUCCCGUACUACGUCGGACCGCUGGCAAGAGGAAA
CAGCAGAUUCGCAUGGAUGACAAGAAAGAGCGAAGAAACAAUCACACCGUGGAACUUCGAAGA
AGUCGUCGACAAGGGAGCAAGCGCACAGAGCUUCAUCGAAAGAAUGACAAACUUCGACAAGAA
CCUGCCGAACGAAAAGGUCCUGCCGAAGCACAGCCUGCUGUACGAAUACUUCACAGUCUACA
ACGAACUGACAAAGGUCAAGUACGUCACAGAAGGAAUGAGAAAGCCGGCAUUCCUGAGCGGA
GAACAGAAGAAGGCAAUCGUCGACCUGCUGUUCAAGACAAACAGAAAGGUCACAGUCAAGCAG
CUGAAGGAAGACUACUUCAAGAAGAUCGAAUGCUUCGACAGCGUCGAAAUCAGCGGAGUCGA
AGACAGAUUCAACGCAAGCCUGGGAACAUACCACGACCUGCUGAAGAUCAUCAAGGACAAGGA
CUUCCUGGACAACGAAGAAAACGAAGACAUCCUGGAAGACAUCGUCCUGACACUGACACUGUU
CGAAGACAGAGAAAUGAUCGAAGAAAGACUGAAGACAUACGCACACCUGUUCGACGACAAGGU
CAUGAAGCAGCUGAAGAGAAGAAGAUACACAGGAUGGGGAAGACUGAGCAGAAAGCUGAUCAA
CGGAAUCAGAGACAAGCAGAGCGGAAAGACAAUCCUGGACUUCCUGAAGAGCGACGGAUUCG
CAAACAGAAACUUCAUGCAGCUGAUCCACGACGACAGCCUGACAUUCAAGGAAGACAUCCAGA
AGGCACAGGUCAGCGGACAGGGAGACAGCCUGCACGAACACAUCGCAAACCUGGCAGGAAGC
CCGGCAAUCAAGAAGGGAAUCCUGCAGACAGUCAAGGUCGUCGACGAACUGGUCAAGGUCAU
GGGAAGACACAAGCCGGAAAACAUCGUCAUCGAAAUGGCAAGAGAAAACCAGACAACACAGAA
GGGACAGAAGAACAGCAGAGAAAGAAUGAAGAGAAUCGAAGAAGGAAUCAAGGAACUGGGAAG
CCAGAUCCUGAAGGAACACCCGGUCGAAAACACACAGCUGCAGAACGAAAAGCUGUACCUGUA
CUACCUGCAGAACGGAAGAGACAUGUACGUCGACCAGGAACUGGACAUCAACAGACUGAGCG
ACUACGACGUCGACCACAUCGUCCCGCAGAGCUUCCUGAAGGACGACAGCAUCGACAACAAG
GUCCUGACAAGAAGCGACAAGAACAGAGGAAAGAGCGACAACGUCCCGAGCGAAGAAGUCGU
CAAGAAGAUGAAGAACUACUGGAGACAGCUGCUGAACGCAAAGCUGAUCACACAGAGAAAGUU
CGACAACCUGACAAAGGCAGAGAGAGGAGGACUGAGCGAACUGGACAAGGCAGGAUUCAUCA
AGAGACAGCUGGUCGAAACAAGACAGAUCACAAAGCACGUCGCACAGAUCCUGGACAGCAGAA
UGAACACAAAGUACGACGAAAACGACAAGCUGAUCAGAGAAGUCAAGGUCAUCACACUGAAGA
GCAAGCUGGUCAGCGACUUCAGAAAGGACUUCCAGUUCUACAAGGUCAGAGAAAUCAACAACU
ACCACCACGCACACGACGCAUACCUGAACGCAGUCGUCGGAACAGCACUGAUCAAGAAGUACC
CGAAGCUGGAAAGCGAAUUCGUCUACGGAGACUACAAGGUCUACGACGUCAGAAAGAUGAUC
GCAAAGAGCGAACAGGAAAUCGGAAAGGCAACAGCAAAGUACUUCUUCUACAGCAACAUCAUG
AACUUCUUCAAGACAGAAAUCACACUGGCAAACGGAGAAAUCAGAAAGAGACCGCUGAUCGAA
ACAAACGGAGAAACAGGAGAAAUCGUCUGGGACAAGGGAAGAGACUUCGCAACAGUCAGAAAG
GUCCUGAGCAUGCCGCAGGUCAACAUCGUCAAGAAGACAGAAGUCCAGACAGGAGGAUUCAG
CAAGGAAAGCAUCCUGCCGAAGAGAAACAGCGACAAGCUGAUCGCAAGAAAGAAGGACUGGGA
CCCGAAGAAGUACGGAGGAUUCGACAGCCCGACAGUCGCAUACAGCGUCCUGGUCGUCGCAA
AGGUCGAAAAGGGAAAGAGCAAGAAGCUGAAGAGCGUCAAGGAACUGCUGGGAAUCACAAUC
AUGGAAAGAAGCAGCUUCGAAAAGAACCCGAUCGACUUCCUGGAAGCAAAGGGAUACAAGGAA
GUCAAGAAGGACCUGAUCAUCAAGCUGCCGAAGUACAGCCUGUUCGAACUGGAAAACGGAAG
AAAGAGAAUGCUGGCAAGCGCAGGAGAACUGCAGAAGGGAAACGAACUGGCACUGCCGAGCA
AGUACGUCAACUUCCUGUACCUGGCAAGCCACUACGAAAAGCUGAAGGGAAGCCCGGAAGAC
AACGAACAGAAGCAGCUGUUCGUCGAACAGCACAAGCACUACCUGGACGAAAUCAUCGAACAG
AUCAGCGAAUUCAGCAAGAGAGUCAUCCUGGCAGACGCAAACCUGGACAAGGUCCUGAGCGC
AUACAACAAGCACAGAGACAAGCCGAUCAGAGAACAGGCAGAAAACAUCAUCCACCUGUUCAC
ACUGACAAACCUGGGAGCACCGGCAGCAUUCAAGUACUUCGACACAACAAUCGACAGAAAGAG
AUACACAAGCACAAAGGAAGUCCUGGACGCAACACUGAUCCACCAGAGCAUCACAGGACUGUA
CGAAACAAGAAUCGACCUGAGCCAGCUGGGAGGAGACGGAGGAGGAAGCCCGAAGAAGAAGA
GAAAGGUCUAG

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a “target sequence” refers to a sequence of nucleic acid in a target gene that has complementarity to the guide sequence of the gRNA, i.e., that is sufficiently complementary to the guide sequence to permit specific binding of the guide sequence. The interaction of the target sequence and the guide sequence directs an RNA-guided DNA binding agent to bind, and potentially nick or cleave (depending on the activity of the agent), within the target sequence.

As used herein, a first sequence is considered to be “identical” or have “100% identity” with a second sequence if an alignment of the first sequence to the second sequence shows that all of the positions of the second sequence in its entirety are matched by the first sequence. For example, the sequence AAG has 100% identity to the sequence AAGA because an alignment would give 100% identity in that there are matches, without gaps, to all three positions of the first sequence. Less than 100% identity can be calculated using routine methods. For example ACG would have 67% identity with AAGA as two of the three positions of the first sequence are matches to the second sequence (⅔=67%). The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity>50% for amino acids or >75% for nucleotides, the Needleman-Wunsch algorithm with default settings of the Needleman-Wunsch algorithm interface provided by the EBI at the www.ebi.ac.uk web server is generally appropriate.

Similarly, as used herein, a first sequence is considered to be “fully complementary” or 100% complementary” to a second sequence when all of the nucleotides of a first sequence are complementary to a second sequence, without gaps. For example, the sequence UCU would be considered to be fully complementary to the sequence AAGA as each of the nucleobases from the first sequence base pair with the nucleotides of the second sequence, without gaps. The sequence UGU would be considered to be 67% complementary to the sequence AAGA as two of the three nucleobases of the first sequence base pair with nucleobases of the second sequence. One skilled in the art will understand that algorithms are available with various parameter settings to determine percent complementarity for any pair of sequences using, e.g., the NCBI BLAST interface (blast.ncbi.nlm.nih.gov/Blast.cgi) or the Needleman-Wunsch algorithm.

“mRNA” is used herein to refer to a polynucleotide that comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Exemplary guide sequences useful in the guide RNA compositions and methods described herein are shown in Table 1 and throughout the application. For example, where Table 1 shows a guide sequence that may be used in a guide RNA to direct a RNA-guided DNA binding agent, e.g., a nuclease, such as a Cas nuclease, such as Cas9, to a target sequence. Target sequences are provided in Table 1 as genomic coordinates, and include both the positive and negative strands of genomic DNA (i.e., the sequence given and the sequence's reverse complement). In some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, “indels” refer to insertion/deletion mutations consisting of a number of nucleotides that are either inserted or deleted at the site of double-stranded breaks (DSBs) in a target nucleic acid.

As used herein, “inhibit expression” and the like refer to a decrease in expression of a particular gene product (e.g., protein, mRNA, or both). Expression of a protein (i.e., gene product) can be measured by detecting total cellular amount of the protein from a tissue or cell population of interest by detecting expression of a protein as individual members of a population of cells, e.g., by cell sorting to define percent of cells expressing a protein, or expression of a protein in cells in aggregate, e.g., by ELISA or western blot. Inhibition of expression can result from genetic modification of a gene sequence, e.g., a genomic sequence, such that the full-length gene product, or any gene product, is no longer expressed, e.g. knockdown of the gene. Certain genetic modifications can result in the introduction of frameshift or nonsense mutations that prevent translation of the full-length gene product. Genetic modifications at a splice site, e.g., at a position sufficiently close to a splice acceptor site or a splice donor site to disrupt splicing, can prevent translation of the full-length protein. Inhibition of expression can result from a genetic modification in a regulatory sequence within the genomic sequence required for the expression of the gene product, e.g., a promoter sequence, a 3′ UTR sequence, e.g., a capping sequence, a 5′ UTR sequence, e.g., a poly A sequence. Inhibition of expression may also result from disrupting expression or activity of regulatory factors required for translation of the gene product, e.g., production of no gene product. For example, a genetic modification in a transcription factor sequence, inhibiting expression of the full-length transcription factor, can have downstream effects and inhibit expression of the expression of one or more gene products controlled by the transcription factor. Therefore, inhibition of expression can be predicted by changes in genomic or mRNA sequences. Therefore, mutations expected to result in inhibition of expression can be detected by known methods including sequencing of mRNA isolated from a tissue or cell population of interest. Inhibition of expression can be determined as the percent of cells in a population having a predetermined level of expression of a protein, i.e., a reduction of the percent or number of cells in a population expressing a protein of interest at least a certain level. Inhibition of expression can also be assessed by determining a decrease in overall protein level, e.g., in a cell or tissue sample, e.g., a biopsy sample. In certain embodiments, inhibition of expression of a secreted protein can be assessed in a fluid sample, e.g., cell culture media or a body fluid. Proteins may be present in a body fluid, e.g., blood or urine, to permit analysis of protein level. In certain embodiments, protein level may be determined by protein activity or the level of a metabolic product, e.g., in urine or blood. In some embodiments, “inhibition of expression” may refer to some loss of expression of a particular gene product, for example a decrease in the amount of mRNA transcribed or a decrease in the amount of protein expressed by a population of cells. In some embodiments, “inhibition” may refer to some loss of expression of a particular gene product, for example a CD38 gene product at the cell surface. It is understood that the level of knockdown is relative to a starting level in the same type of subject sample. For example, routine monitoring of a protein level is more easily performed in a fluid sample from a subject, e.g., blood or urine, than in a tissue sample, e.g., a biopsy sample. It is understood that the level of knockdown is for the sample being assayed. Similarly, in animal studies where serial tissue samples may be obtained, e.g., liver tissue, the knockdown target may be expressed in other tissues. Therefore, the level of knockdown is not necessarily the level of knockdown systemically, but within the tissue, cell type, or fluid being sampled.

As used herein, a “genetic modification” is a change at the DNA level, e.g. a change induced by a CRISPR/Cas9 gRNA and Cas9 system. A genetic modification may comprise an insertion, deletion, or substitution (i.e., base sequence substitution, i.e., mutation), typically within a defined sequence or genomic locus. A genetic modification changes the nucleic acid sequence of the DNA. A genetic modification may be at a single nucleotide position. A genetic modification may be at multiple nucleotides, e.g., 2, 3, 4, 5 or more nucleotides, typically in close proximity to each other, e.g., contiguous nucleotides. A genetic modification can be in a coding sequence, e.g., an exon sequence. A genetic modification can be at a splice site, i.e., sufficiently close to a splice acceptor site or a splice donor site to disrupt splicing. A genetic modification can include insertion of a nucleotide sequence not endogenous to the genomic locus, e.g., insertion of a coding sequence of a heterologous open reading frame or gene. As used herein, preferably a genetic modification prevents translation of a full-length protein having an amino acid sequence of the full-length protein prior to genetic modification of the genomic locus. Prevention of translation of a full-length protein or gene product includes prevention of translation of a protein or gene product of any length. Translation of a full-length protein can be prevented, for example, by a frameshift mutation that results in the generation of a premature stop codon or by generation of a nonsense mutation. Translation of a full-length protein can be prevented by disruption of splicing.

As used herein, a “heterologous coding sequence” refers to a coding sequence that has been introduced as an exogenous source within a cell (e.g., inserted at a genomic locus such as a safe harbor locus including a TCR gene locus). That is, the introduced coding sequence is heterologous with respect to at least its insertion site. A polypeptide expressed from such heterologous coding sequence gene is referred to as a “heterologous polypeptide.” The heterologous coding sequence can be naturally-occurring or engineered and can be wild-type or a variant. The heterologous coding sequence may include nucleotide sequences other than the sequence that encodes the heterologous polypeptide (e.g., an internal ribosomal entry site). The heterologous coding sequence can be a coding sequence that occurs naturally in the genome, as a wild-type or a variant (e.g., mutant). For example, although the cell contains the coding sequence of interest (as a wild-type or as a variant), the same coding sequence or variant thereof can be introduced as an exogenous source, e.g., for expression at a locus that is highly expressed. The heterologous coding sequence can also be a coding sequence that is not naturally occurring in the genome, or that expresses a heterologous polypeptide that does not naturally occur in the genome. “Heterologous coding sequence,” “exogenous coding sequence,” and “transgene” are used interchangeably. In some embodiments, the heterologous coding sequence or transgene includes an exogenous nucleic acid sequence, e.g., a nucleic acid sequence is not endogenous to the recipient cell. In some embodiments, the heterologous coding sequence or transgene includes an exogenous nucleic acid sequence, e.g., a nucleic acid sequence that does not naturally occur in the recipient cell. For example, a heterologous coding sequence may be heterologous with respect to its insertion site and with respect to its recipient cell.

A “safe harbor” locus is a locus within the genome wherein a gene may be inserted without significant deleterious effects on the cell. Non-limiting examples of safe harbor loci that are targeted by nuclease(s) for use herein include AAVS1 (PPP1 R12C), TCR, B2M, or albumin. In some embodiments, insertions at a locus or loci targeted for knockdown such as a TRC gene, e.g., TRAC gene, is advantageous for cells. Other suitable safe harbor loci are known in the art.

As used herein, “targeting receptor” refers to a receptor present on the surface of a cell, e.g., a T cell, to permit binding of the cell to a target site, e.g., a specific cell or tissue in an organism. Targeting receptors include, but are not limited to a chimeric antigen receptor (CAR), a T-cell receptor (TCR), and a receptor comprising a binder for a target (e.g., cell surface molecule or a ligand) operably linked through at least a transmembrane domain in an internal signalling domain capable of activating a T cell upon binding of the extracellular receptor portion of a protein. As used herein, a “receptor” and “ligand” pair includes any binding pair, including an antigen and an antibody that specifically binds the antigen.

As used herein, a “chimeric antigen receptor” refers to an extracellular target recognition domain, e.g., an scFv, VHH, nanobody; operably linked to an intracellular signaling domain, which activates the T cell when a target is bound. CARs are composed of four regions: an target recognition domain, an extracellular hinge region, a transmembrane domain, and an intracellular T-cell signalling domain. Such receptors are well known in the art (see, e.g., WO2020092057, WO2019191114, WO2019147805, WO2018208837, the corresponding portions of the contents of each of which are incorporated herein by reference). A reversed universal CAR that promotes binding of an immune cell to a target cell through an adaptor molecule (see, e.g., WO2019238722, the contents of which are incorporated herein in their entirety) is also contemplated. CARs can be targeted to any target (e.g., antigen) to which a binder (e.g., antibody) can be developed and are typically directed to molecules displayed on the surface of a cell or tissue to be targeted.

As used herein, “treatment” refers to any administration or application of a therapeutic for disease or disorder in a subject, and includes inhibiting the disease, arresting its development, relieving one or more symptoms of the disease, curing the disease, preventing one or more symptoms of the disease, or preventing reoccurrence of one or more symptoms of the disease. Treating an autoimmune or inflammatory response or disorder may comprise alleviating the inflammation associated with the specific disorder resulting in the alleviation of disease-specific symptoms. Treatment with the engineered T cells described herein may be used before, after, or in combination with additional therapeutic agents, e.g., the standard of care for the indication to be treated.

The human wild-type CD38 sequence is available at NCBI Gene ID: 952 (www.ncbi.nlm.nih.gov/gene/952, in the version available on the date of filing the instant application); Ensembl: ENSG00000004468, chr4:15,778,275-15,853,232. Cluster Of Differentiation 38 (CD38) protein is a type II transmembrane glycoprotein that synthesizes and hydrolyzes cyclic adenosine 5′-diphosphate-ribose. The CD38 gene contains 8 exons. ADP-Ribosyl Cyclase/Cyclic ADP-Ribose Hydrolase, and Ecto-Nicotinamide Adenine Dinucleotide Glycohydrolase are gene synonyms for CD38. CD38 increases airway contractility hyper-responsiveness and is increased in the lungs of asthmatic patients, thereby amplifying the inflammatory response of airway smooth muscle of those patients

As used herein, “T cell receptor” or “TCR” refers to a receptor in a T cell. In general, a TCR is a heterodimer receptor molecule that contains two TCR polypeptide chains, α and β. α and β chain TCR polypeptides can complex with various CD3 molecules and elicit immune response(s), including inflammation and autoimmunity, after antigen binding. As used herein, a knockdown of TCR refers to a knockdown of any TCR gene in part or in whole, e.g., deletion of part of the TRBC1 gene, alone or in combination with knockdown of other TCR gene(s) in part or in whole.

“TRAC” is used to refer to the T cell receptor α chain. A human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

“TRBC” is used to refer to the T-cell receptor β-chain, e.g., TRBC1 and TRBC2. “TRBC1” and “TRBC2” refer to two homologous genes encoding the T-cell receptor β-chain, which are the gene products of the TRBC1 or TRBC2 genes.

A human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1.

A human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

A “T cell” plays a central role in the immune response following exposure to an antigen. T cells can be naturally occurring or non-natural, e.g., when T cells are formed by engineering, e.g., from a stem cell or by transdifferentiation, e.g., reprogramming a somatic cell. T cells can be distinguished from other lymphocytes by the presence of a T cell receptor on the cell surface. Included in this definition are conventional adaptive T cells, which include helper CD4+ T cells, cytotoxic CD8+ T cells, memory T cells, and regulatory CD4+ T cells, and innate-like T cells including natural killer T cells, mucosal associated invariant T cells, and gamma delta T cells. In some embodiments, T cells are CD4+. In some embodiments, T cells are CD3+/CD4+.

As used herein, “MHC” or “MHC protein” refers to a major histocompatibility complex molecule (or plural), and includes e.g., MHC class I molecules (e.g., HLA-A, HLA-B, and HLA-C in humans) and MHC class II molecules (e.g., HLA-DP, HLA-DQ, and HLA-DR in humans).

“CIITA,” “CIITA,” or “C2TA,” as used herein, refer to the nucleic acid sequence or protein sequence of “class II major histocompatibility complex transactivator.” The human CIITA gene has accession number NC_000016.10 (range 10866208 . . . 10941562), reference GRCh38.p13. The CIITA protein in the nucleus acts as a positive regulator of MHC class II gene transcription and is required for MHC class II protein expression.

“β2M” or “B2M,” as used herein, refers to nucleic acid sequence or protein sequence of “β-2 microglobulin.” The human B2M gene has accession number NC_000015 (range 44711492 . . . 44718877), reference GRCh38.p13. The B2M protein is associated with MHC class I molecules as a heterodimer on the surface of nucleated cells and is required for MHC class I protein expression.

The term “HLA-A,” as used herein in the context of HLA-A protein, refers to the MHC class I protein molecule, which is a heterodimer consisting of a heavy chain (encoded by the HLA-A gene) and a light chain (i.e., beta-2 microglobulin). The term “HLA-A” or “HLA-A gene,” as used herein in the context of nucleic acids refers to the gene encoding the heavy chain of the HLA-A protein molecule. The HLA-A gene is also referred to as “HLA class I histocompatibility, A alpha chain.;” The human HLA-A gene has accession number NC_000006.12 (29942532 . . . 29945870). The HLA-A gene is known to have thousands of different versions (also referred to as “alleles”) across the population (and an individual may receive two different alleles of the HLA-A gene). A public database for HLA-A alleles, including sequence information, may be accessed at IPD-IMGT/HLA: www.ebi.ac.uk/ipd/imgt/hla/. All alleles of HLA-A are encompassed by the terms “HLA-A” and “HLA-A gene.”

As used herein, the term “within the genomic coordinates” includes the boundaries of the genomic coordinate range given. For example, if chr6:29942854-chr6:29942913 is given, the coordinates chr6:29942854-chr6:29942913 are encompassed. Throughout this application, the referenced genomic coordinates are based on genomic annotations in the GRCh38 (also referred to as hg38) assembly of the human genome from the Genome Reference Consortium, available at www.ncbi.nlm.nih.gov (the National Center for Biotechnology Information website). Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion of an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

A “splice site,” as used herein, refers to the three nucleotides that make up an acceptor splice site or a donor splice site (defined below), or any other nucleotides known in the art that are part of a splice site. See e.g., Burset et al., Nucleic Acids Research 28(21):4364-4375 (2000) (describing canonical and non-canonical splice sites in mammalian genomes). The three nucleotides that make up an “acceptor splice site” are two conserved residues (e.g., AG in humans) at the 3′ of an intron and a boundary nucleotide (i.e., the first nucleotide of the exon 3′ of the AG). The “splice site boundary nucleotide” of an acceptor splice site is designated as “Y” in the diagram below and may also be referred to herein as the “acceptor splice site boundary nucleotide,” or “splice acceptor site boundary nucleotide.” The terms “acceptor splice site,” “splice acceptor site,” “acceptor splice sequence,” or “splice acceptor sequence” may be used interchangeably herein.

The three nucleotides that make up a “donor splice site” are two conserved residues (e.g., GT (gene) or GU (in RNA such as pre-mRNA) in human) at the 5′ end of an intron and a boundary nucleotide (i.e., the first nucleotide of the exon 5′ of the GT). The “splice site boundary nucleotide” of a donor splice site is designated as “X” in the diagram below and may also be referred to herein as the “donor splice site boundary nucleotide,” or “splice donor site boundary nucleotide.” The terms “donor splice site,” “splice donor site,” “donor splice sequence,” or “splice donor sequence” may be used interchangeably herein.

Compositions Comprising Guide RNA (gRNAs)

Provided herein are compositions useful for altering a DNA sequence, e.g., inducing a single-stranded (SSB) or double-stranded break (DSB), within a CD38 gene, e.g., using a guide RNA with an RNA-guided DNA binding agent (e.g., a CRISPR/Cas system). Guide sequences targeting a CD38 gene are shown in Table 1 at SEQ ID NOs: 1-88, as are the genomic coordinates that such guide RNA targets.

Each of the guide sequences shown in Table 1 at SEQ ID NOs: 1-88 may further comprise additional nucleotides to form a crRNA, e.g., with the following exemplary nucleotide sequence following the guide sequence at its 3′ end: GUUUUAGAGCUAUGCUGUUUUG (SEQ ID NO:200) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGC (SEQ ID NO: 201) in 5′ to 3′ orientation.

In the case of a sgRNA, the above guide sequences may further comprise additional nucleotides to form a sgRNA, e.g., with the following exemplary nucleotide sequence following the 3′ end of the guide sequence: GUUUUAGAGCUAGAAAUAGCAAGUUAAAAUAAGGCUAGUCCGUUAUCAACUU GAAAAAGUGGCACCGAGUCGGUGCUUUU (SEQ ID NO: 202) in 5′ to 3′ orientation.

In the case of a sgRNA, the guide sequences may be integrated into the following modified motif. mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

In some embodiments, the sgRNA may comprise a sequence of any one of SEQ ID NO: 1220-1225 (Table 12), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

In the case of a sgRNA, the guide sequences may further comprise a SpyCas9 sgRNA sequence. An example of a SpyCas9 sgRNA sequence is shown in the table below (SEQ ID NO: 201 GUUUUAGAGCUAGAAAUAGCAAGUUAAAAUAAGGCUAGUCCGUUAUCAACUU GAAAAAGUGGCACCGAGUCGGUGC—Exemplary SpyCas9 sgRNA-1”) included at the 3′ end of the guide sequence, and provided with the domains as shown in the table below. LS is lower stem. B is bulge. US is upper stem. H1 and H2 are hairpin 1 and hairpin 2, respectively. Collectively H1 and H2 are referred to as the hairpin region. A model of the structure is provided in FIG. 10A of WO2019237069 which is incorporated herein by reference.

The nucleotide sequence of Exemplary SpyCas9 sgRNA-1 may serve as a template sequence for specific chemical modifications, sequence substitutions and truncations.

In certain embodiments, the gRNA is an sgRNA or a dgRNA, for example, and it optionally comprises a chemical modification. In some embodiments, the modified sgRNA comprises a guide sequence and a SpyCas9 sgRNA sequence, e.g., Exemplary SpyCas9 sgRNA-1. A gRNA, such as an sgRNA, may include modifications on the 5′ end of the guide sequence and/or on the 3′ end of the SpyCas9 sgRNA sequence, such as, e.g., Exemplary SpyCas9 sgRNA-1 at one or more of the terminal nucleotides, e.g., at 1, 2, 3, or 4 of the nucleotides at the 3′ end or at the 5′ end. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide. In certain embodiments, the modified nucleotide includes a PS linkage. In certain embodiments, the modified nucleotide includes a 2′-OMe modified nucleotide and a PS linkage.

In certain embodiments, using SEQ ID NO: 201 (“Exemplary SpyCas9 sgRNA-1”) as an example, (see WO2019237069, the contents of which are incorporated herein by reference) the Exemplary SpyCas9 sgRNA-1 further includes one or more of:
A. a shortened hairpin 1 region, or a substituted and optionally shortened hairpin 1 region, wherein
   1. at least one of the following pairs of nucleotides are substituted in hairpin 1 with Watson-Crick pairing nucleotides: H1-1 and H1-12, H1-2 and H1-11, H1-3 and H1-10, or H1-4 and H1-9, and the hairpin 1 region optionally lacks
      a. any one or two of H1-5 through H1-8,
      b. one, two, or three of the following pairs of nucleotides: H1-1 and H1-12, H1-2 and H1-11, H1-3 and H1-10, and H1-4 and H1-9, or
      c. 1-8 nucleotides of hairpin 1 region; or
   2. the shortened hairpin 1 region lacks 4-8 nucleotides, preferably 4-6 nucleotides; and
      a. one or more of positions H1-1, H1-2, or H1-3 is deleted or substituted relative to Exemplary SpyCas9 sgRNA-1 (SEQ ID NO: 201) or
      b. one or more of positions H1-6 through H1-10 is substituted relative to Exemplary SpyCas9 sgRNA-1 (SEQ ID NO: 201); or
   3. the shortened hairpin 1 region lacks 5-10 nucleotides, preferably 5-6 nucleotides, and one or more of positions N18, H1-12, or n is substituted relative to Exemplary SpyCas9 sgRNA-1 (SEQ ID NO: 201); or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to Exemplary SpyCas9 sgRNA-1 (SEQ ID NO: 201); or
C. a substitution relative to Exemplary SpyCas9 sgRNA-1 (SEQ ID NO: 201) at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. Exemplary SpyCas9 sgRNA-1 (SEQ ID NO: 201) with an upper stem region, wherein the upper stem modification comprises a modification to any one or more of US1-US12 in the upper stem region, wherein
   1. the modified nucleotide is optionally selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof; or
   2. the modified nucleotide optionally includes a 2′-OMe modified nucleotide.

In certain embodiments, Exemplary SpyCas9 sgRNA-1, or an sgRNA, such as an sgRNA comprising Exemplary SpyCas9 sgRNA-1, further includes a 3′ tail, e.g., a 3′ tail of 1, 2, 3, 4, or more nucleotides. In certain embodiments, the tail includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, and an inverted abasic modified nucleotide, or a combination thereof. In certain embodiments, the modified nucleotide includes a 2′-OMe modified nucleotide. In certain embodiments, the modified nucleotide includes a PS linkage between nucleotides. In certain embodiments, the modified nucleotide includes a 2′-OMe modified nucleotide and a PS linkage between nucleotides.

In certain embodiments, the hairpin region includes one or more modified nucleotides. In certain embodiments, the modified nucleotide is selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the upper stem region includes one or more modified nucleotides. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide; or a combination thereof. In certain embodiments, the modified nucleotide includes a 2′-OMe modified nucleotide.

In certain embodiments, the Exemplary SpyCas9 sgRNA-1 comprises one or more YA dinucleotides, wherein Y is a pyrimidine, wherein the YA dinucleotide includes a modified nucleotide. In certain embodiments, the modified nucleotide selected from a 2′-O-methyl (2′-OMe) modified nucleotide, a 2′-O-(2-methoxyethyl) (2′-O-moe) modified nucleotide, a 2′-fluoro (2′-F) modified nucleotide, a phosphorothioate (PS) linkage between nucleotides, an inverted abasic modified nucleotide, or a combination thereof. In certain embodiments, the modified nucleotide includes a 2′-OMe modified nucleotide.

In certain embodiments, the Exemplary SpyCas9 sgRNA-1 comprises, one or more YA dinucleotides, wherein Y is a pyrimidine, wherein the YA dinucleotide includes a substituted nucleotide, i.e., sequence substituted nucleotide, wherein the pyrimidine is substituted for a purine. In certain embodiments, when the pyrimidine forms a Watson-Crick base pair in the single guide, the Watson-Crick based nucleotide of the substituted pyrimidine nucleotide is substituted to maintain Watson-Crick base pairing.

Exemplary spyCas9 sgRNA-1 (SEQ ID NO: 201)

| 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 | 29 | 30 |
|---|---|---|---|---|---|---|---|---|---|---|---|---|---|---|---|---|---|---|---|---|---|---|---|---|---|---|---|---|---|
| G | U | U | U | U | A | G | A | G | C | U | A | G | A | A | A | U | A | G | C | A | A | G | U | U | A | A | A | A | U |
| LS1-LS6 | | | | | | B1-B2 | | US1-US12 | | | | | | | | | | | | B2-B6 | | | | LS7-LS12 | | | | | |
| 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 | 57 | 58 | 59 | 60 |
| A | A | G | G | C | U | A | G | U | C | C | G | U | U | A | U | C | A | A | C | U | U | G | A | A | A | A | A | G | U |

| Nexus | | | | | | | | | H1-1 through H1-12 | | | | | | |
|---|---|---|---|---|---|---|---|---|---|---|---|---|---|---|---|
| 61 | 62 | 63 | 64 | 65 | 66 | 67 | 68 | 69 | 70 | 71 | 72 | 73 | 74 | 75 | 76 |
| G | G | C | A | C | C | G | A | G | U | C | G | G | U | G | C |

| | |
|---|---|
| N | H2-1 through H2-15 |

TABLE 1
CD38 guide sequences and chromosomal coordinates

| | | | SEQ | | Genomic |
|---|---|---|---|---|---|
| SEQ | | | ID NO | | Coordinates |
| ID | Guide | Guide | full | | (hg38) |
| NO: | ID | Sequence | sgRNA | Full sgRNA sequence | |
| 1 | G019761 | GAUCCUCGU | 89 | GAUCCUCGUCGUGGUGCUCGGUUUUAGAGCUAGAAAUAG | chr4: 15778512- |
| | CD38-1 | CGUGGUGCU | | CAAGUUAAAAUAAGGCUAGUCCGUUAUCAACUUGAAAAA | 15778532 |
| | | CG | | GUGGCACCGAGUCGGUGCUUUU | |
| 2 | G019762 | CCUCGUCGU | 90 | CCUCGUCGUGGUGCUCGCGGGUUUUAGAGCUAGAAAUAG | chr4: 15778515- |
| | CD38-2 | GGUGCUCGC | | CAAGUUAAAAUAAGGCUAGUCCGUUAUCAACUUGAAAAA | 15778535 |
| | | GG | | GUGGCACCGAGUCGGUGCUUUU | |
| 3 | G019763 | GCAUCGCGCC | 91 | GCAUCGCGCCAGGACGGUCUGUUUUAGAGCUAGAAAUAG | chr4: 15778595- |
| | CD38-3 | AGGACGGUC | | CAAGUUAAAAUAAGGCUAGUCCGUUAUCAACUUGAAAAA | 15778615 |
| | | U | | GUGGCACCGAGUCGGUGCUUUU | |
| 4 | G019764 | UGUACUUGA | 92 | UGUACUUGACGCAUCGCGCCGUUUUAGAGCUAGAAAUAG | chr4: 15778605- |
| | CD38-4 | CGCAUCGCGC | | CAAGUUAAAAUAAGGCUAGUCCGUUAUCAACUUGAAAAA | 15778625 |
| | | C | | GUGGCACCGAGUCGGUGCUUUU | |
| 5 | G019765 | CACCGGGCU | 93 | CACCGGGCUGAACUCGCAGUGUUUUAGAGCUAGAAAUAG | chr4: 15778421- |
| | CD38-5 | GAACUCGCA | | CAAGUUAAAAUAAGGCUAGUCCGUUAUCAACUUGAAAAA | 15778441 |
| | | GU | | GUGGCACCGAGUCGGUGCUUUU | |
| 6 | G019766 | UCGCGGUGG | 94 | UCGCGGUGGUCGUCCCGAGGGUUUUAGAGCUAGAAAUAG | chr4: 15778529- |
| | CD38-6 | UCGUCCCGA | | CAAGUUAAAAUAAGGCUAGUCCGUUAUCAACUUGAAAAA | 15778549 |
| | | GG | | GUGGCACCGAGUCGGUGCUUUU | |
| 7 | G019767 | CCACCGCGAG | 95 | CCACCGCGAGCACCACGACGGUUUUAGAGCUAGAAAUAG | chr4: 15778518- |
| | CD38-7 | | | CAAGUUAAAAUAAGGCUAGUCCGUUAUCAACUUGAAAAA | 15778538 |
| | | CACCACGACG | | GUGGCACCGAGUCGGUGCUUUU | |
| 8 | G019768 | CAUCGCGCCA | 96 | CAUCGCGCCAGGACGGUCUCGUUUUAGAGCUAGAAAUAG | chr4: 15778594- |
| | CD38-8 | GGACGGUCU | | CAAGUUAAAAUAAGGCUAGUCCGUUAUCAACUUGAAAAA | 15778614 |
| | | C | | GUGGCACCGAGUCGGUGCUUUU | |
| 9 | G019769 | GACGGUCUC | 97 | GACGGUCUCGGGAAAGCGCUGUUUUAGAGCUAGAAAUAG | chr4: 15778583- |
| | CD38-9 | GGGAAAGCG | | CAAGUUAAAAUAAGGCUAGUCCGUUAUCAACUUGAAAAA | 15778603 |
| | | CU | | GUGGCACCGAGUCGGUGCUUUU | |
| 10 | G019770 | GCGCUUUCCC | 98 | GCGCUUUCCCGAGACCGUCCGUUUUAGAGCUAGAAAUAG | chr4: 15778584- |
| | CD38-10 | GAGACCGUC | | CAAGUUAAAAUAAGGCUAGUCCGUUAUCAACUUGAAAAA | 15778604 |
| | | C | | GUGGCACCGAGUCGGUGCUUUU | |
| 11 | G019771 | CUUGACGCA | 99 | CUUGACGCAUCGCGCCAGGAGUUUUAGAGCUAGAAAUAG | chr4: 15778601- |
| | CD38-11 | UCGCGCCAG | | CAAGUUAAAAUAAGGCUAGUCCGUUAUCAACUUGAAAAA | 15778621 |
| | | GA | | GUGGCACCGAGUCGGUGCUUUU | |
| 12 | G019772 | UGCGAGUUC | 100 | UGCGAGUUCAGCCCGGUGUCGUUUUAGAGCUAGAAAUAG | chr4: 15778423- |
| | CD38-12 | AGCCCGGUG | | CAAGUUAAAAUAAGGCUAGUCCGUUAUCAACUUGAAAAA | 15778443 |
| | | UC | | GUGGCACCGAGUCGGUGCUUUU | |
| 13 | G019773 | UGCUCGCGG | 101 | UGCUCGCGGUGGUCGUCCCGGUUUUAGAGCUAGAAAUAG | chr4: 15778526- |
| | CD38-13 | UGGUCGUCC | | CAAGUUAAAAUAAGGCUAGUCCGUUAUCAACUUGAAAAA | 15778546 |
| | | CG | | GUGGCACCGAGUCGGUGCUUUU | |
| 14 | G019774 | AGGGUUUGU | 102 | AGGGUUUGUCCCCGGACACCGUUUUAGAGCUAGAAAUAG | chr4: 15778437- |
| | CD38-14 | CCCCGGACAC | | CAAGUUAAAAUAAGGCUAGUCCGUUAUCAACUUGAAAAA | 15778457 |
| | | C | | GUGGCACCGAGUCGGUGCUUUU | |
| 15 | G019775 | GCGAGUUCA | 103 | GCGAGUUCAGCCCGGUGUCCGUUUUAGAGCUAGAAAUAG | chr4: 15778424- |
| | CD38-15 | GCCCGGUGU | | CAAGUUAAAAUAAGGCUAGUCCGUUAUCAACUUGAAAAA | 15778444 |
| | | CC | | GUGGCACCGAGUCGGUGCUUUU | |
| 16 | G019776 | GGUCUCGGG | 104 | GGUCUCGGGAAAGCGCUUGGGUUUUAGAGCUAGAAAUAG | chr4: 15778580- |
| | CD38-16 | AAAGCGCUU | | CAAGUUAAAAUAAGGCUAGUCCGUUAUCAACUUGAAAAA | 15778600 |
| | | GG | | GUGGCACCGAGUCGGUGCUUUU | |
| 17 | G019777 | CGAGUUCAG | 105 | CGAGUUCAGCCCGGUGUCCGGUUUUAGAGCUAGAAAUAG | chr4: 15778425- |
| | CD38-17 | CCCGGUGUCC | | CAAGUUAAAAUAAGGCUAGUCCGUUAUCAACUUGAAAAA | 15778445 |
| | | G | | GUGGCACCGAGUCGGUGCUUUU | |
| 18 | G019778 | CCGGCAGCA | 106 | CCGGCAGCAGGGUUUGUCCCGUUUUAGAGCUAGAAAUAG | chr4: 15778445- |
| | CD38-18 | GGGUUUGUC | | CAAGUUAAAAUAAGGCUAGUCCGUUAUCAACUUGAAAAA | 15778465 |
| | | CC | | GUGGCACCGAGUCGGUGCUUUU | |
| 19 | G019779 | GUUGGGCUC | 107 | GUUGGGCUCUCCUAGAGAGCGUUUUAGAGCUAGAAAUAG | chr4: 15778464- |
| | CD38-19 | UCCUAGAGA | | CAAGUUAAAAUAAGGCUAGUCCGUUAUCAACUUGAAAAA | 15778484 |
| | | GC | | GUGGCACCGAGUCGGUGCUUUU | |
| 20 | G019780 | CGAGCACCAC | 108 | CGAGCACCACGACGAGGAUCGUUUUAGAGCUAGAAAUAG | chr4: 15778512- |
| | CD38-20 | GACGAGGAU | | CAAGUUAAAAUAAGGCUAGUCCGUUAUCAACUUGAAAAA | 15778532 |
| | | C | | GUGGCACCGAGUCGGUGCUUUU | |
| 21 | G019781 | GGCCAACUG | 109 | GGCCAACUGCGAGUUCAGCCGUUUUAGAGCUAGAAAUAG | chr4: 15778416- |
| | CD38-21 | CGAGUUCAG | | CAAGUUAAAAUAAGGCUAGUCCGUUAUCAACUUGAAAAA | 15778436 |
| | | CC | | GUGGCACCGAGUCGGUGCUUUU | |
| 22 | G019782 | UACUGACGC | 110 | UACUGACGCCAAGACAGAGUGUUUUAGAGCUAGAAAUAG | chr4: 15778482- |
| | CD38-22 | CAAGACAGA | | CAAGUUAAAAUAAGGCUAGUCCGUUAUCAACUUGAAAAA | 15778502 |
| | | GU | | GUGGCACCGAGUCGGUGCUUUU | |
| 23 | G019783 | UCGCCAACCC | 111 | UCGCCAACCCACCUCAUCUCGUUUUAGAGCUAGAAAUAG | chr4: 15778639- |
| | CD38-23 | ACCUCAUCUC | | CAAGUUAAAAUAAGGCUAGUCCGUUAUCAACUUGAAAAA | 15778659 |
| | | | | GUGGCACCGAGUCGGUGCUUUU | |
| 24 | G019784 | CCGGGGACA | 112 | CCGGGGACAAACCCUGCUGCGUUUUAGAGCUAGAAAUAG | chr4: 15778442- |
| | CD38-24 | AACCCUGCU | | CAAGUUAAAAUAAGGCUAGUCCGUUAUCAACUUGAAAAA | 15778462 |
| | | GC | | GUGGCACCGAGUCGGUGCUUUU | |
| 25 | G019785 | GGAAAGCGC | 113 | GGAAAGCGCUUGGUGGUGCCGUUUUAGAGCUAGAAAUAG | chr4: 15778573- |
| | CD38-25 | UUGGUGGUG | | CAAGUUAAAAUAAGGCUAGUCCGUUAUCAACUUGAAAAA | 15778593 |
| | | CC | | GUGGCACCGAGUCGGUGCUUUU | |
| 26 | G019786 | UCUCCUAGA | 114 | UCUCCUAGAGAGCCGGCAGCGUUUUAGAGCUAGAAAUAG | chr4: 15778457- |
| | CD38-26 | GAGCCGGCA | | CAAGUUAAAAUAAGGCUAGUCCGUUAUCAACUUGAAAAA | 15778477 |
| | | GC | | GUGGCACCGAGUCGGUGCUUUU | |
| 27 | G019787 | CGCCAGCAG | 115 | CGCCAGCAGUGGAGCGGUCCGUUUUAGAGCUAGAAAUAG | chr4: 15778552- |
| | CD38-27 | UGGAGCGGU | | CAAGUUAAAAUAAGGCUAGUCCGUUAUCAACUUGAAAAA | 15778572 |
| | | CC | | GUGGCACCGAGUCGGUGCUUUU | |
| 28 | G019788 | CUCCACUGCU | 116 | CUCCACUGCUGGCGCCACCUGUUUUAGAGCUAGAAAUAG | chr4: 15778546- |
| | CD38-28 | GGCGCCACCU | | CAAGUUAAAAUAAGGCUAGUCCGUUAUCAACUUGAAAAA | 15778566 |
| | | | | GUGGCACCGAGUCGGUGCUUUU | |
| 29 | G019789 | CAGGGUUUG | 117 | CAGGGUUUGUCCCCGGACACGUUUUAGAGCUAGAAAUAG | chr4: 15778438- |
| | CD38-29 | UCCCCGGACA | | CAAGUUAAAAUAAGGCUAGUCCGUUAUCAACUUGAAAAA | 15778458 |
| | | C | | GUGGCACCGAGUCGGUGCUUUU | |
| 30 | G019790 | CUGUCUUGG | 118 | CUGUCUUGGCGUCAGUAUCCGUUUUAGAGCUAGAAAUAG | chr4: 15778485- |
| | CD38-30 | CGUCAGUAU | | CAAGUUAAAAUAAGGCUAGUCCGUUAUCAACUUGAAAAA | 15778505 |
| | | CC | | GUGGCACCGAGUCGGUGCUUUU | |
| 31 | G019791 | UGCCCGGACC | 119 | UGCCCGGACCGCUCCACUGCGUUUUAGAGCUAGAAAUAG | chr4: 15778557- |
| | CD38-31 | GCUCCACUGC | | CAAGUUAAAAUAAGGCUAGUCCGUUAUCAACUUGAAAAA | 15778577 |
| | | | | GUGGCACCGAGUCGGUGCUUUU | |
| 32 | G019792 | CUCCUAGAG | 120 | CUCCUAGAGAGCCGGCAGCAGUUUUAGAGCUAGAAAUAG | chr4: 15778456- |
| | CD38-32 | AGCCGGCAG | | CAAGUUAAAAUAAGGCUAGUCCGUUAUCAACUUGAAAAA | 15778476 |
| | | CA | | GUGGCACCGAGUCGGUGCUUUU | |
| 33 | G019793 | CCUGGUCCU | 121 | CCUGGUCCUGAUCCUCGUCGGUUUUAGAGCUAGAAAUAG | chr4: 15778503- |
| | CD38-33 | GAUCCUCGU | | CAAGUUAAAAUAAGGCUAGUCCGUUAUCAACUUGAAAAA | 15778523 |
| | | CG | | GUGGCACCGAGUCGGUGCUUUU | |
| 34 | G019794 | UCCCGAGGU | 122 | UCCCGAGGUGGCGCCAGCAGGUUUUAGAGCUAGAAAUAG | chr4: 15778541- |
| | CD38-34 | GGCGCCAGC | | CAAGUUAAAAUAAGGCUAGUCCGUUAUCAACUUGAAAAA | 15778561 |
| | | AG | | GUGGCACCGAGUCGGUGCUUUU | |
| 35 | G019795 | GCGCCAGCA | 123 | GCGCCAGCAGUGGAGCGGUCGUUUUAGAGCUAGAAAUAG | chr4: 15778551- |
| | CD38-35 | GUGGAGCGG | | CAAGUUAAAAUAAGGCUAGUCCGUUAUCAACUUGAAAAA | 15778571 |
| | | UC | | GUGGCACCGAGUCGGUGCUUUU | |
| 36 | G019796 | UCCACUGCU | 124 | UCCACUGCUGGCGCCACCUCGUUUUAGAGCUAGAAAUAG | chr4: 15778545- |
| | CD38-36 | GGCGCCACCU | | CAAGUUAAAAUAAGGCUAGUCCGUUAUCAACUUGAAAAA | 15778565 |
| | | C | | GUGGCACCGAGUCGGUGCUUUU | |
| 37 | G019797 | AGGAGAGCC | 125 | AGGAGAGCCCAACUCUGUCUGUUUUAGAGCUAGAAAUAG | chr4: 15778471- |
| | CD38-37 | CAACUCUGU | | CAAGUUAAAAUAAGGCUAGUCCGUUAUCAACUUGAAAAA | 15778491 |
| | | CU | | GUGGCACCGAGUCGGUGCUUUU | |
| 38 | G019798 | ACUGACGCC | 126 | ACUGACGCCAAGACAGAGUUGUUUUAGAGCUAGAAAUAG | chr4: 15778481- |
| | CD38-38 | AAGACAGAG | | CAAGUUAAAAUAAGGCUAGUCCGUUAUCAACUUGAAAAA | 15778501 |
| | | UU | | GUGGCACCGAGUCGGUGCUUUU | |
| 39 | G019799 | AACCCUGCU | 127 | AACCCUGCUGCCGGCUCUCUGUUUUAGAGCUAGAAAUAG | chr4: 15778451- |
| | CD38-39 | GCCGGCUCUC | | CAAGUUAAAAUAAGGCUAGUCCGUUAUCAACUUGAAAAA | 15778471 |
| | | U | | GUGGCACCGAGUCGGUGCUUUU | |
| 40 | G019800 | UAUCAGCCA | 128 | UAUCAGCCACUAAUGAAGUUGUUUUAGAGCUAGAAAUAG | chr4: 15816592- |
| | CD38-40 | CUAAUGAAG | | CAAGUUAAAAUAAGGCUAGUCCGUUAUCAACUUGAAAAA | 15816612 |
| | | UU | | GUGGCACCGAGUCGGUGCUUUU | |
| 41 | G019801 | UGAAAGCAU | 129 | UGAAAGCAUCCCAUACACUUGUUUUAGAGCUAGAAAUAG | chr4: 15816525- |
| | CD38-41 | CCCAUACACU | | CAAGUUAAAAUAAGGCUAGUCCGUUAUCAACUUGAAAAA | 15816545 |
| | | U | | GUGGCACCGAGUCGGUGCUUUU | |
| 42 | G019802 | CCCCCAAUUA | 130 | CCCCCAAUUACCUUGUUGCAGUUUUAGAGCUAGAAAUAG | chr4: 15816631- |
| | CD38-42 | CCUUGUUGC | | CAAGUUAAAAUAAGGCUAGUCCGUUAUCAACUUGAAAAA | 15816651 |
| | | A | | GUGGCACCGAGUCGGUGCUUUU | |
| 43 | G019803 | AUGUAGACU | 131 | AUGUAGACUGCCAAAGUGUAGUUUUAGAGCUAGAAAUAG | chr4: 15816512- |
| | CD38-43 | GCCAAAGUG | | CAAGUUAAAAUAAGGCUAGUCCGUUAUCAACUUGAAAAA | 15816532 |
| | | UA | | GUGGCACCGAGUCGGUGCUUUU | |
| 44 | G019804 | AAGUGUAUG | 132 | AAGUGUAUGGGAUGCUUUCAGUUUUAGAGCUAGAAAUA | chr4: 15816525- |
| | CD38-44 | GGAUGCUUU | | GCAAGUUAAAAUAAGGCUAGUCCGUUAUCAACUUGAAAA | 15816545 |
| | | CA | | AGUGGCACCGAGUCGGUGCUUUU | |
| 45 | G019805 | AAUUACCUU | 133 | AAUUACCUUGUUGCAAGGUAGUUUUAGAGCUAGAAAUAG | chr4: 15816626- |
| | CD38-45 | GUUGCAAGG | | CAAGUUAAAAUAAGGCUAGUCCGUUAUCAACUUGAAAAA | 15816646 |
| | | UA | | GUGGCACCGAGUCGGUGCUUUU | |
| 46 | G019806 | UGAGUUCCC | 134 | UGAGUUCCCAACUUCAUUAGGUUUUAGAGCUAGAAAUAG | chr4: 15816601- |
| | CD38-46 | AACUUCAUU | | CAAGUUAAAAUAAGGCUAGUCCGUUAUCAACUUGAAAAA | 15816621 |
| | | AG | | GUGGCACCGAGUCGGUGCUUUU | |
| 47 | G019807 | UGUAGACUG | 135 | UGUAGACUGCCAAAGUGUAUGUUUUAGAGCUAGAAAUAG | chr4: 15816513- |
| | CD38-47 | CCAAAGUGU | | CAAGUUAAAAUAAGGCUAGUCCGUUAUCAACUUGAAAAA | 15816533 |
| | | AU | | GUGGCACCGAGUCGGUGCUUUU | |
| 48 | G019808 | AGUGUAUGG | 136 | AGUGUAUGGGAUGCUUUCAAGUUUUAGAGCUAGAAAUA | chr4: 15816526- |
| | CD38-48 | GAUGCUUUC | | GCAAGUUAAAAUAAGGCUAGUCCGUUAUCAACUUGAAAA | 15816546 |
| | | AA | | AGUGGCACCGAGUCGGUGCUUUU | |
| 49 | G019809 | CUAUCAGCC | 137 | CUAUCAGCCACUAAUGAAGUGUUUUAGAGCUAGAAAUAG | chr4: 15816591- |
| | CD38-49 | ACUAAUGAA | | CAAGUUAAAAUAAGGCUAGUCCGUUAUCAACUUGAAAAA | 15816611 |
| | | GU | | GUGGCACCGAGUCGGUGCUUUU | |
| 50 | G019810 | UCUUCUUCA | 138 | UCUUCUUCAGUAAUGUUGCAGUUUUAGAGCUAGAAAUAG | chr4: 15816571- |
| | CD38-50 | GUAAUGUUG | | CAAGUUAAAAUAAGGCUAGUCCGUUAUCAACUUGAAAAA | 15816591 |
| | | CA | | GUGGCACCGAGUCGGUGCUUUU | |
| 51 | G019811 | UCUGGCCCA | 139 | UCUGGCCCAUCAGUUCACACGUUUUAGAGCUAGAAAUAG | chr4: 15824906- |
| | CD38-51 | UCAGUUCAC | | CAAGUUAAAAUAAGGCUAGUCCGUUAUCAACUUGAAAAA | 15824926 |
| | | AC | | GUGGCACCGAGUCGGUGCUUUU | |
| 52 | G019812 | GCGGGACAU | 140 | GCGGGACAUGUUCACCCUGGGUUUUAGAGCUAGAAAUAG | chr4: 15824933- |
| | CD38-52 | GUUCACCCU | | CAAGUUAAAAUAAGGCUAGUCCGUUAUCAACUUGAAAAA | 15824953 |
| | | GG | | GUGGCACCGAGUCGGUGCUUUU | |
| 53 | G019813 | CCAGCGGGA | 141 | CCAGCGGGACAUGUUCACCCGUUUUAGAGCUAGAAAUAG | chr4: 15824930- |
| | CD38-53 | CAUGUUCAC | | CAAGUUAAAAUAAGGCUAGUCCGUUAUCAACUUGAAAAA | 15824950 |
| | | CC | | GUGGCACCGAGUCGGUGCUUUU | |
| 54 | G019814 | AGCCUAGCA | 142 | AGCCUAGCAGCGUGUCCUCCGUUUUAGAGCUAGAAAUAG | chr4: 15824951- |
| | CD38-54 | GCGUGUCCU | | CAAGUUAAAAUAAGGCUAGUCCGUUAUCAACUUGAAAAA | 15824971 |
| | | CC | | GUGGCACCGAGUCGGUGCUUUU | |
| 55 | G019815 | UGAAUUCAC | 143 | UGAAUUCACCACACCAUGUGGUUUUAGAGCUAGAAAUAG | chr4: 15824987- |
| | CD38-55 | CACACCAUG | | CAAGUUAAAAUAAGGCUAGUCCGUUAUCAACUUGAAAAA | 15825007 |
| | | UG | | GUGGCACCGAGUCGGUGCUUUU | |
| 56 | G019816 | AUCAGUUCA | 144 | AUCAGUUCACACAGGUCCAGGUUUUAGAGCUAGAAAUAG | chr4: 15824914- |
| | CD38-56 | CACAGGUCC | | CAAGUUAAAAUAAGGCUAGUCCGUUAUCAACUUGAAAAA | 15824934 |
| | | AG | | GUGGCACCGAGUCGGUGCUUUU | |
| 57 | G019817 | GCUGAUGAC | 145 | GCUGAUGACCUCACAUGGUGGUUUUAGAGCUAGAAAUAG | chr4: 15824976- |
| | CD38-57 | CUCACAUGG | | CAAGUUAAAAUAAGGCUAGUCCGUUAUCAACUUGAAAAA | 15824996 |
| | | UG | | GUGGCACCGAGUCGGUGCUUUU | |
| 58 | G019818 | GCCUAGCAG | 146 | GCCUAGCAGCGUGUCCUCCAGUUUUAGAGCUAGAAAUAG | chr4: 15824950- |
| | CD38-58 | CGUGUCCUCC | | CAAGUUAAAAUAAGGCUAGUCCGUUAUCAACUUGAAAAA | 15824970 |
| | | A | | GUGGCACCGAGUCGGUGCUUUU | |
| 59 | G019819 | ACCAUGUGA | 147 | ACCAUGUGAGGUCAUCAGCAGUUUUAGAGCUAGAAAUAG | chr4: 15824975- |
| | CD38-59 | GGUCAUCAG | | CAAGUUAAAAUAAGGCUAGUCCGUUAUCAACUUGAAAAA | 15824995 |
| | | CA | | GUGGCACCGAGUCGGUGCUUUU | |
| 60 | G019820 | UCAGUUCAC | 148 | UCAGUUCACACAGGUCCAGCGUUUUAGAGCUAGAAAUAG | chr4: 15824915- |
| | CD38-60 | ACAGGUCCA | | CAAGUUAAAAUAAGGCUAGUCCGUUAUCAACUUGAAAAA | 15824935 |
| | | GC | | GUGGCACCGAGUCGGUGCUUUU | |
| 61 | G019821 | CCAGGGUGA | 149 | CCAGGGUGAACAUGUCCCGCGUUUUAGAGCUAGAAAUAG | chr4: 15824933- |
| | CD38-61 | ACAUGUCCC | | CAAGUUAAAAUAAGGCUAGUCCGUUAUCAACUUGAAAAA | 15824953 |
| | | GC | | GUGGCACCGAGUCGGUGCUUUU | |
| 62 | G019822 | GCUGGACCU | 150 | GCUGGACCUGUGUGAACUGAGUUUUAGAGCUAGAAAUAG | chr4: 15824915- |
| | CD38-62 | GUGUGAACU | | CAAGUUAAAAUAAGGCUAGUCCGUUAUCAACUUGAAAAA | 15824935 |
| | | GA | | GUGGCACCGAGUCGGUGCUUUU | |
| 63 | G019823 | ACCCUGGAG | 151 | ACCCUGGAGGACACGCUGCUGUUUUAGAGCUAGAAAUAG | chr4: 15824946- |
| | CD38-63 | GACACGCUG | | CAAGUUAAAAUAAGGCUAGUCCGUUAUCAACUUGAAAAA | 15824966 |
| | | CU | | GUGGCACCGAGUCGGUGCUUUU | |
| 64 | G019824 | CUGGACCUG | 152 | CUGGACCUGUGUGAACUGAUGUUUUAGAGCUAGAAAUAG | chr4: 15824914- |
| | CD38-64 | UGUGAACUG | | CAAGUUAAAAUAAGGCUAGUCCGUUAUCAACUUGAAAAA | 15824934 |
| | | AU | | GUGGCACCGAGUCGGUGCUUUU | |
| 65 | G019825 | UCUGGAAAA | 153 | UCUGGAAAACGGUUUCCCGCGUUUUAGAGCUAGAAAUAG | chr4: 15834279- |
| | CD38-65 | CGGUUUCCC | | CAAGUUAAAAUAAGGCUAGUCCGUUAUCAACUUGAAAAA | 15834299 |
| | | GC | | GUGGCACCGAGUCGGUGCUUUU | |
| 66 | G019826 | CUACUUGGU | 154 | CUACUUGGUACUUACCCUGCGUUUUAGAGCUAGAAAUAG | chr4: 15834297- |
| | CD38-66 | ACUUACCCU | | CAAGUUAAAAUAAGGCUAGUCCGUUAUCAACUUGAAAAA | 15834317 |
| | | GC | | GUGGCACCGAGUCGGUGCUUUU | |
| 67 | G019827 | ACAACCCUG | 155 | ACAACCCUGUUUCAGUAUUCGUUUUAGAGCUAGAAAUAG | chr4: 15834261- |
| | CD38-67 | UUUCAGUAU | | CAAGUUAAAAUAAGGCUAGUCCGUUAUCAACUUGAAAAA | 15834281 |
| | | UC | | GUGGCACCGAGUCGGUGCUUUU | |
| 68 | G019828 | UUUUCCAGA | 156 | UUUUCCAGAAUACUGAAACAGUUUUAGAGCUAGAAAUAG | chr4: 15834268- |
| | CD38-68 | AUACUGAAA | | CAAGUUAAAAUAAGGCUAGUCCGUUAUCAACUUGAAAAA | 15834288 |
| | | CA | | GUGGCACCGAGUCGGUGCUUUU | |
| 69 | G019829 | GUUUUCCAG | 157 | GUUUUCCAGAAUACUGAAACGUUUUAGAGCUAGAAAUAG | chr4: 15834269- |
| | CD38-69 | AAUACUGAA | | CAAGUUAAAAUAAGGCUAGUCCGUUAUCAACUUGAAAAA | 15834289 |
| | | AC | | GUGGCACCGAGUCGGUGCUUUU | |
| 70 | G019830 | GGGAUCCAU | 158 | GGGAUCCAUUGAGCAUCACAGUUUUAGAGCUAGAAAUAG | chr4: 15838120- |
| | CD38-70 | UGAGCAUCA | | CAAGUUAAAAUAAGGCUAGUCCGUUAUCAACUUGAAAAA | 15838140 |
| | | CA | | GUGGCACCGAGUCGGUGCUUUU | |
| 71 | G019831 | CAUCACAUG | 159 | CAUCACAUGGACCACAUCACGUUUUAGAGCUAGAAAUAG | chr4: 15838107- |
| | CD38-71 | GACCACAUC | | CAAGUUAAAAUAAGGCUAGUCCGUUAUCAACUUGAAAAA | 15838127 |
| | | AC | | GUGGCACCGAGUCGGUGCUUUU | |
| 72 | G019832 | GUGGUCCAU | 160 | GUGGUCCAUGUGAUGCUCAAGUUUUAGAGCUAGAAAUAG | chr4: 15838112- |
| | CD38-72 | GUGAUGCUC | | CAAGUUAAAAUAAGGCUAGUCCGUUAUCAACUUGAAAAA | 15838132 |
| | | AA | | GUGGCACCGAGUCGGUGCUUUU | |
| 73 | G019833 | AGAGAAGGU | 161 | AGAGAAGGUUCAGACACUAGGUUUUAGAGCUAGAAAUAG | chr4: 15840061- |
| | CD38-73 | UCAGACACU | | CAAGUUAAAAUAAGGCUAGUCCGUUAUCAACUUGAAAAA | 15840081 |
| | | AG | | GUGGCACCGAGUCGGUGCUUUU | |
| 74 | G019834 | UCUAGUGUC | 162 | UCUAGUGUCUGAACCUUCUCGUUUUAGAGCUAGAAAUAG | chr4: 15840062- |
| | CD38-74 | UGAACCUUC | | CAAGUUAAAAUAAGGCUAGUCCGUUAUCAACUUGAAAAA | 15840082 |
| | | UC | | GUGGCACCGAGUCGGUGCUUUU | |
| 75 | G019835 | GGUUCAGAC | 163 | GGUUCAGACACUAGAGGCCUGUUUUAGAGCUAGAAAUAG | chr4: 15840067- |
| | CD38-75 | ACUAGAGGC | | CAAGUUAAAAUAAGGCUAGUCCGUUAUCAACUUGAAAAA | 15840087 |
| | | CU | | GUGGCACCGAGUCGGUGCUUUU | |
| 76 | G019836 | CCAUAAUUU | 164 | CCAUAAUUUGCAACCAGAGAGUUUUAGAGCUAGAAAUAG | chr4: 15840046- |
| | CD38-76 | GCAACCAGA | | CAAGUUAAAAUAAGGCUAGUCCGUUAUCAACUUGAAAAA | 15840066 |
| | | GA | | GUGGCACCGAGUCGGUGCUUUU | |
| 77 | G019837 | CCUUCUCUG | 165 | CCUUCUCUGGUUGCAAAUUAGUUUUAGAGCUAGAAAUAG | chr4: 15840049- |
| | CD38-77 | GUUGCAAAU | | CAAGUUAAAAUAAGGCUAGUCCGUUAUCAACUUGAAAAA | 15840069 |
| | | UA | | GUGGCACCGAGUCGGUGCUUUU | |
| 78 | G019838 | AGGUUCAGA | 166 | AGGUUCAGACACUAGAGGCCGUUUUAGAGCUAGAAAUAG | chr4: 15840066- |
| | CD38-78 | CACUAGAGG | | CAAGUUAAAAUAAGGCUAGUCCGUUAUCAACUUGAAAAA | 15840086 |
| | | CC | | GUGGCACCGAGUCGGUGCUUUU | |
| 79 | G019839 | CUAGAGGCC | 167 | CUAGAGGCCUGGGUGAUACAGUUUUAGAGCUAGAAAUAG | chr4: 15840077- |
| | CD38-79 | UGGGUGAUA | | CAAGUUAAAAUAAGGCUAGUCCGUUAUCAACUUGAAAAA | 15840097 |
| | | CA | | GUGGCACCGAGUCGGUGCUUUU | |
| 80 | G019840 | UUUUAGCAC | 168 | UUUUAGCACUUUUGGGAGUGGUUUUAGAGCUAGAAAUA | chr4: 15840019- |
| | CD38-80 | UUUUGGGAG | | GCAAGUUAAAAUAAGGCUAGUCCGUUAUCAACUUGAAAA | 15840039 |
| | | UG | | AGUGGCACCGAGUCGGUGCUUUU | |
| 81 | G019841 | UCUUCCACCA | 169 | UCUUCCACCAUGUAUCACCCGUUUUAGAGCUAGAAAUAG | chr4: 15840087- |
| | CD38-81 | UGUAUCACC | | CAAGUUAAAAUAAGGCUAGUCCGUUAUCAACUUGAAAAA | 15840107 |
| | | C | | GUGGCACCGAGUCGGUGCUUUU | |
| 82 | G019842 | CUUCCCCAGA | 170 | CUUCCCCAGAGACUUAUGCCGUUUUAGAGCUAGAAAUAG | chr4: 15840442- |
| | CD38-82 | GACUUAUGC | | CAAGUUAAAAUAAGGCUAGUCCGUUAUCAACUUGAAAAA | 15840462 |
| | | C | | GUGGCACCGAGUCGGUGCUUUU | |
| 83 | G019843 | UUACCUGUA | 171 | UUACCUGUAGAUAUUCUUGCGUUUUAGAGCUAGAAAUAG | chr4: 15840522- |
| | CD38-83 | GAUAUUCUU | | CAAGUUAAAAUAAGGCUAGUCCGUUAUCAACUUGAAAAA | 15840542 |
| | | GC | | GUGGCACCGAGUCGGUGCUUUU | |
| 84 | G019844 | GCUCUUUUA | 172 | GCUCUUUUAUGGUGGGAUCCGUUUUAGAGCUAGAAAUAG | chr4: 15840463- |
| | CD38-84 | UGGUGGGAU | | CAAGUUAAAAUAAGGCUAGUCCGUUAUCAACUUGAAAAA | 15840483 |
| | | CC | | GUGGCACCGAGUCGGUGCUUUU | |
| 85 | G019845 | GGAUCCCACC | 173 | GGAUCCCACCAUAAAAGAGCGUUUUAGAGCUAGAAAUAG | chr4: 15840463- |
| | CD38-85 | AUAAAAGAG | | CAAGUUAAAAUAAGGCUAGUCCGUUAUCAACUUGAAAAA | 15840483 |
| | | C | | GUGGCACCGAGUCGGUGCUUUU | |
| 86 | G019846 | CGAUUCCAG | 174 | CGAUUCCAGCUCUUUUAUGGGUUUUAGAGCUAGAAAUAG | chr4: 15840471- |
| | CD38-86 | CUCUUUUAU | | CAAGUUAAAAUAAGGCUAGUCCGUUAUCAACUUGAAAAA | 15840491 |
| | | GG | | GUGGCACCGAGUCGGUGCUUUU | |
| 87 | G019847 | GAUUCCAGC | 175 | GAUUCCAGCUCUUUUAUGGUGUUUUAGAGCUAGAAAUAG | chr4: 15840470- |
| | CD38-87 | UCUUUUAUG | | CAAGUUAAAAUAAGGCUAGUCCGUUAUCAACUUGAAAAA | 15840490 |
| | | GU | | GUGGCACCGAGUCGGUGCUUUU | |
| 88 | G019848 | AAUCGAUUC | 176 | AAUCGAUUCCAGCUCUUUUAGUUUUAGAGCUAGAAAUAG | chr4: 15840474- |
| | CD38-88 | CAGCUCUUU | | CAAGUUAAAAUAAGGCUAGUCCGUUAUCAACUUGAAAAA | 15840494 |
| | | UA | | GUGGCACCGAGUCGGUGCUUUU | |

In some embodiments, provided herein is a composition comprising one or more guide RNA (gRNA) comprising guide sequences that direct an RNA-guided DNA binding agent, which can be a nuclease (e.g., a Cas nuclease such as Cas9), to a target DNA sequence in CD38. The gRNA may comprise a crRNA comprising a guide sequence shown in Table 1, optionally SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35. The gRNA may comprise a crRNA comprising 17, 18, 19, or 20 contiguous nucleotides of a guide sequence shown in Table 1. In some embodiments, the gRNA comprises a crRNA comprising a sequence with at least 75%, 80%, 85%, 90%, or 95%, or 100% identity to at least 17, 18, 19, or 20 contiguous nucleotides of a guide sequence shown in Table 1, optionally SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35. In some embodiments, the gRNA comprises a crRNA comprising a sequence with at least 75%, 80%, 85%, 90%, or 95%, or 100% identity to a guide sequence shown in Table 1, optionally SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35. The gRNA may further comprise a trRNA. In each embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In certain embodiments described herein, the guide RNA may comprise two RNA molecules as a “dual guide RNA” or “dgRNA.” The dgRNA comprises a first RNA molecule comprising a crRNA comprising, e.g., a guide sequence shown in Table 1, and a second RNA molecule comprising a trRNA. The first and second RNA molecules may not be covalently linked, but may form an RNA duplex via the base pairing between portions of the crRNA and the trRNA.

In some embodiments, the guide RNA may comprise a single RNA molecule as a “single guide RNA” or “sgRNA”. The sgRNA may comprise a crRNA (or a portion thereof) comprising a guide sequence shown in Table 1, optionally SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35, covalently linked to a trRNA. The sgRNA may comprise 17, 18, 19, or 20 contiguous nucleotides of a guide sequence shown in Table 1, optionally SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, provided herein is a composition comprising one or more guide RNAs comprising a guide sequence of any one of SEQ ID NOs: 1-88, preferably SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35.

In some embodiments, provided herein is a composition comprising one or more sgRNAs comprising any one of SEQ ID NOs: 125, 122, 124, 114, 123, 115, 119, 113, 116, 126, 104, 97, 98, 96, 91, 99, 111, 136, 141, 146, 147, 159, 162, 167, and 169; or 96, 97, 98, 99, 104, 111, 113, 115, 116, 119, 122, 123, 124, and 125; or 96, 97, 98, 99, 104, 113, 115, 116, 119, 122, 123, and 124; or 96, 97, 98, 99, 104, 111, 113, 115, 119, 123, 126, 136, 141, 146, 159, 167, and 169; or 91, 96, 99, 116, 123, and 125; or 96 and 123.

In one aspect, provided herein is a composition comprising a gRNA that comprises a guide sequence that is 100% or at least 95% or 90% identical to any of the nucleic acids of SEQ ID NOs: 1-88, preferably SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35.

In other embodiments, the composition comprises at least one, e.g., at least two gRNA's comprising guide sequences selected from any two or more of the guide sequences of SEQ ID NOs: 1-88, preferably SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35. In some embodiments, the composition comprises at least two gRNA's that each comprise a guide sequence 100%, or at least 95% or 90% identical to any of the nucleic acids of SEQ ID NOs: 1-88, preferably SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35.

In certain embodiments, the guide RNA compositions provided herein are designed to recognize (e.g., hybridize to) a target sequence in a CD38 gene. For example, the CD38 target sequence may be recognized and cleaved by a provided Cas cleavase comprising a guide RNA. In some embodiments, an RNA-guided DNA binding agent, such as a Cas cleavase, may be directed by a guide RNA to a target sequence of a CD38 gene, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within a CD38 gene.

Without being bound by any particular theory, mutations (e.g., frameshift mutations resulting from indels, i.e., insertions or deletions, occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary or having complementarity to a target sequence within CD38 is used to direct the RNA-guided DNA binding agent to a particular location in the appropriate CD38 gene. In some embodiments, gRNAs are designed to have guide sequences that are complementary or have complementarity to target sequences in exon 1, exon 2, exon 3, exon 4, exon 5, exon 6, exon 7, or exon 8 of CD38.

In some embodiments, the guide sequence is 100% or at least 95% or 90% identical to a target sequence present in a human CD38 gene. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, or 95%; or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

Modified gRNAs and mRNAs

In some embodiments, the gRNA is chemically modified. A gRNA comprising one or more modified nucleosides or nucleotides is called a “modified” gRNA or “chemically modified” gRNA, to describe the presence of one or more non-naturally or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar or backbone modification); and (vii) modification or replacement of the sugar (an exemplary sugar modification).

Chemical modifications such as those listed above can be combined to provide modified gRNAs or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates, and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo, and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine, and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., an alkyl, cycloalkyl, aryl, aralkyl, heteroaryl group, or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)n-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH2CH₂— amino (wherein amino can be, e.g., as described herein), —NHC(O)R (wherein R can be, e.g., an alkyl, cycloalkyl, aryl, aralkyl, heteroaryl group, or sugar), cyano; mercapto; alkyl-thio-alkyl; thioalkoxy; and alkyl, cycloalkyl, aryl, alkenyl, and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA or tracr RNA. In embodiments comprising an sgRNA, one or more residues at one or both ends of the sgRNA may be chemically modified, or internal nucleosides may be modified, or the entire sgRNA may be chemically modified. Certain embodiments comprise a 5′ end modification. Certain embodiments comprise a 3′ end modification. Additional embodiments comprise a 5′ end modification and a 3′ end modification.

In some embodiments, the guide RNAs disclosed herein comprise one of the modification patterns disclosed in WO2018/107028 A1, titled “Chemically Modified Guide RNAs” or WO2021119275 titled “Modified Guide RNAs for Gene Editing,” the contents of each are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety.

In some embodiments, the sgRNA comprises any of the modification patterns shown herein, where N is any natural or non-natural nucleotide, and wherein the totality of the N's comprise a CD38 guide sequence as described herein in Table 1. In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise an CD38 guide sequence as described in Table 1. For example, where the N's are replaced with any of the guide sequences disclosed herein in Table 1, optionally wherein the N's are replaced with SEQ ID NO: 37, 34, 36, 26, 35, 27, 31, 25, 28, 38, 16, 9, 10, 8, 3, 11, 23, 48, 53, 58, 59, 71, 74, 79, and 81; or SEQ ID NO: 8, 9, 10, 11, 16, 23, 25, 27, 28, 31, 34, 35, 36, and 37; or SEQ ID NO: 8, 9, 10, 11, 16, 25, 27, 28, 31, 34, 35, and 36; or SEQ ID NO: 8, 9, 10, 11, 16, 23, 25, 27, 31, 35, 38, 48, 53, 58, 71, 79, and 81; or SEQ ID NO: 3, 8, 11, 28, 35, and SEQ ID NO: 37; or SEQ ID NO: 9, 10, 11, 27, and 35; or SEQ ID NO: 10, 11, and 35; or SEQ ID NO: 8 and SEQ ID NO: 35. In some embodiments, the sgRNA listed in Table 1 are modified according to the modification pattern of SEQ ID NO: 300. In some embodiments, the sgRNA may comprise a sequence of any one of SEQ ID NO: 1220-1225 (Table 12), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

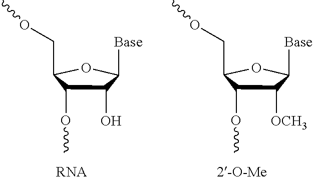

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

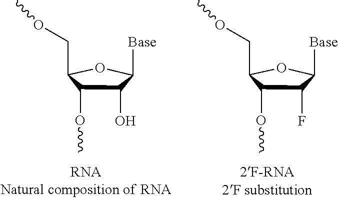

Phosphorothioate (PS) linkage or bond refers to a bond where a sulfur is substituted for one non-bridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S- into a non-bridging phosphate oxygen, generating a PS bond in lieu of a phosphodiester bond:

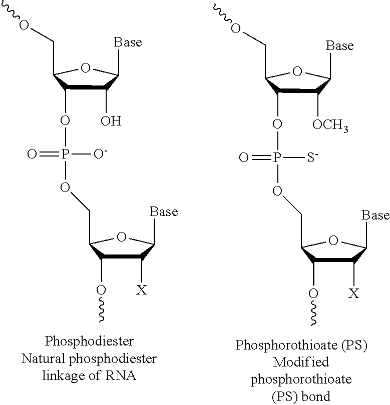

Abasic nucleotides refer to those which lack nitrogenous bases. The figure below depicts an oligonucleotide with an abasic (also known as apurinic) site that lacks a base:

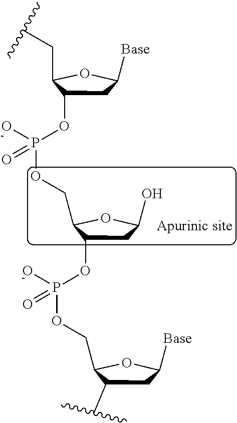

Inverted bases refer to those with linkages that are inverted from the normal 5′ to 3′ linkage (i.e., either a 5′ to 5′ linkage or a 3′ to 3′ linkage). For example:

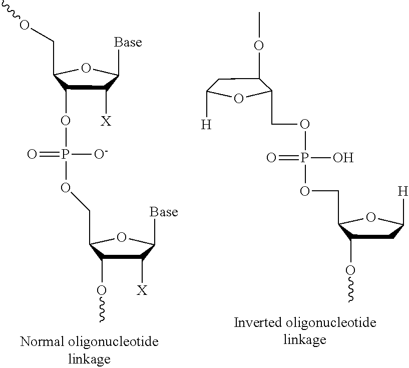

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus and one or more of the last three, four, or five nucleotides at the 3′ terminus are modified. In some embodiments, the modification is a 2′-O-Me, 2′-F, inverted abasic nucleotide, PS bond, or other nucleotide modification well known in the art to increase stability or performance.

In some embodiments, the first four nucleotides at the 5′ terminus and the last four nucleotides at the 3′ terminus are linked with phosphorothioate (PS) bonds.

In some embodiments, the first three nucleotides at the 5′ terminus and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus and the last three nucleotides at the 3′ terminus comprise an inverted abasic nucleotide.

In some embodiments, the guide RNA comprises a modified sgRNA. In some embodiments, the sgRNA comprises the modification pattern shown in mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where N is any natural or non-natural nucleotide, and where the totality of the N's comprise a guide sequence that directs a nuclease to a target sequence in CD38 e.g., the genomic coordinates shown in Table 1. In some embodiments, the sgRNA may comprise a sequence of any one of SEQ ID NO: 1220-1225 (Table 12), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

In some embodiments, the guide RNA comprises a sgRNA comprising any one of the guide sequences of SEQ ID NOs: 1-88 and a conserved portion of an sgRNA, for example, the conserved portion of sgRNA shown as Exemplary SpyCas9 sgRNA-1 or the conserved portions of the gRNAs shown in Table 1 and throughout the specification. In some embodiments, the guide RNA comprises a sgRNA comprising any one of the guide sequences of SEQ ID NOs: 1-88 and the nucleotides of GUUUUAGAGCUAGAAAUAGCAAGUUAAAAUAAGGCUAGUCCGUUAUCAACUU GAAAAAGUGGCACCGAGUCGGUGCUUUU (SEQ ID NO: 202), wherein the nucleotides are on the 3′ end of the guide sequence, and wherein the sgRNA may be modified as shown herein or in the sequence mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300). In some embodiments, the sgRNA comprises Exemplary SpyCas9 sgRNA-1 or the modified versions thereof provided herein, or a version as provided in Table 9 below, where the totality of the N's comprise a guide sequence that directs a nuclease to a target sequence. “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence. Each N is independently modified or unmodified. In certain embodiments, in the absence of an indication of a modification, the nucleotide is an unmodified RNA nucleotide residue, i.e., a ribose sugar and a phosphodiester backbone. In some embodiments, the sgRNA may comprise a sequence of any one of SEQ ID NO: 1220-1225 (Table 12), where “N” may be any natural or non-natural nucleotide, preferably an RNA nucleotide; sugar moieties of the nucleotide can be ribose, deoxyribose, or similar compounds with substitutions; m is a 2′-O-methyl modified nucleotide, and * is a phosphorothioate linkage between nucleotide residues; and wherein the N's are collectively the nucleotide sequence of a guide sequence.

As noted above, in some embodiments, a composition or formulation disclosed herein comprises an mRNA comprising an open reading frame (ORF) encoding an RNA-guided DNA binding agent, such as a Cas nuclease, e.g. Cas9 nuclease, as described herein. In some embodiments, an mRNA comprising an ORF encoding an RNA-guided DNA binding agent, such as a Cas nuclease, e.g. Cas9 nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the mRNA and/or modified ORF may comprise a modified uridine at least at one, a plurality of, or all uridine positions. In some embodiments, the modified uridine is a uridine modified at the 5 position, e.g., with a halogen or a methyl or ethyl group. In some embodiments, the modified uridine is a pseudouridine modified at the 1 position, e.g., with a halogen or a methyl or ethyl group. The modified ORF comprises one or more modified uridines that can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Nat Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

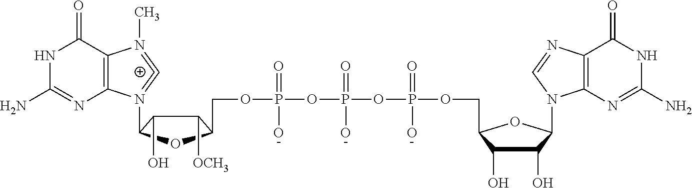

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

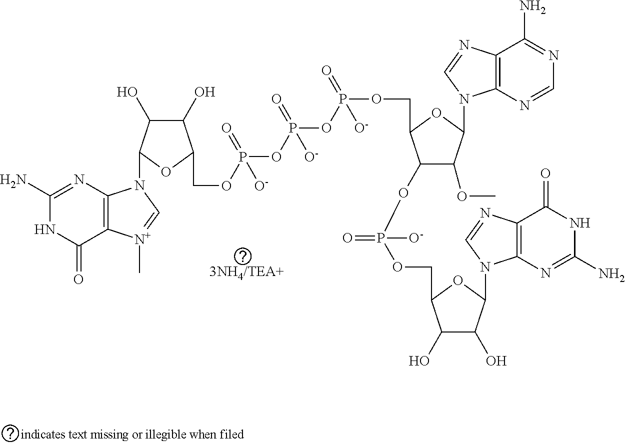 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

### Poly-A Tail

In some embodiments, the mRNA further comprises a poly-adenylated (poly-A) tail. In some embodiments, the poly-A tail sequence comprises 100-400 nucleotides. In some embodiments, the poly-A tail comprises at least 20, 30, 40, 50, 60, 70, 80, 90, or 100 adenines. In some embodiments, the polyA sequence comprises non-adenine nucleotides. In some instances, the poly-A tail is “interrupted” with one or more non-adenine nucleotide “anchors” at one or more locations within the poly-A tail. The poly-A tails may comprise at least 8 consecutive adenine nucleotides, but also comprise one or more non-adenine nucleotide. As used herein, “non-adenine nucleotides” refer to any natural or non-natural nucleotides that do not comprise adenine. Guanine, thymine, and cytosine nucleotides are exemplary non-adenine nucleotides. Thus, the poly-A tails on the mRNA described herein may comprise consecutive adenine nucleotides located 3′ to nucleotides encoding a polypeptide disclosed herein. In some instances, the poly-A tails on mRNA comprise non-consecutive adenine nucleotides located 3′ to nucleotides encoding an RNA-guided DNA-binding agent or a sequence of interest, wherein non-adenine nucleotides interrupt the adenine nucleotides at regular or irregularly spaced intervals.

In some embodiments, the poly-A tail is encoded in the plasmid used for in vitro transcription of mRNA and becomes part of the transcript. The poly-A sequence encoded in the plasmid, i.e., the number of consecutive adenine nucleotides in the poly-A sequence, may not be exact, e.g., a 100 poly-A sequence in the plasmid may not result in a precisely 100 poly-A sequence in the transcribed mRNA. In some embodiments, the poly-A tail is not encoded in the plasmid, and is added by PCR tailing or enzymatic tailing, e.g., using E. coli poly(A) polymerase.

In some embodiments, the one or more non-adenine nucleotides are positioned to interrupt the consecutive adenine nucleotides so that a poly(A) binding protein can bind to a stretch of consecutive adenine nucleotides. In some embodiments, one or more non-adenine nucleotide(s) is located after at least 8, 9, 10, 11, or 12 consecutive adenine nucleotides. In some embodiments, the one or more non-adenine nucleotide is located after at least 8-50 consecutive adenine nucleotides. In some embodiments, the one or more non-adenine nucleotide is located after at least 8-100 consecutive adenine nucleotides. In some embodiments, the non-adenine nucleotide is after one, two, three, four, five, six, or seven adenine nucleotides and is followed by at least 8 consecutive adenine nucleotides.

The poly-A tail of the present disclosure may comprise one sequence of consecutive adenine nucleotides followed by one or more non-adenine nucleotides, optionally followed by additional adenine nucleotides.

In some embodiments, the poly-A tail comprises or contains one non-adenine nucleotide or one consecutive stretch of 2-10 non-adenine nucleotides. In some embodiments, the non-adenine nucleotide(s) is located after at least 8, 9, 10, 11, or 12 consecutive adenine nucleotides. In some instances, the one or more non-adenine nucleotides are located after at least 8-50 consecutive adenine nucleotides. In some embodiments, the one or more non-adenine nucleotides are located after at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive adenine nucleotides.

In some embodiments, the non-adenine nucleotide is guanine, cytosine, or thymine. In some instances, the non-adenine nucleotide is a guanine nucleotide. In some embodiments, the non-adenine nucleotide is a cytosine nucleotide. In some embodiments, the non-adenine nucleotide is a thymine nucleotide. In some instances, where more than one non-adenine nucleotide is present, the non-adenine nucleotide may be selected from: a) guanine and thymine nucleotides; b) guanine and cytosine nucleotides; c) thymine and cytosine nucleotides; or d) guanine, thymine and cytosine nucleotides.

### Ribonucleoprotein Complexes

In some embodiments, a composition provided herein is encompassed comprising one or more gRNAs comprising one or more guide sequences from Table 1 or one or more sgRNAs from Table 1 and an RNA-guided DNA binding agent, e.g., a nuclease, such as a Cas nuclease, such as Cas9. In some embodiments, the RNA-guided DNA-binding agent has cleavase activity, which can also be referred to as double-strand endonuclease activity. In some embodiments, the RNA-guided DNA-binding agent comprises a Cas nuclease. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US20160312198; US 20160312199. Other examples of Cas nucleases include a Csm or Cmr complex of a type III CRISPR system or the Cas10, Csm1, or Cmr2 subunit thereof; and a Cascade complex of a type I CRISPR system, or the Cas3 subunit thereof. In some embodiments, the Cas nuclease may be from a Type-IIA, Type-JIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell Oct 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 20140186958; US 20150166980.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV, or PKKKRRV. In some embodiments, the NLS may be a bipartite sequence, such as the NLS of nucleoplasmin, KRPAATKKAGQAKKKK. In a specific embodiment, a single PKKKRKV NLS may be linked at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called Rubi in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellowl), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression, “Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes, “Cell 154:442-51 (2013). As such, the RNA-guided DNA-binding agent essentially becomes a transcription factor that can be directed to bind a desired target sequence using a guide RNA. In some embodiments, the heterologous functional domain is a deaminase, such as a cytidine deaminase or an adenine deaminase. In certain embodiments, the heterologous functional domain is a C to T base converter (cytidine deaminase), such as an apolipoprotein B mRNA editing enzyme (APOBEC) deaminase.

In some embodiments, the RNA-guided DNA binding agent is selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, Mad7 nuclease, ARCUS nucleases, and CasX. In some embodiments, the RNA-guided DNA binding agent comprises a polypeptide selected from one of: S. pyogenes Cas9, Neisseria meningitidis Cas9, e.g. an Nme2Cas9, S. thermophilus Cas9, S. aureus Cas9, Francisella novicida Cpf1, Acidaminococcus sp. Cpf1, Lachnospiraceae bacterium Cpf1, C-to-T base editor, A-to-G base editor, Cas12a, and CasX.

In some embodiments, the RNA-guided DNA binding agent comprises an editor. An exemplary editor is BC22n, which includes an H. sapiens APOBEC3A fused to S. pyogenes-D10A Cas9 nickase by an XTEN linker, and mRNA encoding BC22n. An mRNA encoding BC22n is provided (SEQ ID NO: 804 or 805).

Determination of Efficacy of gRNAs

In some embodiments, the efficacy of a gRNA is determined when delivered or expressed together with other components forming an RNP. In some embodiments, the gRNA is expressed together with an RNA-guided DNA binding agent, such as a Cas protein, e.g., Cas9. In some embodiments, the gRNA is delivered to or expressed in a cell line that already stably expresses an RNA-guided DNA nuclease, such as a Cas nuclease or nickase, e.g., Cas9 nuclease or nickase. In some embodiments the gRNA is delivered to a cell as part of a RNP. In some embodiments, the gRNA is delivered to a cell along with a mRNA encoding an RNA-guided DNA nuclease, such as a Cas nuclease or nickase, e.g., Cas9 nuclease or nickase.

As described herein, use of an RNA-guided DNA nuclease and a guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein. In some embodiments, the efficacy of particular gRNAs is determined based on in vitro models. In some embodiments, the in vitro model is HEK293 cells stably expressing Cas9 (HEK293_Cas9). In some embodiments the in vitro model is a peripheral blood mononuclear cell (PBMC). In some embodiments, the in vitro model is a T cell, such as primary human T cells. In some embodiments, the in vitro model is a NK cell, such as primary human NK cells. With respect to using primary cells, commercially available primary cells can be used to provide greater consistency between experiments. In some embodiments, the number of off-target sites at which a deletion or insertion occurs in an in vitro model (e.g., in T cells or NK cells) is determined, e.g., by analyzing genomic DNA from transfected cells in vitro with Cas9 mRNA and the guide RNA. In some embodiments, such a determination comprises analyzing genomic DNA from the cells transfected in vitro with Cas9 mRNA, the guide RNA, and a donor oligonucleotide. Exemplary procedures for such determinations are provided in the working examples in which HEK293 cells, PBMCs, human CD3+ T cells, and human NK cells are used.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of a guide RNA is measured by percent indels or percent genetic modifications of CD38. In some embodiments, the efficacy of a guide RNA is measured by percent indels or percent genetic modifications at a CD38 locus. In some embodiments, the efficacy of a guide RNA is measured by percent indels or percent genetic modifications of CD38 at genomic coordinates of Table 1. In some embodiments, the percent editing of CD38 is compared to the percent indels or genetic modifications necessary to achieve knockdown of the CD38 protein products. In some embodiments, the efficacy of a guide RNA is measured by reduced or eliminated expression of CD38 protein. In embodiments, said reduced or eliminated expression of CD38 protein is as measured by flow cytometry, e.g., as described herein.

In some embodiments, the CD38 protein expression is reduced or eliminated in a population of cells using the methods and compositions disclosed herein. In some embodiments, the population of cells is at least 55%, 60%, 65%, 70%, 80%, 90%, 91%, 92%, 93%, 94%, 95%, 96%, 97%, 98%, or 99% CD38 negative as measured by flow cytometry relative to a population of unmodified cells.

An “unmodified cell” (or “unmodified cells”) refers to a control cell (or cells) of the same type of cell in an experiment or test, wherein the “unmodified” control cell has not been contacted with a CD38 guide. Therefore, an unmodified cell (or cells) may be a cell that has not been contacted with a guide RNA, or a cell that has been contacted with a guide RNA that does not target CD38.

In some embodiments, the efficacy of a guide RNA is measured by the number or frequency of indels or genetic modifications at off-target sequences within the genome of the target cell type, such as a T cell or NK cell. In some embodiments, efficacious guide RNAs are provided which produce indels at off target sites at very low frequencies (e.g., <5%) in a cell population or relative to the frequency of indel creation at the target site. Thus, the disclosure provides for guide RNAs which do not exhibit off-target indel formation in the target cell type (e.g., a T cell or NK cell), or which produce a frequency of off-target indel formation of <5% in a cell population or relative to the frequency of indel creation at the target site. In some embodiments, the disclosure provides guide RNAs which do not exhibit any off target indel formation in the target cell type (e.g., T cell or NK cell). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and insertion or homology directed repair (HDR) events in target DNA utilize linear amplification with a tagged primer and isolating the tagged amplification products (herein after referred to as “LAM-PCR,” or “Linear Amplification (LA)” method). In some embodiments, the efficacy of a guide RNA is measured by the levels of functional protein complexes comprising the expressed protein product of the gene. In some embodiments, the efficacy of a guide RNA is measured by flow cytometric analysis of CD38 expression by which the live population of edited cells is analyzed for loss of the CD38.

### T Cell Receptors (TCR)

In some embodiments, the engineered cells or population of cells comprising a genetic modification, e.g., of an endogenous nucleic acid sequence encoding CD38, further comprise a modification, e.g., knockdown, of an endogenous nucleic acid sequence encoding TCR gene sequence(s), e.g., TRAC or TRBC.

In some embodiments, the engineered cells or population of cells comprising a genetic modification, e.g., knockdown, of an endogenous nucleic acid sequence encoding CD38 and insertion into the cell of heterologous sequence(s) encoding a targeting receptor, further comprise a modification, e.g., knockdown, of an endogenous nucleic acid sequence encoding TCR gene sequence(s), e.g., TRAC or TRBC.

Generally, a TCR is a heterodimer receptor molecule that contains two TCR polypeptide chains, α and β. Suitable α and β genomic sequences or loci to target for knockdown are known in the art. In some embodiments, the engineered T cells comprise a modification, e.g., knockdown, of a TCR α-chain gene sequence, e.g., TRAC. See, e.g., NCBI Gene ID: 28755; Ensembl: ENSG00000277734 (T-cell receptor Alpha Constant), US 2018/0362975, and WO2020081613.

In some embodiments, the engineered cells or population of cells comprise a genetic modification of an endogenous nucleic acid sequence encoding CD38, a genetic modification, e.g., knockdown, of an endogenous nucleic acid sequence encoding TCR gene sequence(s), e.g., TRAC or TRBC; and modification, e.g., knockdown of an MHC class I gene, e.g., B2M or HLA-A. In some embodiments, an MHC class I gene is an HLA-B gene or an HLA-C gene.

In some embodiments, the engineered cells or population of cells comprise a genetic modification of an endogenous nucleic acid sequence encoding CD38 and a genetic modification, e.g., knockdown, of an endogenous nucleic acid sequence encoding a TCR, e.g., TRAC or TRBC; and a genetic modification, e.g., knockdown of an MHC class II gene, e.g., CIITA.

In some embodiments, the engineered cells or population of cells comprise a modification of an endogenous nucleic acid sequence encoding CD38, a genetic modification, e.g., knockdown, of an endogenous nucleic acid sequence encoding a TCR, e.g., TRAC or TRBC; and a genetic modification, e.g. knockdown of a checkpoint inhibitor gene, e.g., TIM3, 2B4, LAG3, or PD-1.

In some embodiments, the engineered cells or population of cells comprise a genetic modification of a CD38 gene as assessed by sequencing, e.g., NGS, wherein at least 50%, 55%, 60%, 65%, preferably at least 70%, 75%, 80%, 85%, 90%, 95%, 96%, 97%, 98%, or 99% of cells comprise an insertion, deletion, or substitution in the endogenous CD38 sequence. In some embodiments, at least 50% of cells in the population comprise a modification selected from an insertion, a deletion, and a substitution in the endogenous CD38 sequence. In some embodiments, at least 55% of cells in the population comprise a modification selected from an insertion, a deletion, and a substitution in the endogenous CD38 sequence. In some embodiments, at least 60% of cells in the population comprise a modification selected from an insertion, a deletion, and a substitution in the endogenous CD38 sequence. In some embodiments, at least 65% of cells in the population comprise a modification selected from an insertion, a deletion, and a substitution in the endogenous CD38 sequence. In some embodiments, at least 70% of cells in the population comprise a modification selected from an insertion, a deletion, and a substitution in the endogenous CD38 sequence. In some embodiments, at least 75% of cells in the population comprise a modification selected from an insertion, a deletion, and a substitution in the endogenous CD38 sequence. In some embodiments, at least 85% of cells in the population comprise a modification selected from an insertion, a deletion, and a substitution in the endogenous CD38 sequence. In some embodiments, at least 70% of cells in the population comprise a modification selected from an insertion, a deletion, and a substitution in the endogenous CD38 sequence. In some embodiments, at least 90% of cells in the population comprise a modification selected from an insertion, a deletion, and a substitution in the endogenous CD38 sequence. In some embodiments, at least 95% of cells in the population comprise a modification selected from an insertion, a deletion, and a substitution in the endogenous CD38 sequence. In some embodiments, CD38 is decreased by at least 50%, 55%, 60%, 65%, preferably at least 70%, 75%, 80%, 85%, 90%, 95%, 96%, 97%, 98%, or 99%, or to below the limit of detection of the assay as compared to a suitable control, e.g., wherein the CD38 gene has not been modified. In some embodiments, expression of CD38 is decreased by at least 50% or to below the limit of detection of the assay as compared to a suitable control, e.g., wherein the CD38 gene has not been modified. In some embodiments, expression of CD38 is decreased by at least 55% or to below the limit of detection of the assay as compared to a suitable control, e.g., wherein the CD38 gene has not been modified. In some embodiments, expression of CD38 is decreased by at least 60% or to below the limit of detection of the assay as compared to a suitable control, e.g., wherein the CD38 gene has not been modified. In some embodiments, expression of CD38 is decreased by at least 65% or to below the limit of detection of the assay as compared to a suitable control, e.g., wherein the CD38 gene has not been modified. In some embodiments, expression of CD38 is decreased by at least 70% or to below the limit of detection of the assay as compared to a suitable control, e.g., wherein the CD38 gene has not been modified. In some embodiments, expression of CD38 is decreased by at least 80% or to below the limit of detection of the assay as compared to a suitable control, e.g., wherein the CD38 gene has not been modified. In some embodiments, expression of CD38 is decreased by at least 90% or to below the limit of detection of the assay as compared to a suitable control, e.g., wherein the CD38 gene has not been modified. In some embodiments, expression of CD38 is decreased by at least 95% or to below the limit of detection of the assay as compared to a suitable control, e.g., wherein the CD38 gene has not been modified. Assays for CD38 protein and mRNA expression are known in the art. “Expression of CD38” refers to the expression of encoding transcript (e.g., CD38 mRNA) or expression of a CD38 protein or a portion thereof. Inhibiting expression of CD38 can result in a decreased level of a CD38-encoding transcript (e.g., CD38 mRAN) or a decreased level of a CD38 protein or a portion thereof. Inhibition of CD38 expression can be assessed by detecting or quantifying CD38-encoding transcripts (e.g., mRNA), CD38 proteins, portions of CD38 proteins, or CD38 activity.

In some embodiments, the engineered cells or population of cells comprise a modification, e.g., knockdown, of a TCR gene sequence by gene editing, e.g., as assessed by sequencing, e.g., NGS, wherein at least 50%, 55%, 60%, 65%, preferably at least 70%, 75%, 80%, 85%, 90%, 95%, 96%, 97%, 98%, or 99% of cells comprise an insertion, deletion, or substitution in the endogenous TCR gene sequence. In some embodiments, TCR is decreased by at least 50%, 55%, 60%, 65%, preferably at least 70%, 75%, 80%, 85%, 90%, 95%, 96%, 97%, 98%, 99%, or to below the limit of detection of the assay as compared to a suitable control, e.g., wherein the TCR gene has not been modified. In certain embodiments, the TCR is TRAC or TRBC. Assays for TCR protein and mRNA expression are known in the art.

In some embodiments, the engineered cells or population of cells comprise an insertion of sequence(s) encoding a targeting receptor by gene editing, e.g., as assessed by sequencing, e.g., NGS.

In some embodiments, guide RNAs that specifically target sites within the TCR genes, e.g., TRAC gene, are used to provide a modification, e.g., knockdown, of the TCR genes.

In some embodiments, the TCR gene is modified, e.g., knocked down, in a T cell using a guide RNA with an RNA-guided DNA binding agent. In some embodiments, disclosed herein are T cells engineered by inducing a break (e.g., double-stranded break (DSB) or single-stranded break (nick)) within the TCR genes of a T cell, e.g., using a guide RNA with an RNA-guided DNA-binding agent (e.g., a CRISPR/Cas system). The methods may be used in vitro or ex vivo, e.g., in the manufacture of cell products for suppressing immune response.

In some embodiments, the guide RNAs mediate a target-specific cutting by an RNA-guided DNA-binding agent (e.g., Cas nuclease) at a site described herein within a TCR gene. It will be appreciated that, in some embodiments, the guide RNAs comprise guide sequences that bind to, or are capable of binding to, said regions.

### Methods and Uses Including Therapeutic Methods and Uses and Methods of Preparing Engineered Cells or Immunotherapy Reagents

In certain embodiments, the gRNAs and associated methods and compositions disclosed herein are useful for making cell therapy (e.g., immunotherapy) reagents, such as engineered cells (e.g., engineered T cells and/or engineered NK cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells (NK Cell), cytotoxic T lymphocytes (CTL) can be programmed to act in response to abnormal antigens expressed on the surface of tumor cells. Thus, cancer immunotherapy allows components of the immune system to destroy tumors or other cancerous cells.

Immunotherapy can also be useful for the treatment of chronic infectious disease, e.g., hepatitis B and C virus infection, human immunodeficiency virus (HIV) infection, tuberculosis infection, and malarial infection. Immune effector cells comprising a targeting receptor such as a transgenic TCR or CAR are useful in immunotherapies, such as those described herein.

In some embodiments, the gRNAs comprising the guide sequences of Table 1 together with an RNA-guided DNA nuclease, such as a Cas nuclease, induce double-strand breaks (DSBs) and non-homologous ending joining (NHEJ) during repair leads to a modification, e.g., a mutation in a CD38 gene. In some embodiments, NHEJ leads to a deletion or insertion of a nucleotide(s), which induces a frame shift or nonsense mutation in a CD38 gene. In certain embodiments, gRNAs comprising guide sequences targeted to TCR sequences, e.g., TRAC and TRBC, are also delivered to the cell together with RNA-guided DNA nuclease such as a Cas nuclease, either together or separately, to make a genetic modification in a TCR sequence to inhibit the expression of a full-length TCR sequence. In certain embodiments, the gRNAs are sgRNAs.

In some embodiments, the subject is mammalian. In some embodiments, the subject is human. In some embodiments, the subject is a non-human primate.

In some embodiments, the guide RNAs, compositions, and formulations are used to produce a cell ex vivo, e.g., an immune cell, e.g., a T cell with a genetic modification in a CD38 gene. The modified T cell may be a natural killer (NK) T-cell. The modified T cell may express a T-cell receptor, such as a universal TCR or a modified TCR. The T cell may express a CAR or a CAR construct with a zeta chain signalling motif.

Delivery of gRNA Compositions

Lipid nanoparticles (LNPs) are a well-known means for delivery of nucleotide and protein cargo, and may be used for delivery of the guide RNAs and compositions disclosed herein ex vivo and in vitro. In some embodiments, the LNPs deliver nucleic acid, protein, or nucleic acid together with protein.

In some embodiments, provided herein is a method for delivering any one of the cells or populations of cells disclosed herein to a subject, wherein the gRNA is delivered via an LNP. In some embodiments, the gRNA/LNP is also associated with a Cas9 or an mRNA encoding Cas9.

In some embodiments, provided herein is a composition comprising any one of the gRNAs disclosed and an LNP. In some embodiments, the composition further comprises a Cas9 or an mRNA encoding Cas9.

In some embodiments, LNPs associated with the gRNAs disclosed herein are for use in preparing cells as a medicament for treating a disease or disorder.

Electroporation is a well-known means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, provided herein is a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or an mRNA encoding Cas9.

In some embodiments, the guide RNA compositions described herein, alone or encoded on one or more vectors, are formulated in or administered via a lipid nanoparticle (see e.g., WO2017/173054 and PCT/US2021/29446, the contents of each are hereby incorporated by reference in their entirety).

In certain embodiments, provided herein are DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., SpyCas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced as naked nucleic acid, as nucleic acid complexed with an agent such as a liposome or poloxamer, or they can be delivered by viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

### Combination Therapies

The delivery of gRNAs together with an RNA-guided DNA nuclease that induces double-strand breaks (DSBs) and non-homologous ending joining (NHEJ) during repair resulting in a modification, e.g., a mutation, in a CD38 gene, as described herein, can be combined with one or more additional therapies. In some embodiments, the additional therapy is a cancer therapy. In some embodiments, the additional therapy is a chemotherapy, hormone therapy, immunotherapy, radiation therapy, or a targeted therapy.

The delivery of gRNAs together with an RNA-guided DNA nuclease that induce double-strand breaks (DSBs) and non-homologous ending joining (NHEJ) during repair resulting in a mutation in a CD38 gene, as described herein, can be combined with one or more additional therapies. In some embodiments, the additional therapy is a cancer therapy. In some embodiments, the additional therapy is a chemotherapy, hormone therapy, immunotherapy, radiation therapy, or a targeted therapy.

In some embodiments, the additional therapy can be an anti-CD38 antibody. Combining the gRNA/Cas therapeutic approach that results in at least one mutation in a CD38 gene with another anti-CD38 therapy (e.g., an anti-CD38 targeting therapy) can reduce the CD38 activity in those cells that escaped the gRNA/Cas therapeutic approach. The additional therapy can also be another gRNA/Cas therapy that comprises gRNAs that target other genes (e.g., TCR genes).

Anti-CD38 antibodies are known in the art and have been shown to be effective (or are in clinical trials to confirm their effectiveness) in reducing CD38 activity and treating or preventing certain diseases (e.g., multiple myeloma, diffuse large B cell lymphoma, follicular lymphoma, mantle cell lymphoma, T-cell leukemia). Thus, contemplated herein are antibodies that specifically bind to CD38 and inhibit its activity at least partially. In some embodiments, the anti-CD38 antibody is daratumumab, an IgG1k human monoclonal antibody, that has been shown to be effective (or is in clinical trials to confirm effectiveness) in treating multiple myeloma, diffuse large B cell lymphoma, follicular lymphoma, and mantle cell lymphoma. Additionally, it has been shown that administration of CD38 depleted NK cells reduces or eliminates fratricide caused by daratumumab and boosts the effectiveness of the NK cells (Kararoudi et al., Blood (2020) 136 (21): 2416-2427. In some embodiments, the anti-CD38 antibody is isatuximab (an IgG1 human monoclonal antibody). In some embodiments, the anti-CD38 antibody is a bispecific antibody. In some embodiments, the anti-CD38 antibody is TAK-079 or MOR-202, which are both currently in clinical trials.

In some embodiments, the CD38 inhibitor is a small molecule. In some embodiments, the small molecule is a 4-amino-quinoline. Examples of -amino-quinolines include, but are not limited to, CD38 inhibitor 78c, CD38 inhibitor 1 ah, and CD38 inhibitor 1ai. These types of CD38 inhibitors generally competitively inhibit CD38's NADase activity. CD38 inhibitor 78c (structure shown below), has been shown to be effective in reducing tumor mass in a Lewis lung carcinoma mouse model.

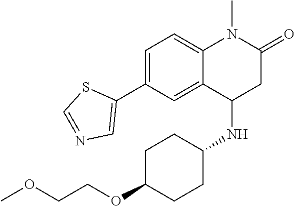 [SMILES: COCCO[C@H]1CC[C@H](NC2CC(=O)N(C)c3ccc(-c4cncs4)cc32)CC1]

NAD+ analogs also inhibit CD38 and are contemplated herein as additional therapeutics that can be combined with the gRNA/Cas system that targets CD38. NAD+ analogs that are CD38 inhibitors include, but are not necessarily limited to, Ara-F-NAD+, Ara-F-NMN, Ara-F-NMN phosphoester/C48, Carba-NAD, and Pseudo-Carba-NAD.

NAD+ analogs also inhibit CD38 and are contemplated herein as additional therapeutics that can be combined with the gRNA/Cas system that targets CD38. NAD+ analogs that are CD38 inhibitors include, but are not necessarily limited to, Ara-F-NAD+, Ara-F-NMN, Ara-F-NMN phosphoester/C48, Carba-NAD, and Pseudo-Carba-NAD.

In some embodiments, the anti-CD38 inhibitor is a flavonoid. Flavonoid CD38 inhibitors are generally not toxic to humans and beneficial effects have been observed in animal models of obesity, heart ischemia, kidney injury, viral infection, and cancer. Flavonoid inhibitors of CD38 include, but are not limited to, Quercetin, Apigenin, Luteolinidin, Kuromanin, and Rhein/K-Rhein. Flavonoids, like NAD+ analogs tend to inhibit CD38's NADase activity, generally by competitive inhibition.

In some embodiments, the additional cancer therapy is CAR-T cell therapy. Chimeric antigen receptors (CAR) are molecules combining antibody-based specificity for tumor-associated surface antigens with T cell receptor-activating intracellular domains with specific anti-tumor cellular immune activity (Eshhar, 1997, Cancer Immunol Immunother 45(3-4) 131-136; Eshhar et al., 1993, Proc Natl Acad Sci USA 90(2):720-724; Brocker and Karjalainen, 1998, Adv Immunol 68:257-269). These CARs allow a T cell to achieve MHC-independent primary activation through single chain Fv (scFv) antigen-specific extracellular regions fused to intracellular domains that provide T cell activation and co-stimulatory signals. Second and third generation CARs also provide appropriate co-stimulatory signals via CD28 and/or CD137 (4-1BB) intracellular activation motifs, which augment cytokine secretion and anti-tumor activity in a variety of solid tumor and leukemia models (Pinthus, et al, 2004, J Clin Invest 114(12):1774-1781; Milone, et al., 2009, Mol Ther 17(8):1453-1464; Sadelain, et al., 2009, Curr Opin Immunol 21(2):215-223). Chimeric Antigen Receptor (CAR) T cell therapy involves genetic modification of patient's autologous T-cells to express a CAR specific for a tumor antigen, following by ex vivo cell expansion and re-infusion back to the patient. CARs are fusion proteins of a selected single-chain fragment variable from a specific monoclonal antibody and one or more T cell receptor intracellular signaling domains. This T cell genetic modification may occur either via viral-based gene transfer methods or nonviral methods, such as DNA-based transposons, CRISPR/Cas9 technology or direct transfer of in vitro transcribed-mRNA by electroporation.

### Indications

In some embodiments, the methods described herein may be used to treat any cancer, including any cancerous or pre-cancerous tumor. Cancers that may be treated by methods and compositions provided herein include, but are not limited to, cancer of the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchioloalveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the cancer being treated is a CD38-expressing cancer.

In some embodiments, the cancer comprises a solid tumor. In some embodiments, the tumor is an adenocarcinoma, an adrenal tumor, an anal tumor, a bile duct tumor, a bladder tumor, a bone tumor, a blood born tumor, a brain/CNS tumor, a breast tumor, a cervical tumor, a colorectal tumor, an endometrial tumor, an esophageal tumor, an Ewing tumor, an eye tumor, a gallbladder tumor, a gastrointestinal, a kidney tumor, a laryngeal or hypopharyngeal tumor, a liver tumor, a lung tumor, a mesothelioma tumor, a multiple myeloma tumor, a muscle tumor, a nasopharyngeal tumor, a neuroblastoma, an oral tumor, an osteosarcoma, an ovarian tumor, a pancreatic tumor, a penile tumor, a pituitary tumor, a primary tumor, a prostate tumor, a retinoblastoma, a Rhabdomyosarcoma, a salivary gland tumor, a soft tissue sarcoma, a melanoma, a metastatic tumor, a basal cell carcinoma, a Merkel cell tumor, a testicular tumor, a thymus tumor, a thyroid tumor, a uterine tumor, a vaginal tumor, a vulvar tumor, or a Wilms tumor.

In certain embodiments, the cancer is Multiple myeloma, Chronic lymphocytic leukemia (CLL), the most common leukemia in adults, lung cancer, prostate cancer, or melanoma.

### EXAMPLES

The following examples are provided to illustrate certain disclosed embodiments and are not to be construed as limiting the scope of this disclosure in any way.

### Example 1. General Methods

### 1.1. Preparation of Lipid Nanoparticles

In general, lipid components were dissolved in 100% ethanol at various molar ratios. The RNA cargos (e.g., Cas9 mRNA and sgRNA) were dissolved in 25 mM citrate buffer, 100 mM NaCl, pH 5.0, resulting in a concentration of RNA cargo of approximately 0.45 mg/mL.

The lipid nucleic acid assemblies contained ionizable Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, 1,2-distearoyl-sn-glycero-3-phosphocholine (DSPC), and 1,2-dimyristoyl-rac-glycero-3-methylpolyoxyethylene glycol 2000 (PEG2k-DMG) in a 50:38:9:3 molar ratio, respectively. The lipid nucleic acid assemblies were formulated with a lipid amine to RNA phosphate (N:P) molar ratio of about 6, and a ratio of gRNA to mRNA of 1:2 by weight, unless otherwise specified.

LNPs were prepared using a cross-flow technique utilizing impinging jet mixing of the lipid in ethanol with two volumes of RNA solutions and one volume of water. The lipids in ethanol were mixed through a mixing cross with the two volumes of RNA solution. A fourth stream of water was mixed with the outlet stream of the cross through an inline tee (See WO2016010840 FIG. 2.). The LNPs were held for 1 hour at room temperature, and further diluted with water (approximately 1:1 v/v). LNPs were concentrated using tangential flow filtration on a flat sheet cartridge (Sartorius, 100 kD MWCO) and buffer exchanged using PD-10 desalting columns (GE) into 50 mM Tris, 45 mM NaCl, 5% (w/v) sucrose, pH 7.5 (TSS). Alternatively, the LNP's were optionally concentrated using 100 kDa Amicon spin filter and buffer exchanged using PD-10 desalting columns (GE) into TSS. The resulting mixture was then filtered using a 0.2 μm sterile filter. The final LNP was stored at 4° C. or −80° C. until further use.

1.2. In Vitro Transcription (“IVT”) of mRNA

Capped and polyadenylated mRNA containing N1-methyl pseudo-U was generated by in vitro transcription using a linearized plasmid DNA template and T7 RNA polymerase. Plasmid DNA containing a T7 promoter, a sequence for transcription, and a polyadenylation region was linearized by incubating at 37° C. for 2 hours with XbaI with the following conditions: 200 ng/μL plasmid, 2 U/μL XbaI (NEB), and 1× reaction buffer. The XbaI was inactivated by heating the reaction at 65° C. for 20 min. The linearized plasmid was purified from enzyme and buffer salts. The IVT reaction to generate modified mRNA was performed by incubating at 37° C. for 1.5-4 hours in the following conditions: 50 ng/μL linearized plasmid; 2-5 mM each of GTP, ATP, CTP, and N1-methyl pseudo-UTP (Trilink); 10-25 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. TURBO DNase (ThermoFisher) was added to a final concentration of 0.01 U/μL, and the reaction was incubated for an additional 30 minutes to remove the DNA template. The mRNA was purified using a MegaClear Transcription Clean-up kit (ThermoFisher) or a RNeasy Maxi kit (Qiagen) per the manufacturers' protocols. Alternatively, the mRNA was purified through a precipitation protocol, which in some cases was followed by HPLC-based purification. Briefly, after the DNase digestion, mRNA is purified using LiCl precipitation, ammonium acetate precipitation and sodium acetate precipitation. For HPLC purified mRNA, after the LiCl precipitation and reconstitution, the mRNA was purified by RP-IP HPLC (see, e.g., Kariko, et al. Nucleic Acids Research, 2011, Vol. 39, No. 21 e142). The fractions chosen for pooling were combined and desalted by sodium acetate/ethanol precipitation as described above. In a further alternative method, mRNA was purified with a LiCl precipitation method followed by further purification by tangential flow filtration. RNA concentrations were determined by measuring the light absorbance at 260 nm (Nanodrop), and transcripts were analyzed by capillary electrophoresis by Bioanalyzer (Agilent).

Streptococcus pyogenes (“Spy”) Cas9 mRNA was generated from plasmid DNA encoding an open reading frame according to SEQ ID NOs: 801-803 (see sequences in Table 9). BC22n mRNA was generated from plasmid DNA encoding an open reading frame according to SEQ ID NO: 804 or 805. UGI mRNA was generated from plasmid DNA encoding an open reading frame according to SEQ ID NOs: 807 or 808. When the sequences cited in this paragraph are referred to below with respect to RNAs, it is understood that Ts should be replaced with Us (which were N1-methyl pseudouridines as described above). Messenger RNAs used in the Examples include a 5′ cap and a 3′ polyadenylation region, e.g., up to 100 nts.

### 1.3. Next-Generation Sequencing (“NGS”) and Analysis for On-Target Editing Efficiency

Genomic DNA was extracted using QuickExtract™ DNA Extraction Solution (Lucigen, Cat. QE09050) according to the manufacturer's protocol. To quantitatively determine the efficiency of editing at the target location in the genome, deep sequencing was utilized to identify the presence of insertions and deletions introduced by gene editing. PCR primers were designed around the target site within the gene of interest (e.g., CD38) and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were re-aligned to the local genome sequence to improve the alignment. Then the number of wild type reads versus the number of reads which contain C-to-T mutations, C-to-A/G mutations or indels was calculated. Insertions and deletions were scored in a 20 bp region centered on the predicted Cas9 cleavage site. Indel percentage is defined as the total number of sequencing reads with one or more base inserted or deleted within the 20 bp scoring region divided by the total number of sequencing reads, including wild type. C-to-T mutations or C-to-A/G mutations were scored in a 40 bp region including 10 bp upstream and 10 bp downstream of the 20 bp sgRNA target sequence. The C-to-T editing percentage is defined as the total number of sequencing reads with either one or more C-to-T mutations within the 40 bp region divided by the total number of sequencing reads, including wild type. The percentage of C-to-A/G mutations are calculated similarly.

### Example 2—CD38 Guide RNA Screening in T Cells with Cas9 and BC22n

### 2.1 T Cell Preparation

T cells were edited at the CD38 locus with either Cas9 or with BC22n and UGI mRNAs to assess the editing outcomes and the corresponding loss of CD38 expression. Guide sequences used and the target regions are listed in Table 1. As shown in Table 1, each sgRNA comprising the guide sequence includes the guide scaffold of SEQ ID NO: 202, and has been modified according to modification pattern of SEQ ID NO: 300.

Healthy human donor apheresis was obtained commercially (Hemacare), and cells were washed and resuspended in CliniMACS PBS/EDTA buffer (Miltenyi Biotec, Cat. No. 130-070-525) on the LOVO device. T cells were isolated via positive selection using CD4 and CD8 magnetic beads (Miltenyi Biotec, Cat. No. 130-030-401/130-030-801) using the CliniMACS Plus and CliniMACS LS disposable kit. T cells were aliquoted into vials and cryopreserved in Cryostor CS10 (StemCell Technologies, Cat. No. 07930) for future use. Upon thaw, T cells were plated at a density of 1.0×10⁶ cells/mL in T cell X-VIVO 15 expansion media composed of X-VIVO 15 (Lonza, Cat. No. BE02-06Q) containing 5% (v/v) of fetal bovine serum (ThermoFisher, Cat. No. A3160902), 50 μM (1×) 2-Mercaptothanol (ThermoFisher, Cat. No. 31350010), 1% of Penicillin-Streptomycin (ThermoFisher, Cat. No. 15140122), 1 M N-acetyl L-cystine (Fisher, Cat. No. ICN19460325) diluted in phosphate buffered saline (PBS) and normalized to pH 7, supplemented with 100 U/mL of recombinant human interleukin-2 (Peprotech, Cat. No. 200-02), 5 ng/mL recombinant human interleukin-7 (Peprotech, Cat. No. 200-07) and 5 ng/mL recombinant human interleukin-15 (Peprotech, Cat. No. 200-15). T cells were activated with TransAct™ (1:100 dilution, Miltenyi Biotec, Cat. No. 130-111-160). Cells were expanded for 72 hours at 37° C. prior to mRNA electroporation.

2.2 T Cell Editing with RNA Electroporation

Solutions containing mRNA encoding Cas9 protein (SEQ ID NO: 801-803), BC22n (SEQ ID NO: 804 or 805), or UGI (SEQ ID NO: 807 or 808) were prepared in sterile water. 50 μM CD38 targeting sgRNAs were removed from their storage plates and denatured for 2 minutes at 95° C. and incubated at room temperature for 5 minutes. Seventy-two hours post activation, T cells were harvested, centrifuged, and resuspended at a concentration of 12.5×10⁶ T cells/mL in P3 electroporation buffer (Lonza). For each well to be electroporated, 1×10⁵ T cells were mixed with 200 ng of Cas9 or BC22n mRNAs, 200 ng of UGI mRNA and 20 pmols of sgRNA as described in Table 2 in a final volume of 20 p L of P3 electroporation buffer. This mix was transferred in duplicate to a 96-well Nucleofector™ plate and electroporated using the manufacturer's pulse code. Electroporated T cells were immediately rested in 80 μL of X-VIVO 15 media without cytokines for 15 minutes before being transferred to new flat-bottom 96-well plates containing an additional 90 μL of X-VIVO 15 media supplemented with 2× cytokines. The resulting plates were incubated at 37° C. for 10 days. To promote expansion, T cells were split at the ratios of 1:4 and 1:3 on days 3 and 6 post-electroporation, respectively, using fresh X-VIVO 15 media with 1× cytokines. On day 9 post-electroporation, cells were split 1:2 in 2 U-bottom plates and one plate was collected for NGS sequencing, while the other plate was used for flow cytometry on Day 10.

### 2.3 Flow Cytometry and NGS Sequencing

On day 10 post-editing, T cells were phenotyped by flow cytometry to determine CD38 receptor expression. Briefly, T cells were incubated for 30 min at 4° C. with a mixture of antibodies against CD3 (BioLegend, Cat. No. 317340), CD4 (BioLegend, Cat. No. 300537), CD8 (BioLegend, Cat. No. 344706) diluted at 1:200 and CD38 (BioLegend, Cat. No. 303546), diluted at 1:100 in cell staining buffer (BioLegend, Cat. No. 420201). Cells were subsequently washed and stained with DAPI (BioLegend, Cat. No. 422801) diluted at 1:10,000 in cell staining buffer. Cells were then processed on a Cytoflex flow cytometer (Beckman Coulter) and analyzed using the FlowJo software package. T cells were gated based on size, shape, viability, and CD38 expression.

On day 9 DNA samples were subjected to PCR and subsequent NGS analysis, as described in Example 1. Table 2 shows CD38 gene editing and CD38 positive results for cells edited with BC22n or Cas9.

TABLE 2
Percent editing and percent of CD38 positive cells following
CD38 editing with Cas9 or BC22n base editor

| | Cas9% Indels | | | Cas9% CD38+ | | | BC22n % C to T | | | BC22% CD38+ | | |
|---|---|---|---|---|---|---|---|---|---|---|---|---|
| Guide ID | Mean | SD | N | Mean | SD | N | Mean | SD | N | Mean | SD | N |
| G019761 | 94.82 | 0.97 | 2 | 22.35 | 0.49 | 2 | 87.21 | 1.57 | 2 | 56.45 | 2.05 | 2 |
| G019762 | 98.19 | 0.63 | 2 | 15.45 | 1.06 | 2 | 85.04 | 0.42 | 2 | 58.55 | 0.07 | 2 |
| G019763 | 91.42 | 1.77 | 2 | 8.17 | 2.38 | 2 | 87.01 | 0.51 | 2 | 2.53 | 0.21 | 2 |
| G019764 | 87.78 | 1.04 | 2 | 11.80 | 0.71 | 2 | 87.00 | 2.33 | 2 | 41.40 | 2.12 | 2 |
| G019765 | 96.43 | 0.40 | 2 | 13.15 | 2.33 | 2 | 81.51 | 5.02 | 2 | 60.40 | 1.27 | 2 |
| G019766 | 97.61 | 0.06 | 2 | 5.31 | 1.15 | 2 | 80.51 | 2.67 | 2 | 61.80 | 0.85 | 2 |
| G019767 | 97.54 | 0.08 | 2 | 17.45 | 0.21 | 2 | 81.92 | 0.46 | 2 | 62.55 | 3.46 | 2 |
| G019768 | 96.65 | 0.96 | 2 | 2.19 | 1.06 | 2 | 89.45 | 0.78 | 2 | 2.49 | 1.18 | 2 |
| G019769 | 95.63 | 2.44 | 2 | 2.58 | 0.35 | 2 | 69.93 | 5.38 | 2 | 14.40 | 4.81 | 2 |
| G019770 | 97.74 | 1.26 | 2 | 1.15 | 0.59 | 2 | 85.06 | 5.81 | 2 | 38.80 | 3.54 | 2 |
| G019771 | 96.90 | 0.54 | 2 | 1.18 | 0.18 | 2 | 87.34 | 0.16 | 2 | 1.39 | 0.26 | 2 |
| G019772 | 96.38 | 0.05 | 2 | 14.50 | 1.98 | 2 | 88.86 | 0.71 | 2 | 70.25 | 1.63 | 2 |
| G019773 | 95.38 | 2.21 | 2 | 6.70 | 0.57 | 2 | 79.83 | 0.15 | 2 | 64.15 | 4.45 | 2 |
| G019774 | 97.12 | 0.07 | 2 | 14.55 | 0.92 | 2 | 71.14 | 2.52 | 2 | 68.60 | 2.83 | 2 |
| G019775 | 95.33 | 3.04 | 2 | 16.75 | 1.63 | 2 | 92.02 | 0.11 | 2 | 74.90 | 0.28 | 2 |
| G019776 | 98.87 | 0.45 | 2 | 1.95 | 1.04 | 2 | 84.25 | 0.57 | 2 | 11.45 | 1.63 | 2 |
| G019777 | 97.87 | 0.54 | 2 | 14.00 | 1.27 | 2 | 86.76 | 1.32 | 2 | 71.75 | 2.62 | 2 |
| G019778 | 95.81 | 1.24 | 2 | 13.75 | 1.48 | 2 | 86.96 | 1.81 | 2 | 62.30 | 2.26 | 2 |
| G019779 | 97.78 | 0.26 | 2 | 10.87 | 2.17 | 2 | 88.77 | 0.53 | 2 | 74.35 | 0.07 | 2 |
| G019780 | 95.75 | 1.84 | 2 | 14.00 | 2.55 | 2 | 89.81 | 0.30 | 2 | 69.05 | 3.18 | 2 |
| G019781 | 93.81 | 2.35 | 2 | 15.90 | 0.85 | 2 | 86.84 | 4.62 | 2 | 68.30 | 0.71 | 2 |
| G019782 | 93.10 | 4.65 | 2 | 10.21 | 2.82 | 2 | 82.19 | 1.92 | 2 | 63.15 | 0.49 | 2 |
| G019783 | 97.88 | 0.30 | 2 | 2.58 | 0.40 | 2 | 77.26 | 4.30 | 2 | 64.10 | 0.14 | 2 |
| G019784 | 97.61 | 0.86 | 2 | 16.80 | 2.69 | 2 | 76.82 | 0.81 | 2 | 69.00 | 0.71 | 2 |
| G019785 | 98.30 | 0.27 | 2 | 1.58 | 0.66 | 2 | 80.95 | 1.14 | 2 | 33.90 | 5.80 | 2 |
| G019786 | 86.22 | 3.34 | 2 | 28.10 | 4.67 | 2 | 88.42 | 2.78 | 2 | 72.35 | 5.02 | 2 |
| G019787 | 98.76 | 0.45 | 2 | 2.06 | 0.86 | 2 | 80.23 | 2.14 | 2 | 1.89 | 0.83 | 2 |
| G019788 | 96.34 | 1.98 | 2 | 11.05 | 0.92 | 2 | 90.88 | 0.06 | 2 | 1.88 | 0.21 | 2 |
| G019789 | 97.68 | 1.33 | 2 | 15.80 | 1.27 | 2 | 67.12 | n.a. | 1 | 52.85 | 1.34 | 2 |
| G019790 | 94.58 | 1.87 | 2 | 12.15 | 0.07 | 2 | 89.81 | 0.22 | 2 | 77.10 | 2.69 | 2 |
| G019791 | 97.79 | 0.90 | 2 | 3.25 | 0.86 | 2 | 79.56 | 3.97 | 2 | 9.51 | 0.62 | 2 |
| G019792 | 96.99 | 0.13 | 2 | 9.19 | 0.11 | 2 | 85.20 | 4.14 | 2 | 64.65 | 0.35 | 2 |
| G019793 | 96.13 | 0.29 | 2 | 19.95 | 2.33 | 2 | 85.55 | 2.40 | 2 | 71.40 | 2.69 | 2 |
| G019794 | 98.30 | 0.28 | 2 | 12.75 | 0.07 | 2 | 76.28 | 0.67 | 2 | 31.40 | 0.85 | 2 |
| G019795 | 97.56 | 0.59 | 2 | 2.52 | 0.56 | 2 | 91.40 | 2.37 | 2 | 1.22 | 0.34 | 2 |
| G019796 | 97.70 | 1.27 | 2 | 7.60 | 2.87 | 2 | 92.29 | 0.28 | 2 | 6.52 | 0.16 | 2 |
| G019797 | 97.04 | 0.26 | 2 | 9.80 | 2.26 | 2 | 87.45 | 0.46 | 2 | 3.07 | 0.84 | 2 |
| G019798 | 97.56 | 1.17 | 2 | 4.92 | 0.93 | 2 | 89.68 | 0.89 | 2 | 59.05 | 1.34 | 2 |
| G019799 | 97.65 | 0.22 | 2 | 8.83 | 1.46 | 2 | 87.59 | 0.90 | 2 | 66.85 | 0.49 | 2 |
| G019800 | 76.71 | 3.24 | 2 | 25.45 | 4.88 | 2 | 74.46 | 2.84 | 2 | 16.00 | 3.54 | 2 |
| G019801 | 92.76 | 3.74 | 2 | 6.10 | 2.28 | 2 | 82.69 | 0.52 | 2 | 33.65 | 4.03 | 2 |
| G019802 | 64.21 | 7.62 | 2 | 33.70 | 5.52 | 2 | 69.14 | 0.45 | 2 | 73.00 | 0.42 | 2 |
| G019803 | 50.46 | 6.67 | 2 | 43.50 | 5.23 | 2 | 45.01 | 1.82 | 2 | 55.40 | 1.98 | 2 |
| G019804 | 89.70 | 3.71 | 2 | 7.05 | 3.23 | 2 | 21.88 | 0.42 | 2 | 67.25 | 2.76 | 2 |
| G019805 | 52.39 | 2.21 | 2 | 40.30 | 0.14 | 2 | 78.69 | 4.52 | 2 | 9.15 | 1.77 | 2 |
| G019806 | 93.47 | 0.40 | 2 | 4.64 | 0.82 | 2 | 72.68 | 1.54 | 2 | 21.30 | 4.95 | 2 |
| G019807 | 86.45 | 2.17 | 2 | 12.70 | 1.84 | 2 | 63.32 | 3.90 | 2 | 65.85 | 0.21 | 2 |
| G019808 | 97.15 | 0.70 | 2 | 2.23 | 1.37 | 2 | 32.87 | 6.70 | 2 | 64.10 | 1.84 | 2 |
| G019809 | 41.04 | 6.38 | 2 | 56.35 | 4.31 | 2 | 68.30 | 3.73 | 2 | 27.50 | 5.94 | 2 |
| G019810 | 46.14 | 4.29 | 2 | 52.05 | 5.16 | 2 | 78.63 | 2.98 | 2 | 10.40 | 2.97 | 2 |
| G019811 | 95.15 | 0.67 | 2 | 14.05 | 1.06 | 2 | 83.21 | 2.11 | 2 | 4.42 | 2.68 | 2 |
| G019812 | 93.95 | 1.06 | 2 | 4.46 | 2.11 | 2 | 84.23 | 5.13 | 2 | 24.90 | 2.55 | 2 |
| G019813 | 96.79 | 1.07 | 2 | 2.51 | 0.60 | 2 | 75.68 | 3.13 | 2 | 5.51 | 1.97 | 2 |
| G019814 | 87.26 | 0.64 | 2 | 11.56 | 2.75 | 2 | 72.31 | 1.65 | 2 | 13.80 | 2.83 | 2 |
| G019815 | 77.53 | 7.29 | 2 | 18.65 | 3.89 | 2 | 86.69 | 0.87 | 2 | 8.47 | 0.74 | 2 |
| G019816 | 76.89 | 1.37 | 2 | 38.25 | 6.86 | 2 | 87.17 | 2.00 | 2 | 8.82 | 2.94 | 2 |
| G019817 | 90.53 | 1.09 | 2 | 5.95 | 1.39 | 2 | 72.59 | 1.92 | 2 | 33.85 | 1.34 | 2 |
| G019818 | 97.71 | 0.21 | 2 | 1.69 | 0.57 | 2 | 77.99 | 3.46 | 2 | 14.15 | 3.75 | 2 |
| G019819 | 94.33 | 1.87 | 2 | 3.70 | 2.29 | 2 | 84.14 | 3.95 | 2 | 2.47 | 0.45 | 2 |
| G019820 | 82.74 | 4.09 | 2 | 21.00 | 3.39 | 2 | 81.28 | 0.66 | 2 | 14.80 | 2.12 | 2 |
| G019821 | 70.98 | 8.64 | 2 | 26.80 | 8.49 | 2 | 73.39 | 3.46 | 2 | 11.09 | 3.27 | 2 |
| G019822 | 46.59 | 2.28 | 2 | 50.80 | 5.09 | 2 | 58.43 | 2.69 | 2 | 73.55 | 0.21 | 2 |
| G019823 | 93.48 | 2.48 | 2 | 4.04 | 1.94 | 2 | 79.65 | 0.80 | 2 | 24.45 | 6.01 | 2 |
| G019824 | 95.90 | 0.76 | 2 | 6.32 | 1.92 | 2 | 89.11 | 1.03 | 2 | 74.75 | 3.18 | 2 |
| G019825 | 29.68 | 7.06 | 2 | 56.10 | 0.57 | 2 | 39.49 | 6.14 | 2 | 66.20 | 3.54 | 2 |
| G019826 | 92.62 | 1.03 | 2 | 3.74 | 1.05 | 2 | 69.37 | 5.37 | 2 | 61.70 | 0.42 | 2 |
| G019827 | 39.32 | 4.26 | 2 | 54.80 | 2.55 | 2 | 48.52 | 0.86 | 2 | 38.15 | 1.20 | 2 |
| G019828 | 87.90 | 3.26 | 2 | 9.98 | 1.16 | 2 | 80.25 | 0.57 | 2 | 7.40 | 2.33 | 2 |
| G019829 | 31.95 | 2.81 | 2 | 63.40 | 1.98 | 2 | 76.10 | 2.57 | 2 | 14.50 | 2.69 | 2 |
| G019830 | 81.01 | 6.21 | 2 | 16.45 | 3.32 | 2 | 83.57 | 1.78 | 2 | 4.29 | 1.12 | 2 |
| G019831 | 98.29 | 0.30 | 2 | 0.91 | 0.25 | 2 | 71.43 | 0.65 | 2 | 2.05 | 0.84 | 2 |
| G019832 | 62.85 | 1.73 | 2 | 34.70 | 3.54 | 2 | 90.87 | 2.45 | 2 | 71.05 | 6.01 | 2 |
| G019833 | 92.66 | 0.94 | 2 | 4.28 | 0.82 | 2 | 75.96 | 0.45 | 2 | 18.40 | 2.26 | 2 |
| G019834 | 94.43 | 0.37 | 2 | 2.75 | 0.76 | 2 | 76.23 | 0.91 | 2 | 69.00 | 2.83 | 2 |
| G019835 | 42.83 | n.a. | 1 | 49.95 | 5.73 | 2 | 51.27 | 8.38 | 2 | 43.95 | 6.72 | 2 |
| G019836 | No data | | | 46.05 | 5.02 | 2 | 31.44 | 6.72 | 2 | 75.10 | 0.00 | 2 |
| G019837 | 43.86 | 6.82 | 2 | 49.85 | 5.44 | 2 | 63.03 | 1.17 | 2 | 51.50 | 1.70 | 2 |
| G019838 | 75.85 | 0.40 | 2 | 24.25 | 0.64 | 2 | 74.87 | 1.14 | 2 | 19.05 | 2.62 | 2 |
| G019839 | 97.44 | n.a. | 1 | 1.82 | 0.21 | 2 | 69.11 | 4.60 | 2 | 40.55 | 2.05 | 2 |
| G019840 | 59.58 | 4.30 | 2 | 37.90 | 0.85 | 2 | 47.06 | 4.04 | 2 | 73.05 | 1.06 | 2 |
| G019841 | 96.14 | n.a. | 1 | 3.60 | 1.68 | 2 | 69.28 | 2.55 | 2 | 27.15 | 2.33 | 2 |
| G019842 | 33.72 | 6.77 | 2 | 58.65 | 2.05 | 2 | 29.32 | 2.31 | 2 | 71.50 | 3.82 | 2 |
| G019843 | 6.02 | 0.04 | 2 | 73.15 | 0.49 | 2 | 20.85 | 1.36 | 2 | 70.75 | 0.35 | 2 |
| G019844 | 85.26 | 12.45 | 2 | 6.30 | 0.47 | 2 | 76.89 | 3.63 | 2 | 39.45 | 3.75 | 2 |
| G019845 | 36.76 | 2.44 | 2 | 55.25 | 3.18 | 2 | 62.90 | 1.02 | 2 | 60.15 | 2.62 | 2 |
| G019846 | 25.85 | 4.33 | 2 | 64.80 | 2.69 | 2 | 57.55 | 1.80 | 2 | 63.95 | 4.88 | 2 |
| G019847 | 58.00 | 5.06 | 2 | 37.85 | 2.19 | 2 | 71.58 | 0.74 | 2 | 42.55 | 5.16 | 2 |
| G019848 | 47.39 | 6.18 | 2 | 46.90 | 5.09 | 2 | 71.78 | 4.09 | 2 | 67.15 | 3.32 | 2 |

“n.a.” indicates standard deviation could not be calculated; “no data” indicates runs were not successful.

### Example 3. Off-Target Analysis

### 3.1 Biochemical Off-Target Analysis

A biochemical method (See, e.g., Cameron et al., Nature Methods. 6, 600-606; 2017) was used to determine potential off-target genomic sites cleaved by Cas9 using specific guides targeting CD38. Twenty-one sgRNAs targeting human CD38 (shown in both Tables 3 and 4) were screened using NA24385 genomic DNA (Coriell Institute) alongside three control guides with known off-target profiles. The number of on target and potential off-target cleavage sites were detected using a guide concentration of 192 nM gRNA and 64 nM Cas9 protein in the biochemical assay for which results are shown in Tables 3 and 4.

TABLE 3
Biochemical Off-Target Analysis

| Guide ID | Target Gene | Sites |
|---|---|---|
| G019768 | CD38 | 10 |
| G019769 | CD38 | 54 |
| G019770 | CD38 | 53 |
| G019771 | CD38 | 6 |
| G019776 | CD38 | 24 |
| G019783 | CD38 | 134 |
| G019785 | CD38 | 11 |
| G019787 | CD38 | 50 |
| G019791 | CD38 | 98 |
| G019795 | CD38 | 60 |
| G019808 | CD38 | 127 |
| G019813 | CD38 | 124 |
| G019818 | CD38 | 208 |
| G019819 | CD38 | 154 |
| G019831 | CD38 | 318 |
| G019834 | CD38 | 45 |
| G019839 | CD38 | 308 |
| G019841 | CD38 | 182 |
| G000644 | EMX1 | 393 |
| G000645 | VEGFA | 4613 |
| G000646 | RAG1B | 70 |

TABLE 4
Biochemical Off-Target Analysis

| Guide ID | Target Gene | Sites |
|---|---|---|
| G019763 | CD38 | 4 |
| G019788 | CD38 | 124 |
| G019797 | CD38 | 205 |
| G000644 | EMX1 | 276 |
| G000645 | VEGFA | 3259 |
| G000646 | RAG1B | 32 |

### 3.2 Targeted Sequencing for Validating Potential Off-Target Sites

Potential off-target sites predicted by detection assays such as the biochemical method used above, may be assessed using targeted sequencing of the identified potential off-target sites to determine whether off-target cleavage at that site is detected.

In one approach, Cas9 and a sgRNA of interest (e.g., a sgRNA having potential off-target sites for evaluation) are introduced to primary T cells. The T cells are then lysed and primers flanking the potential off-target site(s) are used to generate an amplicon for NGS analysis. Identification of indels at a certain level may validate a potential off-target site, whereas the lack of indels found at the potential off-target site may indicate a false positive from the off-target predictive assay that was utilized.

G019771 was further evaluated for possible off-target indel formation using amplicon sequencing at potential off target sites following editing in cells. Potential off target sites were identified by the biochemical assay described above or by in silico prediction.

Samples were prepared in triplicate. T cells were prepared as described in Example 6. Cells were treated simultaneously with 3 LNPs, each formulated with a single RNA cargo of SpyCas9 mRNA, UGI mRNA, or G019771. LNPs were generally prepared as described in Example 1 with lipid molar ratio of 50 Lipid A:38.5 cholesterol:10 DPSC:1.5 PEG. LNPs were pre-incubated in 20 μg/ml of human ApoE3. Approximately 50,000 cells were treated with LNPs measured by RNA weight as follows: 334 ug Cas9 mRNA, 334 ug G019771, 100 ug UGI mRNA. Cells were incubated at 37C for 24 hours then resuspended in fresh media for further growth. Approximately 72 hours after LNP treat, cells were harvested and NGS analysis was performed generally as described in Example 1 or via rhAmpSeq CRISPR Analysis System (IDT) by the manufacturer's protocol using primers designed to identify percent indels at predicted off-target sites. Repair structures were manually inspected at loci with statistically relevant indel rates at the off-target cleavage sites to confirm indel repair structures. Of the 37 potential off target sites examined, one site, which was in an intergenic region, showed less than 1% indels with statistical significance compared to the untreated control. No other sites examined showed statistically significant editing compared to untreated controls.

### Example 4. Dose-Dependent Editing in NK Cells

Natural killer (NK) cells were edited using 2 guides at increasing concentrations. NK cells were isolated from a commercially obtained leukopak using the EasySep Human NK Cell Isolation Kit (STEMCELL, Cat. No. 17955) according to the manufacturers protocol. Following isolation, human primary NK cells were cryopreserved. Upon thaw, cells were cultured in RPMI 1640 media with 10% fetal bovine serum (FBS), 100 U/mL interleukin-2 (IL-2), and 1% Pen-Strep overnight. NK cells were activated by culturing cells 1:1 with irradiated K562 4-1BBL cells in RPMI 1640 media with 10% FBS and 1% Pen-Strep for three days.

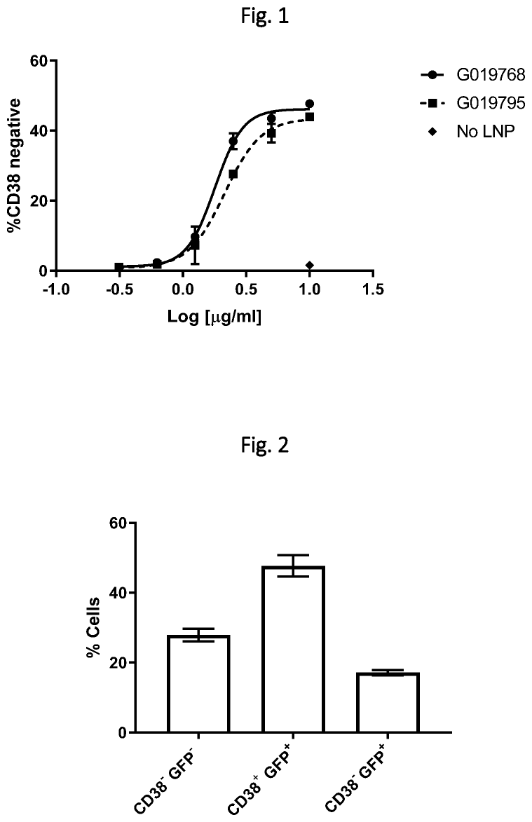

NK cells were treated with LNPs delivering Cas9 mRNA (SEQ ID NO: 802) and a gRNA (G019768 or G019795) as indicated in Table 5 targeting CD38. LNPs were generally prepared as described in Example 1 with the lipid composition with the molar ratio of 50 ionizable lipid A/38.5 cholesterol/10 DSPC/1.5 PEG. The LNPs were formulated with a lipid amine to RNA phosphate (N:P) molar ratio of about 6 and a ratio of gRNA to mRNA of 1:2 by weight. LNPs were preincubated at 37° C. for with 1 μg/ml recombinant human ApoE3 (Peprotech, 350-02) for 15 minutes in RPMI media. The pre-incubated LNPs were added to NK cells in duplicate at total RNA cargo concentrations indicated in Table 5. At 12 days post LNP treatment, cells were assayed by flow cytometry to measure rates of CD38 surface expression. Briefly, NK cells were incubated with antibodies targeting CD3 (Biolegend, Cat. No. 317344), CD56 (Biolegend, Cat. No. 362518) and CD38 (Biolegend, Cat. No. 303510). Cells were subsequently washed, processed on a Cytoflex instrument (Beckman Coulter) and analyzed using the FlowJo software package. NK cells were gated on size and CD3/CD56 status. Table 5 and FIG. 1 show percent of NK cells without CD38 surface expression.

TABLE 5
Mean % CD38 negative cells in edited NK cells

| | | |
|---|---|---|
| LNP | G019768 | G019795 |

| (ug/ml) | Mean | SD | Mean | SD |
|---|---|---|---|---|
| 10.00 | 47.70 | 1.13 | 43.95 | 0.35 |
| 5.00 | 43.55 | 1.63 | 39.30 | 2.69 |
| 2.50 | 37.00 | 2.26 | 27.65 | 0.21 |
| 1.25 | 9.74 | 0.51 | 7.30 | 5.38 |
| 0.63 | 2.36 | 0.63 | 1.88 | 0.18 |
| 0.31 | 0.97 | 0.10 | 1.09 | 0.10 |
| No guide | 1.55 | 0.64 | | |

### Example 5. Multi-Editing with CD38 Disruption and AAVS1 Insertion

Natural killer (NK) cells were sequentially edited to first disrupt CD38 and second insert GFP into the AAVS1 locus. NK cells were isolated from buffy coat using the EasySep Human NK Cell Isolation Kit (STEMCELL, Cat. No. 17955) according to the manufacturers protocol. Following isolation, human primary NK cells were cryopreserved. Upon thaw, human primary NK cells were cultured at 1×106 cells/ml in CTS OpTmizer media (Gibco, A10221-01) containing 5% FBS and 1% Pen-Strep (CTS OpTmizer Complete media) with 500 U/ml IL-2 overnight. NK cells were activated by culturing cells 1:1 with irradiated K562 4-1BBL cells in CTS OpTmizer Complete media for 1 day. Cells were washed and plated in CTS OpTmizer media containing 500 U/ml IL-2 and 5 ng/ml IL-15 at 0.5×106 cells/ml.

NK cells were treated with LNPs delivering Cas9 mRNA (SEQ ID NO: 802) and gRNA G019768 targeting CD38. LNP was generally prepared as described in Example 1 with the lipid with the molar ratio of 50 ionizable lipid A/38.5 cholesterol/10 DSPC/1.5 PEG. LNPs were preincubated at 37° C. for with 5 μg/ml recombinant human ApoE3 (Peprotech, 350-02) for 15 minutes in CTS OpTmizer complete media containing 2.5% human AB serum (GemCell, 100-512). The pre-incubated LNPs were added to NK cells in duplicate at 5 μg/ml total RNA cargo.

One day after CD38 LNP exposure, cells were treated with LNPs and AAV6 for insertion of GFP at the AAVS1 locus. LNP was generally prepared as Example 1 with the lipid composition with the molar ratio of 50 ionizable lipid A/38.5 cholesterol/10 DSPC/1.5 PEG. The LNPs were formulated with a lipid amine to RNA phosphate (N:P) molar ratio of about 6, and a ratio of gRNA to mRNA of 1:2 by weight. LNPs were preincubated with 10 μg/ml APOE3 at 37° C. for about 15 minutes in OpTmizer media with 2.5% human AB serum, 500 U/mL IL-2 and 5 ng/ml IL-15. The pre-incubated LNPs were added to NK cells in duplicate at 10 μg/ml total RNA cargo. Following editing, AAV6 vector encoding a GFP gene driven by its own promoter and flanked homology arms to the AAVS1 sequence (SEQ ID 1001) was added to cells at a multiplicity of infection (MOI) of 600,000 genome copies was added following editing. Cells were incubated for 6 days.

Eight days after activation, cells were assayed by flow cytometry to measure rates of CD38 surface expression and GFP expression. Briefly, NK cells were incubated with antibodies targeting CD3 (Biolegend, Cat. No. 317344), CD56 (Biolegend, Cat. No. 362518), CD38 (Biolegend, Cat. No. 303510) and DAPI. Cells were subsequently washed, processed on a Cytoflex instrument (Beckman Coulter) and analyzed using the FlowJo software package. NK cells were gated on size and CD3/CD56 status. Table 6 and FIG. 2 show percent of NK cells without CD38 surface expression and with GFP expression. Sequential gene disruption and sequence insertion edits were achieved in NK cells using LNPs.

TABLE 6
Percentage of NK cells with expression
phenotype following editing.

| Expression | Mean | SD | n |
|---|---|---|---|
| CD38− GFP− | 27.90 | 1.75 | 3 |
| CD38+ GFP+ | 47.70 | 3.08 | 3 |
| CD38− GFP+ | 17.13 | 0.74 | 3 |

For each crRNA, the indicated 20 nt guide sequence is included within an N20GUUUUAGAGCUAUGCUGUUUUG nucleic acid sequence, where “N20” represents the guide sequence.

Initial guide selection was performed in silico using a human reference genome (e.g., hg38) and user defined genomic regions of interest (e.g., CD38), for identifying PAMs in the regions of interest. For each identified PAM, analyses were performed and statistics reported. gRNA molecules were further selected and rank-ordered based on a number of criteria known in the art (e.g., GC content, predicted on-target activity, and potential off-target activity).

### Example 6. Knockout of CD38 to Prevent Engineered Cell Self-Activation and Fratricide

Healthy human donor T cells were engineered with a targeting receptor that targets the engineered cell to CD38 with or without the disruption of CD38. Following T cell expansion, the engineered cells were characterized for self-activation and fratricide.

### Example 6.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted into vials and cryopreserved in Cryostor® CS10 (StemCell Technologies Cat. No. 07930) for future use.

Upon thaw, T cells were plated at a density of 1.0×10\hat{ }6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were plated for editing by lipid nanoparticles.

### Example 6.2 Multi-Editing T Cells to Target CD38 with Sequential LNP Delivery

T cells were engineered with a series of gene disruptions and insertions. Healthy donor T cells were treated sequentially with up to 3 LNPs, each LNP co-formulated with mRNA encoding Cas9 and a sgRNA targeting either TRBC (G016239) and TRAC (G013006) with or without CD38 (G019771). A transgenic receptor to target the engineered cell to a CD38-expressing cell was integrated into the TRAC cut site by delivering a homology-directed repair template using an adeno-associated virus (AAV).

### Example 6.3. LNP Treatments and Expansion of T Cells

Before each LNP treatment, T cells were centrifuged at 500 g for 5 min and resuspended in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15).

LNPs were generally prepared as described in Example 1. LNPs with TRAC or TRBC gRNAs used a ratio of 50/38.5/10/1.5 Lipid A, cholesterol, DSPC, and PEG2k-DMG. LNPS with CD38 gRNA used a ratio of 50/38/9/3 Lipid A, cholesterol, DSPC, and PEG2k-DMG. LNPs were prepared with a ratio of gRNA to mRNA of 1:2 by weight. LNPs were prepared each day in T cell treatment media (TC™): a version of TCGM containing 20 μg/mL rhApoE3 in the absence of interleukins 2, 5 or 7. LNPs were incubated at 37° C. for 15 minutes and delivered to T cells in a 1:1 ratio by volume.

On day 1, LNPs with Cas9 mRNA and TRBC sgRNA were incubated at a concentration of 5 μg/mL in TC™ containing 20 μg/mL rhApoE3 (Peprotech, Cat. 350-02). Meanwhile, T cells were harvested, washed, and resuspended at a density of 2×10⁶ cells/mL in TCPM with a 1:50 dilution of T Cell TransAct human reagent (Miltenyi, Cat. 130-111-160). T cells and LNP-media were mixed at a 1:1 ratio and T cells plated in a culture flask until day 3.

On day 3, LNPs with Cas9 mRNA and TRAC sgRNA were incubated at a concentration of 5 μg/mL in TC™ containing 20 μg/mL rhApoE3 (Peprotech, Cat. 350-02) and 1 μM of DNA protein kinase inhibitor. Meanwhile, T cells were washed, and resuspended at a density of 1×10⁶ cells/mL in TCPM. T cells and LNP-media were mixed in a culture flask at a 1:1 ratio by volume. Adeno-associated viruses (AAVs) carrying a homology-directed repair template encoding a targeting receptor were added to T cells at a MOI of 3×10⁵ genome copies/cell. T cells were cultured until day 4.

On day 4, two separate treatments were performed. For group 1, LNPs with Cas9 mRNA and CD38 sgRNA were incubated at a concentration of 5 μg/mL in TC™ containing 20 μg/mL rhApoE3 (Peprotech, Cat. 350-02). Meanwhile, T cells were washed, and resuspended at a density of 1×10⁶ cells/mL in TCPM. T cells and LNP-media were mixed in a culture flask at a 1:1 ratio by volume. For group 2, T cells were washed, and resuspended at a density of 1×10⁶ cells/mL in TCPM. T cells were mixed 1:1 with TC™ containing 20 μg/mL rhApoE3 but no LNP.

On day 5, T cells were washed and transferred to a 6M-well GREX plate (Wilson Wolf, Cat. 80660M) in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were expanded for 9 days without media exchanges according to the manufacturer's protocols. T cells were harvested, evaluated by flow cytometry, and cryopreserved in Cryostor® CS10 (StemCell Technologies Cat. No. 07930).

### Example 6.4. Assessments of T Cell Editing by Flow Cytometry

Post expansion, edited T cells were assayed by flow cytometry to evaluate loss of CD38 expression. T cells were incubated with an antibody cocktail targeting the following molecules: CD4 (Biolegend, Cat. 317434), CD8 (Biolegend, Cat. 301046), CD3 (Biolegend, Cat. 317336), and CD38 (Biolegend, Cat. 303516). Cells were subsequently washed, analyzed on a Cytoflex LX instrument (Beckman Coulter) using the FlowJo software package. T cells were gated on size and CD4/CD8 status before expression of any markers was determined. Loss of CD38 expression was confirmed in T cells edited with CD38 LNPs.

### Example 6.5. Evaluation of T Cell Activation by Flow Cytometry

Engineered T cells (CD38+/−) expressing the targeting receptor described herein were co-cultured with target multiple myeloma (MM1.S) cells that express high levels of CD38 at an effector-to-target ratio of 1:2. To evaluate the occurrence of self-activation or fratricide, CD38+/− targeting receptor-expressing T cells were cultured in the absence of target MM1.S cells. Co-cultures were performed in a cytokine-free media composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, and 10 mM HEPES.

After 24 hours, co-cultures were assayed by flow cytometry to evaluate activation of CD8+ effector T cells. For this purpose, cells were incubated with an antibody cocktail targeting the following molecules: CD4 (Biolegend, Cat. 317434), CD8 (Biolegend, Cat. 301046), CD38 (Biolegend, Cat. 303516), CD25 (Biolegend, Cat. 302632), and CD69 CD25 (Biolegend, Cat. 310906). Cells were subsequently washed, analyzed on a Cytoflex LX instrument (Beckman Coulter) using the FlowJo software package. T cells were gated on size and CD4/CD8 status before expression of any markers was determined.

In the presence of MM1.S target cells, both CD38+ and CD38− effector T cells showed robust expression of activation markers CD25 and CD69. More importantly, in the absence of MM1.S target cells, CD38+ effector T cells showed a detectable expression of activation markers, while CD38− effector T cells did not express activation markers above background levels.

### Example 6.6. Luciferase-Based Cytotoxicity Analysis of CD38 KO Effector T Cells

Engineered targeting receptor-expressing T cells with and without disruption of CD38 were co-cultured at an effector-to-target ratio of 1:2 with luciferized multiple myeloma (MM1.S) cells that express high levels of CD38. Co-cultures were performed in a cytokine-free media composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, and 10 mM HEPES.

After 48 hours, the amount of luciferase enzyme produced by live MM1.S cells, which is inversely proportional to engineered T cytotoxicity, was measured by the Bright-Glo assay (Promega Cat. E2620) following the manufacturer's instructions. Luminescence was measured using a Synergy Neo2 Hybrid Multi-Mode Reader (BioTek Instruments).

While both CD38+ and CD38− T cells expressing the targeting receptor showed a cytotoxic response against target MM1.S cells, CD38− T cells showed greater cytotoxicity than CD38+ T cells.

### Example 6.7. Secretion of Pro-Inflammatory Biomarkers by CD38+/− T Cells

To evaluate the occurrence of self-activation or fratricide, targeting receptor-expressing T cells with and without disruption of CD38 were cultured in the absence of target MM1.S cells. Cultures were performed in a cytokine-free media composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, and 10 mM HEPES.

After 24 hours, cultures were centrifuged at 500 g for 5 min and 100 μL of supernatant was collected to assess the levels of pro-inflammatory analytes secreted by CD38+ and CD38− T cells in the absence of target MM1.S cells. The concentration of interleukin-2 (IL2), interferon-γ (IFNG), tumor necrosis factor-α (TNF), granulocyte-macrophage colony-stimulating factor (GM-CSF) and granzyme A (GZMA) was measured using a multiplex immunoassay from Meso Scale Discovery (Cat. K15338K-2) following the manufacturer's protocol and using a MESO QuickPlex SQ 120 instrument.

In the absence of MM1.S target cells, CD38+ T cells showed secreted detectable levels of pro-inflammatory biomarkers, indicating self-activation or fratricide. In comparison, CD38− T cells did not secrete pro-inflammatory biomarkers above background levels, indicating a lack of fratricide.

### Example 7 Editing Human T Cells with BC22n, UGI and 91-Mer sgRNAs

The base editing efficacy of 91-mer sgRNAs as assessed by NGS and/or receptor knockout was compared to that of 100-mer sgRNA controls with the same guides sequences.

### Example 7.1. T Cell Preparation

Healthy human donor apheresis was obtained commercially (Hemacare), and cells were washed, re-suspended in CliniMACS® PBS/EDTA buffer (Miltenyi Biotec Cat. 130-070-525) and processed in a MultiMACS™ Cell 24 Separator Plus device (Miltenyi Biotec). T cells were isolated via positive selection using a Straight from Leukopak® CD4/CD8 MicroBead kit, human (Miltenyi Biotec Cat. 130-122-352). T cells were aliquoted and cryopreserved for future use in Cryostor® CS10 (StemCell Technologies Cat. 07930).

Upon thaw, T cells were plated at a density of 1.0×10^{A}6 cells/mL in T cell growth media (TCGM) composed of CTS OpTmizer T Cell Expansion SFM and T Cell Expansion Supplement (ThermoFisher Cat. A1048501), 5% human AB serum (GeminiBio, Cat. 100-512) 1× Penicillin-Streptomycin, 1× Glutamax, 10 mM HEPES, 200 U/mL recombinant human interleukin-2 (Peprotech, Cat. 200-02), 5 ng/ml recombinant human interleukin 7 (Peprotech, Cat. 200-07), and 5 ng/ml recombinant human interleukin 15 (Peprotech, Cat. 200-15). T cells were rested in this media for 24 hours, at which time they were activated with T Cell TransAct™, human reagent (Miltenyi, Cat. 130-111-160) added at a 1:100 ratio by volume. T cells were activated for 48 hours prior to LNP treatments.

### Example 7.2. T Cell LNP Treatment and Expansion

Forty-eight hours post-activation, T cells were harvested, centrifuged at 500 g for 5 min, and resuspended at a concentration of 1×10\hat{ }6 T cells/mL in T cell plating media (TCPM): a serum-free version of TCGM containing 400 U/mL recombinant human interleukin-2 (Peprotech, Cat. 200-02), 10 ng/ml recombinant human interleukin 7 (Peprotech, Cat. 200-07), and 10 ng/ml recombinant human interleukin 15 (Peprotech, Cat. 200-15). 50 μL of T cells in TCPM (5×10\hat{ }4 T cells) were added per well to be treated in flat-bottom 96-well plates.

LNPs were prepared as described in Example 1 at a ratio of 35/15/47.5/2.5 (Lipid A/cholesterol/DSPC/PEG2k-DMG). The LNPs were formulated with a lipid amine to RNA phosphate (N:P) molar ratio of about 6. LNPs encapsulated a single RNA species: G019771, G023522, BC22n mRNA, or UGI mRNA.

Prior to T cell treatment, LNPs encapsulating a sgRNA were diluted to 6.64 μg/mL in T cell treatment media (TCTM): a version of TCGM containing 20 ug/mL rhApoE3 in the absence of interleukins 2, 5 or 7. These LNPs were incubated at 37° C. for 15 minutes and serially diluted 1:4 using TC™, which resulted in an 8-point dilution series ranging from 6.64 μg/mL to zero. Similarly, single-cargo LNPs with BC22n mRNA or UGI mRNA were diluted in TCTM to 3.32 and 1.67 μg/mL, respectively, incubated at 37° C. for 15 minutes, and mixed 1:1 by volume with sgRNA LNPs serially diluted in the previous step. Last, 50 μL from the resulting mix was added to T cells in 96-well plates at a 1:1 ratio by volume. T cells were incubated at 37° C. for 24 hours, at which time they were harvested, centrifuged at 500 g for 5 min, resuspended in 200 μL of TCGM and returned to the incubator.

### Example 7.3. Evaluation of Editing Outcomes by Next Generation Sequencing (NGS)

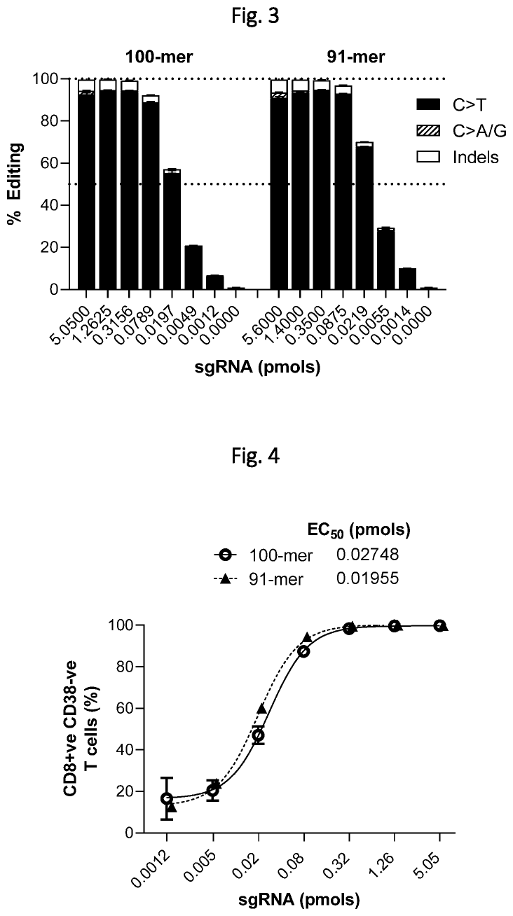

Four days post-LNP treatment, T cells were subjected to lysis, PCR amplification of each targeted locus and subsequent NGS analysis, as described in Example 1. Table 7 and FIG. 3 show editing levels and the C to T editing purity in T cells treated with a decreasing mass of 100-mer or 91-mer sgRNAs targeting CD38.

When compared to their 100-mer versions, 91-mer sgRNAs resulted in higher editing frequencies when delivered at the same concentration. C to T editing purity was observed to be similar between 100-mer and 91-mer sgRNAs.

TABLE 7
Mean percent editing at the CD38 locus in T cells treated with
sgRNAs in the 100-mer (G019771) or 91-mer format (G023522).

| | CD38 | | | | | | | | | | | |
|---|---|---|---|---|---|---|---|---|---|---|---|---|
| | 100-mer | | | | | | 91-mer | | | | | |
| sgRNA | C to T | | C to A/G | | Indels | | C to T | | C to A/G | | Indels | |
| (ng) | Mean | SD | Mean | SD | Mean | SD | Mean | SD | Mean | SD | Mean | SD |
| 166.00 | 92.7 | 0.6 | 1.6 | 0.2 | 5.4 | 0.4 | 91.0 | 0.6 | 2.5 | 0.2 | 6.1 | 0.5 |
| 41.50 | 93.9 | 0.4 | 0.7 | 0.1 | 5.1 | 0.3 | 93.4 | 0.5 | 1.0 | 0.0 | 5.2 | 0.5 |
| 10.38 | 93.7 | 0.1 | 0.7 | 0.1 | 4.8 | 0.2 | 94.0 | 0.4 | 0.6 | 0.1 | 4.6 | 0.2 |
| 2.59 | 88.4 | 0.9 | 0.5 | 0.1 | 3.3 | 0.3 | 92.4 | 0.7 | 0.6 | 0.1 | 3.8 | 0.3 |
| 0.65 | 54.8 | 1.6 | 0.4 | 0.0 | 1.9 | 0.3 | 67.3 | 0.3 | 0.4 | 0.1 | 2.3 | 0.0 |
| 0.16 | 19.7 | 0.4 | 0.5 | 0.1 | 0.7 | 0.1 | 27.8 | 0.4 | 0.5 | 0.1 | 1.0 | 0.2 |
| 0.04 | 5.9 | 0.5 | 0.5 | 0.0 | 0.3 | 0.1 | 9.1 | 0.6 | 0.4 | 0.1 | 0.4 | 0.1 |
| 0.00 | 0.4 | 0.0 | 0.4 | 0.1 | 0.1 | 0.0 | 0.5 | 0.1 | 0.4 | 0.0 | 0.0 | 0.0 |

### Example 7.4. Evaluation of Receptor Knockout by Flow Cytometry

Seven days post LNP treatment, T cells were assayed by flow cytometry to evaluate receptor knockout. T cells were incubated with a fixable viability dye (Beckman Coulter, Cat. C36628) and an antibody cocktail targeting the following molecules: CD3 (Biolegend, Cat. 317336), CD4 (Biolegend, Cat. 317434) and CD8 (Biolegend, Cat. 301046), B2M (Biolegend, Cat. 316306), CD38 (Biolegend, Cat. 303516), HLA-A2 (Biolegend, Cat. 343304) and HLA-DR, DP, DQ (Biolegend, Cat. 361714). Cells were subsequently washed, analyzed on a Cytoflex LX instrument (Beckman Coulter) using the FlowJo software package. T cells were gated on size, viability and CD8 positivity before expression of any markers was determined. The resulting data was plotted on GraphPad Prism v. 9.0.2 and analyzed using a variable slope (four parameter) non-linear regression.

As shown in Table 8 and FIG. 4, the 91-mer sgRNA tested outperformed the 100-mer version.

TABLE 8
Mean percentage of CD8+ T cells that are negative
for CD38 surface receptors following treatment with
sgRNAs targeting CD38 in the 100-mer or 91-mer formats.

| | CD38 (CD38−) | |
|---|---|---|
| sgRNA | 100-mer | 91-mer |

| (ng) | Mean | SD | Mean | SD |
|---|---|---|---|---|
| 166.00 | 99.8 | 0.2 | 99.8 | 0.1 |
| 41.50 | 99.6 | 0.1 | 99.9 | 0.1 |
| 10.38 | 98.4 | 0.3 | 99.5 | 0.1 |
| 2.59 | 87.4 | 1.9 | 94.3 | 0.9 |
| 0.65 | 47.0 | 4.3 | 60.0 | 1.9 |
| 0.16 | 20.5 | 4.9 | 23.8 | 1.6 |
| 0.04 | 16.5 | 10.1 | 12.5 | 0.6 |
| 0.00 | 17.2 | 4.1 | 13.7 | 0.7 |

### Example 8. Dose-Dependent Editing in NK Cells

Natural killer (NK) cells were edited using varying concentrations of either guide RNA or SpyCas9 mRNA. Cryopreserved NK cells from two donors were cultured overnight in NK Growth Media (NKGM): CTS OpTmizer media (Gibco) supplemented with 5% Human AB serum, 10 mM HEPES, 1× Glutamax, and 1% Pen-Strep. NK cells were activated by culturing cells 1:1 with irradiated K562 4-1BBL cells in NKGM with 500 U/ml IL-2 and 5 ng/ml IL-15 for three days.

NK cells were treated with two LNP, one delivering SpyCas9 mRNA (SEQ ID NO: 802) and one delivering gRNA G023522 targeting CD38. LNPs were generally prepared as described in Example 1 with the lipid composition using a molar ratio of 35 Lipid A/15 DSPC/47.5 cholesterol/2.5 PEG. The LNPs were formulated with a lipid amine to RNA phosphate (N:P) molar ratio of about 6. G023522 LNP was 4-fold serially diluted starting from 3.3 μg/ml up to 7 points with a standard concentration of the SpyCas9 mRNA LNP at 0.83 μg/ml and preincubated at 37° C. with 2.5 μg/ml recombinant human ApoE3 (Peprotech, 350-02) for about 10 minutes in NK Editing Media (NKEM): CTS OpTmizer media (Gibco) supplemented with 2.5% Human AB serum, 10 mM HEPES, 1× Glutamax, 1% Pen-Strep, 500 U/ml IL-2 and 5 ng/ml IL-15. SpyCas9 mRNA LNP was also 4-fold serially diluted starting from 3.3 μg/ml with a standard concentration of G023522 at 0.83 μg/ml and pre-incubated with ApoE3 in as directed above.

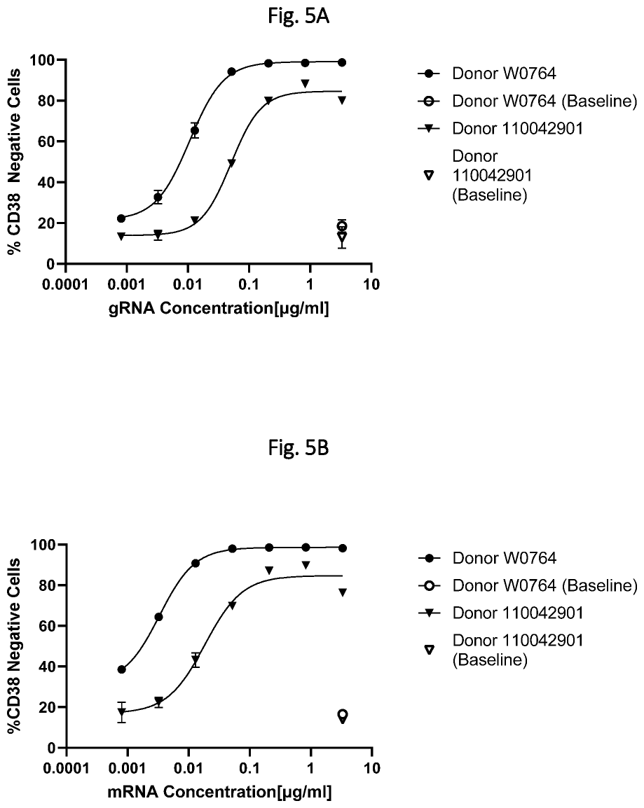

The pre-incubated LNPs were added to 5 e5 NK cells in triplicate at mRNA and gRNA concentrations indicated in Table 9. At 7 days post LNP treatment, cells were assayed by flow cytometry to measure rates of CD38 surface expression. Briefly, NK cells were incubated with antibodies targeting CD3 (Biolegend, Cat. No. 317344), CD56 (Biolegend, Cat. No. 362518) and CD38 (Biolegend, Cat. No. 303510). Cells were subsequently washed, processed on a Cytoflex instrument (Beckman Coulter) and analyzed using the FlowJo software package. NK cells were gated on size and CD3/CD56 status. Table 9 and FIGS. 5A-B show percent of NK cells without CD38 surface expression.

TABLE 9
Mean % CD38 negative cells in edited NK cells

| mRNA | Guide | Donor W0764 | | | Donor 110042901 | | |
|---|---|---|---|---|---|---|---|
| (ug/ml) | (ug/ml) | Mean | SD | N | Mean | SD | N |
| 0.83 | 3.32 | 98.7 | 0.2 | 3 | 80.0 | 1.2 | 3 |
| 0.83 | 0.83 | 98.5 | 0.1 | 3 | 88.2 | 0.6 | 3 |
| 0.83 | 0.2075 | 98.3 | 0.2 | 3 | 79.8 | 0.7 | 3 |
| 0.83 | 0.051875 | 94.2 | 1.2 | 3 | 49.1 | 0.5 | 3 |
| 0.83 | 0.012969 | 65.3 | 3.8 | 3 | 21.1 | 1.9 | 3 |
| 0.83 | 0.003242 | 32.7 | 3.3 | 3 | 14.0 | 2.5 | 3 |
| 0.83 | 0.000811 | 22.2 | 1.9 | 3 | 13.3 | 2.0 | 3 |
| 0.83 | 0 (baseline) | 18.5 | 3.0 | 3 | 13.0 | 5.3 | 3 |
| 3.32 | 0.83 | 98.2 | 0.2 | 3 | 76.3 | 1.9 | 3 |
| 0.83 | 0.83 | 98.6 | 0.2 | 3 | 89.8 | 0.1 | 3 |
| 0.2075 | 0.83 | 98.5 | 0.1 | 3 | 87.1 | 0.6 | 3 |
| 0.051875 | 0.83 | 97.9 | 0.1 | 3 | 69.8 | 1.5 | 3 |
| 0.012969 | 0.83 | 90.8 | 0.5 | 3 | 43.2 | 3.5 | 3 |
| 0.003242 | 0.83 | 64.4 | 1.1 | 3 | 22.3 | 2.6 | 3 |
| 0.0008 | 0.83 | 38.6 | 0.7 | 3 | 17.3 | 5.0 | 3 |
| 0 (baseline) | 0.83 | 16.5 | 1.1 | 3 | 14.0 | 0.0 | 1 |

### Example 9 Additional Biochemical Off-Target Analysis

Guides using a 91 nucleotide format were assessed for potential off-target DNA cleavage using the methods described in Example 3 with the following exceptions. Genomic DNA was treated with calf intestinal alkaline phosphatase (CIP) prior to use. The biochemical assay was performed with 16 nM Cas9 RNP formed using a molar ratio of 3 guide RNA:1 Cas9 protein. The number of cleaved sites detected (including the on-target site) for each guide are shown in Table 10. These potential off target sites and computationally predicted off target sites are validated using targeted sequencing as described in Example 3.

TABLE 10
Biochemically cleaved sites

| | | Total number of unique |
|---|---|---|
| Guide | Target | sites discovered |
| G028179 | CD38 | 47 |
| G028542 | CD38 | 53 |
| G028545 | CD38 | 88 |
| G028546 | CD38 | 53 |
| G028547 | CD38 | 133 |
| G000644 | EMX1 | 113 |
| G000645 | VEGFA | 1254 |
| G000646 | RAG1B | 117 |

### Example 10 Dose Responsive Editing in NK Cells with 91 Nucleotide Guides

Editing efficacy was assessed using 91 nucleotide guides (G028179, G028542, G028543, G028544, G028545—sequences shown in Table 12) delivered to natural killer (NK) cells using lipid nanoparticles (LNPs). Cells were expanded for 5 days from freshly isolated CD3-depleted cord blood mononuclear cells, activated with EBV-LCL feeder cells in NK MACS media (Miltenyi) and human AB serum (hABs) with IL-2 replenished after 2 days in culture. Cells were harvested and resuspended in OpTmizer media with 2.5% hABs, supplemental IL-2 (500 IU/mL) and IL-15 (5 ng/mL) cytokines, and 2.5 ug/ml ApoE3 (Sigma). Cells were aliquoted at 1 e5 cells per well in a 96 well tissue culture plate.

LNPs were generally prepared as described in Example 1 with the lipid composition using a molar ratio of 35 Lipid A/15 DSPC/47.5 cholesterol/2.5 PEG and the cargo using a 1:1 ratio of gRNA:mRNA by weight. LNPs were two-fold serially diluted in OpTmizer media with 2.5% hABs and supplemental IL-2 and IL-15 cytokines. LNPs were added to duplicate samples of NK cells from 3 donors at the concentrations of total RNA cargo weight indicated in Table 11. One day after LNP application, media was exchanged to NK MACS (Miltenyi) supplemented with IL-2 (500 IU/mL) and cells were returned to culture.

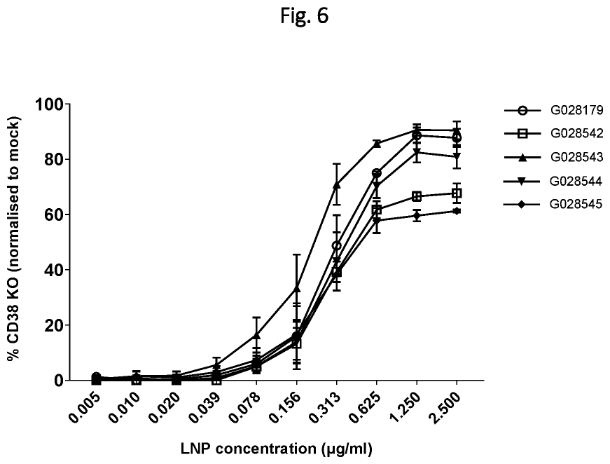

Eight days after LNP treatment, cells were assessed for the presence of CD38 surface antigen by flow cytometry. Briefly, NK cells were incubated with a mixture of antibodies: Anti-human CD56 Brilliant Violet 650 (Biolegend #362532), Anti-human CD16 Alexa Fluor 700 (Biolegend #302026), Anti-human CD38 PerCp-Cy5.5 (Biolegend #356614), Anti-human NKG2D Brilliant Violet 421 (Biolegend #320822), and Anti-human NKG2A APC (Biolegend #375108). Cells were washed then processed on a BD FACSCelesta Cytometer and analyzed using the FlowJo software package. Cells were gated based on FSC/SSC, single cells, viability, absence of feeder cells and CD38 expressing NK cells. Table 11 and FIG. 6 show the mean percent CD38 KG calculated across mean editing in 3 donors. Donor means were calculated across replicates. For each replicate, percent CD38 KO is calculated as 100−[(sample's % CD38 positive cells)/(Mock treatment's % CD38 positive cells for same donor)×100].

TABLE 11
Mean percent CD38 KO assessed by flow cytometry after gene editing (N = 3)

| | G028179 | | G028542 | | G028543 | | G028544 | | G028545 | |
|---|---|---|---|---|---|---|---|---|---|---|
| LNP (ug/ml) | Mean | SD | Mean | SD | Mean | SD | Mean | SD | Mean | SD |
| 0.004883 | 1.3 | 0.8 | −0.1 | 1.7 | 0.4 | 1.4 | 0.1 | 0.2 | 0.4 | 1.0 |
| 0.009766 | −0.2 | 1.1 | −0.6 | 2.0 | 1.5 | 1.9 | 0.7 | 0.7 | 1.5 | 1.7 |
| 0.019531 | 0.5 | 1.2 | −0.2 | 2.3 | 1.7 | 1.5 | −0.1 | 1.0 | 1.0 | 0.6 |
| 0.039063 | 1.9 | 1.5 | −0.1 | 1.9 | 5.6 | 2.7 | 0.9 | 1.0 | 3.0 | 2.2 |
| 0.078125 | 5.9 | 3.0 | 5.0 | 1.8 | 16.4 | 6.3 | 5.1 | 2.6 | 7.3 | 4.4 |
| 0.15625 | 15.9 | 11.9 | 13.2 | 5.8 | 33.4 | 12.2 | 14.0 | 7.9 | 16.6 | 10.2 |
| 0.3125 | 48.7 | 11.1 | 39.3 | 3.7 | 70.9 | 7.4 | 43.1 | 10.5 | 38.4 | 5.9 |
| 0.625 | 75.0 | 0.6 | 61.8 | 3.1 | 85.7 | 1.2 | 70.3 | 4.3 | 57.8 | 4.5 |
| 1.25 | 88.7 | 2.8 | 66.6 | 1.6 | 90.6 | 2.0 | 82.5 | 3.6 | 59.6 | 2.1 |
| 2.5 | 87.7 | 3.3 | 67.7 | 3.5 | 90.4 | 3.3 | 80.9 | 4.2 | 61.3 | 0.5 |

| | | | | | |
|---|---|---|---|---|---|
| EC95 | 1.05 | 0.89 | 0.74 | 1.04 | 0.90 |

TABLE 12
Additional Sequences

| | SEQ | |
|---|---|---|
| Description | ID NO: | SEQUENCE |
| Guide scaffold | 200 | GUUUUAGAGCUAUGCUGUUUUG |
| Guide scaffold | 201 | GUUUUAGAGCUAGAAAUAGCAAGUUAAAAUAAGGCUAGU |
| | | CCGUUAUCAACUUGAAAAAGUGGCACCGAGUCGGUGC |
| Guide scaffold | 202 | GUUUUAGAGCUAGAAAUAGCAAGUUAAAAUAAGGCUAGU |
| | | CCGUUAUCAACUUGAAAAAGUGGCACCGAGUCGGUGCUU |
| | | UU |
| Guide scaffold | 300 | mN*mN*mN*NNNNNNNNNNNNNNNNNGUUUUAGAmGmCm |
| | | UmAmGmAmAmAmUmAmGmCAAGUUAAAAUAAGGCUAGU |
| | | CCGUUAUCAmAmCmUmUmGmAmAmAmAmAmGmUmGmGm |
| | | CmAmCmCmGmAmGmUmCmGmGmUmGmCmU*mU*mU*mU |
| Guide scaffold | 405 | (N)20GUUUUAGAGCUAGAAAUAGCAAGUUAAAAUAAGGC |
| 95 | | UAGUCCGUUAUCAACUUGGCACCGAGUCGGUGC |
| Guide scaffold | 406 | mN*mN*mN*(N)17GUUUUAGAmGmCmUmAmGmAmAmAmU |
| 195 | | mAmGmCAAGUUAAAAUAAGGCUAGUCCGUUAUCAACUUG |
| | | GCACCGAGUCGG*mU*mG*mC |
| Guide scaffold | 407 | (N)20GUUUUAGAGCUAGAAAUAGCAAGUUAAAAUAAGGC |
| 871 | | UAGUCCGUUAUCACGAAAGGGCACCGAGUCGGUGC |
| Guide scaffold | 408 | mN*mN*mN*(N)17mGUUUfUAGmAmGmCmUmAmGmAmAmA |
| 971 | | mUmAmGmCmAmAGUfUmAfAmAfAmUAmAmGmGmCmUmA |
| | | GUmCmCGUfUAmUmCAmCmGmAmAmAmGmGmGmCmAmC |
| | | mCmGmAmGmUmCmGmG*mU*mG*mC |
| Guide scaffold | 410 | mN*mN*mN*(N)17GUUUUAGAmGmCmUmAmGmAmAmAmU |
| 972 | | mAmGmCAAGUUAAAAUAAGGCUAGUCCGUUAUCACGAAA |
| | | GGGCACCGAGUCGG*mU*mG*mC |
| tracrRNA | 411 | AACAGCAUAGCAAGUUAAAAUAAGGCUAGUCCGUUAUCA |
| | | ACUUGAAAAAGUGGCACCGAGUCGGUGCUUUUUUU |
| Guide scaffold | 412 | mN*mN*mN*(N)17GUUUUAGAmGmCmUmAmGmAmAmAmU |
| | | mAmGmCAAGUUAAAAUAAGGCUAGUCCGUUAUCACGAAA |
| | | GGGCACCGAGUCGGmUmGmC*mU |
| Guide scaffold | 413 | CUUGACGCAUCGCGCCAGGAGUUUUAGAGCUAGAAAUAGCAAGUUAA |
| with CD38 | | AAUAAGGCUAGUCCGUUAUCACGAAAGGGCACCGAGUCGGUGCU |
| target sequence | | |
| SEQ ID NO: | | |
| 11 underlined | | |
| Guide scaffold | 414 | mN*mN*mN*NNNNNNNNNNNNNNNNNGUUUUAGAmGmCmUmAm |
| | | GmAmAmAmUmAmGmCAAGUUAAAAUAAGGCUAGUCCGUUAUC |
| | | ACGAAAGGGCACCGAGUCGGmU*mG*mC*mU |
| Recombinant | 800 | MDKKYSIGLDIGTNSVGWAVITDEYKVPSKKFKVLGNTDRHS |
| Cas9-NLS | | IKKNLIGALLFDSGETAEATRLKRTARRRYTRRKNRICYLQEIF |
| amino acid | | SNEMAKVDDSFFHRLEESFLVEEDKKHERHPIFGNIVDEVAYH |
| sequence | | EKYPTIYHLRKKLVDSTDKADLRLIYLALAHMIKFRGHFLIEG |
| | | DLNPDNSDVDKLFIQLVQTYNQLFEENPINASGVDAKAILSAR |
| | | LSKSRRLENLIAQLPGEKKNGLFGNLIALSLGLTPNFKSNFDLA |
| | | EDAKLQLSKDTYDDDLDNLLAQIGDQYADLFLAAKNLSDAIL |
| | | LSDILRVNTEITKAPLSASMIKRYDEHHQDLTLLKALVRQQLP |
| | | EKYKEIFFDQSKNGYAGYIDGGASQEEFYKFIKPILEKMDGTE |
| | | ELLVKLNREDLLRKQRTFDNGSIPHQIHLGELHAILRRQEDFYP |
| | | FLKDNREKIEKILTFRIPYYVGPLARGNSRFAWMTRKSEETITP |
| | | WNFEEVVDKGASAQSFIERMTNFDKNLPNEKVLPKHSLLYEY |
| | | FTVYNELTKVKYVTEGMRKPAFLSGEQKKAIVDLLFKTNRKV |
| | | TVKQLKEDYFKKIECFDSVEISGVEDRFNASLGTYHDLLKIIKD |
| | | KDFLDNEENEDILEDIVLTLTLFEDREMIEERLKTYAHLFDDKV |
| | | MKQLKRRRYTGWGRLSRKLINGIRDKQSGKTILDFLKSDGFA |
| | | NRNFMQLIHDDSLTFKEDIQKAQVSGQGDSLHEHIANLAGSPA |
| | | IKKGILQTVKVVDELVKVMGRHKPENIVIEMARENQTTQKGQ |
| | | KNSRERMKRIEEGIKELGSQILKEHPVENTQLQNEKLYLYYLQ |
| | | NGRDMYVDQELDINRLSDYDVDHIVPQSFLKDDSIDNKVLTR |
| | | SDKNRGKSDNVPSEEVVKKMKNYWRQLLNAKLITQRKFDNL |
| | | TKAERGGLSELDKAGFIKRQLVETRQITKHVAQILDSRMNTKY |
| | | DENDKLIREVKVITLKSKLVSDFRKDFQFYKVREINNYHHAHD |
| | | AYLNAVVGTALIKKYPKLESEFVYGDYKVYDVRKMIAKSEQE |
| | | IGKATAKYFFYSNIMNFFKTEITLANGEIRKRPLIETNGETGEIV |
| | | WDKGRDFATVRKVLSMPQVNIVKKTEVQTGGFSKESILPKRN |
| | | SDKLIARKKDWDPKKYGGFDSPTVAYSVLVVAKVEKGKSKK |
| | | LKSVKELLGITIMERSSFEKNPIDFLEAKGYKEVKKDLIIKLPK |
| | | YSLFELENGRKRMLASAGELQKGNELALPSKYVNFLYLASHY |
| | | EKLKGSPEDNEQKQLFVEQHKHYLDEIIEQISEFSKRVILADAN |
| | | LDKVLSAYNKHRDKPIREQAENIIHLFTLTNLGAPAAFKYFDT |
| | | TIDRKRYTSTKEVLDATLIHQSITGLYETRIDLSQLGGDGGGSP |
| | | KKKRKV |
| ORF encoding | 801 | ATGGACAAGAAGTACAGCATCGGACTGGACATCGGAACAA |
| Sp. Cas9 | | ACAGCGTCGGATGGGCAGTCATCACAGACGAATACAAGGT |
| | | CCCGAGCAAGAAGTTCAAGGTCCTGGGAAACACAGACAGA |
| | | CACAGCATCAAGAAGAACCTGATCGGAGCACTGCTGTTCG |
| | | ACAGCGGAGAAACAGCAGAAGCAACAAGACTGAAGAGAA |
| | | CAGCAAGAAGAAGATACACAAGAAGAAAGAACAGAATCT |
| | | GCTACCTGCAGGAAATCTTCAGCAACGAAATGGCAAAGGT |
| | | CGACGACAGCTTCTTCCACAGACTGGAAGAAAGCTTCCTGG |
| | | TCGAAGAAGACAAGAAGCACGAAAGACACCCGATCTTCGG |
| | | AAACATCGTCGACGAAGTCGCATACCACGAAAAGTACCCG |
| | | ACAATCTACCACCTGAGAAAGAAGCTGGTCGACAGCACAG |
| | | ACAAGGCAGACCTGAGACTGATCTACCTGGCACTGGCACA |
| | | CATGATCAAGTTCAGAGGACACTTCCTGATCGAAGGAGAC |
| | | CTGAACCCGGACAACAGCGACGTCGACAAGCTGTTCATCC |
| | | AGCTGGTCCAGACATACAACCAGCTGTTCGAAGAAAACCC |
| | | GATCAACGCAAGCGGAGTCGACGCAAAGGCAATCCTGAGC |
| | | GCAAGACTGAGCAAGAGCAGAAGACTGGAAAACCTGATCG |
| | | CACAGCTGCCGGGAGAAAAGAAGAACGGACTGTTCGGAAA |
| | | CCTGATCGCACTGAGCCTGGGACTGACACCGAACTTCAAGA |
| | | GCAACTTCGACCTGGCAGAAGACGCAAAGCTGCAGCTGAG |
| | | CAAGGACACATACGACGACGACCTGGACAACCTGCTGGCA |
| | | CAGATCGGAGACCAGTACGCAGACCTGTTCCTGGCAGCAA |
| | | AGAACCTGAGCGACGCAATCCTGCTGAGCGACATCCTGAG |
| | | AGTCAACACAGAAATCACAAAGGCACCGCTGAGCGCAAGC |
| | | ATGATCAAGAGATACGACGAACACCACCAGGACCTGACAC |
| | | TGCTGAAGGCACTGGTCAGACAGCAGCTGCCGGAAAAGTA |
| | | CAAGGAAATCTTCTTCGACCAGAGCAAGAACGGATACGCA |
| | | GGATACATCGACGGAGGAGCAAGCCAGGAAGAATTCTACA |
| | | AGTTCATCAAGCCGATCCTGGAAAAGATGGACGGAACAGA |
| | | AGAACTGCTGGTCAAGCTGAACAGAGAAGACCTGCTGAGA |
| | | AAGCAGAGAACATTCGACAACGGAAGCATCCCGCACCAGA |
| | | TCCACCTGGGAGAACTGCACGCAATCCTGAGAAGACAGGA |
| | | AGACTTCTACCCGTTCCTGAAGGACAACAGAGAAAAGATC |
| | | GAAAAGATCCTGACATTCAGAATCCCGTACTACGTCGGACC |
| | | GCTGGCAAGAGGAAACAGCAGATTCGCATGGATGACAAGA |
| | | AAGAGCGAAGAAACAATCACACCGTGGAACTTCGAAGAAG |
| | | TCGTCGACAAGGGAGCAAGCGCACAGAGCTTCATCGAAAG |
| | | AATGACAAACTTCGACAAGAACCTGCCGAACGAAAAGGTC |
| | | CTGCCGAAGCACAGCCTGCTGTACGAATACTTCACAGTCTA |
| | | CAACGAACTGACAAAGGTCAAGTACGTCACAGAAGGAATG |
| | | AGAAAGCCGGCATTCCTGAGCGGAGAACAGAAGAAGGCAA |
| | | TCGTCGACCTGCTGTTCAAGACAAACAGAAAGGTCACAGTC |
| | | AAGCAGCTGAAGGAAGACTACTTCAAGAAGATCGAATGCT |
| | | TCGACAGCGTCGAAATCAGCGGAGTCGAAGACAGATTCAA |
| | | CGCAAGCCTGGGAACATACCACGACCTGCTGAAGATCATC |
| | | AAGGACAAGGACTTCCTGGACAACGAAGAAAACGAAGACA |
| | | TCCTGGAAGACATCGTCCTGACACTGACACTGTTCGAAGAC |
| | | AGAGAAATGATCGAAGAAAGACTGAAGACATACGCACACC |
| | | TGTTCGACGACAAGGTCATGAAGCAGCTGAAGAGAAGAAG |
| | | ATACACAGGATGGGGAAGACTGAGCAGAAAGCTGATCAAC |
| | | GGAATCAGAGACAAGCAGAGCGGAAAGACAATCCTGGACT |
| | | TCCTGAAGAGCGACGGATTCGCAAACAGAAACTTCATGCA |
| | | GCTGATCCACGACGACAGCCTGACATTCAAGGAAGACATC |
| | | CAGAAGGCACAGGTCAGCGGACAGGGAGACAGCCTGCACG |
| | | AACACATCGCAAACCTGGCAGGAAGCCCGGCAATCAAGAA |
| | | GGGAATCCTGCAGACAGTCAAGGTCGTCGACGAACTGGTC |
| | | AAGGTCATGGGAAGACACAAGCCGGAAAACATCGTCATCG |
| | | AAATGGCAAGAGAAAACCAGACAACACAGAAGGGACAGA |
| | | AGAACAGCAGAGAAAGAATGAAGAGAATCGAAGAAGGAA |
| | | TCAAGGAACTGGGAAGCCAGATCCTGAAGGAACACCCGGT |
| | | CGAAAACACACAGCTGCAGAACGAAAAGCTGTACCTGTAC |
| | | TACCTGCAGAACGGAAGAGACATGTACGTCGACCAGGAAC |
| | | TGGACATCAACAGACTGAGCGACTACGACGTCGACCACAT |
| | | CGTCCCGCAGAGCTTCCTGAAGGACGACAGCATCGACAAC |
| | | AAGGTCCTGACAAGAAGCGACAAGAACAGAGGAAAGAGC |
| | | GACAACGTCCCGAGCGAAGAAGTCGTCAAGAAGATGAAGA |
| | | ACTACTGGAGACAGCTGCTGAACGCAAAGCTGATCACACA |
| | | GAGAAAGTTCGACAACCTGACAAAGGCAGAGAGAGGAGG |
| | | ACTGAGCGAACTGGACAAGGCAGGATTCATCAAGAGACAG |
| | | CTGGTCGAAACAAGACAGATCACAAAGCACGTCGCACAGA |
| | | TCCTGGACAGCAGAATGAACACAAAGTACGACGAAAACGA |
| | | CAAGCTGATCAGAGAAGTCAAGGTCATCACACTGAAGAGC |
| | | AAGCTGGTCAGCGACTTCAGAAAGGACTTCCAGTTCTACAA |
| | | GGTCAGAGAAATCAACAACTACCACCACGCACACGACGCA |
| | | TACCTGAACGCAGTCGTCGGAACAGCACTGATCAAGAAGT |
| | | ACCCGAAGCTGGAAAGCGAATTCGTCTACGGAGACTACAA |
| | | GGTCTACGACGTCAGAAAGATGATCGCAAAGAGCGAACAG |
| | | GAAATCGGAAAGGCAACAGCAAAGTACTTCTTCTACAGCA |
| | | ACATCATGAACTTCTTCAAGACAGAAATCACACTGGCAAAC |
| | | GGAGAAATCAGAAAGAGACCGCTGATCGAAACAAACGGA |
| | | GAAACAGGAGAAATCGTCTGGGACAAGGGAAGAGACTTCG |
| | | CAACAGTCAGAAAGGTCCTGAGCATGCCGCAGGTCAACAT |
| | | CGTCAAGAAGACAGAAGTCCAGACAGGAGGATTCAGCAAG |
| | | GAAAGCATCCTGCCGAAGAGAAACAGCGACAAGCTGATCG |
| | | CAAGAAAGAAGGACTGGGACCCGAAGAAGTACGGAGGATT |
| | | CGACAGCCCGACAGTCGCATACAGCGTCCTGGTCGTCGCAA |
| | | AGGTCGAAAAGGGAAAGAGCAAGAAGCTGAAGAGCGTCA |
| | | AGGAACTGCTGGGAATCACAATCATGGAAAGAAGCAGCTT |
| | | CGAAAAGAACCCGATCGACTTCCTGGAAGCAAAGGGATAC |
| | | AAGGAAGTCAAGAAGGACCTGATCATCAAGCTGCCGAAGT |
| | | ACAGCCTGTTCGAACTGGAAAACGGAAGAAAGAGAATGCT |
| | | GGCAAGCGCAGGAGAACTGCAGAAGGGAAACGAACTGGC |
| | | ACTGCCGAGCAAGTACGTCAACTTCCTGTACCTGGCAAGCC |
| | | ACTACGAAAAGCTGAAGGGAAGCCCGGAAGACAACGAAC |
| | | AGAAGCAGCTGTTCGTCGAACAGCACAAGCACTACCTGGA |
| | | CGAAATCATCGAACAGATCAGCGAATTCAGCAAGAGAGTC |
| | | ATCCTGGCAGACGCAAACCTGGACAAGGTCCTGAGCGCAT |
| | | ACAACAAGCACAGAGACAAGCCGATCAGAGAACAGGCAG |
| | | AAAACATCATCCACCTGTTCACACTGACAAACCTGGGAGCA |
| | | CCGGCAGCATTCAAGTACTTCGACACAACAATCGACAGAA |
| | | AGAGATACACAAGCACAAAGGAAGTCCTGGACGCAACACT |
| | | GATCCACCAGAGCATCACAGGACTGTACGAAACAAGAATC |
| | | GACCTGAGCCAGCTGGGAGGAGACGGAGGAGGAAGCCCG |
| | | AAGAAGAAGAGAAAGGTCTAG |
| ORF encoding | 802 | ATGGACAAGAAGTACTCCATCGGCCTGGACATCGGCACCAACTCC |
| Sp. Cas9 | | GTGGGCTGGGCCGTGATCACCGACGAGTACAAGGTGCCCTCCAA |
| | | GAAGTTCAAGGTGCTGGGCAACACCGACCGGCACTCCATCAAGA |
| | | AGAACCTGATCGGCGCCCTGCTGTTCGACTCCGGCGAGACCGCCG |
| | | AGGCCACCCGGCTGAAGCGGACCGCCCGGCGGCGGTACACCCGG |
| | | CGGAAGAACCGGATCTGCTACCTGCAGGAGATCTTCTCCAACGAG |
| | | ATGGCCAAGGTGGACGACTCCTTCTTCCACCGGCTGGAGGAGTCC |
| | | TTCCTGGTGGAGGAGGACAAGAAGCACGAGCGGCACCCCATCTT |
| | | CGGCAACATCGTGGACGAGGTGGCCTACCACGAGAAGTACCCCA |
| | | CCATCTACCACCTGCGGAAGAAGCTGGTGGACTCCACCGACAAG |
| | | GCCGACCTGCGGCTGATCTACCTGGCCCTGGCCCACATGATCAAG |
| | | TTCCGGGGCCACTTCCTGATCGAGGGCGACCTGAACCCCGACAAC |
| | | TCCGACGTGGACAAGCTGTTCATCCAGCTGGTGCAGACCTACAAC |
| | | CAGCTGTTCGAGGAGAACCCCATCAACGCCTCCGGCGTGGACGCC |
| | | AAGGCCATCCTGTCCGCCCGGCTGTCCAAGTCCCGGCGGCTGGAG |
| | | AACCTGATCGCCCAGCTGCCCGGCGAGAAGAAGAACGGCCTGTT |
| | | CGGCAACCTGATCGCCCTGTCCCTGGGCCTGACCCCCAACTTCAA |
| | | GTCCAACTTCGACCTGGCCGAGGACGCCAAGCTGCAGCTGTCCAA |
| | | GGACACCTACGACGACGACCTGGACAACCTGCTGGCCCAGATCG |
| | | GCGACCAGTACGCCGACCTGTTCCTGGCCGCCAAGAACCTGTCCG |
| | | ACGCCATCCTGCTGTCCGACATCCTGCGGGTGAACACCGAGATCA |
| | | CCAAGGCCCCCCTGTCCGCCTCCATGATCAAGCGGTACGACGAGC |
| | | ACCACCAGGACCTGACCCTGCTGAAGGCCCTGGTGCGGCAGCAG |
| | | CTGCCCGAGAAGTACAAGGAGATCTTCTTCGACCAGTCCAAGAAC |
| | | GGCTACGCCGGCTACATCGACGGCGGCGCCTCCCAGGAGGAGTTC |
| | | TACAAGTTCATCAAGCCCATCCTGGAGAAGATGGACGGCACCGA |
| | | GGAGCTGCTGGTGAAGCTGAACCGGGAGGACCTGCTGCGGAAGC |
| | | AGCGGACCTTCGACAACGGCTCCATCCCCCACCAGATCCACCTGG |
| | | GCGAGCTGCACGCCATCCTGCGGCGGCAGGAGGACTTCTACCCCT |
| | | TCCTGAAGGACAACCGGGAGAAGATCGAGAAGATCCTGACCTTC |
| | | CGGATCCCCTACTACGTGGGCCCCCTGGCCCGGGGCAACTCCCGG |
| | | TTCGCCTGGATGACCCGGAAGTCCGAGGAGACCATCACCCCCTGG |
| | | AACTTCGAGGAGGTGGTGGACAAGGGCGCCTCCGCCCAGTCCTTC |
| | | ATCGAGCGGATGACCAACTTCGACAAGAACCTGCCCAACGAGAA |
| | | GGTGCTGCCCAAGCACTCCCTGCTGTACGAGTACTTCACCGTGTA |
| | | CAACGAGCTGACCAAGGTGAAGTACGTGACCGAGGGCATGCGGA |
| | | AGCCCGCCTTCCTGTCCGGCGAGCAGAAGAAGGCCATCGTGGACC |
| | | TGCTGTTCAAGACCAACCGGAAGGTGACCGTGAAGCAGCTGAAG |
| | | GAGGACTACTTCAAGAAGATCGAGTGCTTCGACTCCGTGGAGATC |
| | | TCCGGCGTGGAGGACCGGTTCAACGCCTCCCTGGGCACCTACCAC |
| | | GACCTGCTGAAGATCATCAAGGACAAGGACTTCCTGGACAACGA |
| | | GGAGAACGAGGACATCCTGGAGGACATCGTGCTGACCCTGACCC |
| | | TGTTCGAGGACCGGGAGATGATCGAGGAGCGGCTGAAGACCTAC |
| | | GCCCACCTGTTCGACGACAAGGTGATGAAGCAGCTGAAGCGGCG |
| | | GCGGTACACCGGCTGGGGCCGGCTGTCCCGGAAGCTGATCAACG |
| | | GCATCCGGGACAAGCAGTCCGGCAAGACCATCCTGGACTTCCTGA |
| | | AGTCCGACGGCTTCGCCAACCGGAACTTCATGCAGCTGATCCACG |
| | | ACGACTCCCTGACCTTCAAGGAGGACATCCAGAAGGCCCAGGTGT |
| | | CCGGCCAGGGCGACTCCCTGCACGAGCACATCGCCAACCTGGCCG |
| | | GCTCCCCCGCCATCAAGAAGGGCATCCTGCAGACCGTGAAGGTG |
| | | GTGGACGAGCTGGTGAAGGTGATGGGCCGGCACAAGCCCGAGAA |
| | | CATCGTGATCGAGATGGCCCGGGAGAACCAGACCACCCAGAAGG |
| | | GCCAGAAGAACTCCCGGGAGCGGATGAAGCGGATCGAGGAGGGC |
| | | ATCAAGGAGCTGGGCTCCCAGATCCTGAAGGAGCACCCCGTGGA |
| | | GAACACCCAGCTGCAGAACGAGAAGCTGTACCTGTACTACCTGCA |
| | | GAACGGCCGGGACATGTACGTGGACCAGGAGCTGGACATCAACC |
| | | GGCTGTCCGACTACGACGTGGACCACATCGTGCCCCAGTCCTTCC |
| | | TGAAGGACGACTCCATCGACAACAAGGTGCTGACCCGGTCCGAC |
| | | AAGAACCGGGGCAAGTCCGACAACGTGCCCTCCGAGGAGGTGGT |
| | | GAAGAAGATGAAGAACTACTGGCGGCAGCTGCTGAACGCCAAGC |
| | | TGATCACCCAGCGGAAGTTCGACAACCTGACCAAGGCCGAGCGG |
| | | GGCGGCCTGTCCGAGCTGGACAAGGCCGGCTTCATCAAGCGGCA |
| | | GCTGGTGGAGACCCGGCAGATCACCAAGCACGTGGCCCAGATCC |
| | | TGGACTCCCGGATGAACACCAAGTACGACGAGAACGACAAGCTG |
| | | ATCCGGGAGGTGAAGGTGATCACCCTGAAGTCCAAGCTGGTGTCC |
| | | GACTTCCGGAAGGACTTCCAGTTCTACAAGGTGCGGGAGATCAAC |
| | | AACTACCACCACGCCCACGACGCCTACCTGAACGCCGTGGTGGGC |
| | | ACCGCCCTGATCAAGAAGTACCCCAAGCTGGAGTCCGAGTTCGTG |
| | | TACGGCGACTACAAGGTGTACGACGTGCGGAAGATGATCGCCAA |
| | | GTCCGAGCAGGAGATCGGCAAGGCCACCGCCAAGTACTTCTTCTA |
| | | CTCCAACATCATGAACTTCTTCAAGACCGAGATCACCCTGGCCAA |
| | | CGGCGAGATCCGGAAGCGGCCCCTGATCGAGACCAACGGCGAGA |
| | | CCGGCGAGATCGTGTGGGACAAGGGCCGGGACTTCGCCACCGTG |
| | | CGGAAGGTGCTGTCCATGCCCCAGGTGAACATCGTGAAGAAGAC |
| | | CGAGGTGCAGACCGGCGGCTTCTCCAAGGAGTCCATCCTGCCCAA |
| | | GCGGAACTCCGACAAGCTGATCGCCCGGAAGAAGGACTGGGACC |
| | | CCAAGAAGTACGGCGGCTTCGACTCCCCCACCGTGGCCTACTCCG |
| | | TGCTGGTGGTGGCCAAGGTGGAGAAGGGCAAGTCCAAGAAGCTG |
| | | AAGTCCGTGAAGGAGCTGCTGGGCATCACCATCATGGAGCGGTCC |
| | | TCCTTCGAGAAGAACCCCATCGACTTCCTGGAGGCCAAGGGCTAC |
| | | AAGGAGGTGAAGAAGGACCTGATCATCAAGCTGCCCAAGTACTC |
| | | CCTGTTCGAGCTGGAGAACGGCCGGAAGCGGATGCTGGCCTCCGC |
| | | CGGCGAGCTGCAGAAGGGCAACGAGCTGGCCCTGCCCTCCAAGT |
| | | ACGTGAACTTCCTGTACCTGGCCTCCCACTACGAGAAGCTGAAGG |
| | | GCTCCCCCGAGGACAACGAGCAGAAGCAGCTGTTCGTGGAGCAG |
| | | CACAAGCACTACCTGGACGAGATCATCGAGCAGATCTCCGAGTTC |
| | | TCCAAGCGGGTGATCCTGGCCGACGCCAACCTGGACAAGGTGCTG |
| | | TCCGCCTACAACAAGCACCGGGACAAGCCCATCCGGGAGCAGGC |
| | | CGAGAACATCATCCACCTGTTCACCCTGACCAACCTGGGCGCCCC |
| | | CGCCGCCTTCAAGTACTTCGACACCACCATCGACCGGAAGCGGTA |
| | | CACCTCCACCAAGGAGGTGCTGGACGCCACCCTGATCCACCAGTC |
| | | CATCACCGGCCTGTACGAGACCCGGATCGACCTGTCCCAGCTGGG |
| | | CGGCGACGGCGGCGGCTCCCCCAAGAAGAAGCGGAAGGTGTGA |
| Open reading | 803 | AUGGACAAGAAGUACUCCAUCGGCCUGGACAUCGGCACCAACU |
| frame for Cas9 | | CCGUGGGCUGGGCCGUGAUCACCGACGAGUACAAGGUGCCCUCC |
| with Hibit tag | | AAGAAGUUCAAGGUGCUGGGCAACACCGACCGGCACUCCAUCA |
| | | AGAAGAACCUGAUCGGCGCCCUGCUGUUCGACUCCGGCGAGACC |
| | | GCCGAGGCCACCCGGCUGAAGCGGACCGCCCGGCGGCGGUACAC |
| | | CCGGCGGAAGAACCGGAUCUGCUACCUGCAGGAGAUCUUCUCC |
| | | AACGAGAUGGCCAAGGUGGACGACUCCUUCUUCCACCGGCUGG |
| | | AGGAGUCCUUCCUGGUGGAGGAGGACAAGAAGCACGAGCGGCA |
| | | CCCCAUCUUCGGCAACAUCGUGGACGAGGUGGCCUACCACGAGA |
| | | AGUACCCCACCAUCUACCACCUGCGGAAGAAGCUGGUGGACUCC |
| | | ACCGACAAGGCCGACCUGCGGCUGAUCUACCUGGCCCUGGCCCA |
| | | CAUGAUCAAGUUCCGGGGCCACUUCCUGAUCGAGGGCGACCUG |
| | | AACCCCGACAACUCCGACGUGGACAAGCUGUUCAUCCAGCUGGU |
| | | GCAGACCUACAACCAGCUGUUCGAGGAGAACCCCAUCAACGCCU |
| | | CCGGCGUGGACGCCAAGGCCAUCCUGUCCGCCCGGCUGUCCAAG |
| | | UCCCGGCGGCUGGAGAACCUGAUCGCCCAGCUGCCCGGCGAGAA |
| | | GAAGAACGGCCUGUUCGGCAACCUGAUCGCCCUGUCCCUGGGCC |
| | | UGACCCCCAACUUCAAGUCCAACUUCGACCUGGCCGAGGACGCC |
| | | AAGCUGCAGCUGUCCAAGGACACCUACGACGACGACCUGGACA |
| | | ACCUGCUGGCCCAGAUCGGCGACCAGUACGCCGACCUGUUCCUG |
| | | GCCGCCAAGAACCUGUCCGACGCCAUCCUGCUGUCCGACAUCCU |
| | | GCGGGUGAACACCGAGAUCACCAAGGCCCCCCUGUCCGCCUCCA |
| | | UGAUCAAGCGGUACGACGAGCACCACCAGGACCUGACCCUGCUG |
| | | AAGGCCCUGGUGCGGCAGCAGCUGCCCGAGAAGUACAAGGAGA |
| | | UCUUCUUCGACCAGUCCAAGAACGGCUACGCCGGCUACAUCGAC |
| | | GGCGGCGCCUCCCAGGAGGAGUUCUACAAGUUCAUCAAGCCCA |
| | | UCCUGGAGAAGAUGGACGGCACCGAGGAGCUGCUGGUGAAGCU |
| | | GAACCGGGAGGACCUGCUGCGGAAGCAGCGGACCUUCGACAAC |
| | | GGCUCCAUCCCCCACCAGAUCCACCUGGGCGAGCUGCACGCCAU |
| | | CCUGCGGCGGCAGGAGGACUUCUACCCCUUCCUGAAGGACAACC |
| | | GGGAGAAGAUCGAGAAGAUCCUGACCUUCCGGAUCCCCUACUA |
| | | CGUGGGCCCCCUGGCCCGGGGCAACUCCCGGUUCGCCUGGAUGA |
| | | CCCGGAAGUCCGAGGAGACCAUCACCCCCUGGAACUUCGAGGAG |
| | | GUGGUGGACAAGGGCGCCUCCGCCCAGUCCUUCAUCGAGCGGA |
| | | UGACCAACUUCGACAAGAACCUGCCCAACGAGAAGGUGCUGCCC |
| | | AAGCACUCCCUGCUGUACGAGUACUUCACCGUGUACAACGAGC |
| | | UGACCAAGGUGAAGUACGUGACCGAGGGCAUGCGGAAGCCCGC |
| | | CUUCCUGUCCGGCGAGCAGAAGAAGGCCAUCGUGGACCUGCUG |
| | | UUCAAGACCAACCGGAAGGUGACCGUGAAGCAGCUGAAGGAGG |
| | | ACUACUUCAAGAAGAUCGAGUGCUUCGACUCCGUGGAGAUCUC |
| | | CGGCGUGGAGGACCGGUUCAACGCCUCCCUGGGCACCUACCACG |
| | | ACCUGCUGAAGAUCAUCAAGGACAAGGACUUCCUGGACAACGA |
| | | GGAGAACGAGGACAUCCUGGAGGACAUCGUGCUGACCCUGACC |
| | | CUGUUCGAGGACCGGGAGAUGAUCGAGGAGCGGCUGAAGACCU |
| | | ACGCCCACCUGUUCGACGACAAGGUGAUGAAGCAGCUGAAGCG |
| | | GCGGCGGUACACCGGCUGGGGCCGGCUGUCCCGGAAGCUGAUC |
| | | AACGGCAUCCGGGACAAGCAGUCCGGCAAGACCAUCCUGGACU |
| | | UCCUGAAGUCCGACGGCUUCGCCAACCGGAACUUCAUGCAGCUG |
| | | AUCCACGACGACUCCCUGACCUUCAAGGAGGACAUCCAGAAGGC |
| | | CCAGGUGUCCGGCCAGGGCGACUCCCUGCACGAGCACAUCGCCA |
| | | ACCUGGCCGGCUCCCCCGCCAUCAAGAAGGGCAUCCUGCAGACC |
| | | GUGAAGGUGGUGGACGAGCUGGUGAAGGUGAUGGGCCGGCACA |
| | | AGCCCGAGAACAUCGUGAUCGAGAUGGCCCGGGAGAACCAGAC |
| | | CACCCAGAAGGGCCAGAAGAACUCCCGGGAGCGGAUGAAGCGG |
| | | AUCGAGGAGGGCAUCAAGGAGCUGGGCUCCCAGAUCCUGAAGG |
| | | AGCACCCCGUGGAGAACACCCAGCUGCAGAACGAGAAGCUGUA |
| | | CCUGUACUACCUGCAGAACGGCCGGGACAUGUACGUGGACCAG |
| | | GAGCUGGACAUCAACCGGCUGUCCGACUACGACGUGGACCACA |
| | | UCGUGCCCCAGUCCUUCCUGAAGGACGACUCCAUCGACAACAAG |
| | | GUGCUGACCCGGUCCGACAAGAACCGGGGCAAGUCCGACAACG |
| | | UGCCCUCCGAGGAGGUGGUGAAGAAGAUGAAGAACUACUGGCG |
| | | GCAGCUGCUGAACGCCAAGCUGAUCACCCAGCGGAAGUUCGAC |
| | | AACCUGACCAAGGCCGAGCGGGGCGGCCUGUCCGAGCUGGACA |
| | | AGGCCGGCUUCAUCAAGCGGCAGCUGGUGGAGACCCGGCAGAU |
| | | CACCAAGCACGUGGCCCAGAUCCUGGACUCCCGGAUGAACACCA |
| | | AGUACGACGAGAACGACAAGCUGAUCCGGGAGGUGAAGGUGAU |
| | | CACCCUGAAGUCCAAGCUGGUGUCCGACUUCCGGAAGGACUUCC |
| | | AGUUCUACAAGGUGCGGGAGAUCAACAACUACCACCACGCCCAC |
| | | GACGCCUACCUGAACGCCGUGGUGGGCACCGCCCUGAUCAAGAA |
| | | GUACCCCAAGCUGGAGUCCGAGUUCGUGUACGGCGACUACAAG |
| | | GUGUACGACGUGCGGAAGAUGAUCGCCAAGUCCGAGCAGGAGA |
| | | UCGGCAAGGCCACCGCCAAGUACUUCUUCUACUCCAACAUCAUG |
| | | AACUUCUUCAAGACCGAGAUCACCCUGGCCAACGGCGAGAUCCG |
| | | GAAGCGGCCCCUGAUCGAGACCAACGGCGAGACCGGCGAGAUC |
| | | GUGUGGGACAAGGGCCGGGACUUCGCCACCGUGCGGAAGGUGC |
| | | UGUCCAUGCCCCAGGUGAACAUCGUGAAGAAGACCGAGGUGCA |
| | | GACCGGCGGCUUCUCCAAGGAGUCCAUCCUGCCCAAGCGGAACU |
| | | CCGACAAGCUGAUCGCCCGGAAGAAGGACUGGGACCCCAAGAA |
| | | GUACGGCGGCUUCGACUCCCCCACCGUGGCCUACUCCGUGCUGG |
| | | UGGUGGCCAAGGUGGAGAAGGGCAAGUCCAAGAAGCUGAAGUC |
| | | CGUGAAGGAGCUGCUGGGCAUCACCAUCAUGGAGCGGUCCUCC |
| | | UUCGAGAAGAACCCCAUCGACUUCCUGGAGGCCAAGGGCUACA |
| | | AGGAGGUGAAGAAGGACCUGAUCAUCAAGCUGCCCAAGUACUC |
| | | CCUGUUCGAGCUGGAGAACGGCCGGAAGCGGAUGCUGGCCUCC |
| | | GCCGGCGAGCUGCAGAAGGGCAACGAGCUGGCCCUGCCCUCCAA |
| | | GUACGUGAACUUCCUGUACCUGGCCUCCCACUACGAGAAGCUG |
| | | AAGGGCUCCCCCGAGGACAACGAGCAGAAGCAGCUGUUCGUGG |
| | | AGCAGCACAAGCACUACCUGGACGAGAUCAUCGAGCAGAUCUC |
| | | CGAGUUCUCCAAGCGGGUGAUCCUGGCCGACGCCAACCUGGACA |
| | | AGGUGCUGUCCGCCUACAACAAGCACCGGGACAAGCCCAUCCGG |
| | | GAGCAGGCCGAGAACAUCAUCCACCUGUUCACCCUGACCAACCU |
| | | GGGCGCCCCCGCCGCCUUCAAGUACUUCGACACCACCAUCGACC |
| | | GGAAGCGGUACACCUCCACCAAGGAGGUGCUGGACGCCACCCUG |
| | | AUCCACCAGUCCAUCACCGGCCUGUACGAGACCCGGAUCGACCU |
| | | GUCCCAGCUGGGCGGCGACGGCGGCGGCUCCCCCAAGAAGAAGC |
| | | GGAAGGUGUCCGAGUCCGCCACCCCCGAGUCCGUGUCCGGCUGG |
| | | CGGCUGUUCAAGAAGAUCUCCUGA |
| Open | 804 | AUGGAGGCCUCCCCCGCCUCCGGCCCCCGGCACCUGAUGGACCCCCACAUC |
| reading | | UUCACCUCCAACUUCAACAACGGCAUCGGCCGGCACAAGACCUACCUGUGC |
| frame for | | UACGAGGUGGAGCGGCUGGACAACGGCACCUCCGUGAAGAUGGACCAGCAC |
| BC22n | | CGGGGCUUCCUGCACAACCAGGCCAAGAACCUGCUGUGCGGCUUCUACGGC |
| | | CGGCACGCCGAGCUGCGGUUCCUGGACCUGGUGCCCUCCCUGCAGCUGGAC |
| | | CCCGCCCAGAUCUACCGGGUGACCUGGUUCAUCUCCUGGUCCCCCUGCUUC |
| | | UCCUGGGGCUGCGCCGGCGAGGUGCGGGCCUUCCUGCAGGAGAACACCCAC |
| | | GUGCGGCUGCGGAUCUUCGCCGCCCGGAUCUACGACUACGACCCCCUGUAC |
| | | AAGGAGGCCCUGCAGAUGCUGCGGGACGCCGGCGCCCAGGUGUCCAUCAUG |
| | | ACCUACGACGAGUUCAAGCACUGCUGGGACACCUUCGUGGACCACCAGGGC |
| | | UGCCCCUUCCAGCCCUGGGACGGCCUGGACGAGCACUCCCAGGCCCUGUCC |
| | | GGCCGGCUGCGGGCCAUCCUGCAGAACCAGGGCAACUCCGGCUCCGAGACC |
| | | CCCGGCACCUCCGAGUCCGCCACCCCCGAGUCCGACAAGAAGUACUCCAUC |
| | | GGCCUGGCCAUCGGCACCAACUCCGUGGGCUGGGCCGUGAUCACCGACGAG |
| | | UACAAGGUGCCCUCCAAGAAGUUCAAGGUGCUGGGCAACACCGACCGGCAC |
| | | UCCAUCAAGAAGAACCUGAUCGGCGCCCUGCUGUUCGACUCCGGCGAGACC |
| | | GCCGAGGCCACCCGGCUGAAGCGGACCGCCCGGCGGCGGUACACCCGGCGG |
| | | AAGAACCGGAUCUGCUACCUGCAGGAGAUCUUCUCCAACGAGAUGGCCAAG |
| | | GUGGACGACUCCUUCUUCCACCGGCUGGAGGAGUCCUUCCUGGUGGAGGAG |
| | | GACAAGAAGCACGAGCGGCACCCCAUCUUCGGCAACAUCGUGGACGAGGUG |
| | | GCCUACCACGAGAAGUACCCCACCAUCUACCACCUGCGGAAGAAGCUGGUG |
| | | GACUCCACCGACAAGGCCGACCUGCGGCUGAUCUACCUGGCCCUGGCCCAC |
| | | AUGAUCAAGUUCCGGGGCCACUUCCUGAUCGAGGGCGACCUGAACCCCGAC |
| | | AACUCCGACGUGGACAAGCUGUUCAUCCAGCUGGUGCAGACCUACAACCAG |
| | | CUGUUCGAGGAGAACCCCAUCAACGCCUCCGGCGUGGACGCCAAGGCCAUC |
| | | CUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCC |
| | | CUGGGCCUGACCCCCAACUUCAAGUCCAACUUCGACCUGGCCGAGGACGCC |
| | | AAGCUGCAGCUGUCCAAGGACACCUACGACGACGACCUGGACAACCUGCUG |
| | | GCCCAGAUCGGCGACCAGUACGCCGACCUGUUCCUGGCCGCCAAGAACCUG |
| | | UCCGACGCCAUCCUGCUGUCCGACAUCCUGCGGGUGAACACCGAGAUCACC |
| | | AAGGCCCCCCUGUCCGCCUCCAUGAUCAAGCGGUACGACGAGCACCACCAG |
| | | GACCUGACCCUGCUGAAGGCCCUGGUGCGGCAGCAGCUGCCCGAGAAGUAC |
| | | AAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAG |
| | | AAGAUGGACGGCACCGAGGAGCUGCUGGUGAAGCUGAACCGGGAGGACCUG |
| | | CUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUC |
| | | CUGAAGGACAACCGGGAGAAGAUCGAGAAGAUCCUGACCUUCCGGAUCCCC |
| | | UACUACGUGGGCCCCCUGGCCCGGGGCAACUCCCGGUUCGCCUGGAUGACC |
| | | CGGAAGUCCGAGGAGACCAUCACCCCCUGGAACUUCGAGGAGGUGGUGGAC |
| | | AAGGGCGCCUCCGCCCAGUCCUUCAUCGAGCGGAUGACCAACUUCGACAAG |
| | | AACCUGCCCAACGAGAAGGUGCUGCCCAAGCACUCCCUGCUGUACGAGUAC |
| | | UUCACCGUGUACAACGAGCUGACCAAGGUGAAGUACGUGACCGAGGGCAUG |
| | | CGGAAGCCCGCCUUCCUGUCCGGCGAGCAGAAGAAGGCCAUCGUGGACCUG |
| | | CUGUUCAAGACCAACCGGAAGGUGACCGUGAAGCAGCUGAAGGAGGACUAC |
| | | UUCAAGAAGAUCGAGUGCUUCGACUCCGUGGAGAUCUCCGGCGUGGAGGAC |
| | | CGGUUCAACGCCUCCCUGGGCACCUACCACGACCUGCUGAAGAUCAUCAAG |
| | | GACAAGGACUUCCUGGACAACGAGGAGAACGAGGACAUCCUGGAGGACAUC |
| | | GUGCUGACCCUGACCCUGUUCGAGGACCGGGAGAUGAUCGAGGAGCGGCUG |
| | | AAGACCUACGCCCACCUGUUCGACGACAAGGUGAUGAAGCAGCUGAAGCGG |
| | | CGGCGGUACACCGGCUGGGGCCGGCUGUCCCGGAAGCUGAUCAACGGCAUC |
| | | CGGGACAAGCAGUCCGGCAAGACCAUCCUGGACUUCCUGAAGUCCGACGGC |
| | | UUCGCCAACCGGAACUUCAUGCAGCUGAUCCACGACGACUCCCUGACCUUC |
| | | AAGGAGGACAUCCAGAAGGCCCAGGUGUCCGGCCAGGGCGACUCCCUGCAC |
| | | GAGCACAUCGCCAACCUGGCCGGCUCCCCCGCCAUCAAGAAGGGCAUCCUG |
| | | CAGACCGUGAAGGUGGUGGACGAGCUGGUGAAGGUGAUGGGCCGGCACAAG |
| | | CCCGAGAACAUCGUGAUCGAGAUGGCCCGGGAGAACCAGACCACCCAGAAG |
| | | GGCCAGAAGAACUCCCGGGAGCGGAUGAAGCGGAUCGAGGAGGGCAUCAAG |
| | | GAGCUGGGCUCCCAGAUCCUGAAGGAGCACCCCGUGGAGAACACCCAGCUG |
| | | CAGAACGAGAAGCUGUACCUGUACUACCUGCAGAACGGCCGGGACAUGUAC |
| | | GUGGACCAGGAGCUGGACAUCAACCGGCUGUCCGACUACGACGUGGACCAC |
| | | AUCGUGCCCCAGUCCUUCCUGAAGGACGACUCCAUCGACAACAAGGUGCUG |
| | | ACCCGGUCCGACAAGAACCGGGGCAAGUCCGACAACGUGCCCUCCGAGGAG |
| | | GUGGUGAAGAAGAUGAAGAACUACUGGCGGCAGCUGCUGAACGCCAAGCUG |
| | | AUCACCCAGCGGAAGUUCGACAACCUGACCAAGGCCGAGCGGGGCGGCCUG |
| | | UCCGAGCUGGACAAGGCCGGCUUCAUCAAGCGGCAGCUGGUGGAGACCCGG |
| | | CAGAUCACCAAGCACGUGGCCCAGAUCCUGGACUCCCGGAUGAACACCAAG |
| | | UACGACGAGAACGACAAGCUGAUCCGGGAGGUGAAGGUGAUCACCCUGAAG |
| | | UCCAAGCUGGUGUCCGACUUCCGGAAGGACUUCCAGUUCUACAAGGUGCGG |
| | | GAGAUCAACAACUACCACCACGCCCACGACGCCUACCUGAACGCCGUGGUG |
| | | GGCACCGCCCUGAUCAAGAAGUACCCCAAGCUGGAGUCCGAGUUCGUGUAC |
| | | GGCGACUACAAGGUGUACGACGUGCGGAAGAUGAUCGCCAAGUCCGAGCAG |
| | | GAGAUCGGCAAGGCCACCGCCAAGUACUUCUUCUACUCCAACAUCAUGAAC |
| | | UUCUUCAAGACCGAGAUCACCCUGGCCAACGGCGAGAUCCGGAAGCGGCCC |
| | | CUGAUCGAGACCAACGGCGAGACCGGCGAGAUCGUGUGGGACAAGGGCCGG |
| | | GACUUCGCCACCGUGCGGAAGGUGCUGUCCAUGCCCCAGGUGAACAUCGUG |
| | | AAGAAGACCGAGGUGCAGACCGGCGGCUUCUCCAAGGAGUCCAUCCUGCCC |
| | | AAGCGGAACUCCGACAAGCUGAUCGCCCGGAAGAAGGACUGGGACCCCAAG |
| | | AAGUACGGCGGCUUCGACUCCCCCACCGUGGCCUACUCCGUGCUGGUGGUG |
| | | GCCAAGGUGGAGAAGGGCAAGUCCAAGAAGCUGAAGUCCGUGAAGGAGCUG |
| | | CUGGGCAUCACCAUCAUGGAGCGGUCCUCCUUCGAGAAGAACCCCAUCGAC |
| | | UUCCUGGAGGCCAAGGGCUACAAGGAGGUGAAGAAGGACCUGAUCAUCAAG |
| | | CUGCCCAAGUACUCCCUGUUCGAGCUGGAGAACGGCCGGAAGCGGAUGCUG |
| | | GCCUCCGCCGGCGAGCUGCAGAAGGGCAACGAGCUGGCCCUGCCCUCCAAG |
| | | UACGUGAACUUCCUGUACCUGGCCUCCCACUACGAGAAGCUGAAGGGCUCC |
| | | CCCGAGGACAACGAGCAGAAGCAGCUGUUCGUGGAGCAGCACAAGCACUAC |
| | | CUGGACGAGAUCAUCGAGCAGAUCUCCGAGUUCUCCAAGCGGGUGAUCCUG |
| | | GCCGACGCCAACCUGGACAAGGUGCUGUCCGCCUACAACAAGCACCGGGAC |
| | | AAGCCCAUCCGGGAGCAGGCCGAGAACAUCAUCCACCUGUUCACCCUGACC |
| | | AACCUGGGCGCCCCCGCCGCCUUCAAGUACUUCGACACCACCAUCGACCGG |
| | | AAGCGGUACACCUCCACCAAGGAGGUGCUGGACGCCACCCUGAUCCACCAG |
| | | UCCAUCACCGGCCUGUACGAGACCCGGAUCGACCUGUCCCAGCUGGGCGGC |
| | | GACGGCGGCGGCUCCCCCAAGAAGAAGCGGAAGGUGUGA |
| Open | 805 | AUGGAGGCCUCCCCCGCCUCCGGCCCCCGGCACCUGAUGGACCCCCACAUC |
| reading | | UUCACCUCCAACUUCAACAACGGCAUCGGCCGGCACAAGACCUACCUGUGC |
| frame for | | UACGAGGUGGAGCGGCUGGACAACGGCACCUCCGUGAAGAUGGACCAGCAC |
| BC22n with | | CGGGGCUUCCUGCACAACCAGGCCAAGAACCUGCUGUGCGGCUUCUACGGC |
| Hibit tag | | CGGCACGCCGAGCUGCGGUUCCUGGACCUGGUGCCCUCCCUGCAGCUGGAC |
| | | CCCGCCCAGAUCUACCGGGUGACCUGGUUCAUCUCCUGGUCCCCCUGCUUC |
| | | UCCUGGGGCUGCGCCGGCGAGGUGCGGGCCUUCCUGCAGGAGAACACCCAC |
| | | GUGCGGCUGCGGAUCUUCGCCGCCCGGAUCUACGACUACGACCCCCUGUAC |
| | | AAGGAGGCCCUGCAGAUGCUGCGGGACGCCGGCGCCCAGGUGUCCAUCAUG |
| | | ACCUACGACGAGUUCAAGCACUGCUGGGACACCUUCGUGGACCACCAGGGC |
| | | UGCCCCUUCCAGCCCUGGGACGGCCUGGACGAGCACUCCCAGGCCCUGUCC |
| | | GGCCGGCUGCGGGCCAUCCUGCAGAACCAGGGCAACUCCGGCUCCGAGACC |
| | | CCCGGCACCUCCGAGUCCGCCACCCCCGAGUCCGACAAGAAGUACUCCAUC |
| | | GGCCUGGCCAUCGGCACCAACUCCGUGGGCUGGGCCGUGAUCACCGACGAG |
| | | UACAAGGUGCCCUCCAAGAAGUUCAAGGUGCUGGGCAACACCGACCGGCAC |
| | | UCCAUCAAGAAGAACCUGAUCGGCGCCCUGCUGUUCGACUCCGGCGAGACC |
| | | GCCGAGGCCACCCGGCUGAAGCGGACCGCCCGGCGGCGGUACACCCGGCGG |
| | | AAGAACCGGAUCUGCUACCUGCAGGAGAUCUUCUCCAACGAGAUGGCCAAG |
| | | GUGGACGACUCCUUCUUCCACCGGCUGGAGGAGUCCUUCCUGGUGGAGGAG |
| | | GACAAGAAGCACGAGCGGCACCCCAUCUUCGGCAACAUCGUGGACGAGGUG |
| | | GCCUACCACGAGAAGUACCCCACCAUCUACCACCUGCGGAAGAAGCUGGUG |
| | | GACUCCACCGACAAGGCCGACCUGCGGCUGAUCUACCUGGCCCUGGCCCAC |
| | | AUGAUCAAGUUCCGGGGCCACUUCCUGAUCGAGGGCGACCUGAACCCCGAC |
| | | AACUCCGACGUGGACAAGCUGUUCAUCCAGCUGGUGCAGACCUACAACCAG |
| | | CUGUUCGAGGAGAACCCCAUCAACGCCUCCGGCGUGGACGCCAAGGCCAUC |
| | | CUGUCCGCCCGGCUGUCCAAGUCCCGGCGGCUGGAGAACCUGAUCGCCCAG |
| | | CUGCCCGGCGAGAAGAAGAACGGCCUGUUCGGCAACCUGAUCGCCCUGUCC |
| | | CUGGGCCUGACCCCCAACUUCAAGUCCAACUUCGACCUGGCCGAGGACGCC |
| | | AAGCUGCAGCUGUCCAAGGACACCUACGACGACGACCUGGACAACCUGCUG |
| | | GCCCAGAUCGGCGACCAGUACGCCGACCUGUUCCUGGCCGCCAAGAACCUG |
| | | UCCGACGCCAUCCUGCUGUCCGACAUCCUGCGGGUGAACACCGAGAUCACC |
| | | AAGGCCCCCCUGUCCGCCUCCAUGAUCAAGCGGUACGACGAGCACCACCAG |
| | | GACCUGACCCUGCUGAAGGCCCUGGUGCGGCAGCAGCUGCCCGAGAAGUAC |
| | | AAGGAGAUCUUCUUCGACCAGUCCAAGAACGGCUACGCCGGCUACAUCGAC |
| | | GGCGGCGCCUCCCAGGAGGAGUUCUACAAGUUCAUCAAGCCCAUCCUGGAG |
| | | AAGAUGGACGGCACCGAGGAGCUGCUGGUGAAGCUGAACCGGGAGGACCUG |
| | | CUGCGGAAGCAGCGGACCUUCGACAACGGCUCCAUCCCCCACCAGAUCCAC |
| | | CUGGGCGAGCUGCACGCCAUCCUGCGGCGGCAGGAGGACUUCUACCCCUUC |
| | | CUGAAGGACAACCGGGAGAAGAUCGAGAAGAUCCUGACCUUCCGGAUCCCC |
| | | UACUACGUGGGCCCCCUGGCCCGGGGCAACUCCCGGUUCGCCUGGAUGACC |
| | | CGGAAGUCCGAGGAGACCAUCACCCCCUGGAACUUCGAGGAGGUGGUGGAC |
| | | AAGGGCGCCUCCGCCCAGUCCUUCAUCGAGCGGAUGACCAACUUCGACAAG |
| | | AACCUGCCCAACGAGAAGGUGCUGCCCAAGCACUCCCUGCUGUACGAGUAC |
| | | UUCACCGUGUACAACGAGCUGACCAAGGUGAAGUACGUGACCGAGGGCAUG |
| | | CGGAAGCCCGCCUUCCUGUCCGGCGAGCAGAAGAAGGCCAUCGUGGACCUG |
| | | CUGUUCAAGACCAACCGGAAGGUGACCGUGAAGCAGCUGAAGGAGGACUAC |
| | | UUCAAGAAGAUCGAGUGCUUCGACUCCGUGGAGAUCUCCGGCGUGGAGGAC |
| | | CGGUUCAACGCCUCCCUGGGCACCUACCACGACCUGCUGAAGAUCAUCAAG |
| | | GACAAGGACUUCCUGGACAACGAGGAGAACGAGGACAUCCUGGAGGACAUC |
| | | GUGCUGACCCUGACCCUGUUCGAGGACCGGGAGAUGAUCGAGGAGCGGCUG |
| | | AAGACCUACGCCCACCUGUUCGACGACAAGGUGAUGAAGCAGCUGAAGCGG |
| | | CGGCGGUACACCGGCUGGGGCCGGCUGUCCCGGAAGCUGAUCAACGGCAUC |
| | | CGGGACAAGCAGUCCGGCAAGACCAUCCUGGACUUCCUGAAGUCCGACGGC |
| | | UUCGCCAACCGGAACUUCAUGCAGCUGAUCCACGACGACUCCCUGACCUUC |
| | | AAGGAGGACAUCCAGAAGGCCCAGGUGUCCGGCCAGGGCGACUCCCUGCAC |
| | | GAGCACAUCGCCAACCUGGCCGGCUCCCCCGCCAUCAAGAAGGGCAUCCUG |
| | | CAGACCGUGAAGGUGGUGGACGAGCUGGUGAAGGUGAUGGGCCGGCACAAG |
| | | CCCGAGAACAUCGUGAUCGAGAUGGCCCGGGAGAACCAGACCACCCAGAAG |
| | | GGCCAGAAGAACUCCCGGGAGCGGAUGAAGCGGAUCGAGGAGGGCAUCAAG |
| | | GAGCUGGGCUCCCAGAUCCUGAAGGAGCACCCCGUGGAGAACACCCAGCUG |
| | | CAGAACGAGAAGCUGUACCUGUACUACCUGCAGAACGGCCGGGACAUGUAC |
| | | GUGGACCAGGAGCUGGACAUCAACCGGCUGUCCGACUACGACGUGGACCAC |
| | | AUCGUGCCCCAGUCCUUCCUGAAGGACGACUCCAUCGACAACAAGGUGCUG |
| | | ACCCGGUCCGACAAGAACCGGGGCAAGUCCGACAACGUGCCCUCCGAGGAG |
| | | GUGGUGAAGAAGAUGAAGAACUACUGGCGGCAGCUGCUGAACGCCAAGCUG |
| | | AUCACCCAGCGGAAGUUCGACAACCUGACCAAGGCCGAGCGGGGCGGCCUG |
| | | UCCGAGCUGGACAAGGCCGGCUUCAUCAAGCGGCAGCUGGUGGAGACCCGG |
| | | CAGAUCACCAAGCACGUGGCCCAGAUCCUGGACUCCCGGAUGAACACCAAG |
| | | UACGACGAGAACGACAAGCUGAUCCGGGAGGUGAAGGUGAUCACCCUGAAG |
| | | UCCAAGCUGGUGUCCGACUUCCGGAAGGACUUCCAGUUCUACAAGGUGCGG |
| | | GAGAUCAACAACUACCACCACGCCCACGACGCCUACCUGAACGCCGUGGUG |
| | | GGCACCGCCCUGAUCAAGAAGUACCCCAAGCUGGAGUCCGAGUUCGUGUAC |
| | | GGCGACUACAAGGUGUACGACGUGCGGAAGAUGAUCGCCAAGUCCGAGCAG |
| | | GAGAUCGGCAAGGCCACCGCCAAGUACUUCUUCUACUCCAACAUCAUGAAC |
| | | UUCUUCAAGACCGAGAUCACCCUGGCCAACGGCGAGAUCCGGAAGCGGCCC |
| | | CUGAUCGAGACCAACGGCGAGACCGGCGAGAUCGUGUGGGACAAGGGCCGG |
| | | GACUUCGCCACCGUGCGGAAGGUGCUGUCCAUGCCCCAGGUGAACAUCGUG |
| | | AAGAAGACCGAGGUGCAGACCGGCGGCUUCUCCAAGGAGUCCAUCCUGCCC |
| | | AAGCGGAACUCCGACAAGCUGAUCGCCCGGAAGAAGGACUGGGACCCCAAG |
| | | AAGUACGGCGGCUUCGACUCCCCCACCGUGGCCUACUCCGUGCUGGUGGUG |
| | | GCCAAGGUGGAGAAGGGCAAGUCCAAGAAGCUGAAGUCCGUGAAGGAGCUG |
| | | CUGGGCAUCACCAUCAUGGAGCGGUCCUCCUUCGAGAAGAACCCCAUCGAC |
| | | UUCCUGGAGGCCAAGGGCUACAAGGAGGUGAAGAAGGACCUGAUCAUCAAG |
| | | CUGCCCAAGUACUCCCUGUUCGAGCUGGAGAACGGCCGGAAGCGGAUGCUG |
| | | GCCUCCGCCGGCGAGCUGCAGAAGGGCAACGAGCUGGCCCUGCCCUCCAAG |
| | | UACGUGAACUUCCUGUACCUGGCCUCCCACUACGAGAAGCUGAAGGGCUCC |
| | | CCCGAGGACAACGAGCAGAAGCAGCUGUUCGUGGAGCAGCACAAGCACUAC |
| | | CUGGACGAGAUCAUCGAGCAGAUCUCCGAGUUCUCCAAGCGGGUGAUCCUG |
| | | GCCGACGCCAACCUGGACAAGGUGCUGUCCGCCUACAACAAGCACCGGGAC |
| | | AAGCCCAUCCGGGAGCAGGCCGAGAACAUCAUCCACCUGUUCACCCUGACC |
| | | AACCUGGGCGCCCCCGCCGCCUUCAAGUACUUCGACACCACCAUCGACCGG |
| | | AAGCGGUACACCUCCACCAAGGAGGUGCUGGACGCCACCCUGAUCCACCAG |
| | | UCCAUCACCGGCCUGUACGAGACCCGGAUCGACCUGUCCCAGCUGGGCGGC |
| | | GACGGCGGCGGCUCCCCCAAGAAGAAGCGGAAGGUGUCCGAGUCCGCCACC |
| | | CCCGAGUCCGUGUCCGGCUGGCGGCUGUUCAAGAAGAUCUCCUGA |
| Amino acid | 806 | MEASPASGPRHLMDPHIFTSNFNNGIGRHKTYLCYEVERLDNGTSVKMDQH |
| sequence | | RGFLHNQAKNLLCGFYGRHAELRFLDLVPSLQLDPAQIYRVTWFISWSPCF |
| for BC22n | | SWGCAGEVRAFLQENTHVRLRIFAARIYDYDPLYKEALQMLRDAGAQVSIM |
| | | TYDEFKHCWDTFVDHQGCPFQPWDGLDEHSQALSGRLRAILQNQGNSGSET |
| | | PGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLV |
| | | DSTDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIALS |
| | | LGLTPNFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLPEKY |
| | | KEIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIP |
| | | YYVGPLARGNSRFAWMTRKSEETITPWNFEEVVDKGASAQSFIERMTNFDK |
| | | NLPNEKVLPKHSLLYEYFTVYNELTKVKYVTEGMRKPAFLSGEQKKAIVDL |
| | | LFKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLKR |
| | | RRYTGWGRLSRKLINGIRDKQSGKTILDFLKSDGFANRNFMQLIHDDSLTF |
| | | KEDIQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENTQL |
| | | QNEKLYLYYLQNGRDMYVDQELDINRLSDYDVDHIVPQSFLKDDSIDNKVL |
| | | TRSDKNRGKSDNVPSEEVVKKMKNYWRQLLNAKLITQRKFDNLTKAERGGL |
| | | SELDKAGFIKRQLVETRQITKHVAQILDSRMNTKYDENDKLIREVKVITLK |
| | | SKLVSDFRKDFQFYKVREINNYHHAHDAYLNAVVGTALIKKYPKLESEFVY |
| | | GDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILP |
| | | KRNSDKLIARKKDWDPKKYGGFDSPTVAYSVLVVAKVEKGKSKKLKSVKEL |
| | | LGITIMERSSFEKNPIDFLEAKGYKEVKKDLIIKLPKYSLFELENGRKRML |
| | | ASAGELQKGNELALPSKYVNFLYLASHYEKLKGSPEDNEQKOLFVEQHKHY |
| | | LDEIIEQISEFSKRVILADANLDKVLSAYNKHRDKPIREQAENIIHLFTLT |
| | | NLGAPAAFKYFDTTIDRKRYTSTKEVLDATLIHQSITGLYETRIDLSQLGG |
| | | DGGGSPKKKRKV |
| Open | 807 | AUGGGACCGAAGAAGAAGAGAAAGGUCGGAGGAGGAAGCACAAACCUGUCG |
| reading | | GACAUCAUCGAAAAGGAAACAGGAAAGCAGCUGGUCAUCCAGGAAUCGAUC |
| frame for | | CUGAUGCUGCCGGAAGAAGUCGAAGAAGUCAUCGGAAACAAGCCGGAAUCG |
| UGI | | GACAUCCUGGUCCACACAGCAUACGACGAAUCGACAGACGAAAACGUCAUG |
| | | CUGCUGACAUCGGACGCACCGGAAUACAAGCCGUGGGCACUGGUCAUCCAG |
| | | GACUCGAACGGAGAAAACAAGAUCAAGAUGCUGUGA |
| Open | 808 | AUGACCAACCUGUCCGACAUCAUCGAGAAGGAGACCGGCAAGCAGCUGGUG |
| reading | | AUCCAGGAGUCCAUCCUGAUGCUGCCCGAGGAGGUGGAGGAGGUGAUCGGC |
| frame for | | AACAAGCCCGAGUCCGACAUCCUGGUGCACACCGCCUACGACGAGUCCACC |
| UGI | | GACGAGAACGUGAUGCUGCUGACCUCCGACGCCCCCGAGUACAAGCCCUGG |
| | | GCCCUGGUGAUCCAGGACUCCAACGGCGAGAACAAGAUCAAGAUGCUGUCC |
| | | GGCGGCUCCAAGCGGACCGCCGACGGCUCCGAGUUCGAGUCCCCCAAGAAG |
| | | AAGCGGAAGGUGGAGUGA |
| Amino acid | 809 | MTNLSDIIEKETGKQLVIQESILMLPEEVEEVIGNKPESDILVHTAYDEST |
| sequence | | DENVMLLTSDAPEYKPWALVIQDSNGENKIKMLSGGSKRTADGSEFESPKK |
| for UGI | | KRKVE |
| eGFP | 1001 | TTGGCCACTCCCTCTCTGCGCGCTCGCTCGCTCACTGAGGCCGGGCGACCA |
| insertion | | AAGGTCGCCCGACGCCCGGGCTTTGCCCGGGCGGCCTCAGTGAGCGAGCGA |
| including | | GCGCGCAGAGAGGGAGTGGCCAACTCCATCACTAGGGGTTCCTagatctat |
| ITRs | | ctctcctccctcacccaaccccatgccgtcttcactcgctgggttcccttt |
| | | tccttctccttctggggcctgtgccatctctcgtttcttaggatggccttc |
| | | tccgacggatgtctcccttgcgtcccgcctccccttcttgtaggcctgcat |
| | | catcaccgtttttctggacaaccccaaagtaccccgtctccctggctttag |
| | | ccacctctccatcctcttgctttctttgcctggacaccccgttctcctgtg |
| | | gattcgggtcacctctcactcctttcatttgggcagctcccctacccccct |
| | | tacctctctagtctgtgctagctcttccagccccctgtcatggcatcttcc |
| | | aggggtccgagagctcagctagtcttcttcctccaacccgggcccctatgt |
| | | ccacttcaggacagcatgtttgctgcctccagggatcctgtgtccccgagc |
| | | tgggaccaccttatattcccagggccggttaatgtggctctggttctgggt |
| | | acttttatctgtcccctccaccccacagtggggccactagggacaggattg |
| | | gtgacagaaaagccccatccttaggcctcctccttccgagtaattcataca |
| | | aaaggactcgcccctgccttggggaatcccagggaccgtcgttaaactccc |
| | | actaacgtagaacccagagatcgctgcgttcccgccccctcacccgcccgc |
| | | tctcgtcatcactgaggtggagaagagcatgcgtgaggctccggtgcccgt |
| | | cagtgggcagagcgcacatcgcccacagtccccgagaagttggggggaggg |
| | | gtcggcaattgaaccggtgcctagagaaggtggcgcggggtaaactgggaa |
| | | agtgatgtcgtgtactggctccgcctttttcccgagggtgggggagaaccg |
| | | tatataagtgcagtagtcgccgtgaacgttctttttcgcaacgggtttgcc |
| | | gccagaacacaggtaagtgccgtgtgtggttcccgcgggcctggcctcttt |
| | | acgggttatggcccttgcgtgccttgaattacttccacgcccctggctgca |
| | | gtacgtgattcttgatcccgagcttcgggttggaagtgggtgggagagttc |
| | | gaggccttgcgcttaaggagccccttcgcctcgtgcttgagttgaggcctg |
| | | gcttgggcgctggggccgccgcgtgcgaatctggtggcaccttcgcgcctg |
| | | tctcgctgctttcgataagtctctagccatttaaaatttttgatgacctgc |
| | | tgcgacgctttttttctggcaagatagtcttgtaaatgcgggccaacatct |
| | | gcacactggtatttcggtttttggggccgcgggcggcgacggggcccgtgc |
| | | gtcccagcgcacatgttcggcgaggcggggcctgcgagcgcggccaccgag |
| | | aatcggacgggggtagtctcaagctggccggcctgctctggtgcctggcct |
| | | cgcgccgccgtgtatcgccccgccctgggcggcaaggctggcccggtcggc |
| | | accagttgcgtgagcggaaagatggccgcttcccggccctgctgcagggag |
| | | ctcaaaatggaggacgcggcgctcgggagagcgggcgggtgagtcacccac |
| | | acaaaggaaaagggcctttccgtcctcagccgtcgcttcatgtgactccac |
| | | ggagtaccgggcgccgtccaggcacctcgattagttctcgagcttttggag |
| | | tacgtcgtctttaggttggggggaggggttttatgcgatggagtttcccca |
| | | cactgagtgggtggagactgaagttaggccagcttggcacttgatgtaatt |
| | | ctccttggaatttgccctttttgagtttggatcttggttcattctcaagcc |
| | | tcagacagtggttcaaagtttttttcttccatttcaggtgtcgtgacgcta |
| | | gcgctaccggactcaatctcgagctcaagcttcgaattctgcagtcgacgg |
| | | taccgcgggcccgggatccaccggtcgccaccatggtgAGCAAGGGCGAGG |
| | | AGCTGTTCACCGGGGTGGTGCCCATCCTGGTCGAGCTGGACGGCGACGTAA |
| | | ACGGCCACAAGTTCAGCGTGTCCGGCGAGGGCGAGGGCGATGCCACCTACG |
| | | GCAAGCTGACCCTGAAGTTCATCTGCACCACCGGCAAGCTGCCCGTGCCCT |
| | | GGCCCACCCTCGTGACCACCCTGACCTACGGCGTGCAGTGCTTCAGCCGCT |
| | | ACCCCGACCACATGAAGCAGCACGACTTCTTCAAGTCCGCCATGCCCGAAG |
| | | GCTACGTCCAGGAGCGCACCATCTTCTTCAAGGACGACGGCAACTACAAGA |
| | | CCCGCGCCGAGGTGAAGTTCGAGGGCGACACCCTGGTGAACCGCATCGAGC |
| | | TGAAGGGCATCGACTTCAAGGAGGACGGCAACATCCTGGGGCACAAGCTGG |
| | | AGTACAACTACAACAGCCACAACGTCTATATCATGGCCGACAAGCAGAAGA |
| | | ACGGCATCAAGGTGAACTTCAAGATCCGCCACAACATCGAGGACGGCAGCG |
| | | TGCAGCTCGCCGACCACTACCAGCAGAACACCCCCATCGGCGACGGCCCCG |
| | | TGCTGCTGCCCGACAACCACTACCTGAGCACCCAGTCCGCCCTGAGCAAAG |
| | | ACCCCAACGAGAAGCGCGATCACATGGTCCTGCTGGAGTTCGTGACCGCCG |
| | | CCGGGATCACTCTCGGCATGGACGAGCTGTACAAGtaatagcggccgcgac |
| | | tctagatcataatcagccataccacatttgtagaggttttacttgctttaa |
| | | aaaacctcccacacctccccctgaacctgaaacataaaatgaatgcaattg |
| | | ttgttgttaacttgtttattgcagcttataatggttacaaataaagcaata |
| | | gcatcacaaatttcacaaataaagcatttttttcactgcattctagttgtg |
| | | gtttgtccaaactcatcaatgtatcttaaggcgttagtctcctgatattgg |
| | | gtctaacccccacctcctgttaggcagattccttatctggtgacacacccc |
| | | catttcctggagccatctctctccttgccagaacctctaaggtttgcttac |
| | | gatggagccagagaggatcctgggagggagagcttggcagggggtgggagg |
| | | gaagggggggatgcgtgacctgcccggttctcagtggccaccctgcgctac |
| | | cctctcccagaacctgagctgctctgacgcggccgtctggtgcgtttcact |
| | | gatcctggtgctgcagcttccttacacttcccaagaggagaagcagtttgg |
| | | aaaaacaaaatcagaataagttggtcctgagttctaactttggctcttcac |
| | | ctttctagtccccaatttatattgttcctccgtgcgtcagttttacctgtg |
| | | agataaggccagtagccagccccgtcctggcagggctgtggtgaggagggg |
| | | ggtgtccgtgtggaaaactccctttgtgagaatggtgcgtcctaggtgttc |
| | | accaggtcgtggccgcctctactccctttctctttctccatccttctttcc |
| | | ttaaagagtccccagtgctatcagatctAGGAACCCCTAGTGATGGAGTTG |
| | | GCCACTCCCTCTCTGCGCGCTCGCTCGCTCACTGAGGCCGCCCGGGCAAAG |
| | | CCCGGGCGTCGGGCGACCTTTGGTCGCCCGGCCTCAGTGAGCGAGCGAGCG |
| | | CGCAGAGAGGGAGTGGCCAA |
| G013006 | 1100 | mC*mU*mC*UCAGCUGGUACACGGCAGUUUUAGAmGmCmUmAmGmAmAmAm |
| gRNA | | UmAmGmCAAGUUAAAAUAAGGCUAGUCCGUUAUCAmAmCmUmUmGmAmAmA |
| targeting | | mAmAmGmUmGmGmCmAmCmCmGmAmGmUmCmGmGmUmGmCmU*mU*mU*mU |
| TRBC | | |
| G016239 | 1101 | mG*mG*mC*CUCGGCGCUGACGAUCUGUUUUAGAmGmCmUmAmGmAmAmAm |
| gRNA | | UmAmGmCAAGUUAAAAUAAGGCUAGUCCGUUAUCAmAmCmUmUmGmAmAmA |
| targeting | | mAmAmGmUmGmGmCmAmCmCmGmAmGmUmCmGmGmUmGmCmU*mU*mU*mU |
| TRAC | | |
| G023522 | 1102 | mC*mU*mU*GACGCAUCGCGCCAGGAGUUUUAGAmGmCmUmAmGmAmAmAm |
| gRNA | | UmAmGmCAAGUUAAAAUAAGGCUAGUCCGUUAUCACGAAAGGGCACCGAGU |
| targeting | | CGGmUmGmC*mU |
| CD38 | | |
| Amino acid | 1201 | MEASPASGPRHLMDPHIFTSNFNNGIGRHKTYLCYEVERLDNGTSVKMDQH |
| sequence | | RGFLHNQAKNLLCGFYGRHAELRFLDLVPSLQLDPAQIYRVTWFISWSPCF |
| for BC22n | | SWGCAGEVRAFLQENTHVRLRIFAARIYDYDPLYKEALQMLRDAGAQVSIM |
| with Hibit | | TYDEFKHCWDTFVDHQGCPFQPWDGLDEHSQALSGRLRAILQNQGNSGSET |
| tag | | PGTSESATPESDKKYSIGLAIGTNSVGWAVITDEYKVPSKKFKVLGNTDRH |
| | | SIKKNLIGALLFDSGETAEATRLKRTARRRYTRRKNRICYLQEIFSNEMAK |
| | | VDDSFFHRLEESFLVEEDKKHERHPIFGNIVDEVAYHEKYPTIYHLRKKLV |
| | | DSTDKADLRLIYLALAHMIKFRGHFLIEGDLNPDNSDVDKLFIQLVQTYNQ |
| | | LFEENPINASGVDAKAILSARLSKSRRLENLIAQLPGEKKNGLFGNLIALS |
| | | LGLTPNFKSNFDLAEDAKLQLSKDTYDDDLDNLLAQIGDQYADLFLAAKNL |
| | | SDAILLSDILRVNTEITKAPLSASMIKRYDEHHQDLTLLKALVRQQLPEKY |
| | | KEIFFDQSKNGYAGYIDGGASQEEFYKFIKPILEKMDGTEELLVKLNREDL |
| | | LRKQRTFDNGSIPHQIHLGELHAILRRQEDFYPFLKDNREKIEKILTFRIP |
| | | YYVGPLARGNSRFAWMTRKSEETITPWNFEEVVDKGASAQSFIERMTNFDK |
| | | NLPNEKVLPKHSLLYEYFTVYNELTKVKYVTEGMRKPAFLSGEQKKAIVDL |
| | | LFKTNRKVTVKQLKEDYFKKIECFDSVEISGVEDRFNASLGTYHDLLKIIK |
| | | DKDFLDNEENEDILEDIVLTLTLFEDREMIEERLKTYAHLFDDKVMKQLKR |
| | | RRYTGWGRLSRKLINGIRDKQSGKTILDFLKSDGFANRNFMQLIHDDSLTF |
| | | KEDIQKAQVSGQGDSLHEHIANLAGSPAIKKGILQTVKVVDELVKVMGRHK |
| | | PENIVIEMARENQTTQKGQKNSRERMKRIEEGIKELGSQILKEHPVENTQL |
| | | QNEKLYLYYLQNGRDMYVDQELDINRLSDYDVDHIVPQSFLKDDSIDNKVL |
| | | TRSDKNRGKSDNVPSEEVVKKMKNYWRQLLNAKLITQRKFDNLTKAERGGL |
| | | SELDKAGFIKRQLVETRQITKHVAQILDSRMNTKYDENDKLIREVKVITLK |
| | | SKLVSDFRKDFQFYKVREINNYHHAHDAYLNAVVGTALIKKYPKLESEFVY |
| | | GDYKVYDVRKMIAKSEQEIGKATAKYFFYSNIMNFFKTEITLANGEIRKRP |
| | | LIETNGETGEIVWDKGRDFATVRKVLSMPQVNIVKKTEVQTGGFSKESILP |
| | | KRNSDKLIARKKDWDPKKYGGFDSPTVAYSVLVVAKVEKGKSKKLKSVKEL |
| | | LGITIMERSSFEKNPIDFLEAKGYKEVKKDLIIKLPKYSLFELENGRKRML |
| | | ASAGELQKGNELALPSKYVNFLYLASHYEKLKGSPEDNEQKQLFVEQHKHY |
| | | LDEIIEQISEFSKRVILADANLDKVLSAYNKHRDKPIREQAENIIHLFTLT |
| | | NLGAPAAFKYFDTTIDRKRYTSTKEVLDATLIHQSITGLYETRIDLSQLGG |
| | | DGGGSPKKKRKVSESATPESVSGWRLFKKIS |
| Amino acid | 1202 | MDKKYSIGLDIGTNSVGWAVITDEYKVPSKKFKVLGNTDRHSIKKNLIGAL |
| sequence | | LFDSGETAEATRLKRTARRRYTRRKNRICYLQEIFSNEMAKVDDSFFHRLE |
| for Cas9 | | ESFLVEEDKKHERHPIFGNIVDEVAYHEKYPTIYHLRKKLVDSTDKADLRL |
| with Hibit | | IYLALAHMIKFRGHFLIEGDLNPDNSDVDKLFIQLVQTYNQLFEENPINAS |
| tag | | GVDAKAILSARLSKSRRLENLIAQLPGEKKNGLFGNLIALSLGLTPNFKSN |
| | | FDLAEDAKLQLSKDTYDDDLDNLLAQIGDQYADLFLAAKNLSDAILLSDIL |
| | | RVNTEITKAPLSASMIKRYDEHHQDLTLLKALVRQQLPEKYKEIFFDQSKN |
| | | GYAGYIDGGASQEEFYKFIKPILEKMDGTEELLVKLNREDLLRKQRTFDNG |
| | | SIPHQIHLGELHAILRRQEDFYPFLKDNREKIEKILTFRIPYYVGPLARGN |
| | | SRFAWMTRKSEETITPWNFEEVVDKGASAQSFIERMTNFDKNLPNEKVLPK |
| | | HSLLYEYFTVYNELTKVKYVTEGMRKPAFLSGEQKKAIVDLLFKTNRKVTV |
| | | KQLKEDYFKKIECFDSVEISGVEDRFNASLGTYHDLLKIIKDKDFLDNEEN |
| | | EDILEDIVLTLTLFEDREMIEERLKTYAHLFDDKVMKQLKRRRYTGWGRLS |
| | | RKLINGIRDKQSGKTILDFLKSDGFANRNFMQLIHDDSLTFKEDIQKAQVS |
| | | GQGDSLHEHIANLAGSPAIKKGILQTVKVVDELVKVMGRHKPENIVIEMAR |
| | | ENQTTQKGQKNSRERMKRIEEGIKELGSQILKEHPVENTQLQNEKLYLYYL |
| | | QNGRDMYVDQELDINRLSDYDVDHIVPQSFLKDDSIDNKVLTRSDKNRGKS |
| | | DNVPSEEVVKKMKNYWRQLLNAKLITQRKFDNLTKAERGGLSELDKAGFIK |
| | | RQLVETRQITKHVAQILDSRMNTKYDENDKLIREVKVITLKSKLVSDFRKD |
| | | FQFYKVREINNYHHAHDAYLNAVVGTALIKKYPKLESEFVYGDYKVYDVRK |
| | | MIAKSEQEIGKATAKYFFYSNIMNFFKTEITLANGEIRKRPLIETNGETGE |
| | | IVWDKGRDFATVRKVLSMPQVNIVKKTEVQTGGFSKESILPKRNSDKLIAR |
| | | KKDWDPKKYGGFDSPTVAYSVLVVAKVEKGKSKKLKSVKELLGITIMERSS |
| | | FEKNPIDFLEAKGYKEVKKDLIIKLPKYSLFELENGRKRMLASAGELQKGN |
| | | ELALPSKYVNFLYLASHYEKLKGSPEDNEQKQLFVEQHKHYLDEIIEQISE |
| | | FSKRVILADANLDKVLSAYNKHRDKPIREQAENIIHLFTLTNLGAPAAFKY |
| | | FDTTIDRKRYTSTKEVLDATLIHQSITGLYETRIDLSQLGGDGGGSPKKKR |
| | | KVSESATPESVSGWRLFKKIS* |
| ORF | 1203 | AUGGACAAGAAGUACAGCAUCGGCCUGGACAUCGGCACGAACAGCGUUGGC |
| encoding | | UGGGCUGUGAUCACGGACGAGUACAAGGUUCCCUCAAAGAAGUUCAAGGUG |
| Sp. Cas9 | | CUGGGCAACACGGACCGGCACAGCAUCAAGAAGAAUCUCAUCGGUGCACUG |
| (I-pair | | CUGUUCGACAGCGGUGAGACGGCCGAAGCCACGCGGCUGAAGCGGACGGCC |
| depleted | | CGCCGGCGGUACACGCGGCGGAAGAACCGGAUCUGCUACCUGCAGGAGAUC |
| and/or I- | | UUCAGCAACGAGAUGGCCAAGGUGGACGACAGCUUCUUCCACCGGCUGGAG |
| single | | GAGAGCUUCCUGGUGGAGGAGGACAAGAAGCACGAGCGGCACCCCAUCUUC |
| depleted | | GGCAACAUCGUGGACGAAGUCGCCUACCACGAGAAGUACCCCACCAUCUAC |
| Cas9 ORF) | | CACCUGCGGAAGAAGCUGGUGGACUCGACUGACAAGGCCGACCUGCGGCUG |
| | | AUCUACCUGGCACUGGCCCACAUGAUAAAGUUCCGGGGCCACUUCCUGAUC |
| | | GAGGGCGACCUGAACCCUGACAACAGCGACGUGGACAAGCUGUUCAUCCAG |
| | | CUGGUGCAGACCUACAACCAGCUGUUCGAGGAGAACCCCAUCAACGCCAGC |
| | | GGCGUGGACGCCAAGGCCAUCCUCAGCGCCCGCCUCAGCAAGAGCCGGCGG |
| | | CUGGAGAAUCUCAUCGCCCAGCUUCCAGGUGAGAAGAAGAAUGGGCUGUUC |
| | | GGCAAUCUCAUCGCACUCAGCCUGGGCCUGACUCCCAACUUCAAGAGCAAC |
| | | UUCGACCUGGCCGAGGACGCCAAGCUGCAGCUCAGCAAGGACACCUACGAC |
| | | GACGACCUGGACAAUCUCCUGGCCCAGAUCGGCGACCAGUACGCCGACCUG |
| | | UUCCUGGCUGCCAAGAAUCUCAGCGACGCCAUCCUGCUCAGCGACAUCCUG |
| | | CGGGUGAACACAGAGAUCACGAAGGCCCCCCUCAGCGCCAGCAUGAUAAAG |
| | | CGGUACGACGAGCACCACCAGGACCUGACGCUGCUGAAGGCACUGGUGCGG |
| | | CAGCAGCUUCCAGAGAAGUACAAGGAGAUCUUCUUCGACCAGAGCAAGAAU |
| | | GGGUACGCCGGGUACAUCGACGGUGGUGCCAGCCAGGAGGAGUUCUACAAG |
| | | UUCAUCAAGCCCAUCCUGGAGAAGAUGGACGGCACAGAGGAGCUGCUGGUG |
| | | AAGCUGAACAGGGAGGACCUGCUGCGGAAGCAGCGGACGUUCGACAAUGGG |
| | | AGCAUCCCCCACCAGAUCCACCUGGGUGAGCUGCACGCCAUCCUGCGGCGG |
| | | CAGGAGGACUUCUACCCCUUCCUGAAGGACAACAGGGAGAAGAUCGAGAAG |
| | | AUCCUGACGUUCCGGAUCCCCUACUACGUUGGCCCCCUGGCCCGCGGCAAC |
| | | AGCCGGUUCGCCUGGAUGACGCGGAAGAGCGAGGAGACGAUCACUCCCUGG |
| | | AACUUCGAGGAAGUCGUGGACAAGGGUGCCAGCGCCCAGAGCUUCAUCGAG |
| | | CGGAUGACGAACUUCGACAAGAAUCUUCCAAACGAGAAGGUGCUUCCAAAG |
| | | CACAGCCUGCUGUACGAGUACUUCACGGUGUACAACGAGCUGACGAAGGUG |
| | | AAGUACGUGACAGAGGGCAUGCGGAAGCCCGCCUUCCUCAGCGGUGAGCAG |
| | | AAGAAGGCCAUCGUGGACCUGCUGUUCAAGACGAACCGGAAGGUGACGGUG |
| | | AAGCAGCUGAAGGAGGACUACUUCAAGAAGAUCGAGUGCUUCGACAGCGUG |
| | | GAGAUCAGCGGCGUGGAGGACCGGUUCAACGCCAGCCUGGGCACCUACCAC |
| | | GACCUGCUGAAGAUCAUCAAGGACAAGGACUUCCUGGACAACGAGGAGAAC |
| | | GAGGACAUCCUGGAGGACAUCGUGCUGACGCUGACGCUGUUCGAGGACAGG |
| | | GAGAUGAUAGAGGAGCGGCUGAAGACCUACGCCCACCUGUUCGACGACAAG |
| | | GUGAUGAAGCAGCUGAAGCGGCGGCGGUACACGGGCUGGGGCCGGCUCAGC |
| | | CGGAAGCUGAUCAAUGGGAUCCGAGACAAGCAGAGCGGCAAGACGAUCCUG |
| | | GACUUCCUGAAGAGCGACGGCUUCGCCAACCGGAACUUCAUGCAGCUGAUC |
| | | CACGACGACAGCCUGACGUUCAAGGAGGACAUCCAGAAGGCCCAGGUCAGC |
| | | GGCCAGGGCGACAGCCUGCACGAGCACAUCGCCAAUCUCGCCGGGAGCCCC |
| | | GCCAUCAAGAAGGGGAUCCUGCAGACGGUGAAGGUGGUGGACGAGCUGGUG |
| | | AAGGUGAUGGGCCGGCACAAGCCAGAGAACAUCGUGAUCGAGAUGGCCAGG |
| | | GAGAACCAGACGACUCAAAAGGGGCAGAAGAACAGCAGGGAGCGGAUGAAG |
| | | CGGAUCGAGGAGGGCAUCAAGGAGCUGGGCAGCCAGAUCCUGAAGGAGCAC |
| | | CCCGUGGAGAACACUCAACUGCAGAACGAGAAGCUGUACCUGUACUACCUG |
| | | CAGAAUGGGCGAGACAUGUACGUGGACCAGGAGCUGGACAUCAACCGGCUC |
| | | AGCGACUACGACGUGGACCACAUCGUUCCCCAGAGCUUCCUGAAGGACGAC |
| | | AGCAUCGACAACAAGGUGCUGACGCGGAGCGACAAGAACCGGGGCAAGAGC |
| | | GACAACGUUCCCUCAGAGGAAGUCGUGAAGAAGAUGAAGAACUACUGGCGG |
| | | CAGCUGCUGAACGCCAAGCUGAUCACUCAACGGAAGUUCGACAAUCUCACG |
| | | AAGGCCGAGCGGGGUGGCCUCAGCGAGCUGGACAAGGCCGGGUUCAUCAAG |
| | | CGGCAGCUGGUGGAGACGCGGCAGAUCACGAAGCACGUGGCCCAGAUCCUG |
| | | GACAGCCGGAUGAACACGAAGUACGACGAGAACGACAAGCUGAUCAGGGAA |
| | | GUCAAGGUGAUCACGCUGAAGAGCAAGCUGGUCAGCGACUUCCGGAAGGAC |
| | | UUCCAGUUCUACAAGGUGAGGGAGAUCAACAACUACCACCACGCCCACGAC |
| | | GCCUACCUGAACGCUGUGGUUGGCACGGCACUGAUCAAGAAGUACCCCAAG |
| | | CUGGAGAGCGAGUUCGUGUACGGCGACUACAAGGUGUACGACGUGCGGAAG |
| | | AUGAUAGCCAAGAGCGAGCAGGAGAUCGGCAAGGCCACGGCCAAGUACUUC |
| | | UUCUACAGCAACAUCAUGAACUUCUUCAAGACAGAGAUCACGCUGGCCAAU |
| | | GGUGAGAUCCGGAAGCGGCCCCUGAUCGAGACGAAUGGUGAGACGGGUGAG |
| | | AUCGUGUGGGACAAGGGGCGAGACUUCGCCACGGUGCGGAAGGUGCUCAGC |
| | | AUGCCCCAGGUGAACAUCGUGAAGAAGACAGAAGUCCAGACGGGUGGCUUC |
| | | AGCAAGGAGAGCAUCCUUCCAAAGCGGAACAGCGACAAGCUGAUCGCCCGC |
| | | AAGAAGGACUGGGACCCCAAGAAGUACGGUGGCUUCGACAGCCCCACCGUG |
| | | GCCUACAGCGUGCUGGUGGUGGCCAAGGUGGAGAAGGGGAAGAGCAAGAAG |
| | | CUGAAGAGCGUGAAGGAGCUGCUGGGCAUCACGAUCAUGGAGCGGAGCAGC |
| | | UUCGAGAAGAACCCCAUCGACUUCCUGGAAGCCAAGGGGUACAAGGAAGUC |
| | | AAGAAGGACCUGAUCAUCAAGCUUCCAAAGUACAGCCUGUUCGAGCUGGAG |
| | | AAUGGGCGGAAGCGGAUGCUGGCCAGCGCCGGUGAGCUGCAGAAGGGGAAC |
| | | GAGCUGGCACUUCCCUCAAAGUACGUGAACUUCCUGUACCUGGCCAGCCAC |
| | | UACGAGAAGCUGAAGGGGAGCCCAGAGGACAACGAGCAGAAGCAGCUGUUC |
| | | GUGGAGCAGCACAAGCACUACCUGGACGAGAUCAUCGAGCAGAUCAGCGAG |
| | | UUCAGCAAGCGGGUGAUCCUGGCCGACGCCAAUCUCGACAAGGUGCUCAGC |
| | | GCCUACAACAAGCACCGAGACAAGCCCAUCAGGGAGCAGGCCGAGAACAUC |
| | | AUCCACCUGUUCACGCUGACGAAUCUCGGUGCCCCCGCUGCCUUCAAGUAC |
| | | UUCGACACGACGAUCGACCGGAAGCGGUACACGUCGACUAAGGAAGUCCUG |
| | | GACGCCACGCUGAUCCACCAGAGCAUCACGGGCCUGUACGAGACGCGGAUC |
| | | GACCUCAGCCAGCUGGGUGGCGACGGUGGUGGCAGCCCCAAGAAGAAGCGG |
| | | AAGGUGUAG |
| ORF | 1204 | AUGGACAAGAAGUACAGCAUCGGCCUCGACAUCGGCACCAACAGCGUCGGC |
| encoding | | UGGGCCGUCAUCACCGACGAGUACAAGGUCCCCAGCAAGAAGUUCAAGGUC |
| Sp. Cas9 | | CUCGGCAACACCGACCGCCACAGCAUCAAGAAGAACCUCAUCGGCGCCCUC |
| (E-pair and | | CUCUUCGACAGCGGCGAGACCGCCGAGGCCACCCGCCUCAAGCGCACCGCC |
| E-single | | CGCCGCCGCUACACCCGCCGCAAGAACCGCAUCUGCUACCUCCAGGAGAUC |
| enriched | | UUCAGCAACGAGAUGGCCAAGGUCGACGACAGCUUCUUCCACCGCCUCGAG |
| Cas9 ORF) | | GAGAGCUUCCUCGUCGAGGAGGACAAGAAGCACGAGCGCCACCCCAUCUUC |
| | | GGCAACAUCGUCGACGAGGUCGCCUACCACGAGAAGUACCCCACCAUCUAC |
| | | CACCUCCGCAAGAAGCUCGUCGACAGCACCGACAAGGCCGACCUCCGCCUC |
| | | AUCUACCUCGCCCUCGCCCACAUGAUCAAGUUCCGCGGCCACUUCCUCAUC |
| | | GAGGGCGACCUCAACCCCGACAACAGCGACGUCGACAAGCUCUUCAUCCAG |
| | | CUCGUCCAGACCUACAACCAGCUCUUCGAGGAGAACCCCAUCAACGCCAGC |
| | | GGCGUCGACGCCAAGGCCAUCCUCAGCGCCCGCCUCAGCAAGAGCCGCCGC |
| | | CUCGAGAACCUCAUCGCCCAGCUCCCCGGCGAGAAGAAGAACGGCCUCUUC |
| | | GGCAACCUCAUCGCCCUCAGCCUCGGCCUCACCCCCAACUUCAAGAGCAAC |
| | | UUCGACCUCGCCGAGGACGCCAAGCUCCAGCUCAGCAAGGACACCUACGAC |
| | | GACGACCUCGACAACCUCCUCGCCCAGAUCGGCGACCAGUACGCCGACCUC |
| | | UUCCUCGCCGCCAAGAACCUCAGCGACGCCAUCCUCCUCAGCGACAUCCUC |
| | | CGCGUCAACACCGAGAUCACCAAGGCCCCCCUCAGCGCCAGCAUGAUCAAG |
| | | CGCUACGACGAGCACCACCAGGACCUCACCCUCCUCAAGGCCCUCGUCCGC |
| | | CAGCAGCUCCCCGAGAAGUACAAGGAGAUCUUCUUCGACCAGAGCAAGAAC |
| | | GGCUACGCCGGCUACAUCGACGGCGGCGCCAGCCAGGAGGAGUUCUACAAG |
| | | UUCAUCAAGCCCAUCCUCGAGAAGAUGGACGGCACCGAGGAGCUCCUCGUC |
| | | AAGCUCAACCGCGAGGACCUCCUCCGCAAGCAGCGCACCUUCGACAACGGC |
| | | AGCAUCCCCCACCAGAUCCACCUCGGCGAGCUCCACGCCAUCCUCCGCCGC |
| | | CAGGAGGACUUCUACCCCUUCCUCAAGGACAACCGCGAGAAGAUCGAGAAG |
| | | AUCCUCACCUUCCGCAUCCCCUACUACGUCGGCCCCCUCGCCCGCGGCAAC |
| | | AGCCGCUUCGCCUGGAUGACCCGCAAGAGCGAGGAGACCAUCACCCCCUGG |
| | | AACUUCGAGGAGGUCGUCGACAAGGGCGCCAGCGCCCAGAGCUUCAUCGAG |
| | | CGCAUGACCAACUUCGACAAGAACCUCCCCAACGAGAAGGUCCUCCCCAAG |
| | | CACAGCCUCCUCUACGAGUACUUCACCGUCUACAACGAGCUCACCAAGGUC |
| | | AAGUACGUCACCGAGGGCAUGCGCAAGCCCGCCUUCCUCAGCGGCGAGCAG |
| | | AAGAAGGCCAUCGUCGACCUCCUCUUCAAGACCAACCGCAAGGUCACCGUC |
| | | AAGCAGCUCAAGGAGGACUACUUCAAGAAGAUCGAGUGCUUCGACAGCGUC |
| | | GAGAUCAGCGGCGUCGAGGACCGCUUCAACGCCAGCCUCGGCACCUACCAC |
| | | GACCUCCUCAAGAUCAUCAAGGACAAGGACUUCCUCGACAACGAGGAGAAC |
| | | GAGGACAUCCUCGAGGACAUCGUCCUCACCCUCACCCUCUUCGAGGACCGC |
| | | GAGAUGAUCGAGGAGCGCCUCAAGACCUACGCCCACCUCUUCGACGACAAG |
| | | GUCAUGAAGCAGCUCAAGCGCCGCCGCUACACCGGCUGGGGCCGCCUCAGC |
| | | CGCAAGCUCAUCAACGGCAUCCGCGACAAGCAGAGCGGCAAGACCAUCCUC |
| | | GACUUCCUCAAGAGCGACGGCUUCGCCAACCGCAACUUCAUGCAGCUCAUC |
| | | CACGACGACAGCCUCACCUUCAAGGAGGACAUCCAGAAGGCCCAGGUCAGC |
| | | GGCCAGGGCGACAGCCUCCACGAGCACAUCGCCAACCUCGCCGGCAGCCCC |
| | | GCCAUCAAGAAGGGCAUCCUCCAGACCGUCAAGGUCGUCGACGAGCUCGUC |
| | | AAGGUCAUGGGCCGCCACAAGCCCGAGAACAUCGUCAUCGAGAUGGCCCGC |
| | | GAGAACCAGACCACCCAGAAGGGCCAGAAGAACAGCCGCGAGCGCAUGAAG |
| | | CGCAUCGAGGAGGGCAUCAAGGAGCUCGGCAGCCAGAUCCUCAAGGAGCAC |
| | | CCCGUCGAGAACACCCAGCUCCAGAACGAGAAGCUCUACCUCUACUACCUC |
| | | CAGAACGGCCGCGACAUGUACGUCGACCAGGAGCUCGACAUCAACCGCCUC |
| | | AGCGACUACGACGUCGACCACAUCGUCCCCCAGAGCUUCCUCAAGGACGAC |
| | | AGCAUCGACAACAAGGUCCUCACCCGCAGCGACAAGAACCGCGGCAAGAGC |
| | | GACAACGUCCCCAGCGAGGAGGUCGUCAAGAAGAUGAAGAACUACUGGCGC |
| | | CAGCUCCUCAACGCCAAGCUCAUCACCCAGCGCAAGUUCGACAACCUCACC |
| | | AAGGCCGAGCGCGGCGGCCUCAGCGAGCUCGACAAGGCCGGCUUCAUCAAG |
| | | CGCCAGCUCGUCGAGACCCGCCAGAUCACCAAGCACGUCGCCCAGAUCCUC |
| | | GACAGCCGCAUGAACACCAAGUACGACGAGAACGACAAGCUCAUCCGCGAG |
| | | GUCAAGGUCAUCACCCUCAAGAGCAAGCUCGUCAGCGACUUCCGCAAGGAC |
| | | UUCCAGUUCUACAAGGUCCGCGAGAUCAACAACUACCACCACGCCCACGAC |
| | | GCCUACCUCAACGCCGUCGUCGGCACCGCCCUCAUCAAGAAGUACCCCAAG |
| | | CUCGAGAGCGAGUUCGUCUACGGCGACUACAAGGUCUACGACGUCCGCAAG |
| | | AUGAUCGCCAAGAGCGAGCAGGAGAUCGGCAAGGCCACCGCCAAGUACUUC |
| | | UUCUACAGCAACAUCAUGAACUUCUUCAAGACCGAGAUCACCCUCGCCAAC |
| | | GGCGAGAUCCGCAAGCGCCCCCUCAUCGAGACCAACGGCGAGACCGGCGAG |
| | | AUCGUCUGGGACAAGGGCCGCGACUUCGCCACCGUCCGCAAGGUCCUCAGC |
| | | AUGCCCCAGGUCAACAUCGUCAAGAAGACCGAGGUCCAGACCGGCGGCUUC |
| | | AGCAAGGAGAGCAUCCUCCCCAAGCGCAACAGCGACAAGCUCAUCGCCCGC |
| | | AAGAAGGACUGGGACCCCAAGAAGUACGGCGGCUUCGACAGCCCCACCGUC |
| | | GCCUACAGCGUCCUCGUCGUCGCCAAGGUCGAGAAGGGCAAGAGCAAGAAG |
| | | CUCAAGAGCGUCAAGGAGCUCCUCGGCAUCACCAUCAUGGAGCGCAGCAGC |
| | | UUCGAGAAGAACCCCAUCGACUUCCUCGAGGCCAAGGGCUACAAGGAGGUC |
| | | AAGAAGGACCUCAUCAUCAAGCUCCCCAAGUACAGCCUCUUCGAGCUCGAG |
| | | AACGGCCGCAAGCGCAUGCUCGCCAGCGCCGGCGAGCUCCAGAAGGGCAAC |
| | | GAGCUCGCCCUCCCCAGCAAGUACGUCAACUUCCUCUACCUCGCCAGCCAC |
| | | UACGAGAAGCUCAAGGGCAGCCCCGAGGACAACGAGCAGAAGCAGCUCUUC |
| | | GUCGAGCAGCACAAGCACUACCUCGACGAGAUCAUCGAGCAGAUCAGCGAG |
| | | UUCAGCAAGCGCGUCAUCCUCGCCGACGCCAACCUCGACAAGGUCCUCAGC |
| | | GCCUACAACAAGCACCGCGACAAGCCCAUCCGCGAGCAGGCCGAGAACAUC |
| | | AUCCACCUCUUCACCCUCACCAACCUCGGCGCCCCCGCCGCCUUCAAGUAC |
| | | UUCGACACCACCAUCGACCGCAAGCGCUACACCAGCACCAAGGAGGUCCUC |
| | | GACGCCACCCUCAUCCACCAGAGCAUCACCGGCCUCUACGAGACCCGCAUC |
| | | GACCUCAGCCAGCUCGGCGGCGACGGCGGCGGCAGCCCCAAGAAGAAGCGC |
| | | AAGGUCUAG |
| ORF | 1205 | ATGGACAAGAAGTACAGCATCGGCCTGGACATCGGCACCAACAGCGTGGGC |
| encoding | | TGGGCCGTGATCACCGACGAGTACAAGGTGCCCAGCAAGAAGTTCAAGGTG |
| Sp. Cas9 | | CTGGGCAACACCGACCGGCACAGCATCAAGAAGAACCTGATCGGCGCCCTG |
| (Cas9 ORF | | CTGTTCGACAGCGGCGAGACCGCCGAGGCCACCCGGCTGAAGCGGACCGCC |
| using low | | CGGCGGCGGTACACCCGGCGGAAGAACCGGATCTGCTACCTGCAGGAGATC |
| A/U codons | | TTCAGCAACGAGATGGCCAAGGTGGACGACAGCTTCTTCCACCGGCTGGAG |
| with start | | GAGAGCTTCCTGGTGGAGGAGGACAAGAAGCACGAGCGGCACCCCATCTTC |
| and stop | | GGCAACATCGTGGACGAGGTGGCCTACCACGAGAAGTACCCCACCATCTAC |
| codons) | | CACCTGCGGAAGAAGCTGGTGGACAGCACCGACAAGGCCGACCTGCGGCTG |
| | | ATCTACCTGGCCCTGGCCCACATGATCAAGTTCCGGGGCCACTTCCTGATC |
| | | GAGGGCGACCTGAACCCCGACAACAGCGACGTGGACAAGCTGTTCATCCAG |
| | | CTGGTGCAGACCTACAACCAGCTGTTCGAGGAGAACCCCATCAACGCCAGC |
| | | GGCGTGGACGCCAAGGCCATCCTGAGCGCCCGGCTGAGCAAGAGCCGGCGG |
| | | CTGGAGAACCTGATCGCCCAGCTGCCCGGCGAGAAGAAGAACGGCCTGTTC |
| | | GGCAACCTGATCGCCCTGAGCCTGGGCCTGACCCCCAACTTCAAGAGCAAC |
| | | TTCGACCTGGCCGAGGACGCCAAGCTGCAGCTGAGCAAGGACACCTACGAC |
| | | GACGACCTGGACAACCTGCTGGCCCAGATCGGCGACCAGTACGCCGACCTG |
| | | TTCCTGGCCGCCAAGAACCTGAGCGACGCCATCCTGCTGAGCGACATCCTG |
| | | CGGGTGAACACCGAGATCACCAAGGCCCCCCTGAGCGCCAGCATGATCAAG |
| | | CGGTACGACGAGCACCACCAGGACCTGACCCTGCTGAAGGCCCTGGTGCGG |
| | | CAGCAGCTGCCCGAGAAGTACAAGGAGATCTTCTTCGACCAGAGCAAGAAC |
| | | GGCTACGCCGGCTACATCGACGGCGGCGCCAGCCAGGAGGAGTTCTACAAG |
| | | TTCATCAAGCCCATCCTGGAGAAGATGGACGGCACCGAGGAGCTGCTGGTG |
| | | AAGCTGAACCGGGAGGACCTGCTGCGGAAGCAGCGGACCTTCGACAACGGC |
| | | AGCATCCCCCACCAGATCCACCTGGGCGAGCTGCACGCCATCCTGCGGCGG |
| | | CAGGAGGACTTCTACCCCTTCCTGAAGGACAACCGGGAGAAGATCGAGAAG |
| | | ATCCTGACCTTCCGGATCCCCTACTACGTGGGCCCCCTGGCCCGGGGCAAC |
| | | AGCCGGTTCGCCTGGATGACCCGGAAGAGCGAGGAGACCATCACCCCCTGG |
| | | AACTTCGAGGAGGTGGTGGACAAGGGCGCCAGCGCCCAGAGCTTCATCGAG |
| | | CGGATGACCAACTTCGACAAGAACCTGCCCAACGAGAAGGTGCTGCCCAAG |
| | | CACAGCCTGCTGTACGAGTACTTCACCGTGTACAACGAGCTGACCAAGGTG |
| | | AAGTACGTGACCGAGGGCATGCGGAAGCCCGCCTTCCTGAGCGGCGAGCAG |
| | | AAGAAGGCCATCGTGGACCTGCTGTTCAAGACCAACCGGAAGGTGACCGTG |
| | | AAGCAGCTGAAGGAGGACTACTTCAAGAAGATCGAGTGCTTCGACAGCGTG |
| | | GAGATCAGCGGCGTGGAGGACCGGTTCAACGCCAGCCTGGGCACCTACCAC |
| | | GACCTGCTGAAGATCATCAAGGACAAGGACTTCCTGGACAACGAGGAGAAC |
| | | GAGGACATCCTGGAGGACATCGTGCTGACCCTGACCCTGTTCGAGGACCGG |
| | | GAGATGATCGAGGAGCGGCTGAAGACCTACGCCCACCTGTTCGACGACAAG |
| | | GTGATGAAGCAGCTGAAGCGGCGGCGGTACACCGGCTGGGGCCGGCTGAGC |
| | | CGGAAGCTGATCAACGGCATCCGGGACAAGCAGAGCGGCAAGACCATCCTG |
| | | GACTTCCTGAAGAGCGACGGCTTCGCCAACCGGAACTTCATGCAGCTGATC |
| | | CACGACGACAGCCTGACCTTCAAGGAGGACATCCAGAAGGCCCAGGTGAGC |
| | | GGCCAGGGCGACAGCCTGCACGAGCACATCGCCAACCTGGCCGGCAGCCCC |
| | | GCCATCAAGAAGGGCATCCTGCAGACCGTGAAGGTGGTGGACGAGCTGGTG |
| | | AAGGTGATGGGCCGGCACAAGCCCGAGAACATCGTGATCGAGATGGCCCGG |
| | | GAGAACCAGACCACCCAGAAGGGCCAGAAGAACAGCCGGGAGCGGATGAAG |
| | | CGGATCGAGGAGGGCATCAAGGAGCTGGGCAGCCAGATCCTGAAGGAGCAC |
| | | CCCGTGGAGAACACCCAGCTGCAGAACGAGAAGCTGTACCTGTACTACCTG |
| | | CAGAACGGCCGGGACATGTACGTGGACCAGGAGCTGGACATCAACCGGCTG |
| | | AGCGACTACGACGTGGACCACATCGTGCCCCAGAGCTTCCTGAAGGACGAC |
| | | AGCATCGACAACAAGGTGCTGACCCGGAGCGACAAGAACCGGGGCAAGAGC |
| | | GACAACGTGCCCAGCGAGGAGGTGGTGAAGAAGATGAAGAACTACTGGCGG |
| | | CAGCTGCTGAACGCCAAGCTGATCACCCAGCGGAAGTTCGACAACCTGACC |
| | | AAGGCCGAGCGGGGCGGCCTGAGCGAGCTGGACAAGGCCGGCTTCATCAAG |
| | | CGGCAGCTGGTGGAGACCCGGCAGATCACCAAGCACGTGGCCCAGATCCTG |
| | | GACAGCCGGATGAACACCAAGTACGACGAGAACGACAAGCTGATCCGGGAG |
| | | GTGAAGGTGATCACCCTGAAGAGCAAGCTGGTGAGCGACTTCCGGAAGGAC |
| | | TTCCAGTTCTACAAGGTGCGGGAGATCAACAACTACCACCACGCCCACGAC |
| | | GCCTACCTGAACGCCGTGGTGGGCACCGCCCTGATCAAGAAGTACCCCAAG |
| | | CTGGAGAGCGAGTTCGTGTACGGCGACTACAAGGTGTACGACGTGCGGAAG |
| | | ATGATCGCCAAGAGCGAGCAGGAGATCGGCAAGGCCACCGCCAAGTACTTC |
| | | TTCTACAGCAACATCATGAACTTCTTCAAGACCGAGATCACCCTGGCCAAC |
| | | GGCGAGATCCGGAAGCGGCCCCTGATCGAGACCAACGGCGAGACCGGCGAG |
| | | ATCGTGTGGGACAAGGGCCGGGACTTCGCCACCGTGCGGAAGGTGCTGAGC |
| | | ATGCCCCAGGTGAACATCGTGAAGAAGACCGAGGTGCAGACCGGCGGCTTC |
| | | AGCAAGGAGAGCATCCTGCCCAAGCGGAACAGCGACAAGCTGATCGCCCGG |
| | | AAGAAGGACTGGGACCCCAAGAAGTACGGCGGCTTCGACAGCCCCACCGTG |
| | | GCCTACAGCGTGCTGGTGGTGGCCAAGGTGGAGAAGGGCAAGAGCAAGAAG |
| | | CTGAAGAGCGTGAAGGAGCTGCTGGGCATCACCATCATGGAGCGGAGCAGC |
| | | TTCGAGAAGAACCCCATCGACTTCCTGGAGGCCAAGGGCTACAAGGAGGTG |
| | | AAGAAGGACCTGATCATCAAGCTGCCCAAGTACAGCCTGTTCGAGCTGGAG |
| | | AACGGCCGGAAGCGGATGCTGGCCAGCGCCGGCGAGCTGCAGAAGGGCAAC |
| | | GAGCTGGCCCTGCCCAGCAAGTACGTGAACTTCCTGTACCTGGCCAGCCAC |
| | | GTGGAGCAGCACAAGCACTACCTGGACGAGATCATCGAGCAGATCAGCGAG |
| | | TACGAGAAGCTGAAGGGCAGCCCCGAGGACAACGAGCAGAAGCAGCTGTTC |
| | | GTGGAGCAGCACAAGCACTACCTGGACGAGATCATCGAGCAGATCAGCGAG |
| | | TTCAGCAAGCGGGTGATCCTGGCCGACGCCAACCTGGACAAGGTGCTGAGC |
| | | GCCTACAACAAGCACCGGGACAAGCCCATCCGGGAGCAGGCCGAGAACATC |
| | | ATCCACCTGTTCACCCTGACCAACCTGGGCGCCCCCGCCGCCTTCAAGTAC |
| | | TTCGACACCACCATCGACCGGAAGCGGTACACCAGCACCAAGGAGGTGCTG |
| | | GACGCCACCCTGATCCACCAGAGCATCACCGGCCTGTACGAGACCCGGATC |
| | | GACCTGAGCCAGCTGGGCGGCGACGGCGGCGGCAGCCCCAAGAAGAAGCGG |
| | | AAGGTGTGA |
| scaffold | 1206 | NNNNNNNNNNNNNNNNNNNNGUUUUAGAGCUAGAAAUAGCAAGUUAAAAUA |
| sequence | | AGGCUAGUCCGUUAUCAACUUGAAAAAGUGGCACCGAGUCGGUGCUUUU |
| modified | 1207 | mN*mN*mN*NNNNNNNNNNNNNNNNNGUUUUAGAmGmCmUmAmGmAmAmAm |
| scaffold | | UmAmGmCAAGUUAAAAUAAGGCUAGUCCGUUAUCAmAmCmUmUmGmAmAmA |
| sequence | | mAmAmGmUmGmGmCmAmCmCmGmAmGmUmCmGmGmUmGmCmU*mU*mU*mU |
| G017275 | 1208 | NNNNNNNNNNNNNNNNNNNNGUUUUAGAGCUAGAAAUAGCAAGUUAAAAUA |
| sgRNA | | AGGCUAGUCCGUUAUCAACUUGGCACCGAGUCGGUGC |
| G017275 | 1209 | mN*mN*mN*NNNNNNNNNNNNNNNNNGUUUUAGAGCUAGAAAUAGCAAGUU |
| sgRNA | | AAAAUAAGGCUAGUCCGUUAUCAACUUGGCACCGAGUCGG*mU*mG*mC |
| modified | | |
| G017276 | 1210 | NNNNNNNNNNNNNNNNNNNNGUUUUAGAGCUAGAAAUAGCAAGUUAAAAUA |
| sgRNA | | AGGCUAGUCCGUUAUCACGAAAGGGCACCGAGUCGGUGC |
| G017276 | 1211 | mN*mN*mN*NNNNNNNNNNNNNNNNNGUUUUAGAmGmCmUmAmGmAmAmAm |
| sgRNA | | UmAmGmCAAGUUAAAAUAAGGCUAGUCCGUUAUCACGAAAGGGCACCGAGU |
| modified | | CGG*mU*mG*mC |
| G017277 | 1212 | NNNNNNNNNNNNNNNNNNNNGUUUUAGAGCUAGAAAUAGCAAGUUAAAAUA |
| sgRNA | | AGGCUAGUCCGUUAUCAAAAAUGGCACCGAGUCGGUGC |
| G017277 | 1213 | mN*mN*mN*NNNNNNNNNNNNNNNNNGUUUUAGAmGmCmUmAmGmAmAmAm |
| sgRNA | | UmAmGmCAAGUUAAAAUAAGGCUAGUCCGUUAUCAAAAAUGGCACCGAGUC |
| modified | | GG*mU*mG*mC |
| G017278 | 1214 | NNNNNNNNNNNNNNNNNNNNGUUUUAGAGCUAGAAAUAGCAAGUUAAAAUA |
| sgRNA | | AGGCUAGUCCGUUAUCACAAGGGCACCGAGUCGGUGC |
| G017278 | 1215 | mN*mN*mN*NNNNNNNNNNNNNNNNNGUUUUAGAmGmCmUmAmGmAmAmAm |
| sgRNA | | UmAmGmCAAGUUAAAAUAAGGCUAGUCCGUUAUCACAAGGGCACCGAGUCG |
| modified | | G*mU*mG*mC |
| G017279 | 1216 | NNNNNNNNNNNNNNNNNNNNGUUUUAGAGCUAGAAAUAGCAAGUUAAAAUA |
| sgRNA | | AGGCUAGUCCGUUAUCAAAAUGGCACCGAGUCGGUGC |
| G017279 | 1217 | mN*mN*mN*NNNNNNNNNNNNNNNNNGUUUUAGAmGmCmUmAmGmAmAmAm |
| sgRNA | | UmAmGmCAAGUUAAAAUAAGGCUAGUCCGUUAUCAAAAUGGCACCGAGUCG |
| modified | | G*mU*mG*mC |
| G017280 | 1218 | NNNNNNNNNNNNNNNNNNNNGUUUUAGAGCGCGAAGCGCAAGUUAAAAUAA |
| sgRNA | | GGCUAGUCCGUUAUCAAAAUGGCACCGAGUCGGUGC |
| G017280 | 1219 | mN*mN*mN*NNNNNNNNNNNNNNNNNGUUUUAGAGCGCGAAGCGCAAGUUA |
| sgRNA | | AAAUAAGGCUAGUCCGUUAUCAAAAUGGCACCGAGUCGG*mU*mG*mC |
| modified | | |
| G028546 | 1220 | mG*mC*mG*CCAGCAGUGGAGCGGUCGUUUUAGAmGmCmUmAmGmAmAmAm |
| | | UmAmGmCAAGUUAAAAUAAGGCUAGUCCGUUAUCACGAAAGGGCACCGAGU |
| | | CGGmU*mG*mC*mU |
| G028179 | 1221 | mC*mU*mU*GACGCAUCGCGCCAGGAGUUUUAGAmGmCmUmAmGmAmAmAm |
| | | UmAmGmCAAGUUAAAAUAAGGCUAGUCCGUUAUCACGAAAGGGCACCGAGU |
| | | CGGmU*mG*mC*mU |
| G028542 | 1222 | mG*mA*mC*GGUCUCGGGAAAGCGCUGUUUUAGAmGmCmUmAmGmAmAmAm |
| | | UmAmGmCAAGUUAAAAUAAGGCUAGUCCGUUAUCACGAAAGGGCACCGAGU |
| | | CGGmU*mG*mC*mU |
| G028543 | 1223 | mG*mC*mG*CUUUCCCGAGACCGUCCGUUUUAGAmGmCmUmAmGmAmAmAm |
| | | UmAmGmCAAGUUAAAAUAAGGCUAGUCCGUUAUCACGAAAGGGCACCGAGU |
| | | CGGmU*mG*mC*mU |
| G028544 | 1224 | mG*mG*mA*AAGCGCUUGGUGGUGCCGUUUUAGAmGmCmUmAmGmAmAmAm |
| | | UmAmGmCAAGUUAAAAUAAGGCUAGUCCGUUAUCACGAAAGGGCACCGAGU |
| | | CGGmU*mG*mC*mU |
| G028545 | 1225 | mC*mG*mC*CAGCAGUGGAGCGGUCCGUUUUAGAmGmCmUmAmGmAmAmAm |
| | | UmAmGmCAAGUUAAAAUAAGGCUAGUCCGUUAUCACGAAAGGGCACCGAGU |
| | | CGGmU*mG*mC*mU |

1. An engineered cell comprising a genetic modification in a human CD38 sequence, within genomic coordinates of chr4: 15766497-15871496.

2. (canceled)

3. (canceled)

4. The engineered cell of claim 1, wherein the genetic modification inhibits expression of a CD38 gene, function of a CD38 gene product, or both.

5. The engineered cell of claim 1, wherein the genetic modification comprises a modification of at least one nucleotide within the genomic coordinates selected from:

| SEQ ID NO. | CD38 NO. | CD38 Genomic Coordinates (hg38) |
|---|---|---|
| 37 | CD38-37 | chr4: 15778471-15778491 |
| 34 | CD38-34 | chr4: 15778541-15778561 |
| 36 | CD38-36 | chr4: 15778545-15778565 |
| 26 | CD38-26 | chr4: 15778546-15778566 |
| 35 | CD38-35 | chr4: 15778551-15778571 |
| 27 | CD38-27 | chr4: 15778552-15778572 |
| 31 | CD38-31 | chr4: 15778557-15778577 |
| 25 | CD38-25 | chr4: 15778573-15778593 |
| 16 | CD38-16 | chr4: 15778580-15778600 |
| 9 | CD38-9 | chr4: 15778583-15778603 |
| 10 | CD38-10 | chr4: 15778584-15778604 |
| 8 | CD38-8 | chr4: 15778594-15778614 |
| 3 | CD38-3 | chr4: 15778595-15778615 |
| 11 | CD38-11 | chr4: 15778601-15778621 |
| 23 | CD38-23 | chr4: 15778639-15778659 |
| 48 | CD38-48 | chr4: 15816526-15816546 |
| 53 | CD38-53 | chr4: 15824930-15824950 |
| 58 | CD38-58 | chr4: 15824950-15824970 |
| 59 | CD38-59 | chr4: 15824975-15824995 |
| 71 | CD38-71 | chr4: 15838107-15838127 |
| 74 | CD38-74 | chr4: 15840062-15840082 |
| 79 | CD38-79 | chr4: 15840077-15840097 |
| 81 | CD38-81 | chr4: 15840087-15840107 |
| 38 | CD38-38 | chr4: 15778481-15778501 |
| 28 | CD38-28 | chr4: 15778546-15778566 |

6.-16. (canceled)

17. The engineered cell of claim 1, wherein the cell comprises an exogenous nucleic acid encoding a targeting receptor that is expressed on the surface of the engineered cell, wherein the targeting receptor is a CAR specific for CD38.

18.-20. (canceled)

21. The engineered cell of claim 1, wherein the engineered cell is an immune cell.

22.-25. (canceled)

26. A pharmaceutical composition comprising the engineered cell of claim 1.

27. (canceled)

28. (canceled)

29. A method of administering the engineered cell of claim 1 to a subject in need thereof.

30. (canceled)

31. (canceled)

32. A CD38 guide RNA that specifically hybridizes to a CD38 sequence comprising a nucleotide sequence selected from:
a. a guide sequence comprising a nucleotide sequence selected from SEQ ID NOs: 3, 8, 9, 10, 11, 16, 23, 25, 26, 27, 28, 31, 34, 35, 36, 37, 38, 48, 53, 58, 59, 71, 74, 79, and 81;
b. a guide sequence comprising a nucleotide sequence of at least 17, 18, 19, or 20 contiguous nucleotides of a nucleotide sequence selected from the sequence of SEQ ID NOs: 3, 8, 9, 10, 11, 16, 23, 25, 26, 27, 28, 31, 34, 35, 36, 37, 38, 48, 53, 58, 59, 71, 74, 79, 81;
c. a guide sequence comprising a nucleotide sequence at least 95% identical or at least 90% identical to a nucleotide sequence selected from SEQ ID NOs 3, 8, 9, 10, 11, 16, 23, 25, 26, 27, 28, 31, 34, 35, 36, 37, 38, 48, 53, 58, 59, 71, 74, 79, 81
d. a guide sequence comprising a nucleotide sequence selected from SEQ ID NOs: 8, 9, 10, 11, 16, 23, 25, 27, 28, 31, 34, 35, 36,and 37;
e. a guide sequence comprising a nucleotide sequence selected from SEQ ID NOs: 8, 9, 10, 11, 16, 25, 27, 28, 31, 34, 35, and 36;
f. a guide sequence comprising a nucleotide sequence selected from SEQ ID NOs: 8, 9, 10, 11, 16, 23, 25, 27, 31, 35, 38, 48, 53, 58, 71, 79, and 81;
g. a guide sequence comprising a nucleotide sequence selected from SEQ ID NOs: 3, 8, 11, 28, 35, and 37;
h. a guide sequence comprising a nucleotide sequence selected from SEQ ID NO: 9, 10, 11, 27, and 35;
i. a guide sequence comprising a nucleotide sequence selected from SEQ ID NO: 10, 11, and 35;
j. a guide sequence comprising a nucleotide sequence set forth in SEQ ID NO: 10;
k. a guide sequence comprising a nucleotide sequence set forth in SEQ ID NO: 11;
l. a guide sequence comprising a nucleotide sequence set forth in SEQ ID NO: 35; and
m. a guide sequence comprising a nucleotide sequence selected from SEQ ID NOs: 8 and 35.

33. A CD38 guide RNA comprising a guide sequence that directs an RNA-guided DNA binding agent to a chromosomal location within the genomic coordinates selected from those targeted by SEQ ID NO: 3, 8, 9, 10, 11, 16, 23, 25, 26, 27, 28, 31, 34, 35, 36, 37, 38, 48, 53, 58, 59, 71, 74, 79, and 81.

34. (canceled)

35. The guide RNA of claim 32, wherein the guide RNA is a single guide RNA (sgRNA).

36. (canceled)

37. The guide RNA of claim 35, further comprising 5′ end modification or a 3′ end modification and a conserved portion of an gRNA comprising one or more of:
A. a shortened hairpin 1 region or a substituted and optionally shortened hairpin 1 region relative to SEQ ID NO: 201, wherein
1. at least one of the following pairs of nucleotides are substituted in the substituted and optionally shortened hairpin 1 with Watson-Crick pairing nucleotides: H1-1 and H1-12, H1-2 and H1-11, H1-3 and H1-10, or H1-4 and H1-9, and the hairpin 1 region optionally lacks
a. any one or two of H1-5 through H1-8,
b. one, two, or three of the following pairs of nucleotides: H1-1 and H1-12, H1-2 and H1-11, H1-3 and H1-10, and H1-4 and H1-9, or
c. 1-8 nucleotides of hairpin 1 region; or
2. the shortened hairpin 1 region lacks 4-8 nucleotides, preferably 4-6 nucleotides; and
a. one or more of positions H1-1, H1-2, or H1-3 is deleted or substituted relative to SEQ ID NO: 201 or
b. one or more of positions H1-6 through H1-10 is substituted relative to SEQ ID NO: 201; or
3. the shortened hairpin 1 region lacks 5-10 nucleotides, preferably 5-6 nucleotides, and one or more of positions N18, H1-12, or n is substituted relative to SEQ ID NO: 201; or
B. a shortened upper stem region, wherein the shortened upper stem region lacks 1-6 nucleotides and wherein the 6, 7, 8, 9, 10, or 11 nucleotides of the shortened upper stem region include less than or equal to 4 substitutions relative to SEQ ID NO: 201; or
C. a substitution relative to SEQ ID NO: 201 at any one or more of LS6, LS7, US3, US10, B3, N7, N15, N17, H2-2 and H2-14, wherein the substituent nucleotide is neither a pyrimidine that is followed by an adenine, nor an adenine that is preceded by a pyrimidine; or
D. a SpyCas9 sgRNA-1 of SEQ ID NO: 201 with an upper stem region, wherein the upper stem modification comprises a modification to any one or more of US1-US12 in the upper stem region.

38. The guide RNA of claim 35, further comprising the nucleotide sequence of GUUUUAGAGCUAUGCUGUUUUG (SEQ ID NO: 200) 3′ to the guide sequence.

39. The guide RNA of claim 35, further comprising the nucleotide sequence of (1) GUUUUAGAGCUAGAAAUAGCAAGUUAAAAUAAGGCUAGUCCGUUAUCAACUUGAAA AAGUGGCACCGAGUCGGUGC (SEQ ID NO: 201) 3′ to the guide sequence, (2) GUUUUAGAGCUAGAAAUAGCAAGUUAAAAUAAGGCUAGUCCGUUAUCAACUUGAAA AAGUGGCACCGAGUCGGUGCUUUU (SEQ ID NO: 202) 3′ to the guide sequence, or (3 GUUUUAGAGCUAGAAAUAGCAAGUUAAAAUAAGGCUAGUCCGUUAUCACGAAAGGG CACCGAGUCGGUGCU 3′ to the guide sequence.

40. The guide RNA of claim 39, wherein the guide RNA is modified according to the pattern mN*mN*mN*NNNNNNNNNNNNNNNNNGUUUUAGAmGmCmUmAmGmAmAmAmUmAm GmCAAGUUAAAAUAAGGCUAGUCCGUUAUCAmAmCmUmUmGmAmAmAmAmAmGmU mGmGmCmAmCmCmGmAmGmUmCmGmGmUmGmCmU*mU*mU*mU (SEQ ID NO: 300) or mN*mN*mN*NNNNNNNNNNNNNNNNNGUUUUAGAmGmCmUmAmGmAmAmAmUmAm GmCAAGUUAAAAUAAGGCUAGUCCGUUAUCACGAAAGGGCACCGAGUCGGmU*mG* mC*mU (SEQ ID NO: 414), where “N” is any natural or non-natural nucleotide, m is a 2′-O-methyl modified nucleotide, and * is a phosphorothioate linkage between nucleotide residues; and wherein the N's are collectively the nucleotide sequence of a guide sequence of claim 39.

41.-50. (canceled)

51. A composition comprising a guide RNA of claim 32 and an RNA guided DNA binding agent wherein the RNA guided DNA binding agent is a polypeptide RNA guided DNA binding agent or a nucleic acid encoding an RNA guided DNA binding agent polypeptide.

52. (canceled)

53. The composition of claim 51, wherein the RNA guided DNA binding agent is a Cas9 nuclease.

54.-58. (canceled)

59. A method of making a genetic modification in a CD38 sequence within a cell, comprising contacting the cell with the guide RNA or composition of claim 32.

60. A method of preparing a population of cells for immunotherapy comprising:
a. making a genetic modification in a CD38 sequence in the cells in the population with a CD38 guide RNA or composition of claim 32; and
b. expanding the population of cells in culture.

61.-68. (canceled)

69. A population of cells comprising a genetic modification of a CD38 gene, wherein at least 40%, 45%, 50%, 55%, 60%, 65%, preferably at least 70%, 75%, 80%, 85%, 90%, or 95% of cells in the population comprise a modification selected from an insertion, a deletion, and a substitution in the endogenous CD38 sequence, wherein the genetic modification is as defined in claim 1.

70.-106. (canceled)

107. A method of treating a cancer in a subject, the method comprising administering the subject the engineered cell of claim 69.

108.-124. (canceled)
